(19) United States
(12) Patent Application Publication
(10) Pub. No.: US 2025/0304951 A1
(43) Pub. Date: Oct. 2, 2025

(54) DNA APTAMERS FOR INHIBITING REVERSE TRANSCRIPTASES

(71) Applicant: Integrated DNA Technologies, Inc., Coralville, IA (US)

(72) Inventors: Tanner Holden Reeb, Coralville, IA (US); Sarah Franz Beaudoin, Iowa City, IA (US); Christopher Anthony Vakulskas, North Liberty, IA (US)

(73) Assignee: Integrated DNA Technologies, Inc., Coralville, IA (US)

(21) Appl. No.: 19/094,585

(22) Filed: Mar. 28, 2025

Related U.S. Application Data
(60) Provisional application No. 63/571,790, filed on Mar. 29, 2024.

(51) Int. Cl. C12N 15/10; C12N 15/115

(52) U.S. Cl. CPC C12N 15/1048; C12N 15/115; C12Y 207/07049; C12N 2310/16

(57) ABSTRACT

Disclosed are DNA aptamers. Related compositions and kits are also provided. Also disclosed are methods for amplifying a target DNA sequence using the DNA aptamers, and methods of identifying the DNA aptamers.

### CROSS-REFERENCE TO PRIOR APPLICATION

This patent application claims benefit to U.S. Provisional Patent Application No. 63/571,790, filed Mar. 29, 2024, which is hereby incorporated by reference in its entirety.

### INCORPORATION-BY-REFERENCE OF MATERIAL SUBMITTED ELECTRONICALLY

Incorporated by reference in its entirety herein is a computer-readable nucleotide/amino acid sequence listing submitted concurrently herewith and identified as follows: One 139,096 Byte Extensible Markup Language (XML) file named “772842.xml,” created on Mar. 28, 2025.

### BACKGROUND OF THE INVENTION

Aptamers are small RNA or DNA molecules that can form secondary and tertiary structures capable of specifically binding proteins or other cellular targets.

Reverse transcriptases (RTases) are enzymes involved with viral genome replication utilizing the RNA-dependent DNA polymerase and RNase H activities of RTase. RTases derived from retroviruses were first discovered in 1970 by David Baltimore and Howard Temin and since have revolutionized molecular biology through many uses such as RT-PCR and sequencing isolated RNA. The three most commonly used RTases for cDNA synthesis are derived from the retroviruses, Moloney murine leukemia virus (MMLV), avian myeloblastosis virus (AMV), and human immunodeficiency virus type 1 (HIV-1) (Coffin, et al., Annu. Rev. Virol., 3 (1): 29-51 (2016)).

There are instances when unwanted reverse transcriptase function is deleterious. Accordingly, there exists an unmet need for controlling reverse transcriptase function.

### BRIEF SUMMARY OF THE INVENTION

An aspect of the invention provides a DNA aptamer comprising a nucleotide sequence that has at least 95% sequence identity to at least one of SEQ ID NOs: 2-101.

Further aspects of the invention provide compositions and kits relating to the DNA aptamers of the invention.

Still further aspects of the invention provide methods of amplifying a target DNA sequence, the method comprising (a) providing a composition of an aspect of the invention, the composition further comprising at least one oligonucleotide primer, and at least one sample comprising at least one target DNA sequence, wherein the composition is at a temperature of from about 20° C. to about 30° C., and wherein the reverse transcriptase is bound to the at least one DNA aptamer, (b) elevating the temperature of the composition to release the reverse transcriptase from the at least one DNA aptamer, (c) hybridizing the at least one oligonucleotide primer to the at least one sample comprising at least one target DNA sequence, and (d) initiating DNA polymerase activity and extending the primer with the DNA polymerase to provide additional target DNA sequences.

An additional aspect of the invention provides methods for identifying at least one DNA aptamer that inhibits at least one reverse transcriptase, the method comprising (a) providing a DNA aptamer pool comprising DNA aptamers, (b) allowing the DNA aptamers to be in proximity to the at least one reverse transcriptase in the presence of a RNA aptamer, (c) separating DNA aptamers with high affinity for the at least one reverse transcriptase from the DNA aptamers with low affinity for the at least one reverse transcriptase, and (d) amplifying the DNA aptamers with high affinity for the at least one reverse transcriptase to obtain an enriched DNA aptamer pool, wherein the at least one DNA aptamer that inhibits at least one reverse transcriptase may be identified from the enriched DNA aptamer pool.

### BRIEF DESCRIPTION OF THE SEVERAL VIEWS OF THE DRAWINGS

FIGS. 1A and 1B are a set of graphs showing the results following sequencing of enriched DNA aptamer pools created using a modified Systematic evolution of ligands by exponential enrichment (SELEX) protocol with (FIG. 1B) and without (FIG. 1A) an RNA aptamer added.

### DETAILED DESCRIPTION OF THE INVENTION

An aspect of the invention provides a DNA aptamer comprising a nucleotide sequence that has at least about 95% sequence identity (i.e., at least about 95%, at least about 96%, at least about 97%, at least about 98%, at least about 99%, or about 100% sequence identity) to at least one of SEQ ID NOs: 2-101. A further aspect provides a DNA aptamer comprising a nucleotide sequence that has at least about 95% sequence identity (i.e., at least about 95%, at least about 96%, at least about 97%, at least about 98%, at least about 99%, or about 100% sequence identity) to at least one SEQ ID NO of a DNA aptamer of Table 1.

TABLE 1

| | SEQ ID | |
|---|---|---|
| Name | NO: | Sequence |
| RNA | SEQ ID | rCrUrUrArCrCrArCrGrCrGrCrUrCrUrUrArArCrUrGrCrUrAr |
| Aptamer | NO: 1 | GrCrGrCrCrArUrGrGrCrCrArArArArCrU |
| M21_1 | SEQ ID | GTTCAGTCCCTACGGCGCTAACTATAGGTGAAGTAAC |
| | NO: 2 | TAAACACGTAATTCTTAATTGACTAGCCACCGTGCTAC |
| | | AACCAAG |
| M21_2 | SEQ ID | GTTCAGTCCCTACGGCGCTAACGAAGGGAGTTAGAGG |
| | NO: 3 | GCGCTAATGATATGCACAACGAAATGCCACCGTGCTA |
| | | CAACCAAG |
| M21_3 | SEQ ID | GTTCAGTCCCTACGGCGCTAACCAAATGGTCGTAATA |
| | NO: 4 | GCCTCAACATGTAGTTCTCAATTTCGCCACCGTGCTAC |
| | | AACCAAG |
| M21_4 | SEQ ID | GTTCAGTCCCTACGGCGCTAACTTTCTCCGACATTTTT |
| | NO: 5 | TTAGGAAGGGGGTAGCTCAGTTGGGCCACCGTGCTAC |
| | | AACCAAG |
| M21_5 | SEQ ID | GTTCAGTCCCTACGGCGCTAACCGGCAGGCGAAACAA |
| | NO: 6 | CTTAAACGAGTAGTTCTTCAATTACGCCACCGTGCTAC |
| | | AACCAAG |
| M21_6 | SEQ ID | GTTCAGTCCCTACGGCGCTAACGACACTGGCGTAGTA |
| | NO: 7 | GATTCACACGAAGATATTATTTGTGGCCACCGTGCTAC |
| | | AACCAAG |
| M21_7 | SEQ ID | GTTCAGTCCCTACGGCGCTAACTTGGCGGATATAGTCA |
| | NO: 8 | CAACATGGAGTTCTCAATTGGCCCGCCACCGTGCTAC |
| | | AACCAAG |
| M21_8 | SEQ ID | GTTCAGTCCCTACGGCGCTAACAGTATGGCGTAATAG |
| | NO: 9 | TAATCCCATGAAGATCTCATTTGTTGCCACCGTGCTAC |
| | | AACCAAG |
| M21_9 | SEQ ID | GTTCAGTCCCTACGGCGCTAACTTGGAATGCATGCAGT |
| | NO: 10 | AACTTGAACACGGAATCCTCGACGGCCACCGTGCTAC |
| | | AACCAAG |
| M21_10 | SEQ ID | GTTCAGTCCCTACGGCGCTAACATGTAGATGCGTAAT |
| | NO: 11 | AAATTCACCATGAAGTTCTTAATTAGCCACCGTGCTAC |
| | | AACCAAG |
| M21_11 | SEQ ID | GTTCAGTCCCTACGGCGCTAACTTAGATACGAATTACG |
| | NO: 12 | GCCGCCGCTACCAGGCCTAGGGCAGCCACCGTGCTAC |
| | | AACCAAG |
| M21_12 | SEQ ID | GTTCAGTCCCTACGGCGCTAACTGGAACGCGGAATAG |
| | NO: 13 | CCAAACATGGAGGTCTTTACTTTGTGCCACCGTGCTAC |
| | | AACCAAG |
| M21_13 | SEQ ID | GTTCAGTCCCTACGGCGCTAACCACCGTGGAGTAGAT |
| | NO: 14 | TAACACGGAATTCTCAATTAGCGATGCCACCGTGCTA |
| | | CAACCAAG |
| M21_14 | SEQ ID | GTTCAGTCCCTACGGCGCTAACCAGCGCAAAAACTCT |
| | NO: 15 | GTCTTCCAATGTTAATTTTAGTAGGGCCACCGTGCTAC |
| | | AACCAAG |
| M21_15 | SEQ ID | GTTCAGTCCCTACGGCGCTAACCTTCATGGCCGTTTAG |
| | NO: 16 | TCAAACAAAAAGGCTCTCAGTTTCGCCACCGTGCTAC |
| | | AACCAAG |
| M21_16 | SEQ ID | GTTCAGTCCCTACGGCGCTAACCTGTGACGAGGGACG |
| | NO: 17 | AACCCTGCCGTGCGGCCGGATTTAAGCCACCGTGCTA |
| | | CAACCAAG |
| M21_17 | SEQ ID | GTTCAGTCCCTACGGCGCTAACAGTTGGTCAAATAGC |
| | NO: 18 | CTCAACATGAGATTCTTAATTGGACGCCACCGTGCTAC |
| | | AACCAAG |
| M21_18 | SEQ ID | GTTCAGTCCCTACGGCGCTAACTGGCATAATAGATAA |
| | NO: 19 | AACATGGCGATCTTATTTGATGCACGCCACCGTGCTAC |
| | | AACCAAG |
| M21_19 | SEQ ID | GTTCAGTCCCTACGGCGCTAACTCTCGAGCGGCACAA |
| | NO: 20 | CTATAACGTGGAGTTCTCAATTAGTGCCACCGTGCTAC |
| | | AACCAAG |
| M21_20 | SEQ ID | GTTCAGTCCCTACGGCGCTAACGAAGGGAGTTAGAGG |
| | NO: 21 | GCGCACATGAAAGACAACAAATAATGCCACCGTGCTA |
| | | CAACCAAG |
| M21_21 | SEQ ID | GTTCAGTCCCTACGGCGCTAACAAGCAAGTACACGGC |
| | NO: 22 | GATTTCAATCGCTGAACCCTCATTAGCCACCGTGCTAC |
| | | AACCAAG |
| M21_22 | SEQ ID | GTTCAGTCCCTACGGCGCTAACTCAGTGGAATCGAGC |
| | NO: 23 | CGCATCGAGACAGGGATCAGCTCAGGCCACCGTGCTA |
| | | CAACCAAG |
| M21_23 | SEQ ID | GTTCAGTCCCTACGGCGCTAACGAAGGGAGTTAGAGG |
| | NO: 24 | GCGCACAAAAACGGTGTATTATGCAGCCACCGTGCTA |
| | | CAACCAAG |
| M21_24 | SEQ ID | GTTCAGTCCCTACGGCGCTAACGAAGGGAGTTAGAGG |
| | NO: 25 | GCGTCATCAAAGTATCAACCAGGTGGCCACCGTGCTA |
| | | CAACCAAG |
| M21_25 | SEQ ID | GTTCAGTCCCTACGGCGCTAACGAAGGGAGTTAGAGG |
| | NO: 26 | GCGCTATTCTGCTTGATCCAACAAGGCCACCGTGCTAC |
| | | AACCAAG |
| M21_26 | SEQ ID | GTTCAGTCCCTACGGCGCTAACCCCTACGAAAAAATA |
| | NO: 27 | TCAGCCTTTCAGTTGTTTGATTCGTGCCACCGTGCTAC |
| | | AACCAAG |
| M21_27 | SEQ ID | GTTCAGTCCCTACGGCGCTAACGAAGGGAGTTAGAGG |
| | NO: 28 | GCGTCCACTACACTGCACCGGCCATGCCACCGTGCTA |
| | | CAACCAAG |
| M21_28 | SEQ ID | GTTCAGTCCCTACGGCGCTAACTGGCTGGCGAAGTAA |
| | NO: 29 | CTATCCACGGAGATCTTTATTTGTGGCCACCGTGCTAC |
| | | AACCAAG |
| M21_29 | SEQ ID | GTTCAGTCCCTACGGCGCTAACCTCAGCATTAAATTGC |
| | NO: 30 | ACACTGCCCAGCGGCCGGATTTAAGCCACCGTGCTAC |
| | | AACCAAG |
| M21_30 | SEQ ID | GTTCAGTCCCTACGGCGCTAACTTAGAAAAAGGCGAA |
| | NO: 31 | GTCGCGACACACAATACTTAACGCCGCCACCGTGCTA |
| | | CAACCAAG |
| M21_31 | SEQ ID | GTTCAGTCCCTACGGCGCTAACAGGCGTTTAGCCAAA |
| | NO: 32 | CAATAAGCATATCATCGACCGGGGGGCCACCGTGCTA |
| | | CAACCAAG |
| M21_32 | SEQ ID | GTTCAGTCCCTACGGCGCTAACCTTCAGGCGAAGTAA |
| | NO: 33 | CCTTGCCACGGAGTTCTTAATTCCCGCCACCGTGCTAC |
| | | AACCAAG |
| M21_33 | SEQ ID | GTTCAGTCCCTACGGCGCTAACTACTCAAGGAAGCAT |
| | NO: 34 | CACTCGCATTTTTCAGTCGCCGATAGCCACCGTGCTAC |
| | | AACCAAG |
| M21_34 | SEQ ID | GTTCAGTCCCTACGGCGCTAACCACTTGGCAAATATTC |
| | NO: 35 | GGAGCATGTCGGTCTTACTTGTATGCCACCGTGCTACA |
| | | ACCAAG |
| M21_35 | SEQ ID | GTTCAGTCCCTACGGCGCTAACATAACTCACAACTGCC |
| | NO: 36 | GAGCGGCCGGATTTAAGCTCTAATGCCACCGTGCTAC |
| | | AACCAAG |
| M21_36 | SEQ ID | GTTCAGTCCCTACGGCGCTAACTAACTCCAGCAGGTGT |
| | NO: 37 | TTAAATAGGGATTAGTCCAGCTCTGCCACCGTGCTACA |
| | | ACCAAG |
| M21_37 | SEQ ID | GTTCAGTCCCTACGGCGCTAACCCTTGGCACAGCAGC |
| | NO: 38 | CTAAACGCGGAGGTCTTACTTAGATGCCACCGTGCTA |
| | | CAACCAAG |
| M21_38 | SEQ ID | GTTCAGTCCCTACGGCGCTAACTACTGCCTAACGGCCG |
| | NO: 39 | GATTTAAGTTATCCAGCTGAGCGGTGCCACCGTGCTAC |
| | | AACCAAG |
| M21_39 | SEQ ID | GTTCAGTCCCTACGGCGCTAACGGGAACAAGAAATCA |
| | NO: 40 | GAGTTTCGATAAGCATTCGGCAAGAGCCACCGTGCTA |
| | | CAACCAAG |
| M21_40 | SEQ ID | GTTCAGTCCCTACGGCGCTAACCTAATAAGGCAAATA |
| | NO: 41 | TATCACATGTCGTTCTCAATTCAGTGCCACCGTGCTAC |
| | | AACCAAG |
| M21_41 | SEQ ID | GTTCAGTCCCTACGGCGCTAACTGGATTTGGCGAAGC |
| | NO: 42 | AGCCCCAACGCGGAGTTATCAATTGGCCACCGTGCTA |
| | | CAACCAAG |
| M21_42 | SEQ ID | GTTCAGTCCCTACGGCGCTAACTGCGGTAATGTTAGTT |
| | NO: NO: | AGAATGCTCTACCGGGTGAGCCAAGCCACCGTGCTAC |
| | 43 | AACCAAG |
| M21_43 | SEQ ID | GTTCAGTCCCTACGGCGCTAACTCTGGTTGCTAGTACA |
| | NO: 44 | ATTAAAACACGTCGATCTCATTTAGCCACCGTGCTACA |
| | | ACCAAG |
| M21_44 | SEQ ID | GTTCAGTCCCTACGGCGCTAACGATTGCCTTTGAGGAC |
| | NO: 45 | GGCTTACTCCTTAGCGGAAGACCAGCCACCGTGCTAC |
| | | AACCAAG |
| M21_45 | SEQ ID | GTTCAGTCCCTACGGCGCTAACGTTTCGAAAAAGGGG |
| | NO: 46 | ATCATTGAGCGTAGGGCAATTCTGAGCCACCGTGCTA |
| | | CAACCAAG |
| M21_46 | SEQ ID | GTTCAGTCCCTACGGCGCTAACCAAACACTACCTGCCC |
| | NO: 47 | TGCGGCCGGATTTAAGCTTTCCTAGCCACCGTGCTACA |
| | | ACCAAG |
| M21_47 | SEQ ID | GTTCAGTCCCTACGGCGCTAACTATATGATGCCTTTGG |
| | NO: 48 | GGACGGCTGAATGCAGAGAGGGACGCCACCGTGCTAC |
| | | AACCAAG |
| M21_48 | SEQ ID | GTTCAGTCCCTACGGCGCTAACGGAGGGAGTTAGAGG |
| | NO: 49 | GCGCCTCCATGCATATTAGTGGTGAGCCACCGTGCTAC |
| | | AACCAAG |
| M21_49 | SEQ ID | GTTCAGTCCCTACGGCGCTAACTGTCGAGTGGGTTAA |
| | NO: 50 | GTATACAACTGCGACAACTAGATTTGCCACCGTGCTA |
| | | CAACCAAG |
| M21_50 | SEQ ID | GTTCAGTCCCTACGGCGCTAACCAGTCGGGCGTAATA |
| | NO: 51 | GCGAACATGAAGTTCTTTAATTAGTGCCACCGTGCTAC |
| | | AACCAAG |
| M21 + | SEQ ID | GTTCAGTCCCTACGGCGCTAACGTCGTGGAGTAGCCCT |
| RNA_1 | NO: 52 | AACACGGAAGTATTACTAGGGACCGCCACCGTGCTAC |
| | | AACCAAG |
| M21 + | SEQ ID | GTTCAGTCCCTACGGCGCTAACGATGTGCAACAAGAT |
| RNA_2 | NO: 53 | TGCCTTTGAGGATGGCTTATATTTTGCCACCGTGCTAC |
| | | AACCAAG |
| M21 + | SEQ ID | GTTCAGTCCCTACGGCGCTAACTACCCGCGAAGTAGC |
| RNA_3 | NO: 54 | TTATACACGTAGTTCTCAATAGTTTGCCACCGTGCTAC |
| | | AACCAAG |
| M21 + | SEQ ID | GTTCAGTCCCTACGGCGCTAACTCGATCCCCACTCTAC |
| RNA_4 | NO: 55 | CGGCAACTGCTCGGTCGGGCATCTGCCACCGTGCTAC |
| | | AACCAAG |
| M21 + | SEQ ID | GTTCAGTCCCTACGGCGCTAACCGTTCGCACTCATTGC |
| RNA_5 | NO: 56 | CTTTGGGGTACGGCTTATGAGATCGCCACCGTGCTACA |
| | | ACCAAG |
| M21 + | SEQ ID | GTTCAGTCCCTACGGCGCTAACTACAAGGGCTTCTACC |
| RNA_6 | NO: 57 | GGACACTTATCGGTTTCTGTATGTGCCACCGTGCTACA |
| | | ACCAAG |
| M21 + | SEQ ID | GTTCAGTCCCTACGGCGCTAACGATTATTAGGTGCTCT |
| RNA_7 | NO: 58 | ATTGCCTTTGGGGACGGCTTATAAGCCACCGTGCTACA |
| | | ACCAAG |
| M21 + | SEQ ID | GTTCAGTCCCTACGGCGCTAACGGCTATGCCGCCTTTT |
| RNA_8 | NO: 59 | GGTATCGTATGTTGAGCGACCTAGGCCACCGTGCTAC |
| | | AACCAAG |
| M21 + | SEQ ID | GTTCAGTCCCTACGGCGCTAACCGATTGCCTTTGAGGT |
| RNA_9 | NO: 60 | ATGGCTTATCGTTTTTGACTCGCCGCCACCGTGCTACA |
| | | ACCAAG |
| M21 + | SEQ ID | GTTCAGTCCCTACGGCGCTAACCAGTTGAGGAGTCTA |
| RNA_10 | NO: 61 | CCGGGGAATATCGGTGCATTTGGTCGCCACCGTGCTA |
| | | CAACCAAG |
| M21 + | SEQ ID | GTTCAGTCCCTACGGCGCTAACGGTGACCTTTTCCTAA |
| RNA_11 | NO: 62 | AAGGAGTTTGGCTTTGCGATTTTGGCCACCGTGCTACA |
| | | ACCAAG |
| M21 + | SEQ ID | GTTCAGTCCCTACGGCGCTAACTTCGATCCTCTCTGCC |
| RNA_12 | NO: 63 | CCTGGTGGGCCCCTGAGTAACGGAGCCACCGTGCTAC |
| | | AACCAAG |
| M21 + | SEQ ID | GTTCAGTCCCTACGGCGCTAACATACCATCGGTTTGCC |
| RNA_13 | NO: 64 | TTTGAGGACGGCTTAACCTATGCTGCCACCGTGCTACA |
| | | ACCAAG |
| M21 + | SEQ ID | GTTCAGTCCCTACGGCGCTAACACGAAACTCCTTCAG |
| RNA_14 | NO: 65 | AGAATTGTTCACATGTCACCTTGGCGCCACCGTGCTAC |
| | | AACCAAG |
| M21 + | SEQ ID | GTTCAGTCCCTACGGCGCTAACAATGGTTTCCTCCTTG |
| RNA_15 | NO: 66 | CCTTTGGGGTACGGCTTAGGAGAAGCCACCGTGCTAC |
| | | AACCAAG |
| M21 + | SEQ ID | GTTCAGTCCCTACGGCGCTAACCTTATTTTCGACGCCT |
| RNA_16 | NO: 67 | TTGGGGTACGGCTTCAAAAATATCGCCACCGTGCTAC |
| | | AACCAAG |
| M21 + | SEQ ID | GTTCAGTCCCTACGGCGCTAACCAAACCGGATGCCGA |
| RNA_17 | NO: 68 | TTGCCTTTGGGGTACGGCCTACTCGGCCACCGTGCTAC |
| | | AACCAAG |
| M21 + | SEQ ID | GTTCAGTCCCTACGGCGCTAACAGAAAGCGAATTGCC |
| RNA_18 | NO: 69 | TTTGGGGTATGGCTTATTGGCTGCCGCCACCGTGCTAC |
| | | AACCAAG |
| M21 + | SEQ ID | GTTCAGTCCCTACGGCGCTAACACACAGAGGAAACAA |
| RNA_19 | NO: 70 | CTGCCCCACCGGCCGGATTTAAGGTGCCACCGTGCTA |
| | | CAACCAAG |
| M21 + | SEQ ID | GTTCAGTCCCTACGGCGCTAACTAAGCGTTTTCCAATT |
| RNA_20 | NO: 71 | TTCGCTAAAATTCGCAGGCATAAGGCCACCGTGCTAC |
| | | AACCAAG |
| M21 + | SEQ ID | GTTCAGTCCCTACGGCGCTAACTCCTCTCTGCCATGCG |
| RNA_21 | NO: 72 | GCCGGATTTAAGCATTCATAAACTGCCACCGTGCTAC |
| | | AACCAAG |
| M21 + | SEQ ID | GTTCAGTCCCTACGGCGCTAACGGTTTGAAATTTTGCC |
| RNA_22 | NO: 73 | TTTGGGGATGGCTTTAATTTTCAGGCCACCGTGCTACA |
| | | ACCAAG |
| M21 + | SEQ ID | GTTCAGTCCCTACGGCGCTAACTGGCCAGTGAAATAG |
| RNA_23 | NO: 74 | CTTTTACATGGAATTCTCAATTTGCGCCACCGTGCTAC |
| | | AACCAAG |
| M21 + | SEQ ID | GTTCAGTCCCTACGGCGCTAACCCTCGCGGAATAGAC |
| RNA_24 | NO: 75 | TTACATGAAGATCTTATTTAGTGACGCCACCGTGCTAC |
| | | AACCAAG |
| M21 + | SEQ ID | GTTCAGTCCCTACGGCGCTAACGTGAACATAGCCTTTG |
| RNA_25 | NO: 76 | GTGTACGGCTTATGCTTCTCTCTTGCCACCGTGCTACA |
| | | ACCAAG |
| M21 + | SEQ ID | GTTCAGTCCCTACGGCGCTAACAAGTCCTGGTACGCA |
| RNA_26 | NO: 77 | CCTTGCCAATGTTACTGGCGCTCTAGCCACCGTGCTAC |
| | | AACCAAG |
| M21 + | SEQ ID | GTTCAGTCCCTACGGCGCTAACGTGCGATTGCCTTTGG |
| RNA_27 | NO: 78 | GGTATGGCTTATCATACGCAGCCGGCCACCGTGCTAC |
| | | AACCAAG |
| M21 + | SEQ ID | GTTCAGTCCCTACGGCGCTAACCATAGCAGGATTAGT |
| RNA_28 | NO: 79 | GGAGTTGCCTTTGGGGATGGCTTAGGCCACCGTGCTA |
| | | CAACCAAG |
| M21 + | SEQ ID | GTTCAGTCCCTACGGCGCTAACGCTTATATGTGGCATG |
| RNA_29 | NO: 80 | GTACAGGGATTGTCCGAGAGTTAGGCCACCGTGCTAC |
| | | AACCAAG |
| M21 + | SEQ ID | GTTCAGTCCCTACGGCGCTAACTCTATCAATGGACTTG |
| RNA_30 | NO: 81 | CCTTTGGGGATGGCTTTGTCTATTGCCACCGTGCTACA |
| | | ACCAAG |
| M21 + | SEQ ID | GTTCAGTCCCTACGGCGCTAACTAAGATTACGGTGATC |
| RNA_31 | NO: 82 | ATTGCCTTTGGGGATGGCTTACTGGCCACCGTGCTACA |
| | | ACCAAG |
| M21 + | SEQ ID | GTTCAGTCCCTACGGCGCTAACACTCCCTGCCCGACGG |
| RNA_32 | NO: 83 | CCGGATTTAAGTCGTCTATGGTGAGCCACCGTGCTACA |
| | | ACCAAG |
| M21 + | SEQ ID | GTTCAGTCCCTACGGCGCTAACGAGGTTCTCCACCTTT |
| RNA_33 | NO: 84 | CCATATAGGTCTCATATAGTGTGCGCCACCGTGCTACA |
| | | ACCAAG |
| M21 + | SEQ ID | GTTCAGTCCCTACGGCGCTAACGTTGGCTAAACTAGCT |
| RNA_34 | NO: 85 | CTGCCTTCCGATGTTAATAGTTGGGCCACCGTGCTACA |
| | | ACCAAG |
| M21 + | SEQ ID | GTTCAGTCCCTACGGCGCTAACTCCTCGAACGACGAT |
| RNA_35 | NO: 86 | AACTTTTGGGGACAGTTTTTCGTAGGCCACCGTGCTAC |
| | | AACCAAG |
| M21 + | SEQ ID | GTTCAGTCCCTACGGCGCTAACGGTTACTCTACTCGTA |
| RNA_36 | NO: 87 | GGCCTTTGAGGATGGCCAAACGAAGCCACCGTGCTAC |
| | | AACCAAG |
| M21 + | SEQ ID | GTTCAGTCCCTACGGCGCTAACCCATTTATACTGCCAC |
| RNA_37 | NO: 88 | CGCGGCCGGATTTAAGCGGCAGAAGCCACCGTGCTAC |
| | | AACCAAG |
| M21 + | SEQ ID | GTTCAGTCCCTACGGCGCTAACATTTCGGGGTATAAGT |
| RNA_38 | NO: 89 | TGGGTCCAGAGAAATGTGACCTTCGCCACCGTGCTAC |
| | | AACCAAG |
| M21 + | SEQ ID | GTTCAGTCCCTACGGCGCTAACCCATACTACTGCCCCA |
| RNA_39 | NO: 90 | CGGCCGGATTTAAGTGTTAGGAATGCCACCGTGCTAC |
| | | AACCAAG |
| M21 + | SEQ ID | GTTCAGTCCCTACGGCGCTAACACTGCCCGACGGCCG |
| RNA_40 | NO: 91 | GATTTAAGTCATAGCTCTTCCTGTGGCCACCGTGCTAC |
| | | AACCAAG |
| M21 + | SEQ ID | GTTCAGTCCCTACGGCGCTAACAGCTTGGAGACAACA |
| RNA_41 | NO: 92 | GAGCTATGATCATCGTTTGTTCAACGCCACCGTGCTAC |
| | | AACCAAG |
| M21 + | SEQ ID | GTTCAGTCCCTACGGCGCTAACCAGCCGTGATCCTGGC |
| RNA_42 | NO: 93 | CTTTGGGGTACGGCCTATGATCTCGCCACCGTGCTACA |
| | | ACCAAG |
| M21 + | SEQ ID | GTTCAGTCCCTACGGCGCTAACAGGATTAGTGGAGCG |
| RNA_43 | NO: 94 | GCCTGTTCGTCCTCGATGGATAACAGCCACCGTGCTAC |
| | | AACCAAG |
| M21 + | SEQ ID | GTTCAGTCCCTACGGCGCTAACCCGCTATCAAATATTT |
| RNA_44 | NO: 95 | GCCTTTGGGGATGGCTTAATATTTGCCACCGTGCTACA |
| | | ACCAAG |
| M21 + | SEQ ID | GTTCAGTCCCTACGGCGCTAACAATTGCCTTTGGGGTA |
| RNA_45 | NO: 96 | TGGCTTATTGTGCATGATCGAAAAGCCACCGTGCTAC |
| | | AACCAAG |
| M21 + | SEQ ID | GTTCAGTCCCTACGGCGCTAACAGCATTGCGAAGTAG |
| RNA_46 | NO: 97 | CTATACACGAAGTTCTTTAATTGGCGCCACCGTGCTAC |
| | | AACCAAG |
| M21 + | SEQ ID | GTTCAGTCCCTACGGCGCTAACTTCTGCCCGACCGGCC |
| RNA_47 | NO: 98 | GGATTTAAGGTCATCCGATTGTTAGCCACCGTGCTACA |
| | | ACCAAG |
| M21 + | SEQ ID | GTTCAGTCCCTACGGCGCTAACAATGTATATCTCGGTA |
| RNA_48 | NO: 99 | CGCTACGAGTACTAACCTTGCCATGCCACCGTGCTACA |
| | | ACCAAG |
| M21 + | SEQ ID | GTTCAGTCCCTACGGCGCTAACTCTCTCCTGCATGAAG |
| RNA_49 | NO: 100 | CACCCGCGCCTATCGGGGTGCGGCGCCACCGTGCTAC |
| | | AACCAAG |
| M21 + | SEQ ID | GTTCAGTCCCTACGGCGCTAACTGCCATGCGGCCGGA |
| RNA_50 | NO: 101 | TTTAAGCATCTTAGTATCAGTCAGAGCCACCGTGCTAC |
| | | AACCAAG |
| InfA | SEQ ID | CAAGACCAATCYTGTCACCTCTGAC |
| Forward 1 | NO: 102 | |
| InfA | SEQ ID | CAAGACCAATYCTGTCACCTYTGAC |
| Forward 2 | NO: 103 | |
| InfA | SEQ ID | GCATTYTGGACAAAVCGTCTACG |
| Reverse 1 | NO: 104 | |
| InfA | SEQ ID | GCATTTTGGATAAAGCGTCTACG |
| Reverse 2 | NO: 105 | |
| InfA Probe | SEQ ID | /FAM/TGCAGTCCT/ZEN/CGCTCACTGGGCACG/3IABkFQ/ |
| | NO: 106 | |
| InfB | SEQ ID | TCCTCAAYTCACTCTTCGAGCG |
| Forward | NO: 107 | |
| InfB | SEQ ID | CGGTGCTCTTGACCAAATTGG |
| Reverse | NO: 108 | |
| InfB Probe | SEQ ID | /YakYel/CCAATTCGA/ZEN/GCAGCTGAAACTGCGGTG/3 |
| | NO: 109 | IABkFQ/ |
| SC2 | SEQ ID | CTGCAGATTTGGATGATTTCTCC |
| Forward | NO: 110 | |
| SC2 | SEQ ID | CCTTGTGTGGTCTGCATGAGTTTAG |
| Reverse | NO: 111 | |
| SC2 | SEQ ID | /TexRd- |
| Probe | NO: 112 | XN/ATTGCAACA/TAO/ATCCATGAGCAGTGCTGACTC/3IAbRQSp/ |
| RNaseP | SEQ ID | AGATTTGGACCTGCGAGCG |
| Forward | NO: 113 | |
| RNaseP | SEQ ID | GAGCGGCTGTCTCCACAAGT |
| Reverse | NO: 114 | |
| RNaseP | SEQ ID | /CY5/TTCTGACCT/TAO/GAAGGCTCTGCGCG/3IAbRQSp/ |
| Probe | NO: 115 | |
| FluA RNA | SEQ ID | rCrArArGrArCrCrArArUrCrUrUrGrUrCrArCrCrUrCrUrGrAr |
| Template | NO: 116 | CrUrArArGrGrGrArArUrUrUrUrArGrGrArUrUrUrGrUrGrUr |
| | | UrCrArCrGrCrUrCrArCrCrGrUrGrCrCrCrArGrUrGrArGrCrG |
| | | rArGrGrArCrUrGrCrArGrCrGrUrArGrArCrGrCrUrUrUrGrUr |
| | | CrCrArArArArUrGrC |
| FluB RNA | SEQ ID | rUrCrCrUrCrArArUrUrCrArCrUrCrUrUrCrGrArGrCrGrUrCr |
| Template | NO: 117 | UrUrArArUrGrArArGrGrArCrArUrUrCrArArArGrCrCrArAr |
| | | UrUrCrGrArGrCrArGrCrUrGrArArArCrUrGrCrGrGrUrGrGrG |
| | | rArGrUrCrUrUrArUrCrCrCrArArUrUrUrGrGrUrCrArArGrAr |
| | | GrCrArCrCrG |
| SC2 RNA | SEQ ID | rCrUrGrCrArGrArUrUrUrGrGrArUrGrArUrUrUrCrUrCrCrAr |
| Template | NO: 118 | ArArCrArArUrUrGrCrArArCrArArUrCrCrArUrGrArGrCrArG |
| | | rUrGrCrUrGrArCrUrCrArArCrUrCrArGrGrCrCrUrArArArCr |
| | | UrCrArUrGrCrArGrArCrCrArCrArCrArArGrG |
| RP RNA | SEQ ID | rArGrArUrUrUrGrGrArCrCrUrGrCrGrArGrCrGrGrGrUrUrCr |
| Template | NO: 119 | UrGrArCrCrUrGrArArGrGrCrUrCrUrGrCrGrCrGrGrArCrUrU |
| | | rGrUrGrGrArGrArCrArGrCrCrGrCrUrC |
| M21 + | SEQ ID | GTTCAGTCCCTACGGCGCTAACCGATTGCCTTTGAGGT |
| RNA_9_unmod | NO: 120 | ATGGCTTATCGTTTTTGACTCGCCGCCACCGTGCTACA |
| | | ACCAAG |
| M21 + | SEQ ID | GTTCAGTCCCTACGGCGCTAACCGATTGCCTTTGAGGT |
| RNA_9_3Phos | NO: 121 | ATGGCTTATCGTTTTTGACTCGCCGCCACCGTGCTACA |
| | | ACCAAG/3Phos/ |
| M21 + | SEQ ID | GTTCAGTCCCTACGGCGCTAACCGATTGCCTTTGAGGT |
| RNA_9_C3 | NO: 122 | ATGGCTTATCGTTTTTGACTCGCCGCCACCGTGCTACA |
| | | ACCAAG/3SpC3/ |
| M21 + | SEQ ID | GTTCAGTCCCTACGGCGCTAACCGATTGCCTTTGAGGT |
| RNA_9_ddC | NO: 123 | ATGGCTTATCGTTTTTGACTCGCCGCCACCGTGCTACA |
| | | ACCAAG/3ddC/ |
| M21 + | SEQ ID | GTTCAGTCCCTACGGCGCTAACCGATTGCCTTTGAGGT |
| RNA_9_InvdT | NO: 124 | ATGGCTTATCGTTTTTGACTCGCCGCCACCGTGCTACA |
| | | ACCAAG/3InvdT/ |
| M21 + | SEQ ID | GTTCAGTCCCTACGGCGCTAACCGATTGCCTTTGAGGT |
| RNA_9_amino | NO: 125 | ATGGCTTATCGTTTTTGACTCGCCGCCACCGTGCTACA |
| | | ACCAAG/3AmMO/ |
| M21 + | SEQ ID | GTTCAGTCCCTACGGCGCTAACGTGCGATTGCCTTTGG |
| RNA_27_unmod | NO: 126 | GGTATGGCTTATCATACGCAGCCGGCCACCGTGCTAC |
| | | AACCAAG |
| M21 + | SEQ ID | GTTCAGTCCCTACGGCGCTAACGTGCGATTGCCTTTGG |
| RNA_27_3Phos | NO: 127 | GGTATGGCTTATCATACGCAGCCGGCCACCGTGCTAC |
| | | AACCAAG/3Phos/ |
| M21 + | SEQ ID | GTTCAGTCCCTACGGCGCTAACGTGCGATTGCCTTTGG |
| RNA_27_C3 | NO: 128 | GGTATGGCTTATCATACGCAGCCGGCCACCGTGCTAC |
| | | AACCAAG/3SpC3/ |
| M21 + | SEQ ID | GTTCAGTCCCTACGGCGCTAACGTGCGATTGCCTTTGG |
| RNA_27_ddC | NO: 129 | GGTATGGCTTATCATACGCAGCCGGCCACCGTGCTAC |
| | | AACCAAG/3ddC/ |
| M21 + | SEQ ID | GTTCAGTCCCTACGGCGCTAACGTGCGATTGCCTTTGG |
| RNA_27_InvdT | NO: 130 | GGTATGGCTTATCATACGCAGCCGGCCACCGTGCTAC |
| | | AACCAAG/3InvdT/ |
| M21 + | SEQ ID | GTTCAGTCCCTACGGCGCTAACGTGCGATTGCCTTTGG |
| RNA_27_amino | NO: 131 | GGTATGGCTTATCATACGCAGCCGGCCACCGTGCTAC |
| | | AACCAAG/3 AmMO/ |
| M21 DNA | SEQ ID | ATGACTTTAAATATTGAGGATGAGCATCGTTTACATGA |
| Sequence | NO: 132 | GACATCAAAAGAACCCGACGTGAGCTTAGGGTCAACG |
| | | TGGCTTTCTGACTTCCCCCAGGCGTGGGCGGAGACTG |
| | | GCGGAATGGGGTTAGCTGTCCGCCAAGCACCGTTGAT |
| | | CATCCCGTTAAAGGCAACGTCTACACCTGTCTCTATCA |
| | | AACAGTACCCCATGAGTCGTGAGGCCCGCCTGGGGAT |
| | | TAAGCCACATATTCGTCGCTTGCTGGACCAGGGGATCT |
| | | TGGTCCCATGTCAATCTCCGTGGAACACCCCCCTTAAA |
| | | CCCGTGAAAAAGCCAGGTACAAACGATTATCGTCCAG |
| | | TTCAAGATCTTCGCGAGGTCAACAAACGCGTAGAAGA |
| | | CATCCATCCGACTGTACCTAATCCTTATAATCTGTTAT |
| | | CAGGCCTGCCCCCATCGCACCAATGGTATACAGTATT |
| | | AGACTTGAAAGACGCGTTCTTTTGCCTGCGTCTGCACC |
| | | CAACGTCTCAGCCGCTGTTTGCGTTCGAATGGCGTGAT |
| | | CCTGAAATGGGAATTTCGGGTCAGTTAACCTGGACTC |
| | | GTCTGCCCCAGGGCTTTAAAAACAGCCCCACATTGTTC |
| | | GATGAAGCACTTCACCGTGACTTAGCAGACTTCCGTAT |
| | | CCAACACCCAGACTTAATTCTGTTACAGTATGTTGACG |
| | | ACCTTTTGTTGGCGGCAACGTCTGAACTTGACTGTCAG |
| | | CAAGGCACACGCGCGTTATTACAAACGTTAGGTAACT |
| | | TAGGATATCGTGCGTCCGCGAAAAAGGCGCAAATTTG |
| | | TCAAAAACAGGTAAAGTACCTTGGGTATTTGCTGAAA |
| | | GAAGGTCAACGTTGGCTGACTGATGCGCGTAAGGAGA |
| | | CCGTAATGGGGCAGCCTACGCCTAAGACGCCACGCCA |
| | | GTTGCGTGAATTTTTGGGCACAGCGGGATTCTGTCGTT |
| | | TATGGATTCCTGGGTTCGCTGAAATGGCTGCACCCCTG |
| | | TACCCCTTAACAAAAACAGGGACGCTTTTCAACTGGG |
| | | GGCCAGACCAGCAAAAGGCGTATCAGGAGATCAAAC |
| | | AAGCTTTGTTGACCGCACCCGCGTTGGGTCTTCCGGAT |
| | | TTAACCAAGCCCTTTGAGCTGTTCGTTGATGAAAAACA |
| | | GGGATATGCAAAAGGAGTATTAACCCAAAAGTTAGGC |
| | | CCGTGGCGTCGCCCTGTTGCTTACTTGAGTAAAAAATT |
| | | GGATCCTGTCGCAGCAGGATGGCCACCGTGCTTGCGT |
| | | ATGGTCGCGGCAATTGCCGTTTTGACAAAGGATGCAG |
| | | GTAAGTTGACGATGGGTCAACCCTTAGTAATCTTGGCT |
| | | CCACATGCTGTAGAAGCGTTAGTAAAGCAGCCCCCAG |
| | | ACCGCTGGCTTTCTAATGCGCGCATGACCCACTATCAG |
| | | GCGCTTCTGCTTGATACGGATCGTGTACAATTTGGACC |
| | | AGTTGTAGCTTTGAATCCAGCTACTTTGCTTCCCCTTC |
| | | CAGAAGAAGGACTTCAGCACAATTGTTTAGATATTCT |
| | | GGCCGAGGCACATGGGACGCGCCCTGATTTGACGGAT |
| | | CAGCCACTGCCTGATGCCGACCATACATGGTATACTG |
| | | GCGGCAGTAGTCTTCTTCAAGAGGGGCAACGCAAGGC |
| | | GGGAGCAGCCGTCACTACGGAGACCGAAGTTATCTGG |
| | | GCCAAAGCGTTACCCGCGGGAACATCCGCGCAACGTG |
| | | CACAGTTAATCGCTCTGACACAGGCCCTGAAGATGGC |
| | | AGAGGGCAAAAAGTTGAATGTCTACACCAACTCACGT |
| | | TATGCTTTTGCAACAGCGCATATCCATGGCGAAATTTA |
| | | CCGCCGCCGTGGTCTGCTGACTAGTGAGGGTAAGGAA |
| | | ATTAAAAATAAAGATGAGATTCTTGCGTTGTTAAAAG |
| | | CTTTATTCTTACCAAAACGCCTTTCGATCATTCATTGC |
| | | CCGGGGCATCAAAAGGGTCACTCAGCGGAGGCTCGTG |
| | | GAAACCGTATGGCGGACCAAGCTGCCCGTAAGGCGGC |
| | | GATCACAGAGACCCCGGATACATCAACGCTGTTGATC |
| | | GAAAACAGCTCTCCCTACACTAGCGAGCATTTT |
| M21 | SEQ ID | MGSSHHHHHHSSGLVPRGSHMTLNIEDEHRLHETSKEPD |
| Protein | NO: 133 | VSLGSTWLSDFPQAWAETGGMGLAVRQAPLIIPLKATST |
| Sequence | | PVSIKQYPMSREARLGIKPHIRRLLDQGILVPCQSPWNTP |
| | | LKPVKKPGTNDYRPVQDLREVNKRVEDIHPTVPNPYNLL |
| | | SGLPPSHQWYTVLDLKDAFFCLRLHPTSQPLFAFEWRDP |
| | | EMGISGQLTWTRLPQGFKNSPTLFDEALHRDLADFRIQH |
| | | PDLILLQYVDDLLLAATSELDCQQGTRALLQTLGNLGYR |
| | | ASAKKAQICQKQVKYLGYLLKEGQRWLTDARKETVMG |
| | | QPTPKTPRQLREFLGTAGFCRLWIPGFAEMAAPLYPLTK |
| | | TGTLFNWGPDQQKAYQEIKQALLTAPALGLPDLTKPFEL |
| | | FVDEKQGYAKGVLTQKLGPWRRPVAYLSKKLDPVAAG |
| | | WPPCLRMVAAIAVLTKDAGKLTMGQPLVILAPHAVEAL |
| | | VKQPPDRWLSNARMTHYQALLLDTDRVQFGPVVALNP |
| | | ATLLPLPEEGLQHNCLDILAEAHGTRPDLTDQPLPDADH |
| | | TWYTGGSSLLQEGQRKAGAAVTTETEVIWAKALPAGTS |
| | | AQRAQLIALTQALKMAEGKKLNVYTNSRYAFATAHIHG |
| | | EIYRRRGLLTSEGKEIKNKDEILALLKALFLPKRLSIIHCP |
| | | GHQKGHSAEARGNRMADQAARKAAITETPDTSTLLIENS |
| | | SPYTSEHF |
| M21 + | SEQ ID | GTTCAGTCCCTACGGCGCTAACGGTGACCTTTTCCTAA |
| RNA_11_3Phos | NO: 134 | AAGGAGTTTGGCTTTGCGATTTTGGCCACCGTGCTACA |
| | | ACCAAG/3Phos/ |
| M21 + | SEQ ID | GTTCAGTCCCTACGGCGCTAACTTCTGCCCGACCGGCC |
| RNA_47_3Phos | NO: 135 | GGATTTAAGGTCATCCGATTGTTAGCCACCGTGCTACA |
| | | ACCAAG/3Phos/ |

In Table 1, and throughout the application, FAM refers to a dye: ZEN refers to N,N-diethyl-4-(4-nitronaphthalen-1-ylazo)-phenylamine: 3IABkFQ refers to Iowa Black™ FQ quencher, which has a broad absorbance spectra ranging from 420 to 620 nm with peak absorbance at 531 nm: YakYel refers to Yakima Yellow™ dye (trademark of ELITechGroup): TexRd-XN refers to 5′ Texas Red™-X (NHS Ester) (trademark of Molecular Probes/Life Technologies): TAO refers to a quencher: 31AbRQSp refers to a quencher: CY5 refers to a red dye: 3Phos refers to 3′ phosphorylation: 3SpC3 refers to 3′ C3 Spacer: 3ddC refers to a 3′ Dideoxy-C: 3InvdT refers to 3′ inverted dT; and 3AmMO refers to a 3′ amino modifier. In Table 1, rC refers to RNA cytosine base, rU refers to RNA uracil base, rA refers to RNA adenine base, and rG refers to RNA guanine base.

The DNA aptamer can be any suitable length, i.e., can comprise any number of amino acids, provided that the DNA aptamers retain their biological activity, e.g., their ability to specifically bind a reverse transcriptase. In an aspect of the invention, the DNA aptamer comprises of from about 50 to about 90 nucleotides (i.e., from about 55 to about 89 nucleotides, from about 60 to about 88 nucleotides, from about 65 to about 87 nucleotides, from about 70 to about 86 nucleotides, from about 72 to about 85 nucleotides, from about 74 to about 85 nucleotides, from about 76 to about 85 nucleotides, from about 78 to about 84 nucleotides, from about 79 to about 84 nucleotides, from about 81 to about 83 nucleotides, or about 82 nucleotides). In this regard, the DNA aptamer comprises about 20, about 25, about 30, about 35, about 40, about 45, about 50, about 55, about 60, about 65, about 70, about 75, about 80, about 81, about 82, about 83, about 84, about 85, or about 90 nucleotides.

The DNA aptamer can comprise adapter sequences. In this regard, the adapter sequence can be any suitable length. For example, the adapter sequence can be from about 5 to about 30 nucleotides, from about 10 to about 25 nucleotides, from about 15 to about 25 nucleotides, from about 20 to about 22 nucleotides, or about 21 nucleotides. The DNA aptamer can comprise about two adapter sequences.

In an aspect of the invention, the DNA aptamer comprises a nucleotide sequence that has at least about 95% (i.e., at least about 95%, at least about 96%, at least about 97%, at least about 98%, at least about 99%, or about 100%) sequence identity to at least one of SEQ ID NOs: 29 (M21_28), 45 (M21_44), 60 (M21+RNA_9), 72 (M21+RNA_21), 76 (M21+RNA_25), 78 (M21+RNA_27), 79 (M21+RNA_28), 81 (M21+RNA_30), 82 (M21+RNA_31), 83 (M21+RNA_32), 90 (M21+RNA_39), 91 (M21+RNA_40), 93 (M21+RNA_42), 94 (M21+RNA_43), 95 (M21+RNA_44), 121 (M21+RNA_9_3Phos), 134 (M21+RNA_11_3Phos), and 135 (M21+RNA_47_3Phos). In an aspect of the invention, the DNA aptamer comprises a nucleotide sequence that has at least about 95% (at least about 95%, at least about 96%, at least about 97%, at least about 98%, at least about 99%, or about 100%) sequence identity to at least one of SEQ ID NOs: 121 (M21+RNA_9_3Phos), 134 (M21+RNA_11_3Phos), and 135 (M21+RNA_47_3Phos).

The DNA aptamer of an aspect of the invention, wherein the DNA aptamer comprises at least one (e.g., 1 or 2) 1-20 bp (1-19, 1-18, 1-17, 1-16, 1-15, 1-14, 1-13, 1-12, 1-11, 1-10, 1-9, 1-8, 1-7, 1-6, 1-5, 1-4, 1-3, 1-2, or 1 bp) truncation. The DNA aptamer may be truncated for any suitable reason, including reduction of the size of the DNA aptamer to the change affinity (e.g., increase affinity) for a reverse transcriptase. In an aspect, the DNA aptamer comprises one about 20 bp truncation.

The DNA aptamer may be modified to increase the stability of the DNA aptamer. The DNA aptamers can be, e.g., glycosylated, amidated, carboxylated, phosphorylated, esterified, N-acylated, cyclized via, e.g., a disulfide bridge, or converted into an acid addition salt and/or optionally dimerized or polymerized, or conjugated. For example, the 3′ or 5′ end can be phosphorylated, or a C3 spacer, dideoxy-C, or invented dT may be added, or an amino modifier could be used. The modification could also prevent or reduce non-specific binding and amplification. In an aspect of the invention, the DNA aptamer comprises a nucleotide sequence that has at least 95% (i.e., at least about 95%, at least about 96%, at least about 97%, at least about 98%, at least about 99%, or about 100%) sequence identity to at least one of SEQ ID NO: NOs: 121-125. In an aspect of the invention, the DNA aptamer comprises 3-phosphorylation.

In an aspect of the invention, the DNA aptamer has binding affinity (K_{d}) for a reverse transcriptase. The binding affinity can be calculated using any suitable means. In some aspects of the invention, the DNA aptamer has high specificity for the reverse transcriptases and/or has high binding affinity for a reverse transcriptase and does not specifically bind proteins that are not reverse transcriptases (e.g., the DNA aptamer binds the reverse transcriptases with at least 10, 100, 1,000, 10,000, or 100,000-fold higher affinity than non-reverse transcriptases).

In a further aspect of the invention, the DNA aptamer specifically binds a reverse transcriptase and has high binding affinity for a reverse transcriptase. In an aspect of the invention, the DNA aptamer binds the reverse transcriptase at from about 20° C. to about 30° C. (e.g., from about 21° C. to about 29° C., from about 22° C. to about 28° C., from about 23° C. to about 27° C., from about 24° C. to about 26° C., or about 25° C.), such that the reverse transcriptase is inhibited. In a further aspect of the invention, the DNA aptamer fails to inhibit a reverse transcriptase at from about 45° C. to about 55° C. (e.g., from about 46° C. to about 54° C., from about 47° C. to about 53° C., from about 48° C. to about 52° C., from about 49° C. to about 51° C., or about 50° C.).

An aspect of the present invention provides a user the ability to tightly control reverse transcriptase, by controlling the DNA aptamer's ability to bind, and therefore, inhibit the function of reverse transcriptase. The DNA aptamer can not only be turned “on” and “off.” by a user by adjusting the temperature, but also the DNA aptamer is stable enough to be sequentially switched between on and off. In this regard, the DNA aptamers of the present invention have the feature of having reversible binding and inhibition. These features of the DNA aptamers of the present invention open new possibilities for new laboratory assays with increased functionality. For example, compositions comprising the DNA aptamers of the present invention can be used in a “master mix” for large scale PCR assays and used with automation because the master mixes inhibit the reverse transcriptase activity at room temperature for a long period of time (e.g., at least 4 hours).

The DNA aptamers of an aspect of the invention are useful for any application where unwanted reverse transcriptase function is problematic. Reverse transcriptase has diverse functions including RNA-dependent DNA polymerase activity, terminal transferase activity, and ability to facilitate template switching in cDNA synthesis. Reverse transcriptases are known to interfere with probe-based qPCR when template is added and that is thought occur through qPCR reagent consumption and the uncharacterized production of PCR-interfering nucleic acid products. Accordingly, the DNA aptamers of an aspect of the invention solve this problem by binding and inhibiting reverse transcriptases, and therefore preventing reverse transcriptase function until it is needed.

It was also found that the DNA aptamers of the present invention reversibly inhibit reverse transcriptase at relatively low DNA aptamer to reverse transcriptase ratios and concentrations. This also provides an efficient and cost effective product.

Any suitable reverse transcriptase may be used. In an aspect of the invention, the reverse transcriptase is derived from Moloney murine leukemia virus (MMLV), avian myeloblastosis virus (AMV), or human immunodeficiency virus type 1 (HIV-1). In an aspect of the invention, the reverse transcriptase is derived MMLV. In a further aspect of the invention, the reverse transcriptase comprises a nucleotide sequence that has at least 95% (i.e., at least about 95%, at least about 96%, at least about 97%, at least about 98%, at least about 99%, or about 100%) sequence identity to SEQ ID NO: 132 (M21).

The M21 protein described herein may include functional variants thereof. The functional variant can, for example, comprise the sequence of the parent protein with at least one conservative amino acid substitution. Conservative amino acid substitutions are known in the art, and include amino acid substitutions in which one amino acid having certain physical and/or chemical properties is exchanged for another amino acid that has the same chemical or physical properties. For instance, the conservative amino acid substitution can be an acidic amino acid substituted for another acidic amino acid (e.g., Asp or Glu), an amino acid with a nonpolar side chain substituted for another amino acid with a nonpolar side chain (e.g., Ala, Gly, Val, Ile, Leu, Met, Phe, Pro, Trp, Val, etc.), a basic amino acid substituted for another basic amino acid (Lys, Arg, etc.), an amino acid with a polar side chain substituted for another amino acid with a polar side chain (Asn, Cys, Gln, Ser, Thr, Tyr, etc.), etc.

Alternatively or additionally, the functional variants can comprise the amino acid sequence of the parent protein with at least one non-conservative amino acid substitution. In this case, it is preferable for the non-conservative amino acid substitution to not interfere with or inhibit the biological activity of the functional variant. Preferably, the non-conservative amino acid substitution enhances the biological activity of the functional variant, such that the biological activity of the functional variant is increased as compared to the parent protein.

The aptamers and reverse transcriptases described herein can comprise, consist essentially of, or consist of the specified nucleic acid sequence or sequences described herein, such that other components of the sequences, e.g., other nucleic acids, do not materially change the biological activity of aptamers and reverse transcriptases.

The aptamers can be produced via any suitable means. For example, the aptamers can be synthetic or recombinant.

The aptamers can be isolated and/or purified. The DNA aptamer compositions may be purified using any suitable means. For example, by polyacrylamide gel electrophoresis (PAGE)-purification. The term “isolated” as used herein means having been removed from its natural environment. The term “purified” as used herein means having been increased in purity, wherein “purity” is a relative term, and not to be necessarily construed as absolute purity. For example, the purity can be at least about 50%, at least about 60%, at least about 70%, at least about 80%, at least about 90%, at least about 95%, or can be about 100%.

Also provided herein are compositions comprising at least one DNA aptamer of an aspect of the invention.

The compositions of the present invention may also include at least one reverse transcriptase. In an aspect of the invention, the reverse transcriptase is M21.

The compositions may comprise any suitable excipient. In an aspect of the invention, the compositions further comprise deoxynucleotide triphosphates (dNTPs), a buffer, or a combination thereof. The excipient can be any of those conventionally used for the particular inventive DNA aptamers under consideration.

In an aspect of the invention, the DNA aptamer is present in the composition at a reverse transcriptase: aptamer ratio of from about 1:2 to about 1:8 (i.e., from about 1:2 to about 1:8, from about 1:3 to about 1:8, from about 1:4 to about 1:8, from about 1:5 to about 1:8, from about 1:5 to about 1:7, from about 1:5.5 to about 1:6.5, or about 1:6).

The compositions can comprise any several DNA aptamers. For example, the composition can comprise three DNA aptamers: a first DNA aptamer (aptamer1), a second DNA aptamer (aptamer2), optionally a third DNA aptamer (aptamer3), and optionally a fourth aptamer (aptamer4) etc. In an aspect of the invention, the composition comprises a reverse transcriptase: aptamer1: aptamer2 ratio of from about 1:2:2 to about 1:8:8 (i.e., from about 1:2:2 to about 1:8:8, from about 1:3:3 to about 1:8:8, from about 1:4:4 to about 1:8:8, from about 1:5:5 to about 1:8:8, from about 1:5:5 to about 1:7:7, from about 1:5.5:5.5 to about 1:6.5:6.5, or about 1:6:6).

In an aspect of the invention, the first DNA aptamer comprises a nucleotide sequence that has at least 95% (i.e., at least about 95%, at least about 96%, at least about 97%, at least about 98%, at least about 99%, or about 100%) sequence identity to SEQ ID NO: 121 (M21+RNA_9_3Phos), the second DNA aptamer comprises a nucleotide sequence that has at least 95% (i.e., at least about 95%, at least about 96%, at least about 97%, at least about 98%, at least about 99%, or about 100%) sequence identity to SEQ ID NO: 134 (M21+RNA_11_3Phos), and the third DNA aptamer comprises a nucleotide sequence that has at least 95% (i.e., at least about 95%, at least about 96%, at least about 97%, at least about 98%, at least about 99%, or about 100%) sequence identity to SEQ ID NO: 135 (M21+RNA_47_3Phos).

In a further aspect of the invention, the first DNA aptamer comprises a nucleotide sequence that has at least 95% (i.e., at least about 95%, at least about 96%, at least about 97%, at least about 98%, at least about 99%, or about 100%) sequence identity to SEQ ID NO: 121 (M21+RNA_9_3Phos), the second DNA aptamer comprises a nucleotide sequence that has at least 95% (i.e., at least about 95%, at least about 96%, at least about 97%, at least about 98%, at least about 99%, or about 100%) sequence identity to SEQ ID NO: 134 (M21+RNA_11_3Phos), and the third DNA aptamer comprises a nucleotide sequence that has at least 95% (i.e., at least about 95%, at least about 96%, at least about 97%, at least about 98%, at least about 99%, or about 100%) sequence identity to SEQ ID NO: 135 (M21+RNA_47_3Phos), and wherein the composition comprises a reverse transcriptase: aptamer 1: aptamer2: aptamer3 ratio of from about 1:2:2:2 to about 1:8:8:8. In an aspect of the invention, the composition comprises a reverse transcriptase: aptamer 1: aptamer2: aptamer3 ratio of about 1:6:6:6.

In an aspect of the invention, the composition comprises a RNA aptamer. Any suitable RNA aptamer may be used. In an aspect of the invention, the RNA aptamer comprises a sequence that has at least 95% (i.e., at least about 95%, at least about 96%, at least about 97%, at least about 98%, at least about 99%, or about 100%) sequence identity to SEQ ID NO: 1.

An aspect of the invention provides a kit comprising a DNA aptamer of the present invention or a composition of the present invention. The kit can contain instructions, a container for holding the DNA aptamer or composition, and any other components necessary or useful for performing the kit.

A further aspect of the invention is using the DNA aptamers in PCRs. In this regard, an aspect of the invention provides a method for amplifying a target DNA sequence, the method comprising: (a) providing a composition of an aspect of the invention, the composition further comprising at least one oligonucleotide primer, and at least one sample comprising at least one target DNA sequence, wherein the composition is at a temperature of from about 20° C. to about 30° C. (e.g., from about 21° C. to about 29° C., from about 22° C. to about 28° C., from about 23° C. to about 27° C., from about 24° C. to about 26° C., or about 25° C.), and wherein the reverse transcriptase is bound to the at least one DNA aptamer, (b) elevating the temperature of the composition to release the reverse transcriptase from the at least one DNA aptamer, (c) hybridizing the at least one oligonucleotide primer to the at least one sample comprising at least one target DNA sequence; and (d) initiating DNA polymerase activity and extending the primer with the DNA polymerase to provide additional target DNA sequences. In an aspect of the invention, the composition is at a temperature of from about 20° C. to about 30° C. for at least about 4 hours (i.e., about 5 hours, about 6 hours, about 7 hours, about 8 hours, about 12 hours, about 16 hours, about 20 hours, about 24 hours, or more) in step (a). In a further aspect, the composition is elevated to a temperature of from about 45° C. to about 55° C. (e.g., from about 46° C. to about 54° C., from about 47° C. to about 53° C., from about 48° C. to about 52° C., from about 49° C. to about 51° C., or about 50° C.) in step (b).

In this regard, a further aspect of the invention provides a method for identifying at least one DNA aptamer that inhibits at least one reverse transcriptase, the method comprising: (a) providing a DNA aptamer pool comprising DNA aptamers, (b) allowing the DNA aptamers to be in proximity to the at least one reverse transcriptase in the presence of a RNA aptamer, (c) separating DNA aptamers with high affinity for the at least one reverse transcriptase from the DNA aptamers with low affinity for the at least one reverse transcriptase, and (d) amplifying the DNA aptamers with high affinity for the at least one reverse transcriptase to obtain an enriched DNA aptamer pool, wherein the at least one DNA aptamer that inhibits at least one reverse transcriptase may be identified from the enriched DNA aptamer pool. Unexpectedly, it was found that the presence of a RNA aptamer was required for identification of the DNA aptamers. One skilled in the art would be able to determine what “proximity” refers to. Similarly, one skilled in the art would be able to determine cut offs for “high affinity” and “low affinity.”

In a further aspect of the invention, the RNA aptamer comprises a sequence that has at least 95% (i.e., at least about 95%, at least about 96%, at least about 97%, at least about 98%, at least about 99%, or about 100%) sequence identity to SEQ ID NO: 1.

In another aspect of the invention, the reverse transcriptase comprises a nucleotide sequence that has at least 95% (i.e., at least about 95%, at least about 96%, at least about 97%, at least about 98%, at least about 99%, or about 100%) sequence identity to SEQ ID NO: 132.

The method disclosed herein may include the use of probes which include dyes/fluophores and quenchers for quantifying binding. Some of the oligonucleotides disclosed herein include a fluorophore, which may include, but is not limited to, 6-carboxyfluorescein (FAM), 2′7′-dimethoxy-4′5′-dichloro-6-carboxyfluorescein (JOE), tetrachlorofluorescein (TET), 6-carboxyrhodamine (R6G), N,N,N;N′-tetramethyl-6-carboxyrhodamine (TAMRA), 6-carboxy-X-rhodamine (ROX): 1-dimethylaminonaphthyl-5-sulfonate, 1-anilino-8-naphthalene sulfonate, 2-p-toluidinyl-6-naphthalene sulfonate, 5-(2′-aminoethyl) aminonaphthalene-1-sulfonic acid (EDANS), a coumarin dye, an acridine dye, indodicarbocyanine 3 (Cy3), indodicarbocyanine 5 (Cy5), indodicarbocyanine 5.5 (Cy5.5), 3-1-carboxy-pentyl)-3′-ethyl-5,5′-dimethyloxacarbocyanine (CyA): 1H,5H,11H,15H-Xantheno [2,3,4-ij: 5,6,7-i′j′] diquinolizin-18-ium, 9-[2 (or 4)-[[[6-[2,5-dioxo-1-pyrrolidinyl)oxy]-6-oxohexyl|amino|sulfonyl]-4 (or 2)-sulfophenyl]-2,3,6, 7, 12, 13, 16, 17-octahydro-inner salt (TR or TexasRed™), a BODIPY™ dye, Yakima Yellow™ dye, benzoxaazole, stilbene and pyrene.

Some of the oligonucleotides disclosed herein include a quencher. Suitable quenchers may include dabcyl, Eclipse™ quencher, BHQ1, BHQ2 and BHQ3, Iowa Black™ FQ, Iowa Black™ RQ-n1, or Iowa Black™ RQ-n2 (Integrated DNA Technologies, Inc.).

### Examples of Non-Limiting Aspects of the Disclosure

Aspects, including embodiments, of the invention described herein may be beneficial alone or in combination, with one or more other aspects or embodiments. Without limiting the foregoing description, certain non-limiting aspects of the disclosure numbered (1)-(30) are provided below. As will be apparent to those of skill in the art upon reading this disclosure, each of the individually numbered aspects may be used or combined with any of the preceding or following individually numbered aspects. This is intended to provide support for all such combinations of aspects and is not limited to combinations of aspects explicitly provided below:

(1) A DNA aptamer comprising a nucleotide sequence that has at least 95% sequence identity to at least one of SEQ ID NOs: 2-101.

(2) The DNA aptamer of aspect 1, wherein the DNA aptamer consists of from about 50 to about 90 nucleotides.

(3) The DNA aptamer of aspect 1 or 2, comprising a nucleotide sequence that has at least 95% sequence identity to at least one of SEQ ID NOs: 29 (M21_28), 45 (M21_44), 60 (M21+RNA_9), 72 (M21+RNA_21), 76 (M21+RNA_25), 78 (M21+RNA_27), 79 (M21+RNA_28), 81 (M21+RNA_30), 82 (M21+RNA_31), 83 (M21+RNA_32), 90 (M21+RNA_39), 91 (M21+RNA_40), 93 (M21+RNA_42), 94 (M21+RNA_43), 95 (M21+RNA_44), 121 (M21+RNA_9_3Phos), 134 (M21+RNA_11_3Phos), and 135 (M21+RNA_47_3Phos).

(4) The DNA aptamer of any one of aspects 1-3, comprising a nucleotide sequence that has at least 95% sequence identity to at least one of SEQ ID NOs: 121 (M21+RNA_9_3Phos), 134 (M21+RNA_11_3Phos), and 135 (M21+RNA_47_3Phos).

(5) The DNA aptamer of any one of aspects 1-4, wherein the DNA aptamer comprises at least one 1-20 bp truncation.

(6) The DNA aptamer of aspect 1 or 2, wherein the DNA aptamer comprises 3′-phosphorylation.

(7) The DNA aptamer of any one of aspects 1-5, wherein the DNA aptamer has binding affinity for a reverse transcriptase.

(8) The DNA aptamer of any one of aspects 1-7, wherein the DNA aptamer inhibits a reverse transcriptase at from about 20° C. to about 30° C.

(9) The DNA aptamer of any one of aspects 1-8, wherein the DNA aptamer fails to inhibit a reverse transcriptase at from about 45° C. to about 55° C.

(10) The DNA aptamer of any one of aspects 7-9, wherein the reverse transcriptase is derived from Moloney murine leukemia virus (MMLV), avian myeloblastosis virus (AMV), or human immunodeficiency virus type 1 (HIV-1).

(11) The DNA aptamer of any one of aspects 7-10, wherein the reverse transcriptase comprises a nucleotide sequence that has at least 95% sequence identity to SEQ ID NO: 132.

(12) A composition comprising at least one DNA aptamer of any one of aspects 1-11.

(13) The composition of aspect 12, wherein the at least one DNA aptamer is polyacrylamide gel electrophoresis (PAGE)-purified.

(14) The composition of aspect 12 or 13, wherein the composition further comprises a reverse transcriptase.

(15) The composition of any one of aspects 12-14, wherein the composition further comprises deoxynucleotide triphosphates (dNTPs), a buffer, or a combination thereof.

(16) The composition of any one of aspects 12-15, wherein the DNA aptamer is present in the composition at a reverse transcriptase: aptamer ratio of from about 1:2 to about 1:8.

(17) The composition of any one of aspects 12-15, wherein the composition comprises a first DNA aptamer (aptamer1), a second DNA aptamer (aptamer2), and optionally a third DNA aptamer (aptamer3).

(18) The composition of aspect 17, wherein the composition comprises a reverse transcriptase:aptamer l:aptamer2 ratio of from about 1:2:2 to about 1:8:8.

(19) The composition of aspect 17, wherein the first DNA aptamer comprises a nucleotide sequence that has at least 95% sequence identity to SEQ ID NO: 121 (M21+RNA_9_3Phos), the second DNA aptamer comprises a nucleotide sequence that has at least 95% sequence identity to SEQ ID NO: 134 (M21+RNA_11_3Phos), and the third DNA aptamer comprises a nucleotide sequence that has at least 95% sequence identity to SEQ ID NO: 135 (M21+RNA_47_3Phos).

(20) The composition of aspect 17, wherein the first DNA aptamer comprises a nucleotide sequence that has at least 95% sequence identity to SEQ ID NO: 121 (M21+RNA_9_3Phos), the second DNA aptamer comprises a nucleotide sequence that has at least 95% sequence identity to SEQ ID NO: 134 (M21+RNA_11_3Phos), and the third DNA aptamer comprises a nucleotide sequence that has at least 95% sequence identity to SEQ ID NO: 135 (M21+RNA_47_3Phos), and wherein the composition comprises a reverse transcriptase: aptamer 1: aptamer2: aptamer3 ratio of from about 1:2:2:2 to about 1:8:8:8.

(21) The composition of aspect 17, wherein first DNA aptamer comprises a nucleotide sequence that has at least 95% sequence identity to SEQ ID NO: 121 (M21+RNA_9_3Phos), the second DNA aptamer comprises a nucleotide sequence that has at least 95% sequence identity to SEQ ID NO: 134 (M21+RNA_11_3Phos), and the third DNA aptamer comprises a nucleotide sequence that has at least 95% sequence identity to SEQ ID NO: 135 (M21+RNA_47_3Phos), and wherein the composition comprises a reverse transcriptase: aptamer 1: aptamer2: aptamer3 ratio of about 1:6:6:6.

(22) The composition of any one of aspects 12-20, further comprising a RNA aptamer, optionally wherein the RNA aptamer comprises a sequence that has at least 95% sequence identity to SEQ ID NO: 1.

(23) A kit comprising the DNA aptamers of any one of aspects 1-11, or the composition of any one of aspects 12-22.

(24) A method for amplifying a target DNA sequence, the method comprising:
a. providing a composition of any one of aspects 12-22, the composition further comprising at least one oligonucleotide primer, and at least one sample comprising at least one target DNA sequence, wherein the composition is at a temperature of from about 20° C. to about 30° C., and wherein the reverse transcriptase is bound to the at least one DNA aptamer;
b. elevating the temperature of the composition to release the reverse transcriptase from the at least one DNA aptamer;
c. hybridizing the at least one oligonucleotide primer to the at least one sample comprising at least one target DNA sequence; and
d. initiating DNA polymerase activity and extending the primer with the DNA polymerase to provide additional target DNA sequences.

(25) The method of aspect 24, wherein the composition is at a temperature of from about 20° C. to about 30° C. for at least 4 hours in step (a).

(26) The method of aspect 24, wherein the composition is at a temperature of from about 20° C. to about 30° C. for at least 12 hours in step (a).

(27) The method of aspect 24, wherein the composition is elevated to a temperature of from about 45° C. to about 55° C. in step (b).

(28) A method for identifying at least one DNA aptamer that inhibits at least one reverse transcriptase, the method comprising:
a. providing a DNA aptamer pool comprising DNA aptamers;
b. allowing the DNA aptamers to be in proximity to the at least one reverse transcriptase in the presence of a RNA aptamer;
c. separating DNA aptamers with high affinity for the at least one reverse transcriptase from the DNA aptamers with low affinity for the at least one reverse transcriptase; and
d. amplifying the DNA aptamers with high affinity for the at least one reverse transcriptase to obtain an enriched DNA aptamer pool, wherein the at least one DNA aptamer that inhibits at least one reverse transcriptase may be identified from the enriched DNA aptamer pool.

(29) The method of aspect 28, wherein the RNA aptamer comprises a sequence that has at least 95% sequence identity to SEQ ID NO: 1.

(30) The method of aspect 28 or 29, wherein the reverse transcriptase comprises a nucleotide sequence that has at least 95% sequence identity to SEQ ID NO: 132.

The following examples further illustrate the invention but, of course, should not be construed as in any way limiting its scope.

### Example 1

This example demonstrates the isolation of DNA aptamers of an aspect of the invention that bind to MMLV reverse transcriptase mutants.

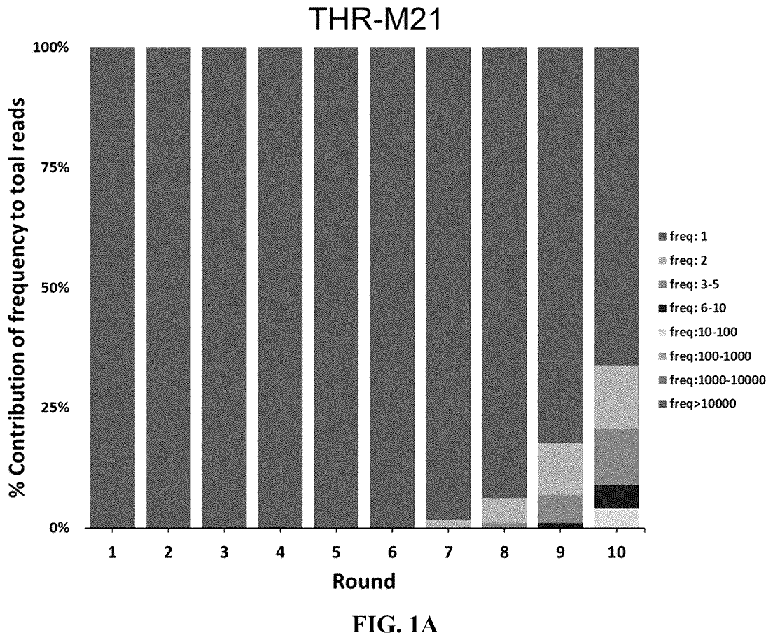

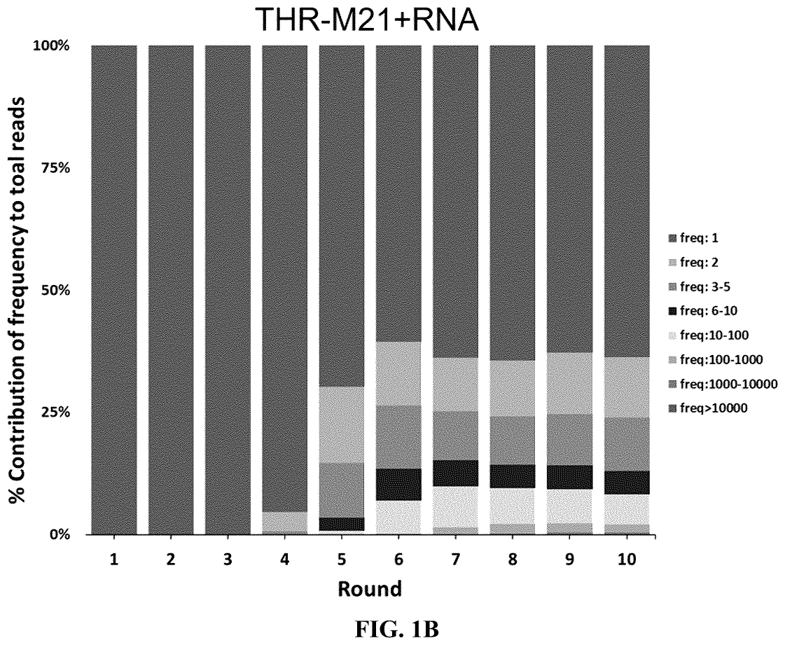

A 40 bp randomized DNA oligo pool flanked by two 21 bp adapter sequences (total aptamer length of 82 bp) was used to perform 10 rounds of SELEX with M21 RTase both in the presence or absence of a previously described reverse transcriptase RNA aptamer (Chen, et al., Biochemistry, 33 (29): 8746-56 (1994)). The enriched aptamer pools were sequenced by NGS for every round after enrichment (FIGS. 1A and 1B and Tables 2A and 2B). These results demonstrated that DNA aptamer enrichment occurred much earlier in experiments containing RNA aptamer. This finding was unexpected. Without being bound to any particular theory, the DNA aptamer enrichment may have occurred earlier due to a conformational change in the reverse transcriptase enzyme resulting in opening of the DNA exit channel when an RNA template is present.

Enriched aptamer sequences obtained from SELEX experiments in the presence and absence of RNA aptamer were rank ordered by percent enrichment (Table 1). The top 50 enriched aptamers for both conditions were chemically synthesized by IDT as 100 nmol DNA oligonucleotides to be used in follow up studies. Typically, “one-step” RT-qPCR reactions allow end-users to convert RNA templates to cDNA and perform quantitative PCR using a single master-mix and cycling program. In these reactions, the presence of reverse transcriptase can decrease qPCR signal in later cycles dramatically if reactions are not run immediately after loading. Evaluation of reverse transcriptase inhibition by DNA aptamers will therefore be evaluated using one-step qPCR reactions where template is added and plates are held at room temperature for several hours before running.

TABLE 2A

| Sample | THR-M21 | | | | | | | | | |
|---|---|---|---|---|---|---|---|---|---|---|
| Round | R1 | R2 | R3 | R4 | R5 | R6 | R7 | R8 | R9 | R10 |
| Total sequence in | 4,951,799 | 4,951,799 | 4,951,799 | 4,951,799 | 4,951,799 | 4,951,799 | 4,951,799 | 4,951,799 | 4,951,799 | 4,951,799 |
| the pool | | | | | | | | | | |
| freq: 0 | 4,201,556 | 4,113,861 | 4,404,391 | 4,189,930 | 4,415,628 | 4,430,254 | 4,375,014 | 4,688,988 | 4,810,125 | 4,818,769 |
| % sequence not in | 85% | 83% | 89% | 85% | 89% | 89% | 88% | 95% | 97% | 97% |
| the sample | | | | | | | | | | |
| freq: 1 | 749,641 | 837,208 | 546,990 | 761,217 | 535,747 | 520,853 | 566,650 | 246,428 | 116,744 | 88,037 |
| freq: 2 | 601 | 730 | 418 | 652 | 424 | 690 | 9,560 | 13,853 | 15,327 | 17,415 |
| freq: 3-5 | 1 | — | — | — | — | 2 | 574 | 2,483 | 8,131 | 15,643 |
| freq: 6-10 | — | — | — | — | — | — | 1 | 47 | 1,304 | 6,590 |
| freq: 10-100 | — | — | — | — | — | — | — | — | 168 | 5,303 |
| freq: 100-1000 | — | — | — | — | — | — | — | — | — | 42 |
| freq: 1000-10000 | — | — | — | — | — | — | — | — | — | — |
| freq > 10000 | — | — | — | — | — | — | — | — | — | — |

TABLE 2B

| | Sample | | | | | | | | | |
|---|---|---|---|---|---|---|---|---|---|---|
| | THR-M21 + RNA | | | | | | | | | |
| Round | R1 | R2 | R3 | R4 | R5 | R6 | R7 | R8 | 89 | R10 |
| Total sequence in | 2,690,140 | 2,690,140 | 2,690,140 | 2,690,140 | 2,690,140 | 2,690,140 | 2,690,140 | 2,690,140 | 2,690,140 | 2,690,140 |
| the pool | | | | | | | | | | |
| freq: 0 | 2,241,190 | 2,060,853 | 1,920,632 | 2,285,415 | 2,378,667 | 2,497,940 | 2,589,602 | 2,638,216 | 2,666,492 | 2,672,389 |
| % sequence not | 83% | 77% | 71% | 85% | 88% | 93% | 96% | 98% | 99% | 99% |
| in the sample | | | | | | | | | | |
| freq: 1 | 448,630 | 628,716 | 768,748 | 386,215 | 217,345 | 116,393 | 54,196 | 33,447 | 14,854 | 11,309 |
| freq: 2 | 320 | 571 | 760 | 15,853 | 48,434 | 25,023 | 11,048 | 5,936 | 2,981 | 2,200 |
| freq: 3-5 | — | — | — | 2,639 | 34,973 | 24,983 | 9,970 | 5,109 | 2,476 | 1,938 |
| freq: 6-10 | — | — | — | 18 | 8,307 | 12,355 | 5,422 | 2,507 | 1,137 | 846 |
| freq: 10-100 | — | — | — | — | 2,414 | 13,131 | 8,364 | 3,796 | 1,661 | 1,091 |
| freq: 100-1000 | — | — | — | — | — | 315 | 1,488 | 1,007 | 448 | 281 |
| freq: 1000-10000 | — | — | — | — | — | — | 50 | 119 | 85 | 76 |
| freq >10000 | — | — | — | — | — | — | — | 3 | 5 | 10 |

### Example 2

This example demonstrates reverse transcriptase inhibition by DNA aptamers of an aspect of the invention using one step RT-qPCR.

DNA aptamers were tested in the context of one step RT-qPCR where a synthetic RNA genome respiratory virus panel (Influenza A, Influenza B, SARS-COV-2, and Human RNase P) was amplified and signal (Ct and fluorescence intensity) was measured running plates on the instrument immediately after template addition (time point=0) or holding plates two hours at 25° C. before running the instrument (time point=2 hrs).

All 100 DNA aptamers were tested with and without RNA aptamer in the context of one step RT-qPCR. For the binding of RNA aptamer to M21 RTase, equal volumes of RNA aptamer (2.4 μM, Table 1) and M21 RTase (0.724 UM, Table 1) were mixed and incubated at room temperature for 15 minutes. Following incubation, a third volume of DNA Aptamer (1.45 μM, Table 1) was added to the RNA aptamer bound RTase and incubated at room temperature for a further 15 minutes. After incubation, the RNA and DNA aptamer bound RTase was placed on ice. For the binding of DNA aptamer to M21 RTase, equal volumes of DNA aptamer (1.45 M, Table 1) and M21 RTase (0.724 μM, Table 1) were mixed and incubated at room temperature for 15 minutes. After incubation, the DNA aptamer bound RTase was placed on ice. For the standard one-step procedure, the RNA and DNA aptamer bound RTases (6.03 nM final) were added to a reaction mixture containing the following: Integrated DNA Technologies PrimeTime™ 1-Step 4X Broad-Range Master Mix formulated without Reverse Transcriptase (1X) and FluVID primers and probe sets (Table 1). The assay master mix and synthetic RNA ultramers (50 copies per well, Table 1) were mixed at a 4:1 ratio for a final volume of 20 μL. The reaction proceeded either immediately or two hours after the addition of template at room temperature on a qPCR (QuantStudio™ 7 Flex) using the following method: 50° C. hold for 15 minutes, 95° C. hold for 30 seconds, followed by 95° C. for 5 seconds and 55° C. for 45 seconds for 45 cycles. The reactions were analyzed and reported by threshold cycle (Ct, with threshold determined by the 0-hour incubation timepoint) and endpoint fluorescence (delta Rn) values (Tables 3 and 4). Of the 100 aptamers tested, 15 aptamers showed a significant increase in on-deck reaction mix stability over the RNA aptamer alone as assessed by endpoint fluorescence and cycle threshold. These 15 aptamers include: M21_28, M21_44, M21+RNA_9, M21+RNA_21, M21+RNA_25, M21+RNA_27, M21+RNA_28, M21+RNA_30, M21+RNA_31, M21+RNA 32, M21+RNA 39, M21+RNA_40, M21+RNA_42, M21+RNA 43, and M21+RNA 44. It is interesting to note that the best aptamers were the same whether RNA aptamer was added or not, and yet the best aptamers were obtained from SELEX experiments where the RNA aptamer was included. Without being bound to any particular theory, these results suggest that RNA was important to isolate the best DNA aptamers, but that the RNA aptamer provides little to no additive benefit over these new DNA aptamers, which was unexpected.

### Example 3

This example demonstrates that the DNA aptamers of an aspect of the invention inhibit reverse transcriptase at different concentrations.

This example provides a procedure used to evaluate a dose range of some of the DNA aptamers from Example 2 and the resulting effect on reverse transcriptase inhibition using one step RT-PCR. To further “challenge” the candidate DNA aptamers, qPCR reactions were held for 0 or 4 hours at 25° C. after template addition before running the instrument. The DNA aptamers were tested by first binding the DNA aptamer at various ratios (1:2, 1:3, 1:5, and 1:10 RTase: Aptamer) to M21 RTase, adding the aptamer bound RTase to a reaction mixture containing a qPCR master mix with multiplexing primers and probes and testing the on-deck rection mix stability of the generated one-step RT-qPCR master mix after template addition.

For the binding of DNA aptamer to M21 RTase, equal volumes of DNA aptamer at varying concentrations (1.45 M, 2.17 μM, 3.62 μM, and 7.24 μM, Table 1) and M21 RTase (0.724 μM, Table 1) were mixed and incubated at room temperature for 15 minutes. After incubation, the DNA aptamer bound RTase was placed on ice. For the standard one-step procedure, the DNA aptamer bound RTases (6.03 nM final) were added to a reaction mixture containing the following: Integrated DNA Technologies PrimeTime™ 1-Step 4X Broad-Range Master Mix formulated without Reverse Transcriptase (1X) and FluVID primers and probe sets (Table 1). The assay master mix and synthetic RNA ultramers (50 copies per well, Table 1) were mixed at a 4:1 ratio for a final volume of 20 μL. The reaction proceeded either immediately or four hours after the addition of template at room temperature on a qPCR (QuantStudio™ 7 Flex) using the following method: 50° C. hold for 15 minutes, 95° C. hold for 30 seconds, followed by 95° C. for 5 seconds and 55° C. for 45 seconds for 45 cycles. The reactions were analyzed and reported by threshold cycle (Ct, with threshold determined by the 0-hour incubation timepoint) and endpoint fluorescence (delta Rn) values (Table 5). While all the conditions with DNA aptamers yielded improved on-deck reaction mix stability over the RNA only control, there wasn't substantial improvement as the concentration of aptamer increased, suggesting that the 1:2 ratio of M21: Aptamer is sufficient to achieve maximum effect.

### Example 4

This example demonstrates reverse transcriptase inhibition by combinations of DNA aptamers of an aspect of the invention.

This example provides a procedure used to evaluate combining the DNA aptamers of an aspect of the invention and looking for an additive effect on reverse transcriptase inhibition by one step qPCR. Aptamers were challenged by holding reactions at 0 or 4 hours at 25° C. after template addition before running the instrument. The DNA aptamers were tested by first binding two DNA aptamers to M21 RTase, adding the aptamer bound RTase to a reaction mixture containing a qPCR master mix with multiplexing primers and probes and testing the on-deck reaction mix stability of the generated one-step RT-qPCR master mix after template addition.

For the binding of DNA aptamers to M21 RTase, equal volumes of two DNA aptamers (1.45 μM, Table 1) and M21 RTase (0.724 μM, Table 1) were mixed and incubated at room temperature for 15 minutes. After incubation, the DNA aptamer bound RTase was placed on ice. For the standard one-step procedure, the DNA aptamer bound RTases (6.03 nM final) were added to a reaction mixture containing the following: Integrated DNA Technologies PrimeTime™ 1-Step 4X Broad-Range Master Mix formulated without Reverse Transcriptase (1X) and FluVID primers and probe sets (Table 1). The assay master mix and synthetic RNA ultramers (50 copies per well, Table 1) were mixed at a 4:1 ratio for a final volume of 20 μL. The reaction proceeded either immediately or four hours after the addition of template at room temperature on a qPCR (QuantStudio™ 7 Flex) using the following method: 50° C. hold for 15 minutes, 95° C. hold for 30 seconds, followed by 95° C. for 5 seconds and 55° C. for 45 seconds for 45 cycles. The reactions were analyzed and reported by threshold cycle (Ct, with threshold determined by the 0-hour incubation timepoint) and endpoint fluorescence (delta Rn) values (Table 5). Of all the combinations tested, 5 were selected as the best based on high endpoint fluorescence, low Ct, and low variance. These combinations were: M21_44+M21+RNA_31, M21+RNA_9+M21+RNA_27, M21+RNA 9+M21+RNA 30, M21+RNA_31+M21+RNA_44, and M21+RNA_43+M21+RNA 44.

### Example 5

This example further demonstrates the effectiveness of combinations of DNA aptamers of the present invention.

In this study, the DNA aptamers were combined at different ratios in the presence or absence of RNA aptamer. These DNA aptamers were challenged by incubation of the qPCR reactions for 0, 4, and 12 hours at 25° C. after template addition before running on the instrument. The DNA aptamers were tested by first binding RNA aptamer to M21 RTase where appropriate, followed by binding one or two DNA aptamers to M21 RTase, adding the aptamer bound RTase to a reaction mixture containing a qPCR master mix with multiplexing primers and probes and testing the on-deck reaction mix stability of the generated one-step RT-qPCR master mix after template addition.

For the conditions that contained RNA aptamer, equal volumes of RNA aptamer (2.4 μM, Table 1) and M21 RTase (0.724 μM, Table 1) were mixed and incubated at room temperature for 15 minutes. Following incubation, a third volume of DNA Aptamer containing either one or two DNA aptamers with final concentrations of 0.724 μM, 1.45 μM, or 3.62 μM for 1:1:3.3, 1:2:3.3, or 1:5:3.3 M21: DNA Aptamer: RNA Aptamer (or 1:1:1:3.3, 1:2:2:3.3, 1:5:5:3.3 M21: DNA Aptamer 1: DNA Aptamer 2: RNA Aptamer for the combinations), respectively (Table 1), were added to the RNA aptamer bound RTase and incubated at room temperature for a further 15 minutes.

For the conditions that didn't contain RNA aptamer, equal volumes of M21 RTase (0.724 μM, Table 1) and DNA Aptamer containing either one or two DNA aptamers with final concentrations of 0.724 μM, 1.45 μM, or 3.62 μM for 1:1, 1:2, or 1:5 (or 1:1:1, 1:2:2, 1:5:5 for the combinations), respectively (Table 1), were added to a third volume of IDTE pH 7.5 and incubated at room temperature for 15 minutes. After incubation, the DNA aptamer bound RTase was placed on ice. For the standard one-step procedure, the DNA aptamer bound RTases (6.03 nM final) were added to a reaction mixture containing the following: Integrated DNA Technologies PrimeTime™ 1-Step 4X Broad-Range Master Mix formulated without Reverse Transcriptase (1X) and FluVID primers and probe sets (Table 1). The assay master mix and synthetic RNA ultramers (50 copies per well, Table 1) were mixed at a 4:1 ratio for a final volume of 20 μL. The reaction proceeded either immediately, four hours, or twelve hours after the addition of template at room temperature on a qPCR (QuantStudio™ 7 Flex) using the following method: 50° C. hold for 15 minutes, 95° C. hold for 30 seconds, followed by 95° C. for 5 seconds and 55° C. for 45 seconds for 45 cycles. The reactions were analyzed and reported by threshold cycle (Ct, with threshold determined by the 0-hour incubation timepoint) and endpoint fluorescence (delta Rn) values (Table 7). Of all the conditions tested, M21+RNA_9+M21+RNA_27 consistently performed as one of the best in terms of based on high endpoint fluorescence, low Ct, and low variance. In fact, M21+RNA 9+M21+RNA 27 was even able to achieve successful amplification of the RNaseP and SARS-COV-2 targets following 12 hours of incubation at room temperature, though the InfA and InfB were not able to clear the cycle threshold from no incubation condition. In nearly all cases, addition of the RNA aptamer yielded lower endpoint fluorescence than the corresponding condition absent the RNA aptamer.

### Example 6

This example demonstrates that extension blocking 3′ modifications do not interfere with combined DNA aptamer inhibition of reverse transcriptase activity.

In this study, 3′ DNA modifications were added to the terminal base of DNA aptamers that prevent extension by a DNA polymerase. The goal of these experiments was to find 3′ modifications that do not interfere with inhibition of reverse transcriptase by the combined DNA aptamers. These chemically modified DNA aptamers were synthesized by IDT as 100 nmol DNA oligonucleotides. These DNA aptamers were challenged by incubation of the qPCR reactions for 4 hours at 25° C. after template addition before running on the instrument. The DNA aptamers were tested by binding one or two DNA aptamers to M21 RTase, adding the aptamer bound RTase to a reaction mixture containing a qPCR master mix with multiplexing primers and probes and testing the on-deck reaction mix stability of the generated one-step RT-qPCR master mix after template addition.

For these experiments, equal volumes of M21 RTase (0.724 μM, Table 1) and DNA Aptamer containing either one or two DNA aptamers with final concentrations of 0.724 μM, 1.45 μM, or 3.62 μM for 1:1, 1:2, or 1:5 (or 1:1: 1, 1:2:2, 1:5:5 for the combinations), respectively (Table 1), were added to a third volume of IDTE pH 7.5 and incubated at room temperature for 15 minutes. After incubation, the DNA aptamer bound RTase was placed on ice. For the standard one-step procedure, the DNA aptamer bound RTases (6.03 nM final) were added to a reaction mixture containing the following: Integrated DNA Technologies PrimeTime™ 1-Step 4X Broad-Range Master Mix formulated without Reverse Transcriptase (1X) and FluVID primers and probe sets (Table 1). The assay master mix and synthetic RNA ultramers (50 copies per well, Table 1) were mixed at a 4:1 ratio for a final volume of 20 μL. The reaction proceeded four hours after the addition of template at room temperature on a qPCR (QuantStudio™ 7 Flex) using the following method: 50° C. hold for 15 minutes, 95° C. hold for 30 seconds, followed by 95° C. for 5 seconds and 55° C. for 45 seconds for 45 cycles. The reactions were analyzed and reported by threshold cycle (Ct) and endpoint fluorescence (delta Rn) values (Table 8). Generally, most 3′ blocking extensions were well-tolerated and the resulting DNA aptamers and DNA aptamer combinations functioned well to inhibit reverse transcriptase activity in one-step qPCR reactions. The DNA aptamers containing 3′ phosphate modifications appeared to produce slightly more consistent data than other modifications or unmodified aptamers.

### Example 7

This example demonstrates the range of binding periods between reverse transcriptases and DNA aptamers of an aspect of the invention.

This example provides a procedure used to determine the optimal binding period between the MMLV RTase and DNA aptamers to maximize the benefit to on-deck reaction mix stability using one step RT-PCR. DNA aptamers were tested in the context of one step RT-qPCR where a synthetic RNA genome respiratory virus panel (Influenza A, Influenza B, SARS-COV-2, and Human RNase P) was amplified and signal (Ct and fluorescence intensity) was measured running plates on the instrument after holding plates six hours at 25° C. before running the instrument (time point=6 hrs).

A single, top-performing DNA aptamer was bound to the M21 RTase for 15, 20 and 30 minutes and the effect on on-deck reaction mix stability was tested in the context of one step RT-qPCR. For the binding of DNA aptamer to M21 RTase, equal volumes of DNA aptamer (2.9 μM, Table 1) and M21 RTase (0.724 UM, Table 1) were mixed and incubated at room temperature for either 15, 20 or 30 minutes. After incubation, the DNA aptamer bound RTase was placed on ice. For the standard one-step procedure, the DNA aptamer bound RTases (6.03 nM final) were added to a reaction mixture containing the following: Integrated DNA Technologies PrimeTime™ 1-Step 4X Broad-Range Master Mix formulated without Reverse Transcriptase (1X) and FluVID primers and probe sets (Table 1). The assay master mix and synthetic RNA ultramers (50 copies per well, Table 1) were mixed at a 4:1 ratio for a final volume of 20 μL. The reaction proceeded six hours after the addition of template at room temperature on a qPCR (QuantStudio™ 7 Flex) using the following method: 50° C. hold for 15 minutes, 95° C. hold for 30 seconds, followed by 95° C. for 5 seconds and 55° C. for 45 seconds for 45 cycles. The reactions were analyzed and reported by threshold cycle (Ct) and endpoint fluorescence (delta Rn) values (Table 9). The 20 and 30 minute binding period showed a marked improvement over the 15 minute binding in terms of on-deck reaction mix stability.

### Example 8

This example demonstrates the reverse transcriptase inhibition by combinations of DNA aptamers of the present invention using one step RT-qPCR.

Pairs of DNA aptamers have been shown to have beneficial effects on improving on-deck reaction mix stability compared to single DNA aptamers. Previous data has demonstrated M21+RNA_9_3Phos as one of the best DNA aptamers as a stand-a-lone aptamer. In response, the DNA aptamers (100 in total) were paired with M21+RNA_9_3Phos, bound to reverse transcriptase and tested in the context of one step RT-qPCR where a synthetic RNA genome respiratory virus panel (Influenza A, Influenza B, SARS-COV-2, and Human RNase P) was amplified and signal (Ct and fluorescence intensity) was measured running plates on the instrument after holding plates six hours at 25° C. before running the instrument (time point=6 hrs).

For the binding of DNA aptamers to M21 RTase, equal volumes of M21+RNA_9_3Phos and an unmodified DNA aptamer (1.45 μM each Table 1) and M21 RTase (0.724 μM, Table 1) were mixed and incubated at room temperature for 1 hour. After incubation, the DNA aptamer bound RTase was placed on ice. For the standard one-step procedure, the DNA aptamer bound RTases (6.03 nM final) were added to a reaction mixture containing the following: Integrated DNA Technologies PrimeTime™ 1-Step 4X Broad-Range Master Mix formulated without Reverse Transcriptase (1X) and FluVID primers and probe sets (Table 1). The assay master mix and synthetic RNA ultramers (50 copies per well, Table 1) were mixed at a 4:1 ratio for a final volume of 20 μL. The reaction proceeded six hours after the addition of template at room temperature on a qPCR (QuantStudio™ 7 Flex) using the following method: 50° C. hold for 15 minutes, 95° C. hold for 30 seconds, followed by 95° C. for 5 seconds and 55° C. for 45 seconds for 45 cycles. The reactions were analyzed and reported by threshold cycle (Ct) and endpoint fluorescence (delta Rn) values (Table 10). Of the 100 aptamers paired with M21+RNA_9_3Phos, two pairs outperformed the others: M21+RNA_9_3Phos with M21+RNA_11 and M21+RNA_9_3Phos with M21+RNA_47. M21+RNA 11 and M21+RNA_47 were ordered with the 3′ Phosphorylation modification for further study.

### Example 9

This example demonstrates reverse transcriptase inhibition by DNA aptamers of an aspect of the invention at different concentrations.

This example provides a procedure used to evaluate a dose range of the best paired DNA aptamers from Example 8 and the resulting effect on reverse transcriptase inhibition using one step RT-PCR. The aptamers were challenged by holding reactions 6 hours at 25° C. after template addition before running the instrument. The DNA aptamers were tested by first binding two DNA aptamers at various ratios (1:2:2, 1:3:3, 1:4:4, 1:6:6 and 1:8:8 RTase: Aptamer 1: Aptamer 2) to M21 RTase, adding the aptamer bound RTase to a reaction mixture containing a qPCR master mix with multiplexing primers and probes and testing the on-deck reaction mix stability of the generated one-step RT-qPCR master mix after template addition.

For the binding of DNA aptamers to M21 RTase, equal volumes of two DNA aptamers (1.45 μM, 2.18 μM, 2.90 μM 4.34 μM, 5.79 μM, Table 1) and M21 RTase (0.724 μM, Table 1) were mixed and incubated at room temperature for 25 minutes. After incubation, the DNA aptamer bound RTase was placed on ice. For the standard one-step procedure, the DNA aptamer bound RTases (6.03 nM final) were added to a reaction mixture containing the following: Integrated DNA Technologies PrimeTime™ 1-Step 4X Broad-Range Master Mix formulated without Reverse Transcriptase (1X) and FluVID primers and probe sets (Table 1). The assay master mix and synthetic RNA ultramers (50 copies per well, Table 1) were mixed at a 4:1 ratio for a final volume of 20 μL. The reaction proceeded six hours after the addition of template at room temperature on a qPCR (QuantStudio™ 7 Flex) using the following method: 50° C. hold for 15 minutes, 95° C. hold for 30 seconds, followed by 95° C. for 5 seconds and 55° C. for 45 seconds for 45 cycles. The reactions were analyzed and reported by threshold cycle (Ct) and endpoint fluorescence (delta Rn) values (Table 11). An improvement of on-deck reaction mix stability was observed by increasing the ratio of RTase to aptamer for both aptamer pairs up to the 1:6:6 dose, at which point further addition of the aptamer pairs (1:8:8) provided no additional benefit. As such, the 1:6:6 ratio was determined to be the optimal concentration of the pair aptamers.

### Example 10

This example demonstrates reverse transcriptase inhibition by combinations of DNA aptamers of an aspect of the invention at different concentrations.

In this study, DNA aptamers were combined and investigated at different ratios to achieve additive benefits to on-deck reaction mix stability. These DNA aptamers were challenged by incubation of the qPCR reactions six hours at 25° C. after template addition before running on the instrument. The DNA aptamers were tested by binding three DNA aptamers at various ratios (1:4:4:4, 1:5:5:5, and 1:6:6:6 RTase: Aptamer 1: Aptamer 2: Aptamer 3) to M21 RTase, adding the aptamer bound RTase to a reaction mixture containing a qPCR master mix with multiplexing primers and probes and testing the on-deck reaction mix stability of the generated one-step RT-qPCR master mix after template addition.

For the binding of pairs DNA aptamers to M21 RTase, equal volumes of two DNA aptamers (4.34 μM, Table 1) and M21 RTase (0.724 μM, Table 1) were mixed and incubated at room temperature for 25 minutes. For the binding of three DNA aptamers to M21 RTase, equal volumes of three DNA aptamers (2.90 μM, 3.62 μM, 4.34 μM, Table 1) and M21 RTase (0.724 μM, Table 1) were mixed and incubated at room temperature for 25 minutes. After incubation, the DNA aptamer bound RTase was placed on ice. For the standard one-step procedure, the DNA aptamer bound RTases (6.03 nM final) were added to a reaction mixture containing the following: Integrated DNA Technologies PrimeTime™ 1-Step 4X Broad-Range Master Mix formulated without Reverse Transcriptase (1X) and FluVID primers and probe sets (Table 1). The assay master mix and synthetic RNA ultramers (50 copies per well, Table 1) were mixed at a 4:1 ratio for a final volume of 20 μL. The reaction proceeded six hours after the addition of template at room temperature on a qPCR (QuantStudio™ 7 Flex) using the following method: 50° C. hold for 15 minutes, 95° C. hold for 30 seconds, followed by 95° C. for 5 seconds and 55° C. for 45 seconds for 45 cycles. The reactions were analyzed and reported by threshold cycle (Ct) and endpoint fluorescence (delta Rn) values (Table 11). Of all conditions tested, the combination of M21+RNA 9_3Phos+M21+RNA_11_3Phos+M21+RNA_47_3Phos (1:6:6:6 ratio) consistently performed the best in terms of based on high endpoint fluorescence, low Ct, and low variance compared to all other conditions for both the two and three aptamer as well as displaying substantial improvement over RNA aptamer, currently used in IDT PrimeTime™ 1-Step 4X Broad-Range Master Mix.

TABLE 3
Evaluation of room temperature stability at 0 hours and 2 hours following template addition
to PrimeTime ™ One-Step RT-qPCR Master Mix (Integrated DNA Technologies) with DNA aptamers.

| | | | 0 h Endpoint | | 0 h Cycle | | 2 h Endpoint | | 2 h Cycle | |
|---|---|---|---|---|---|---|---|---|---|---|
| | | | Fluorescence | | Threshold | | Fluorescence | | Threshold | |
| | RNA | Target | Delta Rn | Delta Rn | Ct | Ct | Delta Rn | Delta Rn | Ct | Ct |
| Aptamer | Aptamer? | Name | Mean | SD | Mean | SD | Mean | SD | Mean | SD |
| M21_1 | No | InfA | 633595 | 64835 | 35.947 | 0.643 | 352869 | 18577 | 35.830 | 0.283 |
| M21_1 | No | InfB | 220446 | 6262 | 35.185 | 0.130 | 102774 | 11286 | 35.200 | 0.402 |
| M21_1 | No | RNaseP | 845164 | 39879 | 35.146 | 0.231 | 660121 | 18258 | 35.150 | 0.336 |
| M21_1 | No | SARS- | 354061 | 26399 | 35.799 | 0.157 | 156426 | 29106 | 35.242 | 0.482 |
| | | CoV-2 | | | | | | | | |
| M21_2 | No | InfA | 637950 | 15859 | 35.139 | 0.208 | 135474 | 16121 | 36.363 | 0.553 |
| M21_2 | No | InfB | 199236 | 34502 | 34.840 | 0.337 | 37059 | 3363 | 35.865 | 0.576 |
| M21_2 | No | RNaseP | 827839 | 98826 | 34.729 | 0.491 | 543134 | 99192 | 34.330 | 0.685 |
| M21_2 | No | SARS- | 375496 | 20649 | 35.360 | 0.745 | 50611 | 13022 | 36.987 | 1.345 |
| | | CoV-2 | | | | | | | | |
| M21_3 | No | InfA | 677604 | 42693 | 35.278 | 0.502 | 265080 | 19149 | 36.103 | 0.324 |
| M21_3 | No | InfB | 228050 | 43650 | 35.038 | 0.587 | 76280 | 5762 | 35.111 | 0.266 |
| M21_3 | No | RNaseP | 882328 | 48628 | 34.985 | 0.343 | 709503 | 31180 | 34.595 | 0.202 |
| M21_3 | No | SARS- | 396714 | 41997 | 35.667 | 0.561 | 150378 | 9071 | 34.959 | 0.058 |
| | | CoV-2 | | | | | | | | |
| M21_4 | No | InfA | 709568 | 77795 | 35.352 | 0.598 | 390854 | 47396 | 35.841 | 0.328 |
| M21_4 | No | InfB | 250841 | 31095 | 34.771 | 0.704 | 110212 | 13799 | 35.374 | 0.427 |
| M21_4 | No | RNaseP | 985378 | 93398 | 34.761 | 0.176 | 755345 | 55748 | 34.673 | 0.243 |
| M21_4 | No | SARS- | 439172 | 11975 | 35.144 | 0.337 | 170187 | 31670 | 35.452 | 0.607 |
| | | CoV-2 | | | | | | | | |
| M21_5 | No | InfA | 675302 | 7884 | 35.410 | 0.186 | 173018 | 16787 | 36.201 | 0.449 |
| M21_5 | No | InfB | 239085 | 20326 | 35.026 | 0.364 | 38501 | 5981 | 36.275 | 0.933 |
| M21_5 | No | RNaseP | 903567 | 66582 | 35.001 | 0.501 | 627588 | 70849 | 34.541 | 0.456 |
| M21_5 | No | SARS- | 390976 | 16758 | 35.392 | 0.461 | 79516 | 4613 | 34.998 | 0.249 |
| | | CoV-2 | | | | | | | | |
| M21_6 | No | InfA | 755796 | 23583 | 35.266 | 0.517 | 366812 | 97689 | 35.741 | 0.948 |
| M21_6 | No | InfB | 249982 | 1722 | 34.913 | 0.509 | 116402 | 14106 | 35.178 | 0.439 |
| M21_6 | No | RNaseP | 937034 | 20789 | 35.176 | 0.206 | 739426 | 13210 | 34.790 | 0.318 |
| M21_6 | No | SARS- | 414561 | 15879 | 35.472 | 0.068 | 153211 | 37100 | 35.646 | 0.631 |
| | | CoV-2 | | | | | | | | |
| M21_7 | No | InfA | 675459 | 21174 | 35.421 | 0.369 | 410372 | 11803 | 35.693 | 0.212 |
| M21_7 | No | InfB | 257765 | 9676 | 34.854 | 0.204 | 92479 | 13794 | 35.780 | 0.748 |
| M21_7 | No | RNaseP | 904582 | 73812 | 34.960 | 0.373 | 693574 | 103913 | 34.777 | 0.511 |
| M21_7 | No | SARS- | 353573 | 37808 | 35.831 | 0.752 | 167743 | 22462 | 35.283 | 0.423 |
| | | CoV-2 | | | | | | | | |
| M21_8 | No | InfA | 635156 | 42697 | 35.604 | 0.705 | 179603 | 9494 | 36.516 | 0.094 |
| M21_8 | No | InfB | 238911 | 11806 | 34.826 | 0.265 | 78350 | 12566 | 34.887 | 0.442 |
| M21_8 | No | RNaseP | 778770 | 63713 | 35.365 | 0.058 | 580867 | 27122 | 34.748 | 0.244 |
| M21_8 | No | SARS- | 400191 | 18479 | 35.405 | 0.744 | 102145 | 4745 | 35.535 | 0.152 |
| | | CoV-2 | | | | | | | | |
| M21_9 | No | InfA | 718714 | 49933 | 35.254 | 0.165 | 288220 | 85650 | 37.257 | 1.230 |
| M21_9 | No | InfB | 253246 | 6567 | 34.916 | 0.311 | 134726 | 18621 | 35.217 | 0.600 |
| M21_9 | No | RNaseP | 917785 | 76325 | 35.183 | 0.109 | 790352 | 16687 | 34.813 | 0.348 |
| M21_9 | No | SARS- | 358002 | 91200 | 35.598 | 0.714 | 233586 | 34166 | 35.344 | 0.158 |
| | | CoV-2 | | | | | | | | |
| M21_10 | No | InfA | 729824 | 90828 | 35.299 | 0.603 | 343021 | 58287 | 36.044 | 0.839 |
| M21_10 | No | InfB | 251380 | 28764 | 35.001 | 0.473 | 105405 | 14531 | 35.214 | 0.516 |
| M21_10 | No | RNaseP | 1007644 | 24163 | 34.904 | 0.578 | 757573 | 36256 | 34.832 | 0.197 |
| M21_10 | No | SARS- | 456726 | 23354 | 35.252 | 0.043 | 184017 | 10716 | 35.435 | 0.116 |
| | | CoV-2 | | | | | | | | |
| M21_11 | No | InfA | 715750 | 65647 | 35.366 | 0.402 | 320041 | 77777 | 36.181 | 0.680 |
| M21_11 | No | InfB | 252077 | 33559 | 34.770 | 0.038 | 88222 | 16019 | 35.442 | 0.543 |
| M21_11 | No | RNaseP | 1024233 | 18638 | 34.595 | 0.169 | 677533 | 22592 | 34.946 | 0.115 |
| M21_11 | No | SARS- | 405794 | 25064 | 35.169 | 0.207 | 172684 | 22871 | 35.250 | 0.364 |
| | | CoV-2 | | | | | | | | |
| M21_12 | No | InfA | 623391 | 127022 | 35.429 | 0.320 | 188086 | 7893 | 36.214 | 0.286 |
| M21_12 | No | InfB | 200136 | 8430 | 35.230 | 0.571 | 56212 | 15171 | 35.716 | 1.474 |
| M21_12 | No | RNaseP | 920629 | 134421 | 34.677 | 0.181 | 656455 | 28219 | 34.581 | 0.091 |
| M21_12 | No | SARS- | 452147 | 43959 | 34.710 | 0.290 | 104018 | 43530 | 36.006 | 2.055 |
| | | CoV-2 | | | | | | | | |
| M21_13 | No | InfA | 726099 | 36369 | 35.254 | 0.293 | 241168 | 45256 | 36.212 | 0.765 |
| M21_13 | No | InfB | 258381 | 23795 | 34.867 | 0.220 | 77739 | 12729 | 35.118 | 0.509 |
| M21_13 | No | RNaseP | 1021542 | 74267 | 34.640 | 0.204 | 749582 | 60539 | 34.507 | 0.472 |
| M21_13 | | SARS- | 463436 | 6762 | 35.201 | 0.302 | 143625 | 51275 | 35.534 | 1.058 |
| | No | CoV-2 | | | | | | | | |
| M21_14 | No | InfA | 674818 | 29633 | 35.859 | 0.330 | 333398 | 68362 | 35.841 | 0.737 |
| M21_14 | No | InfB | 220814 | 33924 | 35.466 | 0.482 | 94458 | 7455 | 34.963 | 0.271 |
| M21_14 | No | RNaseP | 993629 | 30223 | 35.037 | 0.134 | 750388 | 18101 | 34.908 | 0.096 |
| M21_14 | No | SARS- | 431979 | 26066 | 35.301 | 0.471 | 181073 | 32365 | 35.078 | 0.609 |
| | | CoV-2 | | | | | | | | |
| M21_15 | No | InfA | 653168 | 37030 | 35.539 | 0.489 | 316478 | 45840 | 35.747 | 0.509 |
| M21_15 | No | InfB | 236797 | 17885 | 34.828 | 0.622 | 104856 | 13479 | 34.830 | 0.442 |
| M21_15 | No | RNaseP | 944406 | 14713 | 35.039 | 0.175 | 705117 | 7043 | 34.834 | 0.353 |
| M21_15 | No | SARS- | 331217 | 51944 | 36.126 | 0.516 | 147514 | 48390 | 35.319 | 0.619 |
| | | CoV-2 | | | | | | | | |
| M21_16 | No | InfA | 587878 | 86347 | 35.751 | 0.686 | 186299 | 22218 | 36.400 | 0.549 |
| M21_16 | No | InfB | 204527 | 24604 | 35.233 | 0.403 | 55145 | 11767 | 35.586 | 0.730 |
| M21_16 | No | RNaseP | 852969 | 105933 | 34.969 | 0.211 | 597479 | 19108 | 34.711 | 0.200 |
| M21_16 | | SARS- | 365288 | 20231 | 35.407 | 0.080 | 128251 | 34468 | 34.957 | 0.801 |
| | No | CoV-2 | | | | | | | | |
| M21_17 | No | InfA | 611490 | 129285 | 35.651 | 1.229 | 305873 | 75020 | 35.851 | 0.845 |
| M21_17 | No | InfB | 236597 | 21379 | 34.347 | 0.338 | 61394 | 7210 | 35.815 | 0.573 |
| M21_17 | No | RNaseP | 884006 | 64300 | 34.737 | 0.145 | 604806 | 72245 | 34.426 | 0.221 |
| M21_17 | No | SARS- | 436260 | 17473 | 34.978 | 0.148 | 165672 | 43017 | 35.132 | 0.911 |
| | | CoV-2 | | | | | | | | |
| M21_18 | No | InfA | 664340 | 36880 | 35.634 | 0.162 | 302607 | 80234 | 36.067 | 0.787 |
| M21_18 | No | InfB | 267989 | 11224 | 34.613 | 0.816 | 73355 | 6829 | 35.770 | 0.353 |
| M21_18 | No | RNaseP | 982968 | 52016 | 34.548 | 0.138 | 648354 | 43614 | 34.779 | 0.354 |
| M21_18 | No | SARS- | 429377 | 21982 | 35.192 | 0.138 | 161483 | 7544 | 35.189 | |
| | | CoV-2 | | | | | | | | 0.220 |
| M21_19 | No | InfA | 647182 | 94232 | 35.653 | 0.501 | 264289 | 104897 | 36.815 | 1.488 |
| M21_19 | No | InfB | 214506 | 34181 | 35.005 | 0.534 | 92382 | 14497 | 35.283 | 0.442 |
| M21_19 | No | RNaseP | 996654 | 98874 | 34.943 | 0.536 | 780291 | 50922 | 34.781 | 0.178 |
| M21_19 | No | SARS- | 405546 | 39709 | 35.350 | 0.705 | 136210 | 24273 | 35.568 | 0.614 |
| | | CoV-2 | | | | | | | | |
| M21_20 | No | InfA | 623644 | 2641 | 35.090 | 0.239 | 156644 | 48986 | 36.617 | 1.352 |
| M21_20 | No | InfB | 211496 | 31745 | 34.491 | 0.820 | 27648 | 7428 | 39.156 | 2.931 |
| M21_20 | No | RNaseP | 823043 | 53466 | 34.679 | 0.056 | 600772 | 1580 | 34.248 | 0.062 |
| M21_20 | No | SARS- | 470416 | 16638 | 34.852 | 0.463 | 63385 | 25717 | 36.559 | 2.081 |
| | | CoV-2 | | | | | | | | |
| M21_21 | No | InfA | 676241 | 73541 | 35.651 | 0.417 | 438065 | 47384 | 35.668 | 0.366 |
| M21_21 | No | InfB | 236772 | 47052 | 35.567 | 0.746 | 106754 | 15682 | 35.714 | 0.433 |
| M21_21 | No | RNaseP | 1090461 | 26168 | 34.559 | 0.391 | 868679 | 120884 | 34.734 | 0.689 |
| M21_21 | No | SARS- | 505150 | 20743 | 34.605 | 0.077 | 235267 | 53107 | 35.130 | 0.704 |
| | | CoV-2 | | | | | | | | |
| M21_22 | No | InfA | 658113 | 33295 | 35.976 | 0.541 | 202460 | 29177 | 36.684 | 0.422 |
| M21_22 | No | InfB | 218381 | 43317 | 35.182 | 1.300 | 77285 | 8531 | 35.033 | 0.410 |
| M21_22 | No | RNaseP | 1082477 | 30673 | 34.485 | 0.106 | 692888 | 26178 | 34.250 | 0.300 |
| M21_22 | No | SARS- | 349443 | 97732 | 36.138 | 1.142 | 83549 | 22194 | 35.278 | 0.759 |
| | | CoV-2 | | | | | | | | |
| M21_23 | No | InfA | 566824 | 25405 | 35.831 | 0.117 | 111104 | 23863 | 37.572 | 1.458 |
| M21_23 | No | InfB | 199458 | 12860 | 35.072 | 0.273 | 27818 | 2230 | 37.540 | 0.674 |
| M21_23 | No | RNaseP | 855730 | 17202 | 34.739 | 0.132 | 542791 | 41078 | 34.427 | 0.453 |
| M21_23 | No | SARS- | 405880 | 30022 | 35.609 | 0.388 | 62218 | 10820 | 35.978 | 0.840 |
| | | CoV-2 | | | | | | | | |
| M21_24 | No | InfA | 544748 | 41423 | 35.538 | 0.390 | 85734 | 17726 | 39.017 | 1.590 |
| M21_24 | No | InfB | 181816 | 7866 | 35.298 | 0.327 | 30937 | 7854 | 37.797 | 3.399 |
| M21_24 | No | RNaseP | 769826 | 50058 | 34.982 | 0.266 | 567173 | 31544 | 34.017 | 0.285 |
| M21_24 | No | SARS- | 406760 | 18482 | 35.252 | 0.424 | 59584 | 9456 | 36.160 | 1.037 |
| | | CoV-2 | | | | | | | | |
| M21_25 | No | InfA | 629415 | 31582 | 34.730 | 0.370 | 158556 | 16153 | 36.441 | 0.519 |
| M21_25 | No | InfB | 202127 | 19637 | 34.287 | 0.208 | 35438 | 11523 | 37.543 | 2.428 |
| M21_25 | No | RNaseP | 839803 | 35335 | 34.110 | 0.300 | 618324 | 99751 | 34.507 | 0.634 |
| M21_25 | No | SARS- | 403340 | 21415 | 34.262 | 0.350 | 84662 | 4229 | 35.642 | 0.166 |
| | | CoV-2 | | | | | | | | |
| M21_26 | No | InfA | 747924 | 36702 | 35.113 | 0.485 | 440140 | 81701 | 35.999 | 0.936 |
| M21_26 | No | InfB | 260333 | 23094 | 34.962 | 0.855 | 140403 | 10450 | 35.162 | 0.341 |
| M21_26 | No | RNaseP | 1014251 | 81699 | 34.580 | 0.688 | 855290 | 59234 | 34.893 | 0.410 |
| M21_26 | No | SARS- | 471505 | 31193 | 34.841 | 0.181 | 210284 | 18067 | 35.428 | 0.383 |
| | | CoV-2 | | | | | | | | |
| M21_27 | No | InfA | 578315 | 28505 | 35.077 | 0.303 | 185223 | 9935 | 36.214 | 0.568 |
| M21_27 | No | InfB | 192197 | 29187 | 34.943 | 0.102 | 48987 | 8633 | 35.713 | 0.748 |
| M21_27 | No | RNaseP | 838003 | 48054 | 34.518 | 0.507 | 709848 | 63089 | 34.154 | 0.253 |
| M21_27 | No | SARS- | 425002 | 46172 | 34.988 | 0.631 | 95796 | 11617 | 35.310 | 0.538 |
| | | CoV-2 | | | | | | | | |
| M21_28 | No | InfA | 747165 | 55034 | 35.104 | 0.579 | 447024 | 60557 | 35.611 | 0.452 |
| M21_28 | No | InfB | 242586 | 29425 | 34.724 | 0.460 | 130411 | 10684 | 35.002 | 0.312 |
| M21_28 | No | RNaseP | 1033181 | 42832 | 34.440 | 0.401 | 808389 | 15798 | 34.470 | 0.217 |
| M21_28 | No | SARS- | 473128 | 28057 | 35.118 | 0.598 | 228915 | 45977 | 35.478 | 0.754 |
| | | CoV-2 | | | | | | | | |
| M21_29 | No | InfA | 724103 | 29080 | 35.132 | 0.041 | 275705 | 42853 | 36.306 | 0.461 |
| M21_29 | No | InfB | 215310 | 3985 | 35.048 | 0.282 | 57402 | 5859 | 35.722 | 0.282 |
| M21_29 | No | RNaseP | 1039851 | 6927 | 34.282 | 0.154 | 844691 | 13699 | 34.174 | 0.127 |
| M21_29 | No | SARS- | 281440 | 13064 | 34.768 | 0.559 | 26638 | 1146 | 40.443 | 0.857 |
| | | CoV-2 | | | | | | | | |
| M21_30 | No | InfA | 674107 | 39783 | 35.366 | 0.166 | 230981 | 69719 | 36.456 | 1.305 |
| M21_30 | No | InfB | 239910 | 32233 | 34.849 | 0.676 | 66655 | 1117 | 35.410 | 0.200 |
| M21_30 | No | RNaseP | 1012798 | 79205 | 34.717 | 0.269 | 744794 | 99313 | 34.546 | 0.696 |
| M21_30 | No | SARS- | 461096 | 46522 | 34.913 | 0.480 | 135446 | 13651 | 35.186 | 0.184 |
| | | CoV-2 | | | | | | | | |
| M21_31 | No | InfA | 719356 | 5344 | 35.069 | 0.241 | 228335 | 67344 | 36.360 | 1.359 |
| M21_31 | No | InfB | 234583 | 26267 | 34.924 | 0.587 | 66108 | 11160 | 35.358 | 0.555 |
| M21_31 | No | RNaseP | 973262 | 58114 | 34.513 | 0.320 | 668078 | 41325 | 34.452 | 0.210 |
| M21_31 | No | SARS- | 401083 | 49615 | 35.275 | 0.181 | 114432 | 24109 | 35.595 | 0.687 |
| | | CoV-2 | | | | | | | | |
| M21_32 | No | InfA | 627661 | 21781 | 35.652 | 0.555 | 462060 | 44750 | 35.050 | 0.309 |
| M21_32 | No | InfB | 237477 | 7068 | 34.920 | 0.312 | 89312 | 9339 | 35.637 | 0.342 |
| M21_32 | No | RNaseP | 986099 | 55221 | 34.780 | 0.435 | 691787 | 74368 | 35.085 | 0.292 |
| M21_32 | | SARS- | 435382 | 6322 | 35.143 | 0.363 | 168673 | 28986 | 35.451 | 0.488 |
| | No | CoV-2 | | | | | | | | |
| M21_33 | No | InfA | 683374 | 67746 | 35.107 | 0.436 | 338320 | 27641 | 35.646 | 0.336 |
| M21_33 | No | InfB | 256108 | 26215 | 34.372 | 0.525 | 81505 | 1394 | 35.321 | 0.054 |
| M21_33 | No | RNaseP | 925668 | 53414 | 34.592 | 0.180 | 732458 | 26351 | 34.657 | 0.066 |
| M21_33 | No | SARS- | 408290 | 24885 | 35.227 | 0.675 | 143094 | 18082 | 35.163 | 0.578 |
| | | CoV-2 | | | | | | | | |
| M21_34 | No | InfA | 716596 | 83862 | 34.978 | 0.133 | 255452 | 57664 | 36.802 | 0.522 |
| M21_34 | No | InfB | 227901 | 30082 | 34.820 | 1.260 | 93545 | 32573 | 35.454 | 1.214 |
| M21_34 | No | RNaseP | 1070609 | 58426 | 34.253 | 0.321 | 752726 | 45723 | 34.761 | 0.041 |
| M21_34 | | SARS- | 476873 | 50638 | 34.782 | 0.701 | 197693 | 6521 | 35.175 | 0.201 |
| | No | CoV-2 | | | | | | | | |
| M21_35 | No | InfA | 781560 | 31534 | 34.938 | 0.499 | 517174 | 23144 | 36.322 | 0.293 |
| M21_35 | No | InfB | 288499 | 34284 | 34.145 | 0.652 | 136989 | 15895 | 35.966 | 0.106 |
| M21_35 | No | RNaseP | 1039935 | 67737 | 34.086 | 0.283 | 852092 | 53866 | 34.648 | 0.279 |
| M21_35 | No | SARS- | 421795 | 18309 | 34.637 | 0.477 | 270574 | 31811 | 35.047 | 0.307 |
| | | CoV-2 | | | | | | | | |
| M21_36 | No | InfA | 691778 | 35101 | 35.130 | 0.373 | 270727 | 35426 | 36.077 | 0.527 |
| M21_36 | No | InfB | 237044 | 9163 | 34.918 | 0.384 | 68617 | 11256 | 35.300 | 0.519 |
| M21_36 | No | RNaseP | 1097188 | 74362 | 34.095 | 0.281 | 811613 | 29260 | 34.407 | 0.091 |
| M21_36 | No | SARS- | 453732 | 48387 | 34.472 | 0.193 | 99117 | 12830 | 34.710 | |
| | | CoV-2 | | | | | | | | 0.305 |
| M21_37 | No | InfA | 721928 | 78753 | 35.213 | 0.295 | 294109 | 68594 | 36.278 | 0.797 |
| M21_37 | No | InfB | 299940 | 19770 | 34.099 | 0.504 | 87364 | 11573 | 35.343 | 0.450 |
| M21_37 | No | RNaseP | 1029301 | 165626 | 34.522 | 0.676 | 755496 | 54577 | 34.538 | 0.092 |
| M21_37 | No | SARS- | 463230 | 15853 | 34.834 | 0.662 | 164341 | 9585 | 34.879 | 0.364 |
| | | CoV-2 | | | | | | | | |
| M21_38 | No | InfA | 738788 | 44511 | 35.198 | 0.228 | 235069 | 121538 | 38.069 | 3.680 |
| M21_38 | No | InfB | 263611 | 12702 | 34.456 | 0.070 | 73722 | 717 | 35.782 | 0.214 |
| M21_38 | No | RNaseP | 1112138 | 36838 | 34.259 | 0.419 | 839117 | 40850 | 34.380 | 0.272 |
| M21_38 | No | SARS- | 470115 | 53737 | 34.899 | 1.104 | 177763 | 3415 | 35.331 | 0.271 |
| | | CoV-2 | | | | | | | | |
| M21_39 | No | InfA | 691178 | 4649 | 35.467 | 0.504 | 288199 | 39792 | 35.734 | 0.569 |
| M21_39 | No | InfB | 246729 | 39099 | 34.775 | 1.069 | 76699 | 9099 | 35.150 | 0.405 |
| M21_39 | No | RNaseP | 989286 | 82221 | 34.712 | 0.418 | 680799 | 10342 | 34.396 | 0.093 |
| M21_39 | No | SARS- | 476627 | 10343 | 34.874 | 0.569 | 173493 | 6574 | 35.079 | 0.176 |
| | | CoV-2 | | | | | | | | |
| M21_40 | No | InfA | 677093 | 25893 | 35.089 | 0.511 | 300214 | 33327 | 35.899 | 0.148 |
| M21_40 | No | InfB | 250404 | 29428 | 34.778 | 0.691 | 93739 | 16233 | 35.319 | 0.459 |
| M21_40 | No | RNaseP | 954717 | 85001 | 34.558 | 0.248 | 763185 | 55073 | 34.647 | 0.155 |
| M21_40 | No | SARS- | 414900 | 14219 | 35.339 | 0.630 | 128028 | 32874 | 35.808 | 0.705 |
| | | CoV-2 | | | | | | | | |
| M21_41 | No | InfA | 795368 | 24413 | 34.955 | 0.212 | 495447 | 110560 | 35.992 | 0.525 |
| M21_41 | No | InfB | 267788 | 17204 | 34.366 | 0.160 | 157363 | 9718 | 35.270 | 0.434 |
| M21_41 | No | RNaseP | 1076287 | 57475 | 34.583 | 0.406 | 784035 | 29313 | 35.221 | 0.191 |
| M21_41 | No | SARS- | 461421 | 26482 | 34.965 | 0.255 | 274606 | 40419 | 35.326 | 0.294 |
| | | CoV-2 | | | | | | | | |
| M21_42 | No | InfA | 803364 | 33462 | 34.690 | 0.320 | 479048 | 11869 | 35.735 | 0.102 |
| M21_42 | No | InfB | 284296 | 9584 | 34.402 | 0.046 | 120390 | 34121 | 35.533 | 0.897 |
| M21_42 | No | RNaseP | 1139966 | 35564 | 33.977 | 0.086 | 833125 | 29339 | 35.143 | 0.264 |
| M21_42 | No | SARS- | 447062 | 4108 | 35.034 | 0.376 | 216632 | 10446 | 35.716 | 0.119 |
| | | CoV-2 | | | | | | | | |
| M21_43 | No | InfA | 737496 | 48193 | 35.195 | 0.516 | 329003 | 83834 | 36.115 | 1.018 |
| M21_43 | No | InfB | 241692 | 70428 | 34.939 | 1.434 | 121589 | 22680 | 34.712 | 0.680 |
| M21_43 | No | RNaseP | 1072048 | 67954 | 34.142 | 0.115 | 800227 | 41910 | 34.581 | 0.193 |
| M21_43 | No | SARS- | 478331 | 19804 | 34.593 | 0.120 | 154045 | 12150 | 35.644 | 0.254 |
| | | CoV-2 | | | | | | | | |
| M21_44 | No | InfA | 783683 | 52098 | 35.010 | 0.175 | 459452 | 129306 | 35.920 | 0.945 |
| M21_44 | No | InfB | 275661 | 37365 | 34.689 | 1.089 | 144946 | 22996 | 34.884 | 0.583 |
| M21_44 | No | RNaseP | 1137165 | 66667 | 34.540 | 0.267 | 926036 | 45502 | 34.381 | 0.349 |
| M21_44 | No | SARS- | 445169 | 60852 | 34.884 | 0.518 | 223529 | 17070 | 35.069 | 0.247 |
| | | CoV-2 | | | | | | | | |
| M21_45 | No | InfA | 698833 | 27576 | 34.976 | 0.225 | 264652 | 54740 | 35.643 | 0.590 |
| M21_45 | No | InfB | 248093 | 10219 | 34.324 | 0.374 | 62655 | 9103 | 35.383 | 0.504 |
| M21_45 | No | RNaseP | 1046774 | 59138 | 34.319 | 0.602 | 737642 | 53670 | 34.138 | 0.227 |
| M21_45 | No | SARS- | 504193 | 27843 | 34.450 | 0.453 | 170906 | 50074 | 34.822 | 0.795 |
| | | CoV-2 | | | | | | | | |
| M21_46 | No | InfA | 701294 | 28384 | 35.562 | 0.307 | 402909 | 41041 | 35.846 | 0.153 |
| M21_46 | No | InfB | 282751 | 10530 | 34.217 | 0.755 | 134497 | 9809 | 35.529 | 0.265 |
| M21_46 | No | RNaseP | 1102749 | 16541 | 34.296 | 0.410 | 849739 | 25814 | 34.445 | 0.293 |
| M21_46 | No | SARS- | 441344 | 31735 | 35.047 | 0.113 | 162048 | 28007 | 35.497 | 0.603 |
| | | CoV-2 | | | | | | | | |
| M21_47 | No | InfA | 703874 | 60797 | 35.105 | 0.452 | 287068 | 43125 | 35.608 | 0.696 |
| M21_47 | No | InfB | 267364 | 29520 | 34.739 | 0.734 | 77144 | 14898 | 34.981 | 0.817 |
| M21_47 | No | RNaseP | 931298 | 36275 | 34.584 | 0.293 | 765091 | 41644 | 34.324 | 0.163 |
| M21_47 | No | SARS- | 411566 | 13264 | 35.238 | 0.331 | 116119 | 25570 | 35.539 | 0.692 |
| | | CoV-2 | | | | | | | | |
| M21_48 | No | InfA | 629488 | 54933 | 35.023 | 0.270 | 124295 | 41412 | 37.331 | 1.992 |
| M21_48 | No | InfB | 222251 | 32928 | 34.264 | 0.052 | 28929 | 4530 | 37.606 | 1.945 |
| M21_48 | No | RNaseP | 824840 | 44243 | 34.494 | 0.406 | 477436 | 85885 | 34.837 | 1.043 |
| M21_48 | No | SARS- | 394363 | 57669 | 35.146 | 0.564 | 63201 | 11823 | 35.891 | 0.685 |
| | | CoV-2 | | | | | | | | |
| M21_49 | No | InfA | 740409 | 42511 | 34.943 | 0.318 | 292801 | 12975 | 36.440 | 0.354 |
| M21_49 | No | InfB | 270323 | 34440 | 34.176 | 0.524 | 97928 | 17190 | 35.412 | 0.643 |
| M21_49 | No | RNaseP | 977211 | 106086 | 34.765 | 0.373 | 775089 | 32453 | 34.481 | 0.291 |
| M21_49 | No | SARS- | 426542 | 22258 | 35.010 | 0.301 | 213664 | 16002 | 34.834 | 0.088 |
| | | CoV-2 | | | | | | | | |
| M21_50 | No | InfA | 627109 | 151340 | 35.798 | 1.505 | 381587 | 15978 | 35.595 | 0.275 |
| M21_50 | No | InfB | 271366 | 14121 | 34.308 | 0.403 | 99554 | 12923 | 35.101 | 0.397 |
| M21_50 | No | RNaseP | 989400 | 15687 | 34.669 | 0.466 | 803308 | 44972 | 34.601 | 0.290 |
| M21_50 | No | SARS- | 481502 | 24003 | 34.610 | 0.127 | 189479 | 58970 | 35.288 | 0.831 |
| | | CoV-2 | | | | | | | | |
| M21 + RNA_1 | No | InfA | 745831 | 36916 | 35.318 | 0.085 | 560451 | 60759 | 35.609 | 0.251 |
| M21 + RNA_1 | No | InfB | 260282 | 56032 | 35.168 | 1.253 | 141186 | 25837 | 35.539 | 0.566 |
| M21 + RNA_1 | No | RNaseP | 822092 | 49153 | 35.410 | 0.052 | 776207 | 23882 | 34.723 | 0.267 |
| M21 + RNA_1 | No | SARS- | 414087 | 18171 | 35.271 | 0.411 | 212376 | 41569 | 35.741 | 0.626 |
| | | CoV-2 | | | | | | | | |
| M21 + RNA_2 | No | InfA | 722556 | 67841 | 35.531 | 0.436 | 533078 | 61835 | 35.668 | 0.286 |
| M21 + RNA_2 | No | InfB | 266243 | 12029 | 35.097 | 0.278 | 156858 | 16053 | 35.206 | 0.282 |
| M21 + RNA_2 | No | RNaseP | 1025323 | 79903 | 34.675 | 0.133 | 873168 | 47525 | 34.645 | 0.277 |
| M21 + RNA_2 | No | SARS- | 412715 | 21442 | 35.310 | 0.314 | 142086 | 21153 | 36.308 | 0.610 |
| | | CoV-2 | | | | | | | | |
| M21 + RNA_3 | No | InfA | 639520 | 49747 | 35.786 | 0.537 | 462255 | 47811 | 35.766 | 0.559 |
| M21 + RNA_3 | No | InfB | 242837 | 2364 | 35.288 | 0.417 | 133614 | 12466 | 35.135 | 0.280 |
| M21 + RNA_3 | No | RNaseP | 979256 | 80502 | 34.689 | 0.371 | 693600 | 72262 | 35.006 | 0.343 |
| M21 + RNA_3 | No | SARS- | 415242 | 41534 | 35.345 | 0.352 | 184841 | 27904 | 35.791 | 0.510 |
| | | CoV-2 | | | | | | | | |
| M21 + RNA_4 | No | InfA | 798868 | 24299 | 34.846 | 0.194 | 567451 | 19505 | 35.667 | 0.428 |
| M21 + RNA_4 | No | InfB | 278479 | 23976 | 34.813 | 0.753 | 124121 | 39546 | 36.074 | 1.324 |
| M21 + RNA_4 | No | RNaseP | 966929 | 41285 | 34.932 | 0.081 | 807865 | 55025 | 35.033 | 0.169 |
| M21 + RNA_4 | No | SARS- | 470795 | 28485 | 34.972 | 0.078 | 234475 | 38482 | 35.588 | 0.621 |
| | | CoV-2 | | | | | | | | |
| M21 + RNA_5 | No | InfA | 761489 | 12300 | 35.455 | 0.140 | 502535 | 79043 | 36.503 | 0.623 |
| M21 + RNA_5 | No | InfB | 268440 | 18233 | 34.805 | 0.191 | 137848 | 37261 | 36.052 | 0.788 |
| M21 + RNA_5 | No | RNaseP | 988845 | 60439 | 34.983 | 0.393 | 747813 | 31176 | 35.045 | 0.347 |
| M21 + RNA_5 | No | SARS- | 383152 | 23991 | 35.702 | 0.428 | 198535 | 32465 | 35.510 | 0.381 |
| | | CoV-2 | | | | | | | | |
| M21 + RNA_6 | No | InfA | 722387 | 57122 | 35.356 | 0.566 | 481667 | 39929 | 36.023 | 0.411 |
| M21 + RNA_6 | No | InfB | 239424 | 10043 | 35.182 | 0.137 | 150678 | 16651 | 35.241 | 0.214 |
| M21 + RNA_6 | No | RNaseP | 930764 | 45872 | 35.012 | 0.234 | 766464 | 112299 | 35.235 | 0.920 |
| M21 + RNA_6 | No | SARS- | 394099 | 18346 | 35.496 | 0.274 | 243520 | 33820 | 35.570 | 0.474 |
| | | CoV-2 | | | | | | | | |
| M21 + RNA_7 | No | InfA | 712185 | 27911 | 35.489 | 0.501 | 457965 | 86162 | 36.333 | 0.738 |
| M21 + RNA_7 | No | InfB | 270320 | 7777 | 35.087 | 0.234 | 152602 | 22462 | 35.465 | 0.643 |
| M21 + RNA_7 | No | RNaseP | 946167 | 17204 | 34.974 | 0.326 | 749392 | 30760 | 35.197 | 0.273 |
| M21 + RNA_7 | No | SARS- | 375936 | 26243 | 35.354 | 0.051 | 167331 | 719 | 34.829 | 0.129 |
| | | CoV-2 | | | | | | | | |
| M21 + RNA_8 | No | InfA | 667822 | 16291 | 35.418 | 0.309 | 439240 | 30810 | 35.484 | 0.246 |
| M21 + RNA_8 | No | InfB | 210257 | 6526 | 35.136 | 0.415 | 119071 | 15998 | 35.436 | 0.424 |
| M21 + RNA_8 | No | RNaseP | 873751 | 27415 | 34.779 | 0.200 | 698800 | 30939 | 34.793 | 0.037 |
| M21 + RNA_8 | No | SARS- | 360006 | 19640 | 35.605 | 0.102 | 180022 | 54688 | 35.815 | 1.074 |
| | | CoV-2 | | | | | | | | |
| M21 + RNA_9 | No | InfA | 767046 | 84497 | 35.280 | 0.715 | 639440 | 50058 | 35.453 | 0.332 |
| M21 + RNA_9 | No | InfB | 257616 | 4359 | 35.072 | 0.151 | 181153 | 26449 | 35.344 | 0.742 |
| M21 + RNA_9 | No | RNaseP | 984221 | 87612 | 35.132 | 0.447 | 837727 | 20043 | 35.031 | 0.173 |
| M21 + RNA_9 | No | SARS- | 396755 | 45295 | 35.404 | 0.377 | 249936 | 63134 | 35.553 | 0.963 |
| | | CoV-2 | | | | | | | | |
| M21 + RNA_10 | No | InfA | 758227 | 77546 | 35.319 | 0.634 | 451066 | 80959 | 35.511 | 0.670 |
| M21 + RNA_10 | No | InfB | 273200 | 14834 | 34.929 | 0.487 | 104321 | 19599 | 35.445 | 0.403 |
| M21 + RNA_10 | No | RNaseP | 969013 | 92087 | 34.871 | 0.504 | 784355 | 4154 | 35.047 | 0.344 |
| M21 + RNA_10 | No | SARS- | 433939 | 13838 | 35.324 | 0.256 | 207501 | 45143 | 35.451 | 0.676 |
| | | CoV-2 | | | | | | | | |
| M21 + RNA_11 | No | InfA | 687105 | 11653 | 35.525 | 0.293 | 264925 | 94976 | 37.297 | 1.660 |
| M21 + RNA_11 | No | InfB | 241163 | 24180 | 35.155 | 0.401 | 90563 | 3648 | 36.131 | 0.158 |
| M21 + RNA_11 | No | RNaseP | 1034270 | 37163 | 34.879 | 0.358 | 793897 | 27314 | 34.766 | 0.161 |
| M21 + RNA_11 | No | SARS- | 451442 | 31164 | 35.047 | 0.241 | 225849 | 62326 | 35.450 | 0.976 |
| | | CoV-2 | | | | | | | | |
| M21 + RNA_12 | No | InfA | 671484 | 18262 | 35.856 | 0.261 | 293310 | 12567 | 35.912 | 0.175 |
| M21 + RNA_12 | No | InfB | 236078 | 31852 | 35.359 | 0.484 | 82290 | 11122 | 35.164 | 0.359 |
| M21 + RNA_12 | No | RNaseP | 951175 | 118122 | 35.011 | 0.732 | 759735 | 61191 | 34.607 | 0.237 |
| M21 + RNA_12 | No | SARS- | 452887 | 37166 | 35.092 | 0.283 | 144853 | 43833 | 35.267 | 0.693 |
| | | CoV-2 | | | | | | | | |
| M21 + RNA_13 | No | InfA | 755831 | 55602 | 35.421 | 0.168 | 467192 | 38236 | 35.988 | 0.276 |
| M21 + RNA_13 | No | InfB | 229318 | 60352 | 35.651 | 1.292 | 143809 | 14188 | 35.308 | 0.253 |
| M21 + RNA_13 | No | RNaseP | 980878 | 82171 | 34.890 | 0.313 | 826891 | 25491 | 34.809 | 0.060 |
| M21 + RNA_13 | No | SARS- | 440767 | 39798 | 35.535 | 0.198 | 185639 | 15993 | 35.366 | 0.237 |
| | | CoV-2 | | | | | | | | |
| M21 + RNA_14 | No | InfA | 656087 | 51874 | 35.459 | 0.501 | 357592 | 98976 | 35.380 | 1.024 |
| M21 + RNA_14 | No | InfB | 217078 | 4130 | 35.445 | 0.298 | 85281 | 27258 | 35.455 | 1.301 |
| M21 + RNA_14 | No | RNaseP | 923863 | 40070 | 35.093 | 0.338 | 730443 | 40237 | 34.595 | 0.088 |
| M21 + RNA_14 | No | SARS- | 393036 | 45067 | 35.045 | 0.215 | 140946 | 42432 | 35.415 | 0.604 |
| | | CoV-2 | | | | | | | | |
| M21 + RNA_15 | No | InfA | 656929 | 106712 | 35.874 | 0.733 | 479948 | 77217 | 36.010 | 0.609 |
| M21 + RNA_15 | No | InfB | 219717 | 24663 | 35.647 | 0.804 | 148009 | 2281 | 35.207 | 0.206 |
| M21 + RNA_15 | No | RNaseP | 1007275 | 37632 | 34.826 | 0.138 | 848219 | 98335 | 34.905 | 0.502 |
| M21 + RNA_15 | No | SARS- | 397042 | 29718 | 35.729 | 0.217 | 208340 | 35277 | 35.572 | 0.433 |
| | | CoV-2 | | | | | | | | |
| M21 + RNA_16 | No | InfA | 666542 | 5779 | 35.641 | 0.280 | 538063 | 58788 | 35.568 | 0.239 |
| M21 + RNA_16 | No | InfB | 252194 | 19374 | 35.081 | 0.281 | 135539 | 14575 | 35.791 | 0.318 |
| M21 + RNA_16 | No | RNaseP | 963241 | 35492 | 34.856 | 0.238 | 774405 | 16740 | 34.869 | 0.138 |
| M21 + RNA_16 | No | SARS- | 365575 | 26278 | 35.604 | 0.246 | 228565 | 34396 | 35.620 | 0.285 |
| | | CoV-2 | | | | | | | | |
| M21 + RNA_17 | No | InfA | 662599 | 42963 | 35.724 | 0.318 | 426252 | 34978 | 36.088 | 0.095 |
| M21 + RNA_17 | No | InfB | 282042 | 26486 | 34.697 | 0.668 | 167642 | 10450 | 35.229 | 0.370 |
| M21 + RNA_17 | No | RNaseP | 1035157 | 15050 | 34.579 | 0.378 | 793551 | 49588 | 35.365 | 0.245 |
| M21 + RNA_17 | No | SARS- | 442891 | 16510 | 35.314 | 0.192 | 213135 | 25307 | 34.951 | 0.313 |
| | | CoV-2 | | | | | | | | |
| M21 + RNA_18 | No | InfA | 789479 | 8127 | 35.066 | 0.329 | 356820 | 99000 | 36.933 | 1.060 |
| M21 + RNA_18 | No | InfB | 248010 | 15299 | 35.178 | 0.135 | 121074 | 8321 | 35.594 | 0.191 |
| M21 + RNA_18 | No | RNaseP | 1073710 | 51798 | 34.839 | 0.272 | 880721 | 71579 | 34.721 | 0.361 |
| M21 + RNA_18 | No | SARS- | 466915 | 25500 | 34.950 | 0.173 | 227470 | 43439 | 35.336 | 0.643 |
| | | CoV-2 | | | | | | | | |
| M21 + RNA_19 | No | InfA | 731987 | 21341 | 35.400 | 0.132 | 530844 | 26587 | 35.794 | 0.418 |
| M21 + RNA_19 | No | InfB | 281439 | 27334 | 34.493 | 0.663 | 163542 | 9669 | 35.239 | 0.481 |
| M21 + RNA_19 | No | RNaseP | 1005829 | 29555 | 34.859 | 0.495 | 908454 | 30532 | 34.697 | 0.143 |
| M21 + RNA_19 | No | SARS- | 466386 | 19189 | 34.767 | 0.241 | 134624 | 855 | 36.153 | 0.280 |
| | | CoV-2 | | | | | | | | |
| M21 + RNA_20 | No | InfA | 557954 | 34592 | 35.558 | 0.390 | 145217 | 29294 | 37.014 | 1.218 |
| M21 + RNA_20 | No | InfB | 197002 | 23203 | 35.215 | 0.666 | 40487 | 9933 | 36.324 | 1.487 |
| M21 + RNA_20 | No | RNaseP | 858633 | 73685 | 34.655 | 0.239 | 591022 | 34258 | 34.489 | 0.211 |
| M21 + RNA_20 | No | SARS- | 409831 | 34371 | 35.139 | 0.209 | 100091 | 15790 | 35.276 | 0.700 |
| | | CoV-2 | | | | | | | | |
| M21 + RNA_21 | No | InfA | 762375 | 20608 | 35.299 | 0.451 | 534227 | 16376 | 36.074 | 0.191 |
| M21 + RNA_21 | No | InfB | 274353 | 14762 | 35.118 | 0.629 | 159608 | 21057 | 35.737 | 0.616 |
| M21 + RNA_21 | No | RNaseP | 1102732 | 30441 | 34.551 | 0.158 | 954011 | 128424 | 34.918 | 0.621 |
| M21 + RNA_21 | No | SARS- | 447198 | 34809 | 35.304 | 0.180 | 250215 | 42010 | 35.826 | 0.660 |
| | | CoV-2 | | | | | | | | |
| M21 + RNA_22 | No | InfA | 738512 | 36302 | 35.425 | 0.468 | 400181 | 93779 | 36.626 | 0.897 |
| M21 + RNA_22 | No | InfB | 246023 | 18916 | 35.454 | 0.513 | 156403 | 10478 | 35.447 | 0.327 |
| M21 + RNA_22 | No | RNaseP | 1088278 | 59098 | 34.563 | 0.317 | 851030 | 71312 | 35.287 | 0.289 |
| M21 + RNA_22 | No | SARS- | 471643 | 43243 | 35.393 | 0.667 | 226804 | 24341 | 35.784 | 0.355 |
| | | CoV-2 | | | | | | | | |
| M21 + RNA_23 | No | InfA | 687582 | 48716 | 35.470 | 0.321 | 260146 | 72642 | 36.592 | 0.664 |
| M21 + RNA_23 | No | InfB | 238862 | 13411 | 35.091 | 0.183 | 74476 | 9837 | 35.923 | 0.443 |
| M21 + RNA_23 | No | RNaseP | 1000877 | 20282 | 34.625 | 0.068 | 703077 | 7396 | 34.873 | 0.340 |
| M21 + RNA_23 | No | SARS- | 424267 | 6257 | 34.910 | 0.221 | 178135 | 24902 | 35.301 | 0.376 |
| | | CoV-2 | | | | | | | | |
| M21 + RNA_24 | No | InfA | 641389 | 48328 | 35.627 | 0.561 | 220653 | 64969 | 36.747 | 0.812 |
| M21 + RNA_24 | No | InfB | 222836 | 10898 | 35.412 | 0.504 | 81820 | 24212 | 35.409 | 0.857 |
| M21 + RNA_24 | No | RNaseP | 917734 | 68754 | 34.973 | 0.566 | 726489 | 51544 | 34.697 | 0.249 |
| M21 + RNA_24 | No | SARS- | 371912 | 11156 | 35.750 | 0.345 | 152795 | 6010 | 35.170 | 0.276 |
| | | CoV-2 | | | | | | | | |
| M21 + RNA_25 | No | InfA | 688254 | 29424 | 35.546 | 0.231 | 487387 | 86674 | 36.012 | 0.426 |
| M21 + RNA_25 | No | InfB | 277267 | 24413 | 34.368 | 0.482 | 118332 | 12677 | 35.876 | 0.500 |
| M21 + RNA_25 | No | RNaseP | 1008462 | 65910 | 34.501 | 0.171 | 923013 | 66749 | 34.581 | 0.335 |
| M21 + RNA_25 | No | SARS- | 446490 | 9199 | 34.686 | 0.511 | 288565 | 6650 | 35.294 | 0.277 |
| | | CoV-2 | | | | | | | | |
| M21 + RNA_26 | No | InfA | 735173 | 20947 | 35.208 | 0.232 | 539080 | 97232 | 35.684 | 0.552 |
| M21 + RNA_26 | No | InfB | 286991 | 12706 | 33.959 | 0.270 | 137547 | 3501 | 35.132 | 0.235 |
| M21 + RNA_26 | No | RNaseP | 1065067 | 17045 | 34.120 | 0.201 | 629349 | 33394 | 34.859 | 0.345 |
| M21 + RNA_26 | No | SARS- | 478467 | 15531 | 34.577 | 0.144 | 249441 | 42954 | 35.065 | 0.454 |
| | | CoV-2 | | | | | | | | |
| M21 + RNA_27 | No | InfA | 701667 | 95875 | 35.783 | 0.833 | 558900 | 130225 | 35.681 | 0.809 |
| M21 + RNA_27 | No | InfB | 263354 | 38754 | 34.963 | 0.330 | 182808 | 26391 | 35.004 | 0.541 |
| M21 + RNA_27 | No | RNaseP | 1081620 | 19679 | 34.714 | 0.151 | 967181 | 56463 | 34.709 | 0.250 |
| M21 + RNA_27 | No | SARS- | 463472 | 100298 | 35.120 | 0.571 | 208107 | 59467 | 36.158 | 0.796 |
| | | CoV-2 | | | | | | | | |
| M21 + RNA_28 | No | InfA | 725978 | 112336 | 35.477 | 1.138 | 481320 | 74654 | 36.044 | 0.619 |
| M21 + RNA_28 | No | InfB | 244887 | 50439 | 34.895 | 0.992 | 139733 | 7541 | 35.091 | 0.148 |
| M21 + RNA_28 | No | RNaseP | 1104257 | 33485 | 34.774 | 0.471 | 991843 | 15399 | 34.330 | 0.227 |
| M21 + RNA_28 | No | SARS- | 476213 | 45248 | 34.705 | 0.137 | 196372 | 23645 | 34.569 | 0.262 |
| | | CoV-2 | | | | | | | | |
| M21 + RNA_29 | No | InfA | 822719 | 52431 | 35.346 | 0.763 | 570372 | 55095 | 36.005 | 0.428 |
| M21 + RNA_29 | No | InfB | 273336 | 7979 | 35.247 | 0.536 | 166874 | 3512 | 35.821 | 0.197 |
| M21 + RNA_29 | No | RNaseP | 1225231 | 88649 | 34.482 | 0.177 | 1056902 | 52942 | 34.592 | 0.205 |
| M21 + RNA_29 | No | SARS- | 577422 | 36366 | 34.820 | 0.284 | 321355 | 47194 | 35.514 | 0.447 |
| | | CoV-2 | | | | | | | | |
| M21 + RNA_30 | No | InfA | 795627 | 52081 | 35.061 | 0.382 | 593863 | 56184 | 35.573 | 0.463 |
| M21 + RNA_30 | No | InfB | 297423 | 24087 | 34.326 | 0.209 | 150713 | 4750 | 35.147 | 0.194 |
| M21 + RNA_30 | No | RNaseP | 1130145 | 55517 | 34.502 | 0.585 | 944074 | 44015 | 34.683 | 0.103 |
| M21 + RNA_30 | No | SARS- | 420612 | 43826 | 35.352 | 0.506 | 111956 | 6290 | 35.313 | 0.274 |
| | | CoV-2 | | | | | | | | |
| M21 + RNA_31 | No | InfA | 721138 | 27367 | 35.226 | 0.392 | 440169 | 172714 | 36.917 | 2.094 |
| M21 + RNA_31 | No | InfB | 225070 | 34147 | 36.059 | 0.677 | 138993 | 17264 | 35.868 | 0.655 |
| M21 + RNA_31 | No | RNaseP | 1027131 | 45539 | 34.949 | 0.096 | 948358 | 71743 | 34.579 | 0.362 |
| M21 + RNA_31 | No | SARS- | 460180 | 37394 | 35.129 | 0.551 | 189907 | 38502 | 35.403 | 0.589 |
| | | CoV-2 | | | | | | | | |
| M21 + RNA_32 | No | InfA | 696799 | 59273 | 35.345 | 0.512 | 414731 | 40345 | 36.512 | 0.412 |
| M21 + RNA_32 | No | InfB | 256273 | 33053 | 35.222 | 0.713 | 155679 | 14578 | 35.378 | 0.218 |
| M21 + RNA_32 | No | RNaseP | 999683 | 66013 | 34.982 | 0.382 | 785140 | 39435 | 34.919 | 0.189 |
| M21 + RNA_32 | No | SARS- | 439887 | 49129 | 35.199 | 0.488 | 276221 | 25837 | 35.500 | 0.429 |
| | | CoV-2 | | | | | | | | |
| M21 + RNA_33 | No | InfA | 725338 | 77677 | 34.987 | 0.473 | 472510 | 16110 | 35.782 | 0.328 |
| M21 + RNA_33 | No | InfB | 245953 | 26023 | 34.533 | 0.566 | 126960 | 40836 | 35.478 | 0.913 |
| M21 + RNA_33 | No | RNaseP | 1033546 | 126680 | 34.547 | 0.362 | 787707 | 25292 | 34.583 | 0.349 |
| M21 + RNA_33 | No | SARS- | 439750 | 33785 | 34.953 | 0.383 | 202492 | 20446 | 35.560 | 0.300 |
| | | CoV-2 | | | | | | | | |
| M21 + RNA_34 | No | InfA | 754022 | 44684 | 35.461 | 0.196 | 577016 | 41984 | 35.891 | 0.413 |
| M21 + RNA_34 | No | InfB | 293239 | 50120 | 34.104 | 1.017 | 128240 | 23582 | 35.966 | 0.668 |
| M21 + RNA_34 | No | RNaseP | 1055956 | 62821 | 34.558 | 0.341 | 890746 | 35067 | 35.136 | 0.221 |
| M21 + RNA_34 | No | SARS- | 490560 | 19562 | 34.014 | 0.741 | 265408 | 21377 | 35.326 | 0.403 |
| | | CoV-2 | | | | | | | | |
| M21 + RNA_35 | No | InfA | 718348 | 70683 | 35.258 | 0.183 | 394859 | 53370 | 36.124 | 0.582 |
| M21 + RNA_35 | No | InfB | 246714 | 17472 | 34.225 | 0.209 | 115074 | 18740 | 34.847 | 0.514 |
| M21 + RNA_35 | No | RNaseP | 845370 | 83478 | 34.610 | 0.254 | 707408 | 5697 | 34.354 | 0.246 |
| M21 + RNA_35 | No | SARS- | 464811 | 52259 | 34.875 | 0.896 | 150503 | 31793 | 35.101 | 0.580 |
| | | CoV-2 | | | | | | | | |
| M21 + RNA_36 | No | InfA | 607236 | 45893 | 36.083 | 0.946 | 377474 | 134362 | 36.568 | 1.770 |
| M21 + RNA_36 | No | InfB | 246331 | 16398 | 34.898 | 0.308 | 124607 | 14320 | 35.298 | 0.372 |
| M21 + RNA_36 | No | RNaseP | 1004200 | 35207 | 34.426 | 0.121 | 887302 | 51704 | 34.859 | 0.221 |
| M21 + RNA_36 | No | SARS- | 498067 | 28059 | 34.730 | 0.583 | 259396 | 45123 | 35.235 | 0.340 |
| | | CoV-2 | | | | | | | | |
| M21 + RNA_37 | No | InfA | 803556 | 65978 | 35.058 | 0.481 | 558274 | 34618 | 35.775 | 0.330 |
| M21 + RNA_37 | No | InfB | 313255 | 15044 | 34.397 | 0.473 | 115399 | 47632 | 36.596 | 1.717 |
| M21 + RNA_37 | No | RNaseP | 1106419 | 65511 | 34.632 | 0.389 | 819051 | 70117 | 35.559 | 0.470 |
| M21 + RNA_37 | No | SARS- | 548321 | 25393 | 34.392 | 0.097 | 267891 | 12039 | 35.877 | 0.073 |
| | | CoV-2 | | | | | | | | |
| M21 + RNA_38 | No | InfA | 589916 | 52410 | 35.426 | 0.166 | 195268 | 97038 | 36.679 | 1.964 |
| M21 + RNA_38 | No | InfB | 238046 | 1825 | 34.220 | 0.427 | 43498 | 10852 | 36.447 | 1.600 |
| M21 + RNA_38 | No | RNaseP | 965733 | 125132 | 34.572 | 0.179 | 686440 | 70927 | 34.280 | 0.461 |
| M21 + RNA_38 | No | SARS- | 431065 | 11791 | 35.125 | 0.853 | 118181 | 52498 | 35.557 | 1.750 |
| | | CoV-2 | | | | | | | | |
| M21 + RNA_39 | No | InfA | 706932 | 94629 | 35.328 | 0.458 | 516671 | 80740 | 35.331 | 0.470 |
| M21 + RNA_39 | No | InfB | 249108 | 35187 | 35.008 | 0.855 | 149698 | 24966 | 35.210 | 0.650 |
| M21 + RNA_39 | No | RNaseP | 1038820 | 45272 | 34.488 | 0.206 | 876532 | 25286 | 34.689 | 0.487 |
| M21 + RNA_39 | No | SARS- | 445316 | 31294 | 35.261 | 0.269 | 280386 | 27651 | 35.119 | 0.324 |
| | | CoV-2 | | | | | | | | |
| M21 + RNA_40 | No | InfA | 737784 | 31128 | 35.088 | 0.185 | 440420 | 56353 | 36.263 | 0.534 |
| M21 + RNA_40 | No | InfB | 229846 | 25675 | 35.552 | 0.395 | 127203 | 27473 | 35.998 | 1.049 |
| M21 + RNA_40 | No | RNaseP | 1031494 | 33049 | 35.030 | 0.156 | 818073 | 59963 | 34.999 | 0.272 |
| M21 + RNA_40 | No | SARS- | 437203 | 43533 | 35.339 | 0.381 | 289240 | 21624 | 35.416 | 0.443 |
| | | CoV-2 | | | | | | | | |
| M21 + RNA_41 | No | InfA | 789033 | 21453 | 34.845 | 0.341 | 397648 | 108649 | 36.104 | 0.989 |
| M21 + RNA_41 | No | InfB | 288685 | 15225 | 34.143 | 0.264 | 108560 | 33239 | 35.620 | 1.153 |
| M21 + RNA_41 | No | RNaseP | 1018206 | 30102 | 34.696 | 0.146 | 848668 | 55375 | 34.439 | 0.125 |
| M21 + RNA_41 | No | SARS- | 427091 | 16529 | 34.711 | 0.465 | 174667 | 30760 | 35.596 | 0.276 |
| | | CoV-2 | | | | | | | | |
| M21 + RNA_42 | No | InfA | 817858 | 16521 | 34.513 | 0.088 | 438828 | 65910 | 36.701 | 0.843 |
| M21 + RNA_42 | No | InfB | 303762 | 5291 | 34.048 | 0.299 | 135944 | 29865 | 35.991 | 0.924 |
| M21 + RNA_42 | No | RNaseP | 1073897 | 77088 | 34.615 | 0.373 | 880329 | 32392 | 34.888 | 0.391 |
| M21 + RNA_42 | No | SARS- | 451151 | 23677 | 34.723 | 0.189 | 310797 | 37893 | 35.261 | 0.266 |
| | | CoV-2 | | | | | | | | |
| M21 + RNA_43 | No | InfA | 668376 | 22606 | 35.757 | 0.271 | 536915 | 88651 | 36.001 | 0.895 |
| M21 + RNA_43 | No | InfB | 273326 | 21984 | 34.218 | 0.525 | 129948 | 3753 | 35.884 | 0.260 |
| M21 + RNA_43 | No | RNaseP | 952349 | 40169 | 34.624 | 0.477 | 831838 | 49762 | 35.038 | 0.191 |
| M21 + RNA_43 | No | SARS- | 470823 | 29267 | 34.545 | 0.681 | 213101 | 29513 | 35.920 | 0.350 |
| | | CoV-2 | | | | | | | | |
| M21 + RNA_44 | No | InfA | 772547 | 58876 | 35.444 | 0.819 | 410648 | 198018 | 37.632 | 1.993 |
| M21 + RNA_44 | No | InfB | 299465 | 13840 | 34.305 | 0.776 | 165779 | 12702 | 35.390 | 0.175 |
| M21 + RNA_44 | No | RNaseP | 1174071 | 10919 | 34.153 | 0.129 | 930483 | 48553 | 34.888 | 0.363 |
| M21 + RNA_44 | No | SARS- | 502448 | 16648 | 34.892 | 0.341 | 224740 | 15297 | 35.298 | 0.283 |
| | | CoV-2 | | | | | | | | |
| M21 + RNA_45 | No | InfA | 811270 | 29502 | 34.871 | 0.467 | 447999 | 128322 | 36.416 | 0.896 |
| M21 + RNA_45 | No | InfB | 285056 | 11962 | 34.651 | 0.462 | 116888 | 28345 | 35.863 | 0.639 |
| M21 + RNA_45 | No | RNaseP | 1089966 | 60206 | 34.489 | 0.345 | 869200 | 53061 | 34.970 | 0.169 |
| M21 + RNA_45 | No | SARS- | 477006 | 5735 | 34.990 | 0.539 | 256793 | 41917 | 35.172 | 0.106 |
| | | CoV-2 | | | | | | | | |
| M21 + RNA_46 | No | InfA | 714184 | 35086 | 35.505 | 0.440 | 350619 | 77868 | 36.257 | 0.623 |
| M21 + RNA_46 | No | InfB | 273797 | 18912 | 34.606 | 0.217 | 112233 | 29021 | 35.325 | 0.915 |
| M21 + RNA_46 | No | RNaseP | 1042465 | 27215 | 34.486 | 0.264 | 744909 | 42217 | 34.815 | 0.238 |
| M21 + RNA_46 | No | SARS- | 457928 | 17244 | 34.690 | 0.603 | 206311 | 18215 | 35.372 | 0.363 |
| | | CoV-2 | | | | | | | | |
| M21 + RNA_47 | No | InfA | 722054 | 15276 | 35.185 | 0.212 | 494699 | 68912 | 36.210 | 0.228 |
| M21 + RNA_47 | No | InfB | 272656 | 30726 | 34.617 | 0.960 | 158955 | 30198 | 35.713 | 0.932 |
| M21 + RNA_47 | No | RNaseP | 1025883 | 55002 | 34.771 | 0.309 | 806411 | 21915 | 34.983 | 0.149 |
| M21 + RNA_47 | No | SARS- | 412073 | 2890 | 34.972 | 0.489 | 228136 | 39405 | 36.112 | 0.557 |
| | | CoV-2 | | | | | | | | |
| M21 + RNA_48 | No | InfA | 663982 | 50449 | 35.194 | 0.547 | 294774 | 50694 | 35.784 | 0.622 |
| M21 + RNA_48 | No | InfB | 222961 | 6838 | 35.405 | 0.334 | 57078 | 12279 | 36.241 | 1.127 |
| M21 + RNA_48 | No | RNaseP | 952626 | 20720 | 34.566 | 0.226 | 656032 | 77790 | 34.951 | 0.474 |
| M21 + RNA_48 | No | SARS- | 392526 | 5102 | 34.889 | 0.328 | 127498 | 32234 | 35.282 | 0.464 |
| | | CoV-2 | | | | | | | | |
| M21 + RNA_49 | No | InfA | 756579 | 39961 | 34.698 | 0.513 | 385579 | 16745 | 35.745 | 0.010 |
| M21 + RNA_49 | No | InfB | 269958 | 26876 | 34.349 | 0.840 | 92721 | 9831 | 35.536 | 0.147 |
| M21 + RNA_49 | No | RNaseP | 991152 | 65358 | 34.725 | 0.303 | 828358 | 36795 | 34.414 | 0.388 |
| M21 + RNA_49 | No | SARS- | 435624 | 37497 | 35.126 | 0.967 | 196994 | 30159 | 34.882 | 0.187 |
| | | CoV-2 | | | | | | | | |
| M21 + RNA_50 | No | InfA | 652626 | 165101 | 35.952 | 1.706 | 481349 | 42799 | 35.975 | 0.108 |
| M21 + RNA_50 | No | InfB | 288244 | 11786 | 34.471 | 0.150 | 124989 | 21799 | 35.407 | 0.528 |
| M21 + RNA_50 | No | RNaseP | 1006647 | 44430 | 34.678 | 0.175 | 851786 | 42454 | 34.789 | 0.312 |
| M21 + RNA_50 | No | SARS- | 441394 | 35518 | 34.938 | 0.599 | 220550 | 31652 | 35.428 | 0.473 |
| | | CoV-2 | | | | | | | | |
| RNA Aptamer | Yes | InfA | 710468 | 60071 | 35.629 | 0.602 | 278165 | 28917 | 37.049 | 0.657 |
| Only | | | | | | | | | | |
| RNA Aptamer | Yes | InfB | 232111 | 6852 | 34.711 | 0.390 | 49729 | 14070 | 36.534 | 2.416 |
| Only | | | | | | | | | | |
| RNA Aptamer | Yes | RNaseP | 978469 | 74258 | 34.747 | 0.404 | 527584 | 28424 | 34.429 | 0.271 |
| Only | | | | | | | | | | |
| RNA Aptamer | Yes | SARS- | 304423 | 40733 | 35.633 | 0.668 | 90707 | 24890 | 35.746 | 1.066 |
| Only | | CoV-2 | | | | | | | | |
| No Aptamer | No | InfA | 553584 | 56992 | 35.183 | 0.382 | 127093 | 29335 | 36.791 | 1.049 |
| No Aptamer | No | InfB | 207036 | 20435 | 34.759 | 0.590 | 48768 | 9392 | 35.029 | 0.789 |
| No Aptamer | No | RNaseP | 803290 | 46812 | 34.322 | 0.162 | 529163 | 30650 | 34.514 | 0.149 |
| No Aptamer | No | SARS- | 412688 | 50592 | 34.738 | 0.385 | 74009 | 25811 | 35.649 | 1.048 |
| | | CoV-2 | | | | | | | | |
| No Template | No | InfA | 3432 | 341 | NA | NA | 2646 | 166 | NA | NA |
| Control | | | | | | | | | | |
| No Template | No | InfB | 1625 | 181 | NA | NA | 964 | 186 | NA | NA |
| Control | | | | | | | | | | |
| No Template | No | RNaseP | 2507 | 438 | NA | NA | 1661 | 36 | NA | NA |
| Control | | | | | | | | | | |
| No Template | No | SARS- | 1558 | 189 | NA | NA | 1107 | 156 | NA | NA |
| Control | | CoV-2 | | | | | | | | |

For Tables 2-11, data was reported by threshold cycle (Ct) and endpoint fluorescence (delta Rn) values. Samples that did not surpass the amplification threshold are marked as “No Amplification” (NA).

TABLE 4
Evaluation of room temperature stability at 0 hours and 2 hours following template addition to
PrimeTime ™ One-Step RT-qPCR Master Mix (Integrated DNA Technologies) with DNA and RNA aptamers.

| | | | 0 h Endpoint | | 0 h Cycle | | 2 h Endpoint | | 2 h Cycle | |
|---|---|---|---|---|---|---|---|---|---|---|
| | | | Fluorescence | | Threshold | | Fluorescence | | Threshold | |
| | RNA | | Delta | Delta | Ct | Ct | Delta | Delta | Ct | Ct |
| Sample Name | Aptamer? | Target Name | Rn Mean | Rn SD | Mean | SD | Rn Mean | Rn SD | Mean | SD |
| M21_1 | Yes | InfA | 759,060 | 38,617 | 35.186 | 0.263 | 437,284 | 66,399 | 35.468 | 0.599 |
| M21_1 | Yes | InfB | 222,815 | 23,530 | 34.123 | 0.735 | 67,780 | 8,329 | 34.415 | 0.330 |
| M21_1 | Yes | RNaseP | 965,021 | 41,989 | 34.488 | 0.084 | 526,458 | 43,255 | 34.561 | 0.506 |
| M21_1 | Yes | SARS-CoV-2 | 349,678 | 41,238 | 34.561 | 0.372 | 89,014 | 9,743 | 34.953 | 0.376 |
| M21_2 | Yes | InfA | 688,624 | 51,518 | 34.943 | 0.536 | 125,700 | 24,272 | 37.302 | 1.201 |
| M21_2 | Yes | InfB | 196,137 | 6,697 | 33.920 | 0.289 | 20,284 | 3,467 | 36.742 | 1.157 |
| M21_2 | Yes | RNaseP | 869,577 | 28,177 | 34.363 | 0.193 | 410,888 | 14,612 | 34.049 | 0.155 |
| M21_2 | Yes | SARS-CoV-2 | 351,008 | 22,155 | 34.363 | 0.137 | 40,817 | 5,966 | 35.052 | 0.599 |
| M21_3 | Yes | InfA | 731,189 | 13,568 | 35.672 | 0.227 | 351,477 | 71,271 | 36.256 | 0.602 |
| M21_3 | Yes | InfB | 219,788 | 29,994 | 34.667 | 0.584 | 74,075 | 20,064 | 34.563 | 1.010 |
| M21_3 | Yes | RNaseP | 981,680 | 82,334 | 34.983 | 0.355 | 614,058 | 25,091 | 34.630 | 0.292 |
| M21_3 | Yes | SARS-CoV-2 | 358,926 | 21,829 | 34.787 | 0.359 | 75,064 | 17,945 | 35.637 | 0.665 |
| M21_4 | Yes | InfA | 715,708 | 47,522 | 35.506 | 0.363 | 379,812 | 70,018 | 36.174 | 0.687 |
| M21_4 | Yes | InfB | 243,537 | 25,146 | 33.958 | 0.432 | 63,631 | 14,722 | 34.740 | 0.754 |
| M21_4 | Yes | RNaseP | 890,593 | 73,731 | 34.937 | 0.607 | 640,626 | 64,087 | 34.157 | 0.313 |
| M21_4 | Yes | SARS-CoV-2 | 388,882 | 29,279 | 34.582 | 0.531 | 122,569 | 33,740 | 34.547 | 1.148 |
| M21_5 | Yes | InfA | 547,411 | 92,694 | 34.574 | 0.339 | 45,228 | 26,571 | 39.538 | 1.714 |
| M21_5 | Yes | InfB | 114,592 | 6,670 | 34.233 | 0.328 | 5,709 | 3,797 | 44.692 | NA |
| M21_5 | Yes | RNaseP | 718,115 | 12,710 | 33.944 | 0.224 | 239,110 | 5,708 | 34.581 | 0.185 |
| M21_5 | Yes | SARS-CoV-2 | 142,371 | 81,873 | 34.807 | 1.190 | 7,569 | 5,919 | 44.918 | NA |
| M21_6 | Yes | InfA | 726,289 | 71,738 | 35.050 | 0.632 | 385,712 | 32,356 | 35.421 | 0.148 |
| M21_6 | Yes | InfB | 217,029 | 25,082 | 34.141 | 0.506 | 49,253 | 21,559 | 35.238 | 1.664 |
| M21_6 | Yes | RNaseP | 937,502 | 17,498 | 34.784 | 0.104 | 567,744 | 44,013 | 33.939 | 0.418 |
| M21_6 | Yes | SARS-CoV-2 | 369,138 | 22,171 | 34.535 | 0.065 | 76,472 | 18,404 | 35.162 | 0.945 |
| M21_7 | Yes | InfA | 593,704 | 90,957 | 35.890 | 0.236 | 425,494 | 78,461 | 35.883 | 0.486 |
| M21_7 | Yes | InfB | 194,972 | 50,286 | 34.669 | 0.677 | 71,328 | 18,671 | 34.754 | 0.921 |
| M21_7 | Yes | RNaseP | 849,878 | 87,811 | 34.850 | 0.109 | 548,642 | 42,868 | 35.078 | 0.357 |
| M21_7 | Yes | SARS-CoV-2 | 357,338 | 43,828 | 34.795 | 0.443 | 127,267 | 11,810 | 34.548 | 0.341 |
| M21_8 | Yes | InfA | 672,963 | 86,939 | 35.383 | 0.716 | 315,952 | 89,761 | 36.029 | 1.190 |
| M21_8 | Yes | InfB | 179,209 | 10,752 | 34.756 | 0.348 | 43,522 | 14,747 | 35.510 | 1.473 |
| M21_8 | Yes | RNaseP | 822,383 | 81,497 | 34.966 | 0.279 | 534,101 | 66,806 | 34.087 | 0.610 |
| M21_8 | Yes | SARS-CoV-2 | 341,487 | 52,729 | 34.568 | 0.274 | 64,406 | 29,313 | 36.300 | 2.688 |
| M21_9 | Yes | InfA | 639,578 | 37,528 | 35.122 | 0.586 | 166,891 | 22,329 | 36.195 | 0.667 |
| M21_9 | Yes | InfB | 193,626 | 15,797 | 33.619 | 0.452 | 22,726 | 7,070 | 36.577 | 1.719 |
| M21_9 | Yes | RNaseP | 849,350 | 49,132 | 34.379 | 0.508 | 375,498 | 10,813 | 34.011 | 0.128 |
| M21_9 | Yes | SARS-CoV-2 | 339,005 | 25,879 | 34.342 | 0.231 | 22,545 | 6,950 | 39.005 | 3.289 |
| M21_10 | Yes | InfA | 733,053 | 45,030 | 35.159 | 0.558 | 274,059 | 113,921 | 36.606 | 1.560 |
| M21_10 | Yes | InfB | 240,488 | 3,260 | 33.894 | 0.338 | 42,099 | 4,722 | 35.054 | 0.438 |
| M21_10 | Yes | RNaseP | 1,002,390 | 58,833 | 34.479 | 0.258 | 551,912 | 11,528 | 34.020 | 0.124 |
| M21_10 | Yes | SARS-CoV-2 | 366,151 | 36,954 | 34.784 | 0.317 | 87,450 | 19,860 | 34.484 | 0.756 |
| M21_11 | Yes | InfA | 741,074 | 14,068 | 34.906 | 0.454 | 368,697 | 66,957 | 35.897 | 0.760 |
| M21_11 | Yes | InfB | 222,994 | 13,744 | 33.971 | 0.331 | 64,640 | 7,908 | 34.235 | 0.490 |
| M21_11 | Yes | RNaseP | 1,039,630 | 24,630 | 34.552 | 0.091 | 601,607 | 65,451 | 34.052 | 0.440 |
| M21_11 | Yes | SARS-CoV-2 | 387,195 | 10,176 | 34.463 | 0.247 | 78,249 | 13,220 | 34.967 | 0.540 |
| M21_12 | Yes | InfA | 731,450 | 36,046 | 35.316 | 0.508 | 293,286 | 85,080 | 36.630 | 1.216 |
| M21_12 | Yes | InfB | 223,132 | 18,053 | 34.267 | 0.671 | 51,466 | 6,768 | 34.797 | 0.366 |
| M21_12 | Yes | RNaseP | 1,046,114 | 51,380 | 34.494 | 0.091 | 555,302 | 30,938 | 34.380 | 0.272 |
| M21_12 | Yes | SARS-CoV-2 | 423,019 | 29,544 | 34.568 | 0.107 | 84,975 | 14,984 | 34.827 | 0.706 |
| M21_13 | Yes | InfA | 721,196 | 39,552 | 34.783 | 0.512 | 113,029 | 13,430 | 37.416 | 0.640 |
| M21_13 | Yes | InfB | 202,179 | 16,812 | 33.772 | 0.237 | 20,207 | 7,842 | 37.225 | 2.890 |
| M21_13 | Yes | RNaseP | 852,290 | 27,230 | 34.274 | 0.230 | 386,753 | 18,465 | 33.908 | 0.172 |
| M21_13 | Yes | SARS-CoV-2 | 382,876 | 12,007 | 33.894 | 0.102 | 24,875 | 1,939 | 36.899 | 0.686 |
| M21_14 | Yes | InfA | 741,945 | 129,437 | 35.188 | 1.077 | 407,533 | 6,284 | 35.289 | 0.026 |
| M21_14 | Yes | InfB | 230,358 | 1,757 | 33.820 | 0.212 | 40,569 | 15,586 | 35.658 | 2.057 |
| M21_14 | Yes | RNaseP | 956,361 | 38,809 | 34.644 | 0.488 | 546,717 | 23,303 | 34.030 | 0.307 |
| M21_14 | Yes | SARS-CoV-2 | 375,734 | 32,419 | 34.482 | 0.345 | 84,960 | 34,218 | 34.569 | 1.056 |
| M21_15 | Yes | InfA | 686,686 | 55,371 | 35.306 | 0.523 | 380,866 | 59,460 | 35.529 | 0.464 |
| M21_15 | Yes | InfB | 197,836 | 3,891 | 34.411 | 0.254 | 60,300 | 13,089 | 34.327 | 0.845 |
| M21_15 | Yes | RNaseP | 968,311 | 20,177 | 34.762 | 0.218 | 602,939 | 47,162 | 34.018 | 0.346 |
| M21_15 | Yes | SARS-CoV-2 | 395,520 | 23,420 | 34.417 | 0.166 | 88,234 | 8,075 | 34.681 | 0.306 |
| M21_16 | Yes | InfA | 666,145 | 109,327 | 35.426 | 1.113 | 327,158 | 107,190 | 36.053 | 1.175 |
| M21_16 | Yes | InfB | 174,247 | 35,139 | 34.838 | 0.771 | 55,460 | 8,767 | 34.487 | 0.465 |
| M21_16 | Yes | RNaseP | 954,259 | 61,547 | 34.640 | 0.383 | 525,983 | 38,906 | 34.607 | 0.439 |
| M21_16 | Yes | SARS-CoV-2 | 380,730 | 11,173 | 34.439 | 0.108 | 64,499 | 10,078 | 35.474 | 0.450 |
| M21_17 | Yes | InfA | 764,798 | 21,287 | 34.331 | 0.326 | 176,881 | 67,566 | 36.616 | 1.687 |
| M21_17 | Yes | InfB | 199,013 | 6,169 | 33.679 | 0.193 | 19,866 | 3,776 | 36.938 | 1.519 |
| M21_17 | Yes | RNaseP | 881,530 | 40,369 | 34.098 | 0.125 | 388,824 | 31,666 | 33.922 | 0.406 |
| M21_17 | Yes | SARS-CoV-2 | 349,123 | 23,219 | 34.324 | 0.193 | 32,616 | 10,563 | 36.426 | 1.747 |
| M21_18 | Yes | InfA | 724,036 | 44,893 | 34.980 | 0.411 | 123,458 | 30,746 | 38.092 | 1.005 |
| M21_18 | Yes | InfB | 187,906 | 19,309 | 34.399 | 0.422 | 19,123 | 1,432 | 37.285 | 0.431 |
| M21_18 | Yes | RNaseP | 923,772 | 62,554 | 34.413 | 0.252 | 443,821 | 13,431 | 34.098 | 0.155 |
| M21_18 | Yes | SARS-CoV-2 | 442,072 | 10,374 | 33.981 | 0.161 | 50,038 | 6,901 | 35.067 | 0.429 |
| M21_19 | Yes | InfA | 699,762 | 85,032 | 35.289 | 1.117 | 356,697 | 66,186 | 35.847 | 0.624 |
| M21_19 | Yes | InfB | 224,505 | 28,035 | 34.161 | 0.499 | 51,729 | 10,975 | 34.632 | 0.911 |
| M21_19 | Yes | RNaseP | 989,059 | 77,558 | 34.515 | 0.509 | 597,706 | 15,255 | 33.924 | 0.344 |
| M21_19 | Yes | SARS-CoV-2 | 410,213 | 51,596 | 34.307 | 0.499 | 70,706 | 13,600 | 34.890 | 0.714 |
| M21_20 | Yes | InfA | 800,660 | 7,635 | 34.842 | 0.268 | 343,397 | 65,343 | 35.716 | 0.562 |
| M21_20 | Yes | InfB | 230,015 | 17,006 | 34.082 | 0.437 | 38,886 | 10,477 | 35.464 | 0.941 |
| M21_20 | Yes | RNaseP | 1,025,915 | 72,809 | 34.704 | 0.568 | 667,849 | 53,710 | 33.613 | 0.354 |
| M21_20 | Yes | SARS-CoV-2 | 384,031 | 35,614 | 34.642 | 0.295 | 77,079 | 20,795 | 34.995 | 0.947 |
| M21_21 | Yes | InfA | 571,674 | 68,157 | 34.755 | 0.608 | 79,629 | 7,195 | 38.372 | 0.705 |
| M21_21 | Yes | InfB | 144,195 | 9,608 | 33.918 | 0.184 | 6,933 | 3,022 | NA | NA |
| M21_21 | Yes | RNaseP | 774,086 | 15,019 | 33.902 | 0.049 | 250,573 | 34,601 | 34.787 | 0.768 |
| M21_21 | Yes | SARS-CoV-2 | 278,602 | 36,002 | 34.054 | 0.378 | 11,845 | 1,335 | 44.380 | NA |
| M21_22 | Yes | InfA | 678,972 | 100,886 | 35.660 | 0.627 | 273,166 | 62,857 | 36.163 | 0.942 |
| M21_22 | Yes | InfB | 228,071 | 3,841 | 34.013 | 0.320 | 44,894 | 4,731 | 34.714 | 0.572 |
| M21_22 | Yes | RNaseP | 1,014,997 | 44,355 | 34.458 | 0.288 | 620,375 | 9,932 | 33.575 | 0.097 |
| M21_22 | Yes | SARS-CoV-2 | 428,328 | 30,373 | 34.053 | 0.342 | 61,955 | 7,022 | 34.147 | 0.194 |
| M21_23 | Yes | InfA | 695,352 | 38,605 | 35.072 | 0.376 | 192,733 | 35,123 | 36.012 | 0.817 |
| M21_23 | Yes | InfB | 171,107 | 4,327 | 34.291 | 0.212 | 21,143 | 8,144 | 35.426 | 0.611 |
| M21_23 | Yes | RNaseP | 935,711 | 19,376 | 34.219 | 0.098 | 429,763 | 27,677 | 34.088 | 0.163 |
| M21_23 | Yes | SARS-CoV-2 | 380,225 | 31,623 | 34.523 | 0.498 | 45,846 | 4,983 | 35.098 | 0.268 |
| M21_24 | Yes | InfA | 671,412 | 38,310 | 35.559 | 0.312 | 266,730 | 95,227 | 36.248 | 1.209 |
| M21_24 | Yes | InfB | 217,883 | 6,438 | 33.885 | 0.140 | 43,139 | 14,141 | 34.974 | 1.055 |
| M21_24 | Yes | RNaseP | 972,061 | 23,369 | 34.743 | 0.172 | 562,497 | 29,908 | 34.140 | 0.296 |
| M21_24 | Yes | SARS-CoV-2 | 394,919 | 26,071 | 34.424 | 0.240 | 66,515 | 10,341 | 35.246 | 0.491 |
| M21_25 | Yes | InfA | 600,712 | 67,399 | 34.875 | 0.433 | 91,391 | 38,651 | 36.882 | 0.074 |
| M21_25 | Yes | InfB | 167,157 | 9,258 | 33.755 | 0.104 | 12,671 | 8,971 | 36.943 | 2.384 |
| M21_25 | Yes | RNaseP | 853,540 | 48,103 | 33.932 | 0.506 | 381,007 | 17,236 | 33.882 | 0.242 |
| M21_25 | Yes | SARS-CoV-2 | 386,601 | 34,699 | 33.221 | 0.846 | 24,974 | 5,035 | 37.240 | 1.955 |
| M21_26 | Yes | InfA | 684,205 | 82,610 | 35.149 | 0.447 | 362,184 | 64,007 | 35.655 | 0.784 |
| M21_26 | Yes | InfB | 209,503 | 26,871 | 33.869 | 0.603 | 52,228 | 5,871 | 34.613 | 0.332 |
| M21_26 | Yes | RNaseP | 1,024,374 | 41,018 | 34.176 | 0.288 | 574,727 | 36,537 | 33.832 | 0.314 |
| M21_26 | Yes | SARS-CoV-2 | 418,724 | 23,507 | 33.948 | 0.242 | 71,258 | 15,545 | 34.884 | 0.655 |
| M21_27 | Yes | InfA | 622,960 | 99,911 | 34.843 | 0.361 | 112,029 | 27,641 | 37.851 | 0.911 |
| M21_27 | Yes | InfB | 159,088 | 20,451 | 34.323 | 0.295 | 20,071 | 5,202 | 37.162 | 2.961 |
| M21_27 | Yes | RNaseP | 892,163 | 23,946 | 33.885 | 0.250 | 425,288 | 21,314 | 33.850 | 0.242 |
| M21_27 | Yes | SARS-CoV-2 | 380,281 | 16,273 | 33.877 | 0.408 | 30,234 | 14,971 | 37.550 | 3.760 |
| M21_28 | Yes | InfA | 686,966 | 119,115 | 35.620 | 0.413 | 400,337 | 39,974 | 36.342 | 0.287 |
| M21_28 | Yes | InfB | 218,788 | 29,566 | 33.941 | 0.102 | 70,210 | 9,949 | 34.682 | 0.520 |
| M21_28 | Yes | RNaseP | 1,083,554 | 146,882 | 34.334 | 0.464 | 752,211 | 64,595 | 33.933 | 0.430 |
| M21_28 | Yes | SARS-CoV-2 | 366,877 | 97,139 | 34.412 | 0.953 | 173,742 | 20,965 | 33.927 | 0.359 |
| M21_29 | Yes | InfA | 701,671 | 58,623 | 34.773 | 0.267 | 143,058 | 70,950 | 38.194 | 3.101 |
| M21_29 | Yes | InfB | 203,713 | 11,314 | 33.494 | 0.181 | 16,805 | 5,561 | 38.998 | 4.289 |
| M21_29 | Yes | RNaseP | 944,419 | 108,620 | 34.107 | 0.121 | 525,422 | 44,904 | 34.029 | 0.435 |
| M21_29 | Yes | SARS-CoV-2 | 252,590 | 23,655 | 33.751 | 0.231 | 3,448 | 3,252 | NA | NA |
| M21_30 | Yes | InfA | 650,081 | 54,729 | 35.181 | 0.649 | 226,703 | 45,710 | 35.971 | 0.801 |
| M21_30 | Yes | InfB | 203,705 | 13,124 | 33.841 | 0.352 | 22,241 | 6,704 | 37.420 | 3.028 |
| M21_30 | Yes | RNaseP | 942,257 | 44,934 | 34.064 | 0.110 | 515,076 | 15,867 | 33.556 | 0.105 |
| M21_30 | Yes | SARS-CoV-2 | 376,732 | 18,103 | 34.158 | 0.354 | 32,169 | 1,814 | 36.337 | 0.309 |
| M21_31 | Yes | InfA | 718,133 | 30,609 | 35.196 | 0.382 | 276,933 | 42,986 | 36.248 | 0.691 |
| M21_31 | Yes | InfB | 214,348 | 11,069 | 34.168 | 0.523 | 40,890 | 10,634 | 35.440 | 0.900 |
| M21_31 | Yes | RNaseP | 1,034,657 | 15,045 | 34.360 | 0.210 | 616,120 | 7,635 | 33.897 | 0.077 |
| M21_31 | Yes | SARS-CoV-2 | 335,620 | 12,507 | 35.007 | 0.334 | 99,721 | 18,607 | 34.288 | 0.477 |
| M21_32 | Yes | InfA | 725,756 | 103,926 | 35.364 | 1.294 | 417,565 | 21,915 | 35.760 | 0.240 |
| M21_32 | Yes | InfB | 225,503 | 34,371 | 34.300 | 0.463 | 69,879 | 10,759 | 34.538 | 0.516 |
| M21_32 | Yes | RNaseP | 984,724 | 45,736 | 34.848 | 0.098 | 651,310 | 58,430 | 34.272 | 0.343 |
| M21_32 | Yes | SARS-CoV-2 | 391,197 | 18,571 | 34.606 | 0.145 | 122,548 | 9,422 | 34.259 | 0.319 |
| M21_33 | Yes | InfA | 712,973 | 50,346 | 35.109 | 0.043 | 190,430 | 7,526 | 37.200 | 0.219 |
| M21_33 | Yes | InfB | 209,664 | 30,604 | 33.699 | 0.929 | 39,972 | 4,689 | 34.821 | 0.265 |
| M21_33 | Yes | RNaseP | 979,084 | 70,917 | 34.150 | 0.589 | 542,924 | 37,017 | 33.576 | 0.321 |
| M21_33 | Yes | SARS-CoV-2 | 373,328 | 32,212 | 34.284 | 0.336 | 47,992 | 7,461 | 35.100 | 0.666 |
| M21_34 | Yes | InfA | 773,742 | 35,931 | 34.666 | 0.163 | 243,161 | 93,722 | 36.227 | 1.797 |
| M21_34 | Yes | InfB | 196,912 | 27,655 | 33.828 | 0.924 | 28,788 | 918 | 35.388 | 0.196 |
| M21_34 | Yes | RNaseP | 979,290 | 47,201 | 33.944 | 0.733 | 502,056 | 28,079 | 33.775 | 0.268 |
| M21_34 | Yes | SARS-CoV-2 | 437,933 | 48,586 | 33.642 | 1.158 | 42,726 | 4,039 | 35.340 | 0.474 |
| M21_35 | Yes | InfA | 746,996 | 41,856 | 35.140 | 0.438 | 445,238 | 44,555 | 35.736 | 0.328 |
| M21_35 | Yes | InfB | 256,874 | 16,205 | 33.026 | 0.406 | 70,158 | 14,445 | 34.365 | 0.544 |
| M21_35 | Yes | RNaseP | 997,145 | 110,049 | 33.827 | 0.043 | 609,231 | 31,120 | 33.824 | 0.339 |
| M21_35 | Yes | SARS-CoV-2 | 465,930 | 28,922 | 32.884 | 0.528 | 123,179 | 23,157 | 34.028 | 0.667 |
| M21_36 | Yes | InfA | 772,781 | 39,129 | 34.896 | 0.500 | 369,865 | 100,593 | 36.440 | 1.186 |
| M21_36 | Yes | InfB | 218,334 | 8,934 | 33.421 | 0.423 | 36,340 | 9,018 | 36.118 | 0.968 |
| M21_36 | Yes | RNaseP | 1,067,156 | 83,109 | 34.528 | 0.370 | 679,606 | 106,127 | 34.216 | 0.748 |
| M21_36 | Yes | SARS-CoV-2 | 435,646 | 12,374 | 33.945 | 0.261 | 81,416 | 7,419 | 34.437 | 0.093 |
| M21_37 | Yes | InfA | 658,322 | 18,624 | 34.934 | 0.608 | 189,001 | 60,092 | 35.998 | 1.102 |
| M21_37 | Yes | InfB | 186,528 | 18,501 | 33.631 | 0.512 | 16,946 | 2,159 | 37.700 | 1.484 |
| M21_37 | Yes | RNaseP | 869,844 | 49,758 | 34.208 | 0.281 | 383,403 | 55,673 | 33.950 | 0.382 |
| M21_37 | Yes | SARS-CoV-2 | 367,242 | 57,845 | 33.828 | 0.468 | 24,019 | 4,404 | 36.605 | 2.065 |
| M21_38 | Yes | InfA | 685,197 | 57,536 | 35.086 | 0.473 | 280,546 | 46,632 | 35.332 | 0.469 |
| M21_38 | Yes | InfB | 203,239 | 14,800 | 33.888 | 0.475 | 29,152 | 5,169 | 35.525 | 0.756 |
| M21_38 | Yes | RNaseP | 974,279 | 23,905 | 34.342 | 0.283 | 501,381 | 24,624 | 33.588 | 0.050 |
| M21_38 | Yes | SARS-CoV-2 | 457,241 | 25,863 | 33.736 | 0.176 | 44,556 | 15,043 | 35.300 | 1.150 |
| M21_39 | Yes | InfA | 676,301 | 71,984 | 35.319 | 0.614 | 291,001 | 91,014 | 35.954 | 1.345 |
| M21_39 | Yes | InfB | 198,774 | 21,246 | 34.191 | 0.463 | 32,297 | 2,243 | 35.466 | 0.030 |
| M21_39 | Yes | RNaseP | 1,015,247 | 47,890 | 34.388 | 0.400 | 525,492 | 36,779 | 33.834 | 0.186 |
| M21_39 | Yes | SARS-CoV-2 | 407,083 | 29,601 | 34.114 | 0.508 | 49,218 | 15,622 | 35.819 | 1.484 |
| M21_40 | Yes | InfA | 779,683 | 13,761 | 35.115 | 0.401 | 420,725 | 91,890 | 36.185 | 1.271 |
| M21_40 | Yes | InfB | 227,329 | 22,000 | 34.287 | 0.416 | 69,061 | 11,236 | 34.585 | 0.374 |
| M21_40 | Yes | RNaseP | 1,029,028 | 18,746 | 34.562 | 0.098 | 612,793 | 19,184 | 34.403 | 0.280 |
| M21_40 | Yes | SARS-CoV-2 | 394,167 | 20,281 | 34.439 | 0.483 | 134,052 | 5,808 | 34.158 | 0.222 |
| M21_41 | Yes | InfA | 688,152 | 37,551 | 34.967 | 0.475 | 392,895 | 16,825 | 35.135 | 0.341 |
| M21_41 | Yes | InfB | 236,157 | 4,571 | 33.209 | 0.292 | 43,634 | 4,984 | 34.899 | 0.481 |
| M21_41 | Yes | RNaseP | 938,857 | 56,477 | 34.191 | 0.091 | 501,829 | 52,522 | 33.986 | 0.352 |
| M21_41 | Yes | SARS-CoV-2 | 341,807 | 33,232 | 34.036 | 0.157 | 56,084 | 22,383 | 35.827 | 2.523 |
| M21_42 | Yes | InfA | 684,446 | 45,251 | 35.422 | 0.023 | 332,546 | 46,863 | 36.140 | 0.337 |
| M21_42 | Yes | InfB | 243,344 | 28,432 | 33.305 | 0.684 | 49,168 | 18,879 | 35.251 | 1.456 |
| M21_42 | Yes | RNaseP | 1,097,369 | 40,767 | 33.813 | 0.400 | 641,005 | 46,643 | 33.644 | 0.385 |
| M21_42 | Yes | SARS-CoV-2 | 424,513 | 12,339 | 33.603 | 0.150 | 65,602 | 8,748 | 35.173 | 0.409 |
| M21_43 | Yes | InfA | 792,909 | 24,913 | 34.701 | 0.169 | 406,193 | 38,672 | 35.346 | 0.455 |
| M21_43 | Yes | InfB | 220,142 | 21,937 | 33.811 | 0.772 | 41,864 | 8,311 | 35.321 | 0.665 |
| M21_43 | Yes | RNaseP | 1,023,507 | 16,810 | 34.043 | 0.221 | 590,864 | 62,688 | 34.118 | 0.576 |
| M21_43 | Yes | SARS-CoV-2 | 469,196 | 14,384 | 33.756 | 0.150 | 88,863 | 25,764 | 34.541 | 0.980 |
| M21_44 | Yes | InfA | 797,293 | 37,298 | 35.131 | 0.337 | 540,738 | 85,601 | 35.522 | 0.810 |
| M21_44 | Yes | InfB | 233,688 | 21,683 | 34.072 | 0.459 | 63,097 | 15,920 | 35.201 | 0.965 |
| M21_44 | Yes | RNaseP | 1,094,267 | 57,595 | 34.500 | 0.252 | 716,653 | 46,498 | 34.438 | 0.473 |
| M21_44 | Yes | SARS-CoV-2 | 420,067 | 34,688 | 34.287 | 0.395 | 156,826 | 35,624 | 34.230 | 0.611 |
| M21_45 | Yes | InfA | 664,336 | 83,604 | 35.137 | 0.628 | 183,664 | 31,855 | 35.786 | 0.786 |
| M21_45 | Yes | InfB | 190,089 | 17,205 | 33.791 | 0.106 | 20,563 | 5,642 | 37.069 | 2.379 |
| M21_45 | Yes | RNaseP | 896,125 | 50,232 | 34.110 | 0.349 | 474,977 | 8,311 | 33.423 | 0.217 |
| M21_45 | Yes | SARS-CoV-2 | 409,142 | 77,480 | 33.569 | 1.048 | 28,585 | 13,469 | 38.118 | 4.155 |
| M21_46 | Yes | InfA | 730,933 | 9,577 | 35.073 | 0.090 | 231,189 | 82,726 | 36.412 | 1.310 |
| M21_46 | Yes | InfB | 242,410 | 11,996 | 33.691 | 0.506 | 42,770 | 12,511 | 35.148 | 1.046 |
| M21_46 | Yes | RNaseP | 1,054,516 | 59,831 | 34.400 | 0.087 | 600,980 | 30,773 | 33.830 | 0.139 |
| M21_46 | Yes | SARS-CoV-2 | 415,275 | 27,774 | 33.930 | 0.221 | 55,980 | 3,772 | 34.463 | 0.346 |
| M21_47 | Yes | InfA | 631,010 | 74,833 | 35.819 | 0.704 | 274,912 | 138,600 | 36.788 | 2.572 |
| M21_47 | Yes | InfB | 202,885 | 13,535 | 34.426 | 0.357 | 53,731 | 17,588 | 34.305 | 1.123 |
| M21_47 | Yes | RNaseP | 986,233 | 30,020 | 34.573 | 0.217 | 557,826 | 44,658 | 34.028 | 0.223 |
| M21_47 | Yes | SARS-CoV-2 | 384,622 | 59,403 | 34.634 | 0.677 | 74,942 | 19,632 | 34.664 | 0.992 |
| M21_48 | Yes | InfA | 660,523 | 7,161 | 35.637 | 0.318 | 285,446 | 78,705 | 36.265 | 0.726 |
| M21_48 | Yes | InfB | 213,640 | 8,323 | 33.998 | 0.415 | 35,883 | 1,445 | 35.445 | 0.343 |
| M21_48 | Yes | RNaseP | 963,096 | 83,819 | 34.885 | 0.786 | 602,326 | 36,324 | 33.952 | 0.209 |
| M21_48 | Yes | SARS-CoV-2 | 366,783 | 11,135 | 34.698 | 0.193 | 101,951 | 33,866 | 34.274 | 0.826 |
| M21_49 | Yes | InfA | 736,782 | 65,941 | 34.981 | 0.419 | 353,267 | 43,403 | 35.466 | 0.493 |
| M21_49 | Yes | InfB | 211,441 | 11,353 | 34.523 | 0.650 | 54,517 | 6,061 | 34.365 | 0.304 |
| M21_49 | Yes | RNaseP | 957,280 | 74,429 | 34.258 | 0.210 | 516,131 | 22,779 | 34.048 | 0.373 |
| M21_49 | Yes | SARS-CoV-2 | 401,849 | 22,802 | 34.397 | 0.382 | 74,423 | 11,656 | 34.573 | 0.352 |
| M21_50 | Yes | InfA | 679,353 | 79,142 | 35.687 | 0.779 | 350,342 | 99,034 | 36.305 | 1.279 |
| M21_50 | Yes | InfB | 217,503 | 39,208 | 34.124 | 0.550 | 60,709 | 5,913 | 34.592 | 0.297 |
| M21_50 | Yes | RNaseP | 1,061,209 | 20,187 | 34.331 | 0.089 | 543,823 | 96,407 | 34.765 | 1.110 |
| M21_50 | Yes | SARS-CoV-2 | 417,982 | 12,872 | 34.240 | 0.450 | 108,427 | 39,363 | 34.545 | 1.241 |
| M21 + RNA_1 | Yes | InfA | 795,256 | 48,267 | 34.822 | 0.632 | 450,325 | 71,500 | 35.421 | 0.708 |
| M21 + RNA_1 | Yes | InfB | 221,524 | 24,149 | 34.434 | 0.449 | 63,173 | 3,588 | 34.508 | 0.217 |
| M21 + RNA_1 | Yes | RNaseP | 915,898 | 33,473 | 34.553 | 0.367 | 561,520 | 17,090 | 33.987 | 0.061 |
| M21 + RNA_1 | Yes | SARS-CoV-2 | 395,469 | 13,276 | 34.495 | 0.093 | 103,522 | 15,429 | 34.356 | 0.547 |
| M21 + RNA_2 | Yes | InfA | 642,933 | 83,139 | 35.614 | 0.690 | 379,690 | 73,554 | 35.412 | 0.669 |
| M21 + RNA_2 | Yes | InfB | 179,962 | 40,893 | 34.693 | 0.745 | 44,239 | 4,450 | 34.814 | 0.526 |
| M21 + RNA_2 | Yes | RNaseP | 915,292 | 112,983 | 34.607 | 0.390 | 467,393 | 25,428 | 34.282 | 0.074 |
| M21 + RNA_2 | Yes | SARS-CoV-2 | 350,619 | 52,565 | 34.578 | 0.609 | 42,792 | 12,793 | 36.100 | 1.578 |
| M21 + RNA_3 | Yes | InfA | 732,568 | 109,099 | 35.180 | 1.149 | 211,535 | 161,459 | 36.176 | 1.073 |
| M21 + RNA_3 | Yes | InfB | 233,558 | 27,245 | 34.077 | 0.716 | 39,557 | 14,600 | 36.191 | 2.254 |
| M21 + RNA_3 | Yes | RNaseP | 941,398 | 30,294 | 34.225 | 0.055 | 407,034 | 122,679 | 35.071 | 1.515 |
| M21 + RNA_3 | Yes | SARS-CoV-2 | 402,179 | 12,728 | 34.444 | 0.226 | 71,845 | 48,120 | 33.954 | 0.351 |
| M21 + RNA_4 | Yes | InfA | 780,940 | 52,848 | 35.452 | 0.304 | 519,451 | 52,709 | 35.573 | 0.379 |
| M21 + RNA_4 | Yes | InfB | 248,484 | 15,147 | 33.973 | 0.119 | 84,494 | 7,311 | 34.404 | 0.272 |
| M21 + RNA_4 | Yes | RNaseP | 1,019,192 | 25,457 | 34.770 | 0.162 | 672,596 | 36,021 | 34.322 | 0.188 |
| M21 + RNA_4 | Yes | SARS-CoV-2 | 405,208 | 41,079 | 34.646 | 0.243 | 141,018 | 3,457 | 34.241 | 0.174 |
| M21 + RNA_5 | Yes | InfA | 794,000 | 41,879 | 35.146 | 0.359 | 326,214 | 35,454 | 36.886 | 0.409 |
| M21 + RNA_5 | Yes | InfB | 263,661 | 16,307 | 33.863 | 0.413 | 57,604 | 27,893 | 35.624 | 2.169 |
| M21 + RNA_5 | Yes | RNaseP | 1,010,686 | 25,897 | 34.411 | 0.325 | 637,456 | 34,971 | 33.931 | 0.245 |
| M21 + RNA_5 | Yes | SARS-CoV-2 | 389,260 | 60,737 | 34.609 | 0.430 | 118,903 | 12,950 | 34.015 | 0.244 |
| M21 + RNA_6 | Yes | InfA | 622,841 | 180,170 | 35.825 | 1.481 | 342,428 | 40,615 | 35.970 | 0.389 |
| M21 + RNA_6 | Yes | InfB | 232,156 | 9,937 | 33.723 | 0.201 | 53,605 | 19,816 | 35.049 | 1.048 |
| M21 + RNA_6 | Yes | RNaseP | 975,402 | 137,395 | 34.443 | 0.602 | 511,250 | 35,652 | 34.343 | 0.325 |
| M21 + RNA_6 | Yes | SARS-CoV-2 | 408,101 | 46,404 | 34.430 | 0.425 | 80,202 | 33,884 | 35.126 | 1.402 |
| M21 + RNA_7 | Yes | InfA | 747,521 | 14,200 | 35.076 | 0.029 | 438,560 | 8,582 | 35.573 | 0.255 |
| M21 + RNA_7 | Yes | InfB | 210,057 | 38,207 | 34.564 | 0.837 | 50,522 | 7,310 | 35.142 | 0.602 |
| M21 + RNA_7 | Yes | RNaseP | 886,678 | 64,535 | 34.712 | 0.427 | 539,559 | 43,089 | 34.313 | 0.413 |
| M21 + RNA_7 | Yes | SARS-CoV-2 | 301,632 | 18,340 | 35.393 | 0.356 | 73,286 | 12,580 | 34.792 | 0.661 |
| M21 + RNA_8 | Yes | InfA | 673,687 | 26,803 | 35.836 | 0.165 | 439,981 | 79,818 | 35.974 | 0.804 |
| M21 + RNA_8 | Yes | InfB | 196,641 | 23,478 | 34.817 | 0.519 | 67,450 | 26,836 | 35.313 | 1.617 |
| M21 + RNA_8 | Yes | RNaseP | 901,912 | 49,277 | 35.053 | 0.269 | 610,557 | 31,209 | 34.457 | 0.281 |
| M21 + RNA_8 | Yes | SARS-CoV-2 | 319,390 | 22,957 | 35.275 | 0.352 | 142,875 | 20,583 | 34.394 | 0.335 |
| M21 + RNA_9 | Yes | InfA | 792,912 | 31,850 | 35.129 | 0.230 | 538,048 | 22,670 | 35.247 | 0.042 |
| M21 + RNA_9 | Yes | InfB | 255,541 | 34,941 | 33.874 | 0.208 | 63,467 | 7,655 | 35.288 | 0.518 |
| M21 + RNA_9 | Yes | RNaseP | 1,054,425 | 68,892 | 34.708 | 0.413 | 665,985 | 23,749 | 34.057 | 0.143 |
| M21 + RNA_9 | Yes | SARS-CoV-2 | 416,775 | 39,419 | 34.543 | 0.261 | 149,209 | 18,849 | 34.037 | 0.429 |
| M21 + RNA_10 | Yes | InfA | 730,925 | 56,119 | 35.101 | 0.371 | 279,174 | 56,911 | 36.183 | 0.712 |
| M21 + RNA_10 | Yes | InfB | 223,851 | 13,234 | 33.860 | 0.441 | 34,026 | 8,896 | 35.490 | 1.418 |
| M21 + RNA_10 | Yes | RNaseP | 964,564 | 61,475 | 34.579 | 0.321 | 515,985 | 27,966 | 33.975 | 0.552 |
| M21 + RNA_10 | Yes | SARS-CoV-2 | 419,726 | 34,356 | 34.260 | 0.394 | 62,987 | 10,823 | 34.625 | 0.585 |
| M21 + RNA_11 | Yes | InfA | 724,748 | 56,866 | 35.242 | 0.184 | 371,690 | 39,873 | 36.083 | 0.208 |
| M21 + RNA_11 | Yes | InfB | 230,722 | 17,358 | 34.415 | 0.206 | 89,621 | 8,473 | 34.334 | 0.489 |
| M21 + RNA_11 | Yes | RNaseP | 1,100,660 | 40,800 | 34.349 | 0.288 | 695,159 | 44,374 | 34.028 | 0.394 |
| M21 + RNA_11 | Yes | SARS-CoV-2 | 431,484 | 51,800 | 34.198 | 0.488 | 143,714 | 10,342 | 34.335 | 0.082 |
| M21 + RNA_12 | Yes | InfA | 738,036 | 21,882 | 35.454 | 0.349 | 407,770 | 41,510 | 36.057 | 0.251 |
| M21 + RNA_12 | Yes | InfB | 237,766 | 5,368 | 33.938 | 0.298 | 58,716 | 10,694 | 34.809 | 0.571 |
| M21 + RNA_12 | Yes | RNaseP | 1,097,552 | 47,493 | 34.587 | 0.184 | 636,017 | 18,084 | 34.170 | 0.181 |
| M21 + RNA_12 | Yes | SARS-CoV-2 | 402,405 | 24,561 | 34.642 | 0.522 | 116,452 | 10,983 | 34.223 | 0.251 |
| M21 + RNA_13 | Yes | InfA | 776,979 | 58,344 | 34.935 | 0.252 | 461,320 | 58,703 | 35.272 | 0.442 |
| M21 + RNA_13 | Yes | InfB | 206,132 | 24,146 | 34.382 | 0.517 | 51,125 | 12,918 | 34.899 | 1.047 |
| M21 + RNA_13 | Yes | RNaseP | 1,071,602 | 47,669 | 34.177 | 0.209 | 569,268 | 22,596 | 34.140 | 0.370 |
| M21 + RNA_13 | Yes | SARS-CoV-2 | 418,020 | 47,801 | 34.191 | 0.482 | 41,837 | 5,342 | 36.143 | 0.810 |
| M21 + RNA_14 | Yes | InfA | 804,134 | 20,404 | 34.596 | 0.152 | 356,670 | 19,509 | 35.363 | 0.191 |
| M21 + RNA_14 | Yes | InfB | 229,627 | 46,695 | 34.022 | 0.815 | 36,355 | 9,017 | 35.327 | 1.178 |
| M21 + RNA_14 | Yes | RNaseP | 1,023,451 | 60,676 | 34.499 | 0.270 | 506,909 | 23,828 | 34.184 | 0.373 |
| M21 + RNA_14 | Yes | SARS-CoV-2 | 410,970 | 37,606 | 34.403 | 0.643 | 73,636 | 18,279 | 34.587 | 0.927 |
| M21 + RNA_15 | Yes | InfA | 783,563 | 28,758 | 35.082 | 0.498 | 422,000 | 37,796 | 36.070 | 0.569 |
| M21 + RNA_15 | Yes | InfB | 212,193 | 17,471 | 34.652 | 0.271 | 69,069 | 9,283 | 34.849 | 0.460 |
| M21 + RNA_15 | Yes | RNaseP | 1,094,681 | 56,818 | 34.662 | 0.405 | 670,922 | 35,500 | 34.312 | 0.153 |
| M21 + RNA_15 | Yes | SARS-CoV-2 | 388,966 | 24,132 | 34.742 | 0.505 | 134,313 | 10,609 | 34.456 | 0.233 |
| M21 + RNA_16 | Yes | InfA | 653,472 | 63,854 | 35.597 | 0.583 | 459,425 | 90,930 | 35.681 | 0.878 |
| M21 + RNA_16 | Yes | InfB | 223,211 | 28,756 | 34.369 | 0.363 | 56,361 | 11,285 | 35.372 | 0.804 |
| M21 + RNA_16 | Yes | RNaseP | 955,850 | 50,304 | 34.815 | 0.077 | 634,626 | 48,183 | 34.368 | 0.517 |
| M21 + RNA_16 | Yes | SARS-CoV-2 | 358,305 | 27,809 | 34.407 | 0.445 | 133,172 | 10,447 | 34.288 | 0.110 |
| M21 + RNA_17 | Yes | InfA | 771,849 | 13,200 | 35.103 | 0.127 | 345,967 | 37,235 | 36.153 | 0.353 |
| M21 + RNA_17 | Yes | InfB | 235,555 | 16,326 | 34.148 | 0.391 | 55,870 | 17,832 | 35.078 | 1.237 |
| M21 + RNA_17 | Yes | RNaseP | 1,095,922 | 103,213 | 34.217 | 0.571 | 629,665 | 47,607 | 34.126 | 0.368 |
| M21 + RNA_17 | Yes | SARS-CoV-2 | 427,706 | 20,242 | 34.081 | 0.318 | 89,080 | 11,243 | 34.569 | 0.507 |
| M21 + RNA_18 | Yes | InfA | 782,955 | 23,739 | 34.818 | 0.457 | 356,989 | 17,739 | 35.709 | 0.230 |
| M21 + RNA_18 | Yes | InfB | 230,557 | 8,044 | 33.853 | 0.032 | 49,010 | 10,581 | 34.611 | 0.735 |
| M21 + RNA_18 | Yes | RNaseP | 1,011,771 | 36,160 | 34.363 | 0.218 | 571,161 | 49,128 | 33.893 | 0.509 |
| M21 + RNA_18 | Yes | SARS-CoV-2 | 421,360 | 49,989 | 34.120 | 0.726 | 46,653 | 15,483 | 36.114 | 1.312 |
| M21 + RNA_19 | Yes | InfA | 732,548 | 55,005 | 35.321 | 0.447 | 435,871 | 51,025 | 35.750 | 0.524 |
| M21 + RNA_19 | Yes | InfB | 228,724 | 19,076 | 34.222 | 0.370 | 51,382 | 13,643 | 35.211 | 0.970 |
| M21 + RNA_19 | Yes | RNaseP | 1,106,581 | 35,670 | 34.448 | 0.241 | 693,441 | 58,118 | 33.865 | 0.327 |
| M21 + RNA_19 | Yes | SARS-CoV-2 | 424,922 | 36,546 | 34.273 | 0.334 | 88,503 | 3,501 | 34.262 | 0.173 |
| M21 + RNA_20 | Yes | InfA | 704,612 | 66,596 | 35.726 | 1.054 | 390,513 | 15,803 | 35.497 | 0.182 |
| M21 + RNA_20 | Yes | InfB | 240,511 | 21,986 | 34.104 | 0.514 | 47,950 | 7,448 | 34.965 | 0.390 |
| M21 + RNA_20 | Yes | RNaseP | 1,081,620 | 79,406 | 34.661 | 0.352 | 583,124 | 105,263 | 34.368 | 0.789 |
| M21 + RNA_20 | Yes | SARS-CoV-2 | 437,955 | 14,582 | 34.548 | 0.119 | 76,001 | 19,958 | 35.319 | 0.859 |
| M21 + RNA_21 | Yes | InfA | 752,722 | 49,652 | 35.232 | 0.611 | 439,816 | 116,471 | 35.698 | 0.850 |
| M21 + RNA_21 | Yes | InfB | 238,780 | 2,239 | 33.753 | 0.010 | 68,398 | 8,644 | 34.643 | 0.293 |
| M21 + RNA_21 | Yes | RNaseP | 1,114,637 | 88,792 | 34.265 | 0.535 | 713,593 | 62,331 | 33.704 | 0.495 |
| M21 + RNA_21 | Yes | SARS-CoV-2 | 421,423 | 38,446 | 34.352 | 0.429 | 107,737 | 13,970 | 34.467 | 0.504 |
| M21 + RNA_22 | Yes | InfA | 743,328 | 27,042 | 35.220 | 0.409 | 407,364 | 68,739 | 35.568 | 0.630 |
| M21 + RNA_22 | Yes | InfB | 259,307 | 5,444 | 33.511 | 0.031 | 62,302 | 7,678 | 34.506 | 0.330 |
| M21 + RNA_22 | Yes | RNaseP | 1,017,982 | 11,508 | 34.669 | 0.233 | 602,739 | 85,930 | 34.261 | 0.432 |
| M21 + RNA_22 | Yes | SARS-CoV-2 | 413,689 | 28,330 | 34.367 | 0.367 | 132,948 | 12,823 | 33.781 | 0.265 |
| M21 + RNA_23 | Yes | InfA | 730,107 | 26,051 | 35.259 | 0.354 | 374,866 | 67,340 | 35.609 | 0.593 |
| M21 + RNA_23 | Yes | InfB | 220,667 | 12,118 | 34.010 | 0.440 | 46,978 | 5,760 | 34.886 | 0.505 |
| M21 + RNA_23 | Yes | RNaseP | 1,033,021 | 50,460 | 34.648 | 0.459 | 582,283 | 85,416 | 34.331 | 0.803 |
| M21 + RNA_23 | Yes | SARS-CoV-2 | 398,940 | 20,303 | 34.467 | 0.291 | 114,667 | 24,712 | 33.897 | 0.546 |
| M21 + RNA_24 | Yes | InfA | 711,673 | 37,340 | 35.348 | 0.366 | 401,598 | 80,296 | 35.528 | 0.597 |
| M21 + RNA_24 | Yes | InfB | 220,849 | 10,377 | 34.023 | 0.066 | 50,340 | 5,060 | 34.903 | 0.399 |
| M21 + RNA_24 | Yes | RNaseP | 906,632 | 165,882 | 35.115 | 0.661 | 590,506 | 24,606 | 34.224 | 0.197 |
| M21 + RNA_24 | Yes | SARS-CoV-2 | 357,927 | 23,813 | 34.765 | 0.186 | 90,764 | 9,257 | 34.719 | 0.137 |
| M21 + RNA_25 | Yes | InfA | 730,840 | 30,656 | 35.420 | 0.541 | 419,334 | 82,529 | 35.768 | 1.054 |
| M21 + RNA_25 | Yes | InfB | 225,567 | 19,582 | 34.055 | 0.313 | 73,118 | 7,686 | 34.251 | 0.343 |
| M21 + RNA_25 | Yes | RNaseP | 1,081,016 | 111,672 | 34.153 | 0.711 | 633,115 | 34,606 | 33.980 | 0.244 |
| M21 + RNA_25 | Yes | SARS-CoV-2 | 470,475 | 23,152 | 34.069 | 0.352 | 119,803 | 27,138 | 34.232 | 0.494 |
| M21 + RNA_26 | Yes | InfA | 781,741 | 25,163 | 34.871 | 0.110 | 441,724 | 103,182 | 35.390 | 1.032 |
| M21 + RNA_26 | Yes | InfB | 264,437 | 16,133 | 33.418 | 0.610 | 52,237 | 23,067 | 35.372 | 1.818 |
| M21 + RNA_26 | Yes | RNaseP | 1,068,906 | 106,235 | 34.470 | 0.697 | 589,865 | 29,453 | 33.919 | 0.181 |
| M21 + RNA_26 | Yes | SARS-CoV-2 | 466,040 | 74,563 | 33.940 | 0.855 | 105,720 | 40,460 | 34.127 | 0.843 |
| M21 + RNA_27 | Yes | InfA | 779,399 | 83,989 | 35.362 | 0.656 | 516,935 | 58,370 | 35.588 | 0.463 |
| M21 + RNA_27 | Yes | InfB | 263,800 | 3,898 | 33.717 | 0.365 | 62,748 | 6,657 | 34.986 | 0.476 |
| M21 + RNA_27 | Yes | RNaseP | 1,159,254 | 110,881 | 34.470 | 0.542 | 717,732 | 44,083 | 34.160 | 0.488 |
| M21 + RNA_27 | Yes | SARS-CoV-2 | 438,659 | 5,901 | 34.521 | 0.156 | 132,121 | 38,740 | 34.788 | 0.986 |
| M21 + RNA_28 | Yes | InfA | 811,131 | 43,859 | 34.938 | 0.403 | 361,743 | 89,956 | 36.678 | 1.045 |
| M21 + RNA_28 | Yes | InfB | 241,463 | 24,197 | 34.184 | 0.372 | 68,121 | 13,368 | 34.580 | 0.616 |
| M21 + RNA_28 | Yes | RNaseP | 1,169,723 | 61,816 | 34.615 | 0.451 | 802,987 | 34,499 | 33.855 | 0.327 |
| M21 + RNA_28 | Yes | SARS-CoV-2 | 416,365 | 57,095 | 34.773 | 0.422 | 137,106 | 2,370 | 33.956 | 0.063 |
| M21 + RNA_29 | Yes | InfA | 785,163 | 29,498 | 35.041 | 0.183 | 495,429 | 90,004 | 35.642 | 0.688 |
| M21 + RNA_29 | Yes | InfB | 244,440 | 13,726 | 33.952 | 0.103 | 67,054 | 23,105 | 35.146 | 1.363 |
| M21 + RNA_29 | Yes | RNaseP | 1,135,790 | 45,419 | 34.250 | 0.179 | 721,969 | 54,416 | 34.135 | 0.214 |
| M21 + RNA_29 | Yes | SARS-CoV-2 | 403,243 | 102,617 | 34.676 | 1.081 | 141,624 | 31,323 | 34.419 | 0.394 |
| M21 + RNA_30 | Yes | InfA | 737,544 | 27,542 | 35.344 | 0.098 | 380,779 | 105,317 | 36.107 | 1.052 |
| M21 + RNA_30 | Yes | InfB | 207,422 | 18,393 | 34.922 | 0.712 | 62,920 | 4,045 | 34.585 | 0.197 |
| M21 + RNA_30 | Yes | RNaseP | 1,039,413 | 51,103 | 34.470 | 0.267 | 719,176 | 7,431 | 33.674 | 0.141 |
| M21 + RNA_30 | Yes | SARS-CoV-2 | 423,795 | 36,281 | 34.447 | 0.395 | 70,991 | 2,289 | 34.286 | 0.191 |
| M21 + RNA_31 | Yes | InfA | 735,020 | 48,918 | 35.352 | 0.374 | 509,091 | 50,763 | 35.644 | 0.417 |
| M21 + RNA_31 | Yes | InfB | 238,992 | 13,517 | 34.142 | 0.222 | 89,605 | 12,114 | 34.223 | 0.601 |
| M21 + RNA_31 | Yes | RNaseP | 1,060,575 | 26,695 | 34.784 | 0.342 | 720,774 | 59,475 | 34.035 | 0.311 |
| M21 + RNA_31 | Yes | SARS-CoV-2 | 411,141 | 33,283 | 34.507 | 0.195 | 161,759 | 2,577 | 33.856 | 0.026 |
| M21 + RNA_32 | Yes | InfA | 700,809 | 31,100 | 35.313 | 0.146 | 351,348 | 118,794 | 36.667 | 1.265 |
| M21 + RNA_32 | Yes | InfB | 232,054 | 24,016 | 34.236 | 0.330 | 92,444 | 17,283 | 34.005 | 0.567 |
| M21 + RNA_32 | Yes | RNaseP | 1,043,706 | 92,313 | 34.682 | 0.215 | 686,425 | 30,510 | 34.192 | 0.417 |
| M21 + RNA_32 | Yes | SARS-CoV-2 | 406,227 | 30,318 | 34.510 | 0.526 | 102,168 | 27,935 | 35.189 | 0.816 |
| M21 + RNA_33 | Yes | InfA | 757,372 | 8,635 | 35.289 | 0.232 | 451,126 | 103,717 | 35.882 | 0.915 |
| M21 + RNA_33 | Yes | InfB | 208,597 | 17,667 | 34.065 | 0.463 | 80,302 | 14,135 | 34.245 | 0.562 |
| M21 + RNA_33 | Yes | RNaseP | 1,030,663 | 43,847 | 34.355 | 0.119 | 698,352 | 69,242 | 33.743 | 0.478 |
| M21 + RNA_33 | Yes | SARS-CoV-2 | 367,206 | 73,608 | 34.586 | 0.593 | 107,899 | 42,957 | 35.140 | 1.554 |
| M21 + RNA_34 | Yes | InfA | 759,313 | 46,473 | 35.280 | 0.591 | 453,395 | 149,178 | 35.816 | 1.174 |
| M21 + RNA_34 | Yes | InfB | 261,144 | 14,845 | 33.223 | 0.688 | 67,987 | 5,061 | 34.416 | 0.108 |
| M21 + RNA_34 | Yes | RNaseP | 1,108,666 | 51,292 | 34.076 | 0.334 | 625,995 | 68,911 | 34.138 | 0.269 |
| M21 + RNA_34 | Yes | SARS-CoV-2 | 470,836 | 13,459 | 33.732 | 0.179 | 127,405 | 29,543 | 34.294 | 0.588 |
| M21 + RNA_35 | Yes | InfA | 782,728 | 14,492 | 35.231 | 0.386 | 398,108 | 91,242 | 36.203 | 0.869 |
| M21 + RNA_35 | Yes | InfB | 224,877 | 6,866 | 33.356 | 0.249 | 57,784 | 6,091 | 34.665 | 0.364 |
| M21 + RNA_35 | Yes | RNaseP | 954,924 | 48,799 | 34.272 | 0.433 | 559,783 | 25,728 | 34.181 | 0.276 |
| M21 + RNA_35 | Yes | SARS-CoV-2 | 469,867 | 28,123 | 34.158 | 0.419 | 76,976 | 22,425 | 35.222 | 1.072 |
| M21 + RNA_36 | Yes | InfA | 761,339 | 62,662 | 35.114 | 0.551 | 441,945 | 48,953 | 36.226 | 0.366 |
| M21 + RNA_36 | Yes | InfB | 234,072 | 32,576 | 33.956 | 0.500 | 82,242 | 6,254 | 34.345 | 0.401 |
| M21 + RNA_36 | Yes | RNaseP | 1,085,950 | 85,591 | 34.435 | 0.546 | 753,486 | 56,767 | 34.178 | 0.412 |
| M21 + RNA_36 | Yes | SARS-CoV-2 | 444,788 | 33,243 | 33.579 | 0.710 | 181,987 | 12,445 | 33.846 | 0.149 |
| M21 + RNA_37 | Yes | InfA | 749,656 | 44,344 | 35.118 | 0.428 | 463,892 | 56,567 | 36.040 | 0.572 |
| M21 + RNA_37 | Yes | InfB | 211,033 | 36,281 | 34.255 | 0.282 | 62,644 | 17,923 | 34.725 | 0.762 |
| M21 + RNA_37 | Yes | RNaseP | 1,032,449 | 42,820 | 34.712 | 0.139 | 647,230 | 62,708 | 34.220 | 0.546 |
| M21 + RNA_37 | Yes | SARS-CoV-2 | 445,539 | 46,858 | 34.031 | 0.465 | 142,727 | 13,905 | 33.890 | 0.242 |
| M21 + RNA_38 | Yes | InfA | 688,028 | 37,321 | 35.846 | 0.250 | 271,388 | 52,267 | 36.735 | 0.816 |
| M21 + RNA_38 | Yes | InfB | 239,863 | 37,572 | 34.314 | 0.530 | 51,531 | 12,275 | 34.846 | 1.017 |
| M21 + RNA_38 | Yes | RNaseP | 1,109,603 | 10,777 | 34.347 | 0.172 | 580,964 | 20,930 | 33.961 | 0.120 |
| M21 + RNA_38 | Yes | SARS-CoV-2 | 389,419 | 34,598 | 34.821 | 0.522 | 91,956 | 14,421 | 34.598 | 0.657 |
| M21 + RNA_39 | Yes | InfA | 764,407 | 21,972 | 35.130 | 0.355 | 467,272 | 43,609 | 35.496 | 0.629 |
| M21 + RNA_39 | Yes | InfB | 233,506 | 28,884 | 34.085 | 0.554 | 62,970 | 6,084 | 34.698 | 0.254 |
| M21 + RNA_39 | Yes | RNaseP | 1,034,619 | 94,080 | 34.708 | 0.213 | 654,519 | 42,826 | 34.220 | 0.323 |
| M21 + RNA_39 | Yes | SARS-CoV-2 | 383,773 | 17,929 | 35.086 | 0.400 | 123,567 | 43,273 | 34.459 | 0.911 |
| M21 + RNA_40 | Yes | InfA | 730,697 | 40,244 | 35.339 | 0.588 | 451,183 | 49,453 | 35.875 | 0.801 |
| M21 + RNA_40 | Yes | InfB | 228,046 | 9,590 | 34.239 | 0.724 | 80,494 | 6,362 | 34.195 | 0.260 |
| M21 + RNA_40 | Yes | RNaseP | 1,050,637 | 19,655 | 34.658 | 0.139 | 650,720 | 65,176 | 34.248 | 0.522 |
| M21 + RNA_40 | Yes | SARS-CoV-2 | 411,902 | 8,214 | 34.562 | 0.389 | 149,397 | 15,355 | 34.102 | 0.191 |
| M21 + RNA_41 | Yes | InfA | 768,735 | 17,318 | 35.090 | 0.225 | 562,619 | 25,297 | 35.054 | 0.356 |
| M21 + RNA_41 | Yes | InfB | 271,822 | 3,760 | 33.830 | 0.038 | 81,223 | 7,375 | 34.406 | 0.319 |
| M21 + RNA_41 | Yes | RNaseP | 1,081,741 | 37,259 | 34.334 | 0.129 | 731,208 | 11,108 | 33.909 | 0.155 |
| M21 + RNA_41 | Yes | SARS-CoV-2 | 423,486 | 26,356 | 34.537 | 0.493 | 137,137 | 19,791 | 34.298 | 0.413 |
| M21 + RNA_42 | Yes | InfA | 681,952 | 123,747 | 35.407 | 0.690 | 528,196 | 36,990 | 35.199 | 0.308 |
| M21 + RNA_42 | Yes | InfB | 235,518 | 5,815 | 33.775 | 0.390 | 68,782 | 5,262 | 34.454 | 0.147 |
| M21 + RNA_42 | Yes | RNaseP | 1,002,420 | 150,604 | 34.436 | 0.313 | 691,054 | 6,363 | 33.841 | 0.145 |
| M21 + RNA_42 | Yes | SARS-CoV-2 | 415,577 | 35,413 | 33.831 | 0.403 | 128,251 | 8,176 | 34.193 | 0.229 |
| M21 + RNA_43 | Yes | InfA | 775,458 | 52,890 | 35.189 | 0.369 | 474,053 | 67,571 | 35.736 | 0.706 |
| M21 + RNA_43 | Yes | InfB | 215,036 | 28,004 | 34.837 | 0.645 | 75,517 | 19,992 | 34.595 | 0.957 |
| M21 + RNA_43 | Yes | RNaseP | 1,081,238 | 97,687 | 34.338 | 0.051 | 684,860 | 24,050 | 33.949 | 0.051 |
| M21 + RNA_43 | Yes | SARS-CoV-2 | 500,966 | 26,118 | 33.947 | 0.455 | 138,301 | 18,563 | 34.582 | 0.245 |
| M21 + RNA_44 | Yes | InfA | 775,576 | 25,032 | 35.418 | 0.428 | 524,480 | 28,361 | 35.883 | 0.342 |
| M21 + RNA_44 | Yes | InfB | 216,931 | 39,953 | 34.684 | 1.007 | 69,491 | 22,462 | 35.373 | 1.385 |
| M21 + RNA_44 | Yes | RNaseP | 1,105,172 | 54,813 | 34.701 | 0.612 | 748,041 | 12,986 | 34.510 | 0.144 |
| M21 + RNA_44 | Yes | SARS-CoV-2 | 500,382 | 27,212 | 33.640 | 0.894 | 159,633 | 35,010 | 34.197 | 0.656 |
| M21 + RNA_45 | Yes | InfA | 725,786 | 25,904 | 35.350 | 0.195 | 418,993 | 56,131 | 36.040 | 0.578 |
| M21 + RNA_45 | Yes | InfB | 235,964 | 36,608 | 34.017 | 0.465 | 60,177 | 4,785 | 34.728 | 0.278 |
| M21 + RNA_45 | Yes | RNaseP | 1,112,448 | 45,018 | 34.462 | 0.220 | 564,877 | 20,922 | 34.563 | 0.200 |
| M21 + RNA_45 | Yes | SARS-CoV-2 | 450,097 | 36,616 | 34.105 | 0.215 | 111,835 | 51,743 | 34.871 | 1.392 |
| M21 + RNA_46 | Yes | InfA | 745,773 | 49,594 | 34.953 | 0.280 | 319,158 | 10,077 | 36.533 | 0.098 |
| M21 + RNA_46 | Yes | InfB | 196,749 | 22,647 | 34.341 | 0.196 | 45,182 | 12,121 | 35.416 | 0.931 |
| M21 + RNA_46 | Yes | RNaseP | 1,039,891 | 114,072 | 34.167 | 0.265 | 661,159 | 31,638 | 33.871 | 0.131 |
| M21 + RNA_46 | Yes | SARS-CoV-2 | 434,315 | 36,840 | 34.169 | 0.728 | 105,551 | 31,588 | 34.501 | 0.944 |
| M21 + RNA_47 | Yes | InfA | 713,200 | 68,818 | 35.351 | 0.017 | 407,176 | 141,386 | 36.500 | 1.549 |
| M21 + RNA_47 | Yes | InfB | 230,385 | 34,628 | 34.352 | 0.697 | 67,264 | 21,885 | 35.183 | 1.325 |
| M21 + RNA_47 | Yes | RNaseP | 1,003,411 | 40,924 | 34.805 | 0.112 | 743,003 | 7,510 | 34.067 | 0.064 |
| M21 + RNA_47 | Yes | SARS-CoV-2 | 400,734 | 46,522 | 34.425 | 0.712 | 112,031 | 32,210 | 35.107 | 0.851 |
| M21 + RNA_48 | Yes | InfA | 709,731 | 38,220 | 35.184 | 0.432 | 356,153 | 27,351 | 35.950 | 0.302 |
| M21 + RNA_48 | Yes | InfB | 225,157 | 31,242 | 33.905 | 0.450 | 57,487 | 3,768 | 34.488 | 0.281 |
| M21 + RNA_48 | Yes | RNaseP | 973,249 | 3,805 | 34.771 | 0.244 | 563,274 | 27,567 | 34.495 | 0.420 |
| M21 + RNA_48 | Yes | SARS-CoV-2 | 362,345 | 15,593 | 34.689 | 0.049 | 108,053 | 10,413 | 34.212 | 0.282 |
| M21 + RNA_49 | Yes | InfA | 717,741 | 38,769 | 35.196 | 0.642 | 269,133 | 59,146 | 36.841 | 0.915 |
| M21 + RNA_49 | Yes | InfB | 207,318 | 35,794 | 34.509 | 0.614 | 49,039 | 3,021 | 34.736 | 0.166 |
| M21 + RNA_49 | Yes | RNaseP | 999,452 | 28,507 | 34.419 | 0.258 | 553,375 | 79,297 | 33.980 | 0.788 |
| M21 + RNA_49 | Yes | SARS-CoV-2 | 425,358 | 10,812 | 34.181 | 0.184 | 75,557 | 17,555 | 34.742 | 0.774 |
| M21 + RNA_50 | Yes | InfA | 701,816 | 134,292 | 35.320 | 1.018 | 365,110 | 68,123 | 35.895 | 0.774 |
| M21 + RNA_50 | Yes | InfB | 223,907 | 17,748 | 33.931 | 0.217 | 46,172 | 10,630 | 34.730 | 0.618 |
| M21 + RNA_50 | Yes | RNaseP | 955,460 | 43,608 | 34.477 | 0.559 | 569,565 | 23,690 | 34.002 | 0.053 |
| M21 + RNA_50 | Yes | SARS-CoV-2 | 378,013 | 70,292 | 34.258 | 0.637 | 73,230 | 22,334 | 34.905 | 1.315 |
| RNA Only | Yes | InfA | 748,436 | 22,332 | 34.922 | 0.377 | 367,609 | 85,606 | 35.935 | 1.303 |
| RNA Only | Yes | InfB | 194,650 | 13,996 | 34.470 | 0.389 | 28,760 | 13,347 | 37.763 | 2.984 |
| RNA Only | Yes | RNaseP | 970,899 | 15,176 | 34.730 | 0.403 | 516,129 | 81,247 | 34.271 | 0.742 |
| RNA Only | Yes | SARS-CoV-2 | 357,428 | 48,450 | 34.635 | 0.687 | 54,964 | 29,900 | 36.450 | 1.822 |
| No Aptamer | No | InfA | 607,776 | 22,346 | 34.712 | 0.183 | 57,302 | 12,745 | 40.604 | 2.692 |
| No Aptamer | No | InfB | 218,241 | 12,125 | 34.128 | 0.100 | 24,555 | 11,616 | 34.312 | 0.575 |
| No Aptamer | No | RNaseP | 760,792 | 31,832 | 34.719 | 0.250 | 495,127 | 50,824 | 33.930 | 0.158 |
| No Aptamer | No | SARS-CoV-2 | 373,875 | 7,031 | 34.799 | 0.366 | 57,791 | 7,947 | 34.333 | 0.407 |
| No Template Control | No | InfA | 1,718 | 214 | NA | NA | 1,729 | 227 | NA | NA |
| No Template Control | No | InfB | 239 | 38 | NA | NA | 599 | 90 | NA | NA |
| No Template Control | No | RNaseP | 915 | 102 | NA | NA | 1,192 | 96 | NA | NA |
| No Template Control | No | SARS-CoV-2 | 534 | 133 | NA | NA | 585 | 148 | NA | NA |

TABLE 5
Evaluation of room temperature stability at 0 hours and 4 hours following template addition to PrimeTime ™ One-
Step RT-qPCR Master Mix (Integrated DNA Technologies) with varying concentrations DNA aptamers.

| | | | 0 h Endpoint | | 0 h Cycle | | 4 h Endpoint | | | |
|---|---|---|---|---|---|---|---|---|---|---|
| | | | Fluorescence | | Threshold | | Fluorescence | | 4 h Cycle | |
| | Protein:Aptamer | Target | Delta Rn | Delta Rn | Ct | | Delta Rn | Delta Rn | Threshold | |
| Aptamer | Ratio | Name | Mean | SD | Mean | Ct SD | Mean | SD | Ct Mean | Ct SD |
| M21_9 | 1:2 | InfA | 277031 | 208174 | 38.501 | 4.451 | 141945 | 90098 | 37.921 | 4.563 |
| M21_9 | 1:2 | InfB | 155061 | 83639 | 36.021 | 1.894 | 47409 | 26252 | 38.210 | 5.347 |
| M21_9 | 1:2 | RNaseP | 590204 | 294082 | 35.643 | 2.037 | 534425 | 31553 | 34.185 | 0.279 |
| M21_9 | 1:2 | SARS- | 210849 | 97035 | 36.026 | 1.428 | 86770 | 10797 | 34.874 | 0.392 |
| | | CoV-2 | | | | | | | | |
| M21_9 | 1:3 | InfA | 747644 | 60383 | 33.753 | 0.268 | 193022 | 65007 | 35.702 | 0.832 |
| M21_9 | 1:3 | InfB | 276074 | 10950 | 34.002 | 0.364 | 40034 | 12749 | 37.202 | 1.850 |
| M21_9 | 1:3 | RNaseP | 989879 | 112315 | 33.220 | 0.273 | 635615 | 55503 | 33.794 | 0.357 |
| M21_9 | 1:3 | SARS- | 356071 | 116623 | 33.997 | 0.431 | 84953 | 16224 | 35.477 | 0.373 |
| | | CoV-2 | | | | | | | | |
| M21_9 | 1:5 | InfA | 776586 | 41068 | 33.673 | 0.315 | 174980 | 86637 | 36.376 | 1.457 |
| M21_9 | 1:5 | InfB | 264007 | 42375 | 34.463 | 0.721 | 56476 | 7754 | 36.010 | 0.355 |
| M21_9 | 1:5 | RNaseP | 1074335 | 60226 | 32.885 | 0.243 | 615024 | 60860 | 34.065 | 0.272 |
| M21_9 | 1:5 | SARS- | 481323 | 34568 | 33.832 | 0.305 | 114698 | 15005 | 35.073 | 0.527 |
| | | CoV-2 | | | | | | | | |
| M21_9 | 1:10 | InfA | 761784 | 49622 | 33.900 | 0.454 | 275543 | 84765 | 35.585 | 1.000 |
| M21_9 | 1:10 | InfB | 277179 | 17314 | 34.371 | 0.076 | 83815 | 14927 | 35.836 | 0.540 |
| M21_9 | 1:10 | RNaseP | 1023362 | 7380 | 33.587 | 0.323 | 646726 | 45897 | 34.296 | 0.304 |
| M21_9 | 1:10 | SARS- | 410248 | 1860 | 34.139 | 0.425 | 146360 | 21622 | 35.314 | 0.426 |
| | | CoV-2 | | | | | | | | |
| M21_44 | 1:2 | InfA | 588527 | 76233 | 34.717 | 0.833 | 214999 | 56449 | 35.614 | 0.829 |
| M21_44 | 1:2 | InfB | 232447 | 38628 | 34.691 | 0.656 | 69256 | 30029 | 36.033 | 2.560 |
| M21_44 | 1:2 | RNaseP | 926090 | 23892 | 33.779 | 0.844 | 613530 | 52148 | 34.172 | 0.303 |
| M21_44 | 1:2 | SARS- | 382927 | 36309 | 34.387 | 0.499 | 79563 | 13623 | 35.240 | 0.609 |
| | | CoV-2 | | | | | | | | |
| M21_44 | 1:3 | InfA | 731732 | 34195 | 33.500 | 0.260 | 231470 | 60313 | 35.925 | 0.436 |
| M21_44 | 1:3 | InfB | 240792 | 7266 | 34.471 | 0.406 | 53313 | 38578 | 35.295 | 0.265 |
| M21_44 | 1:3 | RNaseP | 954049 | 114173 | 33.615 | 0.713 | 760589 | 40515 | 33.855 | 0.045 |
| M21_44 | 1:3 | SARS- | 412922 | 55051 | 33.611 | 0.073 | 87955 | 21131 | 35.350 | 0.433 |
| | | CoV-2 | | | | | | | | |
| M21_44 | 1:5 | InfA | 712153 | 40498 | 33.990 | 0.214 | 196182 | 90449 | 36.908 | 1.252 |
| M21_44 | 1:5 | InfB | 248674 | 14153 | 34.309 | 0.150 | 76379 | 17663 | 35.914 | 0.704 |
| M21_44 | 1:5 | RNaseP | 1075640 | 56554 | 33.520 | 0.177 | 777960 | 17964 | 33.833 | 0.187 |
| M21_44 | 1:5 | SARS- | 381839 | 54449 | 33.973 | 0.250 | 114931 | 32191 | 35.193 | 0.841 |
| | | CoV-2 | | | | | | | | |
| M21_44 | 1:10 | InfA | 729146 | 68472 | 33.844 | 0.736 | 462778 | 1162 | 34.804 | 0.369 |
| M21_44 | 1:10 | InfB | 263315 | 47520 | 34.867 | 1.091 | 104175 | 26171 | 35.766 | 0.648 |
| M21_44 | 1:10 | RNaseP | 995788 | 92008 | 33.386 | 0.740 | 751033 | 90077 | 33.881 | 0.210 |
| M21_44 | 1:10 | SARS- | 398281 | 14764 | 33.929 | 0.180 | 126009 | 30809 | 35.238 | 0.292 |
| | | CoV-2 | | | | | | | | |
| M21 + RNA_9 | 1:2 | InfA | 736033 | 27800 | 33.775 | 0.258 | 388682 | 87430 | 35.733 | 0.852 |
| M21 + RNA_9 | 1:2 | InfB | 266197 | 23682 | 34.270 | 0.638 | 103449 | 7314 | 36.228 | 0.475 |
| M21 + RNA_9 | 1:2 | RNaseP | 1009094 | 39725 | 33.387 | 0.149 | 765441 | 65673 | 34.371 | 0.723 |
| M21 + RNA_9 | 1:2 | SARS- | 425158 | 41476 | 33.999 | 0.700 | 151498 | 24525 | 35.536 | 0.523 |
| | | CoV-2 | | | | | | | | |
| M21 + RNA_9 | 1:3 | InfA | 738549 | 42221 | 33.532 | 0.514 | 397159 | 55218 | 35.552 | 0.445 |
| M21 + RNA_9 | 1:3 | InfB | 277260 | 36743 | 33.844 | 0.497 | 128476 | 2132 | 35.288 | 0.244 |
| M21 + RNA_9 | 1:3 | RNaseP | 1011078 | 72880 | 33.397 | 0.172 | 836478 | 87306 | 33.909 | 0.377 |
| M21 + RNA_9 | 1:3 | SARS- | 438067 | 30605 | 33.604 | 0.183 | 147150 | 22845 | 35.424 | 0.487 |
| | | CoV-2 | | | | | | | | |
| M21 + RNA_9 | 1:5 | InfA | 672852 | 54433 | 34.357 | 0.709 | 484054 | 68539 | 35.629 | 0.659 |
| M21 + RNA_9 | 1:5 | InfB | 232410 | 18047 | 34.703 | 0.432 | 116397 | 24904 | 36.086 | 0.587 |
| M21 + RNA_9 | 1:5 | RNaseP | 1033551 | 26861 | 33.339 | 0.383 | 859418 | 47621 | 34.372 | 0.642 |
| M21 + RNA_9 | 1:5 | SARS- | 491775 | 30729 | 33.486 | 0.629 | 234711 | 14948 | 35.167 | 0.168 |
| | | CoV-2 | | | | | | | | |
| M21 + RNA_9 | 1:10 | InfA | 680232 | 76516 | 34.585 | 0.647 | 347913 | 70472 | 36.809 | 0.576 |
| M21 + RNA_9 | 1:10 | InfB | 260605 | 24102 | 34.214 | 0.502 | 145270 | 17875 | 35.919 | 0.491 |
| M21 + RNA_9 | 1:10 | RNaseP | 1020270 | 45230 | 33.558 | 0.147 | 841346 | 83989 | 34.513 | 0.326 |
| M21 + RNA_9 | 1:10 | SARS- | 431771 | 41626 | 34.109 | 0.591 | 247181 | 25575 | 35.285 | 0.085 |
| | | CoV-2 | | | | | | | | |
| M21 + RNA_21 | 1:2 | InfA | 699237 | 34664 | 34.133 | 0.395 | 226069 | 12157 | 35.947 | 0.182 |
| M21 + RNA_21 | 1:2 | InfB | 275666 | 5755 | 34.212 | 0.100 | 73929 | 16795 | 35.868 | 0.634 |
| M21 + RNA_21 | 1:2 | RNaseP | 969088 | 42835 | 33.439 | 0.282 | 652561 | 48609 | 33.961 | 0.138 |
| M21 + RNA_21 | 1:2 | SARS- | 403702 | 19428 | 33.872 | 0.359 | 116399 | 49619 | 35.457 | 1.327 |
| | | CoV-2 | | | | | | | | |
| M21 + RNA_21 | 1:3 | InfA | 687885 | 21780 | 34.103 | 0.402 | 319929 | 37736 | 35.418 | 0.442 |
| M21 + RNA_21 | 1:3 | InfB | 274113 | 18642 | 34.148 | 0.194 | 81067 | 22905 | 36.088 | 0.639 |
| M21 + RNA_21 | 1:3 | RNaseP | 1083224 | 84149 | 33.026 | 0.274 | 733316 | 55044 | 33.856 | 0.275 |
| M21 + RNA_21 | 1:3 | SARS- | 456178 | 28201 | 33.486 | 0.126 | 124373 | 8578 | 35.030 | 0.051 |
| | | CoV-2 | | | | | | | | |
| M21 + RNA_21 | 1:5 | InfA | 749633 | 30401 | 33.755 | 0.136 | 340467 | 28909 | 35.519 | 0.331 |
| M21 + RNA_21 | 1:5 | InfB | 269818 | 4085 | 34.630 | 0.093 | 116717 | 6510 | 35.194 | 0.247 |
| M21 + RNA_21 | 1:5 | RNaseP | 989436 | 46829 | 33.459 | 0.675 | 762495 | 22579 | 34.059 | 0.292 |
| M21 + RNA_21 | 1:5 | SARS- | 447939 | 43230 | 33.939 | 0.351 | 122386 | 27099 | 35.442 | 0.539 |
| | | CoV-2 | | | | | | | | |
| M21 + RNA_21 | 1:10 | InfA | 757502 | 78184 | 34.012 | 0.680 | 326085 | 175181 | 36.443 | 2.216 |
| M21 + RNA_21 | 1:10 | InfB | 283347 | 21777 | 34.413 | 0.313 | 118803 | 33911 | 35.588 | 1.019 |
| M21 + RNA_21 | 1:10 | RNaseP | 1076041 | 11251 | 33.202 | 0.391 | 693993 | 27369 | 34.018 | 0.271 |
| M21 + RNA_21 | 1:10 | SARS- | 428930 | 31163 | 34.103 | 0.270 | 119796 | 31924 | 35.488 | 0.843 |
| | | CoV-2 | | | | | | | | |
| M21 + RNA_25 | 1:2 | InfA | 742460 | 57776 | 33.904 | 0.576 | 243379 | 79450 | 35.504 | 1.046 |
| M21 + RNA_25 | 1:2 | InfB | 281104 | 24357 | 34.076 | 0.416 | 68252 | 8242 | 35.718 | 0.476 |
| M21 + RNA_25 | 1:2 | RNaseP | 998188 | 57796 | 33.282 | 0.122 | 669817 | 42670 | 34.028 | 0.310 |
| M21 + RNA_25 | 1:2 | SARS- | 369489 | 10266 | 34.386 | 0.625 | 120250 | 9441 | 35.278 | 0.329 |
| | | CoV-2 | | | | | | | | |
| M21 + RNA_25 | 1:3 | InfA | 651067 | 91637 | 34.322 | 0.367 | 327182 | 93303 | 35.005 | 0.828 |
| M21 + RNA_25 | 1:3 | InfB | 244814 | 21803 | 34.096 | 0.437 | 69991 | 8976 | 36.030 | 0.377 |
| M21 + RNA_25 | 1:3 | RNaseP | 1070382 | 27338 | 33.234 | 0.321 | 824387 | 11264 | 33.759 | 0.215 |
| M21 + RNA_25 | 1:3 | SARS- | 474448 | 13823 | 33.564 | 0.388 | 123429 | 38008 | 35.492 | 0.870 |
| | | CoV-2 | | | | | | | | |
| M21 + RNA_25 | 1:5 | InfA | 680674 | 15436 | 34.117 | 0.340 | 288571 | 46099 | 35.601 | 0.525 |
| M21 + RNA_25 | 1:5 | InfB | 259598 | 25707 | 34.047 | 0.208 | 109300 | 17352 | 34.984 | 0.385 |
| M21 + RNA_25 | 1:5 | RNaseP | 1012892 | 68050 | 33.454 | 0.305 | 711908 | 38058 | 34.094 | 0.228 |
| M21 + RNA_25 | 1:5 | SARS- | 428192 | 17757 | 33.290 | 0.472 | 164191 | 46135 | 35.067 | 0.581 |
| | | CoV-2 | | | | | | | | |
| M21 + RNA_25 | 1:10 | InfA | 735384 | 17216 | 34.209 | 0.411 | 307170 | 27565 | 35.522 | 0.301 |
| M21 + RNA_25 | 1:10 | InfB | 265094 | 26061 | 34.411 | 0.661 | 84246 | 4356 | 35.846 | 0.138 |
| M21 + RNA_25 | 1:10 | RNaseP | 1086733 | 21307 | 33.413 | 0.249 | 768645 | 70305 | 33.982 | 0.449 |
| M21 + RNA_25 | 1:10 | SARS- | 453282 | 25619 | 33.932 | 0.544 | 130454 | 53524 | 35.937 | 1.275 |
| | | CoV-2 | | | | | | | | |
| M21 + RNA_27 | 1:2 | InfA | 763128 | 63847 | 33.867 | 0.226 | 455490 | 57798 | 34.943 | 0.520 |
| M21 + RNA_27 | 1:2 | InfB | 267967 | 33436 | 34.457 | 0.270 | 94302 | 43300 | 36.513 | 2.068 |
| M21 + RNA_27 | 1:2 | RNaseP | 1037722 | 140662 | 33.281 | 0.693 | 720355 | 25552 | 34.324 | 0.130 |
| M21 + RNA_27 | 1:2 | SARS- | 443456 | 50303 | 33.904 | 0.460 | 151152 | 35065 | 35.383 | 0.530 |
| | | CoV-2 | | | | | | | | |
| M21 + RNA_27 | 1:3 | InfA | 712883 | 57873 | 33.751 | 0.204 | 339182 | 51625 | 35.715 | 0.435 |
| M21 + RNA_27 | 1:3 | InfB | 260353 | 32296 | 34.245 | 0.188 | 110232 | 16808 | 35.549 | 0.463 |
| M21 + RNA_27 | 1:3 | RNaseP | 1124303 | 30857 | 33.157 | 0.182 | 890441 | 24109 | 34.035 | 0.195 |
| M21 + RNA_27 | 1:3 | SARS- | 437421 | 43813 | 33.761 | 0.256 | 182514 | 11896 | 35.036 | 0.203 |
| | | CoV-2 | | | | | | | | |
| M21 + RNA_27 | 1:5 | InfA | 709248 | 44477 | 34.063 | 0.651 | 388122 | 40821 | 35.638 | 0.279 |
| M21 + RNA_27 | 1:5 | InfB | 225121 | 32917 | 34.881 | 0.871 | 93446 | 28709 | 36.293 | 0.855 |
| M21 + RNA_27 | 1:5 | RNaseP | 1127315 | 64511 | 33.062 | 0.142 | 876983 | 12709 | 34.034 | 0.318 |
| M21 + RNA_27 | 1:5 | SARS- | 465828 | 27503 | 33.660 | 0.279 | 155681 | 14902 | 35.872 | 0.146 |
| | | CoV-2 | | | | | | | | |
| M21 + RNA_27 | 1:10 | InfA | 700124 | 93812 | 34.308 | 0.842 | 476564 | 31379 | 35.377 | 0.357 |
| M21 + RNA_27 | 1:10 | InfB | 274818 | 1085 | 34.736 | 0.241 | 79717 | 22713 | 37.246 | 0.922 |
| M21 + RNA_27 | 1:10 | RNaseP | 1013947 | 85631 | 33.626 | 0.589 | 827911 | 4020 | 34.071 | 0.396 |
| M21 + RNA_27 | 1:10 | SARS- | 441171 | 11398 | 33.760 | 0.513 | 170700 | 33852 | 35.738 | 0.578 |
| | | CoV-2 | | | | | | | | |
| M21 + RNA_28 | 1:2 | InfA | 689259 | 125560 | 33.867 | 0.883 | 211608 | 90220 | 35.984 | 1.897 |
| M21 + RNA_28 | 1:2 | InfB | 278140 | 37687 | 34.475 | 0.557 | 48765 | 4149 | 36.400 | 0.408 |
| M21 + RNA_28 | 1:2 | RNaseP | 982568 | 22707 | 33.286 | 0.328 | 611158 | 44188 | 33.967 | 0.329 |
| M21 + RNA_28 | 1:2 | SARS- | 349955 | 52940 | 34.581 | 0.895 | 43638 | 2660 | 35.314 | 0.223 |
| | | CoV-2 | | | | | | | | |
| M21 + RNA_28 | 1:3 | InfA | 678481 | 32614 | 34.198 | 0.457 | 213637 | 52616 | 35.839 | 0.795 |
| M21 + RNA_28 | 1:3 | InfB | 256065 | 23213 | 34.427 | 0.572 | 45325 | 27715 | 36.290 | 1.707 |
| M21 + RNA_28 | 1:3 | RNaseP | 1025239 | 20646 | 33.155 | 0.057 | 728233 | 37613 | 33.634 | 0.178 |
| M21 + RNA_28 | 1:3 | SARS- | 425463 | 35587 | 33.988 | 0.660 | 30811 | 12096 | 35.688 | 0.190 |
| | | CoV-2 | | | | | | | | |
| M21 + RNA_28 | 1:5 | InfA | 675379 | 25472 | 33.941 | 0.569 | 311794 | 86197 | 35.481 | 0.850 |
| M21 + RNA_28 | 1:5 | InfB | 210977 | 9384 | 34.629 | 0.760 | 57274 | 16801 | 37.055 | 1.016 |
| M21 + RNA_28 | 1:5 | RNaseP | 1001414 | 132364 | 33.186 | 0.343 | 800110 | 23777 | 34.066 | 0.232 |
| M21 + RNA_28 | 1:5 | SARS- | 374218 | 38679 | 34.658 | 0.852 | 36512 | 3326 | 36.106 | 0.430 |
| | | CoV-2 | | | | | | | | |
| M21 + RNA_28 | 1:10 | InfA | 685649 | 24601 | 34.052 | 0.946 | 293656 | 57520 | 35.848 | 0.733 |
| M21 + RNA_28 | 1:10 | InfB | 273658 | 6968 | 34.392 | 0.788 | 79064 | 14467 | 36.211 | 0.658 |
| M21 + RNA_28 | 1:10 | RNaseP | 1003355 | 29034 | 33.145 | 0.292 | 710737 | 6301 | 34.177 | 0.303 |
| M21 + RNA_28 | 1:10 | SARS- | 389913 | 10979 | 33.989 | 0.764 | 45742 | 20742 | 35.201 | 0.340 |
| | | CoV-2 | | | | | | | | |
| M21 + RNA_30 | 1:2 | InfA | 707715 | 26037 | 33.754 | 0.341 | 290673 | 35480 | 35.776 | 0.264 |
| M21 + RNA_30 | 1:2 | InfB | 229560 | 22297 | 34.355 | 0.466 | 72694 | 7004 | 35.816 | 0.240 |
| M21 + RNA_30 | 1:2 | RNaseP | 929248 | 47661 | 33.679 | 0.274 | 708163 | 88313 | 34.083 | 0.526 |
| M21 + RNA_30 | 1:2 | SARS- | 344689 | 41088 | 34.198 | 0.488 | 36562 | 13417 | 37.352 | 2.932 |
| | | CoV-2 | | | | | | | | |
| M21 + RNA_30 | 1:3 | InfA | 656521 | 25650 | 33.954 | 0.368 | 168474 | 20535 | 37.088 | 0.360 |
| M21 + RNA_30 | 1:3 | InfB | 233587 | 42025 | 34.534 | 1.052 | 80025 | 17996 | 36.000 | 0.537 |
| M21 + RNA_30 | 1:3 | RNaseP | 988792 | 13495 | 33.338 | 0.353 | 785917 | 42394 | 33.710 | 0.211 |
| M21 + RNA_30 | 1:3 | SARS- | 308370 | 79298 | 34.992 | 0.974 | 57775 | 10779 | 35.111 | 0.530 |
| | | CoV-2 | | | | | | | | |
| M21 + RNA_30 | 1:5 | InfA | 719720 | 23451 | 34.274 | 0.082 | 337662 | 169670 | 36.369 | 1.979 |
| M21 + RNA_30 | 1:5 | InfB | 279861 | 23108 | 34.242 | 0.514 | 76050 | 24577 | 36.567 | 1.321 |
| M21 + RNA_30 | 1:5 | RNaseP | 1038633 | 76466 | 33.234 | 0.140 | 780940 | 65800 | 34.254 | 0.499 |
| M21 + RNA_30 | 1:5 | SARS- | 410180 | 8774 | 33.537 | 0.297 | 42528 | 7700 | 36.250 | 0.656 |
| | | CoV-2 | | | | | | | | |
| M21 + RNA_30 | 1:10 | InfA | 734277 | 48866 | 33.970 | 0.393 | 378855 | 44490 | 35.551 | 0.498 |
| M21 + RNA_30 | 1:10 | InfB | 268150 | 12904 | 34.507 | 0.366 | 100354 | 15581 | 35.901 | 0.687 |
| M21 + RNA_30 | 1:10 | RNaseP | 1020301 | 46895 | 33.601 | 0.075 | 810897 | 70025 | 34.062 | 0.584 |
| M21 + RNA_30 | 1:10 | SARS- | 364607 | 22144 | 33.950 | 0.879 | 66923 | 10056 | 35.294 | 0.552 |
| | | CoV-2 | | | | | | | | |
| M21 + RNA_31 | 1:2 | InfA | 634723 | 64056 | 34.457 | 0.334 | 320459 | 81818 | 35.538 | 0.564 |
| M21 + RNA_31 | 1:2 | InfB | 224986 | 48997 | 34.803 | 0.299 | 83309 | 26605 | 36.046 | 0.985 |
| M21 + RNA_31 | 1:2 | RNaseP | 841828 | 166262 | 33.735 | 0.326 | 733304 | 40025 | 34.056 | 0.196 |
| M21 + RNA_31 | 1:2 | SARS- | 361480 | 53247 | 34.500 | 0.146 | 51543 | 26117 | 36.831 | 1.843 |
| | | CoV-2 | | | | | | | | |
| M21 + RNA_31 | 1:3 | InfA | 775018 | 49012 | 33.806 | 0.424 | 382977 | 80527 | 35.198 | 0.800 |
| M21 + RNA_31 | 1:3 | InfB | 267421 | 29656 | 34.268 | 0.737 | 73162 | 14495 | 36.620 | 0.716 |
| M21 + RNA_31 | 1:3 | RNaseP | 1070984 | 19418 | 33.643 | 0.278 | 811226 | 38358 | 33.919 | 0.169 |
| M21 + RNA_31 | 1:3 | SARS- | 422000 | 26731 | 34.750 | 0.610 | 86529 | 14101 | 35.601 | 0.494 |
| | | CoV-2 | | | | | | | | |
| M21 + RNA_31 | 1:5 | InfA | 761497 | 33624 | 33.862 | 0.170 | 368704 | 96006 | 35.815 | 0.573 |
| M21 + RNA_31 | 1:5 | InfB | 301815 | 14165 | 34.295 | 0.150 | 124134 | 26010 | 35.501 | 0.536 |
| M21 + RNA_31 | 1:5 | RNaseP | 1064248 | 48964 | 33.888 | 0.155 | 866743 | 23553 | 33.716 | 0.178 |
| M21 + RNA_31 | 1:5 | SARS- | 492199 | 10023 | 33.695 | 0.101 | 114526 | 10067 | 35.250 | 0.170 |
| | | CoV-2 | | | | | | | | |
| M21 + RNA_31 | 1:10 | InfA | 713936 | 38119 | 34.522 | 0.681 | 288768 | 62367 | 36.531 | 0.464 |
| M21 + RNA_31 | 1:10 | InfB | 287241 | 31356 | 34.456 | 0.582 | 134102 | 12331 | 35.518 | 0.507 |
| M21 + RNA_31 | 1:10 | RNaseP | 981275 | 75963 | 33.967 | 0.160 | 771420 | 31547 | 34.015 | 0.250 |
| M21 + RNA_31 | 1:10 | SARS- | 371297 | 98296 | 34.682 | 1.067 | 116939 | 13219 | 35.558 | 0.402 |
| | | CoV-2 | | | | | | | | |
| M21 + RNA_32 | 1:2 | InfA | 718823 | 12450 | 34.244 | 0.252 | 240921 | 117339 | 35.998 | 1.469 |
| M21 + RNA_32 | 1:2 | InfB | 245950 | 21618 | 34.804 | 0.720 | 53813 | 20402 | 36.719 | 1.079 |
| M21 + RNA_32 | 1:2 | RNaseP | 969487 | 60846 | 33.737 | 0.653 | 639866 | 22639 | 33.954 | 0.121 |
| M21 + RNA_32 | 1:2 | SARS- | 434466 | 29489 | 33.938 | 0.073 | 117337 | 16847 | 35.259 | 0.532 |
| | | CoV-2 | | | | | | | | |
| M21 + RNA_32 | 1:3 | InfA | 691732 | 45153 | 33.996 | 0.354 | 264184 | 20947 | 35.868 | 0.402 |
| M21 + RNA_32 | 1:3 | InfB | 236965 | 45358 | 34.330 | 0.616 | 99438 | 22366 | 35.544 | 0.805 |
| M21 + RNA_32 | 1:3 | RNaseP | 1074991 | 64063 | 33.381 | 0.475 | 713576 | 43513 | 34.071 | 0.353 |
| M21 + RNA_32 | 1:3 | SARS- | 446005 | 15573 | 33.980 | 0.309 | 181399 | 39397 | 34.941 | 0.373 |
| | | CoV-2 | | | | | | | | |
| M21 + RNA_32 | 1:5 | InfA | 643153 | 84242 | 34.657 | 0.728 | 347482 | 42634 | 35.374 | 0.456 |
| M21 + RNA_32 | 1:5 | InfB | 281894 | 3898 | 34.006 | 0.303 | 89834 | 25305 | 35.936 | 1.192 |
| M21 + RNA_32 | 1:5 | RNaseP | 1026592 | 39654 | 33.490 | 0.411 | 777867 | 72044 | 33.934 | 0.210 |
| M21 + RNA_32 | 1:5 | SARS- | 454850 | 28622 | 34.217 | 0.472 | 119432 | 32359 | 35.776 | 0.348 |
| | | CoV-2 | | | | | | | | |
| M21 + RNA_32 | 1:10 | InfA | 514663 | 339608 | 37.595 | 5.456 | 284547 | 83988 | 36.068 | 0.737 |
| M21 + RNA_32 | 1:10 | InfB | 200524 | 114653 | 36.353 | 2.903 | 69045 | 29855 | 37.183 | 2.145 |
| M21 + RNA_32 | 1:10 | RNaseP | 794241 | 271741 | 35.272 | 2.695 | 719997 | 38535 | 34.175 | 0.459 |
| M21 + RNA_32 | 1:10 | SARS- | 326408 | 138811 | 35.734 | 2.091 | 144312 | 38818 | 35.713 | 0.800 |
| | | CoV-2 | | | | | | | | |
| M21 + RNA_39 | 1:2 | InfA | 667710 | 40163 | 34.452 | 0.466 | 199897 | 58754 | 35.656 | 1.007 |
| M21 + RNA_39 | 1:2 | InfB | 245953 | 40235 | 35.164 | 0.593 | 68045 | 8593 | 35.315 | 0.349 |
| M21 + RNA_39 | 1:2 | RNaseP | 978015 | 49047 | 33.703 | 0.495 | 608622 | 25879 | 34.382 | 0.316 |
| M21 + RNA_39 | 1:2 | SARS- | 410968 | 33447 | 34.685 | 0.729 | 87810 | 27267 | 35.304 | 0.912 |
| | | CoV-2 | | | | | | | | |
| M21 + RNA_39 | 1:3 | InfA | 723699 | 41104 | 33.939 | 0.428 | 230728 | 65706 | 35.757 | 0.933 |
| M21 + RNA_39 | 1:3 | InfB | 281746 | 22602 | 34.088 | 0.235 | 66539 | 14034 | 35.936 | 0.788 |
| M21 + RNA_39 | 1:3 | RNaseP | 1106073 | 78199 | 33.038 | 0.218 | 714976 | 10050 | 33.922 | 0.054 |
| M21 + RNA_39 | 1:3 | SARS- | 450561 | 21757 | 34.141 | 0.535 | 96668 | 18689 | 35.445 | 0.309 |
| | | CoV-2 | | | | | | | | |
| M21 + RNA_39 | 1:5 | InfA | 726074 | 32095 | 34.264 | 0.351 | 232450 | 51068 | 35.859 | 0.618 |
| M21 + RNA_39 | 1:5 | InfB | 230198 | 34389 | 35.106 | 0.605 | 97200 | 5525 | 35.405 | 0.259 |
| M21 + RNA_39 | 1:5 | RNaseP | 1122980 | 34440 | 33.202 | 0.712 | 751171 | 33771 | 34.119 | 0.453 |
| M21 + RNA_39 | 1:5 | SARS- | 513720 | 10143 | 33.666 | 0.209 | 128095 | 30027 | 35.272 | 0.591 |
| | | CoV-2 | | | | | | | | |
| M21 + RNA_39 | 1:10 | InfA | 763449 | 36526 | 34.123 | 0.432 | 193701 | 153873 | 36.011 | 1.691 |
| M21 + RNA_39 | 1:10 | InfB | 269390 | 34632 | 34.708 | 0.923 | 45231 | 24315 | 36.849 | 0.776 |
| M21 + RNA_39 | 1:10 | RNaseP | 983237 | 38929 | 33.625 | 0.315 | 543016 | 253897 | 36.079 | 3.358 |
| M21 + RNA_39 | 1:10 | SARS- | 375434 | 27975 | 34.836 | 0.273 | 82964 | 32050 | 36.534 | 1.446 |
| | | CoV-2 | | | | | | | | |
| M21 + RNA_40 | 1:2 | InfA | 750904 | 27655 | 33.877 | 0.265 | 280354 | 56542 | 36.170 | 0.971 |
| M21 + RNA_40 | 1:2 | InfB | 277892 | 19643 | 34.382 | 0.492 | 74238 | 17249 | 36.093 | 0.678 |
| M21 + RNA_40 | 1:2 | RNaseP | 963731 | 83906 | 33.667 | 0.302 | 689422 | 48993 | 34.439 | 0.453 |
| M21 + RNA_40 | 1:2 | SARS- | 439007 | 56145 | 34.312 | 0.646 | 162524 | 37604 | 35.489 | 0.431 |
| | | CoV-2 | | | | | | | | |
| M21 + RNA_40 | 1:3 | InfA | 750615 | 26361 | 33.996 | 0.405 | 336253 | 30064 | 35.580 | 0.274 |
| M21 + RNA_40 | 1:3 | InfB | 245616 | 8486 | 35.070 | 0.415 | 118642 | 24118 | 35.298 | 0.327 |
| M21 + RNA_40 | 1:3 | RNaseP | 1154469 | 31159 | 33.374 | 0.155 | 776236 | 10626 | 34.025 | 0.275 |
| M21 + RNA_40 | 1:3 | SARS- | 507106 | 15575 | 34.255 | 0.232 | 181634 | 31841 | 35.440 | 0.474 |
| | | CoV-2 | | | | | | | | |
| M21 + RNA_40 | 1:5 | InfA | 735251 | 35393 | 33.996 | 0.224 | 360510 | 28212 | 35.667 | 0.357 |
| M21 + RNA_40 | 1:5 | InfB | 234897 | 51630 | 34.590 | 0.774 | 99913 | 21648 | 35.875 | 0.810 |
| M21 + RNA_40 | 1:5 | RNaseP | 1051906 | 62740 | 33.470 | 0.435 | 791821 | 64043 | 34.042 | 0.457 |
| M21 + RNA_40 | 1:5 | SARS- | 491548 | 54421 | 34.340 | 0.915 | 220637 | 46166 | 35.046 | 0.334 |
| | | CoV-2 | | | | | | | | |
| M21 + RNA_40 | 1:10 | InfA | 746878 | 70902 | 34.428 | 1.055 | 368378 | 81620 | 35.580 | 0.690 |
| M21 + RNA_40 | 1:10 | InfB | 278317 | 51865 | 34.450 | 1.033 | 116894 | 8989 | 35.609 | 0.345 |
| M21 + RNA_40 | 1:10 | RNaseP | 958047 | 26552 | 33.718 | 0.152 | 765411 | 54483 | 34.231 | 0.436 |
| M21 + RNA_40 | 1:10 | SARS- | 435718 | 36222 | 34.280 | 0.268 | 179906 | 25624 | 35.324 | 0.470 |
| | | CoV-2 | | | | | | | | |
| M21 + RNA_42 | 1:2 | InfA | 750743 | 56237 | 34.047 | 0.206 | 312792 | 11559 | 35.531 | 0.327 |
| M21 + RNA_42 | 1:2 | InfB | 265698 | 23948 | 34.353 | 0.704 | 102182 | 21663 | 35.121 | 0.693 |
| M21 + RNA_42 | 1:2 | RNaseP | 1061179 | 97579 | 33.140 | 0.450 | 746562 | 80652 | 33.821 | 0.496 |
| M21 + RNA_42 | 1:2 | SARS- | 390504 | 35069 | 34.459 | 0.109 | 119845 | 24325 | 35.209 | 0.634 |
| | | CoV-2 | | | | | | | | |
| M21 + RNA_42 | 1:3 | InfA | 724729 | 98105 | 34.151 | 0.545 | 314891 | 97640 | 35.730 | 0.497 |
| M21 + RNA_42 | 1:3 | InfB | 259600 | 52395 | 34.211 | 0.875 | 56280 | 13864 | 36.966 | 1.027 |
| M21 + RNA_42 | 1:3 | RNaseP | 1126896 | 104576 | 33.335 | 0.244 | 810958 | 28651 | 33.780 | 0.419 |
| M21 + RNA_42 | 1:3 | SARS- | 448974 | 57136 | 33.783 | 0.722 | 119821 | 20404 | 35.268 | 0.388 |
| | | CoV-2 | | | | | | | | |
| M21 + RNA_42 | 1:5 | InfA | 725247 | 129705 | 34.059 | 1.040 | 344660 | 149493 | 36.073 | 1.878 |
| M21 + RNA_42 | 1:5 | InfB | 267450 | 12447 | 34.242 | 0.244 | 118430 | 12556 | 35.478 | 0.568 |
| M21 + RNA_42 | 1:5 | RNaseP | 1086503 | 47036 | 33.385 | 0.449 | 814441 | 11813 | 34.111 | 0.264 |
| M21 + RNA_42 | 1:5 | SARS- | 437249 | 9920 | 33.871 | 0.387 | 92829 | 23330 | 36.298 | 0.834 |
| | | CoV-2 | | | | | | | | |
| M21 + RNA_42 | 1:10 | InfA | 759634 | 24532 | 34.196 | 0.255 | 405040 | 58063 | 35.400 | 0.361 |
| M21 + RNA_42 | 1:10 | InfB | 292813 | 28809 | 34.689 | 1.042 | 115387 | 25167 | 35.839 | 0.933 |
| M21 + RNA_42 | 1:10 | RNaseP | 1056418 | 46862 | 33.587 | 0.409 | 782455 | 36608 | 34.278 | 0.409 |
| M21 + RNA_42 | 1:10 | SARS- | 417345 | 41554 | 34.185 | 0.435 | 203596 | 23900 | 34.633 | 0.417 |
| | | CoV-2 | | | | | | | | |
| M21 + RNA_43 | 1:2 | InfA | 774277 | 19875 | 33.682 | 0.303 | 255493 | 38550 | 35.356 | 0.361 |
| M21 + RNA_43 | 1:2 | InfB | 246977 | 45132 | 34.638 | 0.570 | 80610 | 6299 | 35.039 | 0.222 |
| M21 + RNA_43 | 1:2 | RNaseP | 1038195 | 100634 | 33.267 | 0.439 | 516292 | 84249 | 34.206 | 0.775 |
| M21 + RNA_43 | 1:2 | SARS- | 501508 | 33046 | 33.755 | 0.223 | 101231 | 25710 | 35.369 | 0.507 |
| | | CoV-2 | | | | | | | | |
| M21 + RNA_43 | 1:3 | InfA | 590644 | 86690 | 35.014 | 0.771 | 253438 | 59678 | 35.646 | 0.792 |
| M21 + RNA_43 | 1:3 | InfB | 268767 | 53906 | 34.466 | 1.217 | 51762 | 28217 | 37.637 | 2.811 |
| M21 + RNA_43 | 1:3 | RNaseP | 1018535 | 129311 | 33.316 | 0.463 | 619334 | 44594 | 33.804 | 0.112 |
| M21 + RNA_43 | 1:3 | SARS- | 462903 | 27496 | 34.393 | 0.235 | 105543 | 23878 | 35.646 | 0.541 |
| | | CoV-2 | | | | | | | | |
| M21 + RNA_43 | 1:5 | InfA | 758752 | 34670 | 34.037 | 0.089 | 240637 | 52648 | 36.305 | 0.518 |
| M21 + RNA_43 | 1:5 | InfB | 277076 | 44597 | 34.422 | 0.849 | 94793 | 5261 | 35.247 | 0.363 |
| M21 + RNA_43 | 1:5 | RNaseP | 1027438 | 16600 | 33.367 | 0.124 | 687689 | 35665 | 33.790 | 0.233 |
| M21 + RNA_43 | 1:5 | SARS- | 414505 | 83712 | 34.591 | 0.785 | 115892 | 44402 | 36.170 | 1.001 |
| | | CoV-2 | | | | | | | | |
| M21 + RNA_43 | 1:10 | InfA | 734080 | 27152 | 34.119 | 0.303 | 281494 | 69160 | 36.060 | 0.781 |
| M21 + RNA_43 | 1:10 | InfB | 274349 | 13669 | 34.546 | 0.320 | 89736 | 17691 | 35.957 | 0.458 |
| M21 + RNA_43 | 1:10 | RNaseP | 947890 | 65832 | 34.075 | 0.598 | 663168 | 11885 | 33.832 | 0.244 |
| M21 + RNA_43 | 1:10 | SARS- | 417858 | 55872 | 34.752 | 0.586 | 129592 | 54120 | 35.691 | 1.189 |
| | | CoV-2 | | | | | | | | |
| M21 + RNA_44 | 1:2 | InfA | 780899 | 48568 | 33.875 | 0.698 | 173145 | 191266 | 36.732 | 2.989 |
| M21 + RNA_44 | 1:2 | InfB | 247649 | 32241 | 34.615 | 0.570 | 70052 | 30456 | 36.621 | 2.099 |
| M21 + RNA_44 | 1:2 | RNaseP | 1011647 | 56134 | 33.519 | 0.304 | 727727 | 54859 | 33.920 | 0.278 |
| M21 + RNA_44 | 1:2 | SARS- | 437111 | 21744 | 34.625 | 0.128 | 84043 | 21479 | 35.310 | 0.842 |
| | | CoV-2 | | | | | | | | |
| M21 + RNA_44 | 1:3 | InfA | 742688 | 20571 | 34.252 | 0.279 | 349679 | 75538 | 35.789 | 0.651 |
| M21 + RNA_44 | 1:3 | InfB | 286876 | 20637 | 34.488 | 0.517 | 88871 | 25832 | 36.532 | 0.924 |
| M21 + RNA_44 | 1:3 | RNaseP | 1193301 | 49960 | 33.072 | 0.293 | 791839 | 84091 | 34.244 | 0.418 |
| M21 + RNA_44 | 1:3 | SARS- | 449125 | 22861 | 34.157 | 0.507 | 81783 | 10407 | 35.912 | 0.267 |
| | | CoV-2 | | | | | | | | |
| M21 + RNA_44 | 1:5 | InfA | 730552 | 28930 | 34.437 | 0.205 | 490191 | 57311 | 35.280 | 0.505 |
| M21 + RNA_44 | 1:5 | InfB | 273052 | 31329 | 34.709 | 0.670 | 110575 | 6911 | 35.873 | 0.095 |
| M21 + RNA_44 | 1:5 | RNaseP | 1122354 | 58740 | 33.321 | 0.279 | 778263 | 59630 | 34.588 | 0.343 |
| M21 + RNA_44 | 1:5 | SARS- | 424727 | 25995 | 33.873 | 0.044 | 89175 | 13628 | 36.055 | 0.537 |
| | | CoV-2 | | | | | | | | |
| M21 + RNA_44 | 1:10 | InfA | 688290 | 23600 | 34.642 | 0.613 | 431710 | 94228 | 36.008 | 0.922 |
| M21 + RNA_44 | 1:10 | InfB | 272934 | 61408 | 34.488 | 0.583 | 109944 | 39759 | 36.550 | 1.595 |
| M21 + RNA_44 | 1:10 | RNaseP | 908770 | 179279 | 34.101 | 0.354 | 863339 | 45062 | 34.343 | 0.387 |
| M21 + RNA_44 | 1:10 | SARS- | 422155 | 53132 | 34.169 | 0.485 | 100758 | 32422 | 36.277 | 1.119 |
| | | CoV-2 | | | | | | | | |
| RNA Aptamer Only | 1:15 | InfA | 627250 | 37136 | 34.161 | 0.901 | 111376 | 13843 | 37.294 | 0.527 |
| RNA Aptamer Only | 1:15 | InfB | 191503 | 46367 | 34.509 | 0.596 | 5668 | 2383 | NA | NA |
| RNA Aptamer Only | 1:15 | RNaseP | 809823 | 68690 | 33.484 | 0.208 | 231525 | 2227 | 34.287 | 0.042 |
| RNA Aptamer Only | 1:15 | SARS- | 369173 | 29218 | 33.532 | 0.356 | 13862 | 1973 | NA | NA |
| | | CoV-2 | | | | | | | | |
| No Aptamer | | InfA | 448615 | 55557 | 35.409 | 0.588 | 1888 | 490 | NA | NA |
| No Aptamer | | InfB | 171827 | 23476 | 34.971 | 0.037 | 316 | 72 | NA | NA |
| No Aptamer | | RNaseP | 732356 | 34459 | 34.503 | 0.191 | 37185 | 51557 | 39.532 | NA |
| No Aptamer | | SARS- | 313130 | 6117 | 34.866 | 0.355 | 359 | 115 | NA | NA |
| | | CoV-2 | | | | | | | | |
| No Template Control | | InfA | 135651 | 129749 | 38.145 | 0.500 | 2671 | 896 | NA | NA |
| No Template Control | | InfB | 4537 | 3746 | NA | NA | 396 | 68 | NA | NA |
| No Template Control | | RNaseP | 2585 | 1047 | NA | NA | 2516 | 271 | NA | NA |
| No Template Control | | SARS- | 1266 | 748 | NA | NA | 494 | 63 | NA | NA |
| | | CoV-2 | | | | | | | | |

TABLE 6
Evaluation of room temperature stability at 0 hours and 4 hours following template addition to PrimeTime ™ One-
Step RT-qPCR Master Mix (Integrated DNA Technologies) with combinations of DNA aptamers.

| | | | | | | | 4 h Endpoint | | | |
|---|---|---|---|---|---|---|---|---|---|---|
| | | | 0 h Endpoint | | 0 h Cycle | | Fluorescence | | 4 h Cycle | |
| | | | Fluorescence | | Threshold | | Delta | | Threshold | |
| | | Target | Delta Rn | Delta Rn | Ct | | Rn | Delta Rn | Ct | Ct |
| Aptamer 1 | Aptamer 2 | Name | Mean | SD | Mean | Ct SD | Mean | SD | Mean | SD |
| M21_28 | M21_44 | InfA | 841,284 | 16,549 | 35.026 | 0.360 | 290,120 | 65,209 | 37.264 | 0.533 |
| M21_28 | M21_44 | InfB | 301,390 | 7,993 | 35.081 | 0.339 | 118,071 | 13,354 | 35.789 | 0.281 |
| M21_28 | M21_44 | RNaseP | 1,019,772 | 120,778 | 35.200 | 0.980 | 713,593 | 38,786 | 35.173 | 0.218 |
| M21_28 | M21_44 | SARS-CoV- | 430,505 | 10,819 | 35.106 | 0.256 | 99,192 | 30,600 | 36.567 | 1.054 |
| | | 2 | | | | | | | | |
| M21_28 | M21 + RNA_9 | InfA | 749,212 | 33,936 | 35.672 | 0.322 | 461,963 | 83,506 | 36.526 | 0.661 |
| M21_28 | M21 + RNA_9 | InfB | 304,529 | 18,551 | 35.134 | 0.481 | 114,432 | 20,590 | 36.896 | 0.480 |
| M21_28 | M21 + RNA_9 | RNaseP | 1,043,202 | 83,933 | 35.583 | 0.550 | 799,687 | 7,550 | 35.483 | 0.165 |
| M21_28 | M21 + RNA_9 | SARS-CoV- | 408,627 | 21,546 | 35.277 | 0.610 | 174,087 | 11,825 | 35.735 | 0.308 |
| | | 2 | | | | | | | | |
| M21_28 | M21 + RNA_21 | InfA | 732,838 | 89,981 | 35.638 | 0.925 | 270,018 | 171,385 | 36.531 | 0.071 |
| M21_28 | M21 + RNA_21 | InfB | 282,019 | 22,299 | 35.546 | 0.312 | 95,947 | 14,447 | 36.543 | 0.589 |
| M21_28 | M21 + RNA_21 | RNaseP | 1,072,799 | 41,205 | 35.108 | 0.271 | 774,859 | 101,813 | 34.970 | 0.551 |
| M21_28 | M21 + RNA_21 | SARS-CoV- | 379,493 | 54,761 | 35.794 | 0.654 | 95,184 | 32,554 | 36.160 | 0.623 |
| | | 2 | | | | | | | | |
| M21_28 | M21 + RNA_25 | InfA | 744,726 | 88,568 | 35.472 | 0.735 | 404,284 | 55,050 | 36.257 | 0.427 |
| M21_28 | M21 + RNA_25 | InfB | 270,755 | 13,382 | 34.972 | 0.180 | 105,434 | 38,956 | 36.527 | 1.862 |
| M21_28 | M21 + RNA_25 | RNaseP | 1,123,468 | 29,154 | 35.000 | 0.283 | 672,814 | 118,566 | 35.723 | 1.124 |
| | | SARS-CoV- | 436,100 | 24,761 | 35.194 | 0.349 | 153,632 | 32,951 | 35.720 | 0.330 |
| M21_28 | M21 + RNA_25 | 2 | | | | | | | | |
| M21_28 | M21 + RNA_27 | InfA | 755,071 | 13,470 | 35.679 | 0.189 | 340,598 | 5,310 | 36.873 | 0.150 |
| M21_28 | M21 + RNA_27 | InfB | 325,670 | 21,068 | 34.745 | 0.361 | 120,005 | 16,880 | 35.984 | 0.422 |
| M21_28 | M21 + RNA_27 | RNaseP | 1,115,896 | 23,408 | 34.983 | 0.265 | 800,384 | 85,736 | 35.100 | 0.362 |
| | M21 + RNA_27 | SARS-CoV- | 422,968 | 29,015 | 35.457 | 0.574 | 125,598 | 31,682 | 36.145 | 0.739 |
| M21_28 | | 2 | | | | | | | | |
| M21_28 | M21 + RNA_28 | InfA | 774,436 | 20,397 | 35.716 | 0.823 | 284,427 | 28,350 | 37.315 | 0.259 |
| M21_28 | M21 + RNA_28 | InfB | 306,625 | 21,646 | 34.942 | 0.390 | 97,042 | 24,838 | 36.320 | 0.792 |
| M21_28 | M21 + RNA_28 | RNaseP | 1,017,589 | 65,216 | 35.461 | 0.108 | 767,091 | 48,546 | 35.173 | 0.304 |
| | | SARS-CoV- | | | | | | | | |
| M21_28 | M21 + RNA_28 | 2 | 428,128 | 36,722 | 35.370 | 0.351 | 87,708 | 29,683 | 35.753 | 0.845 |
| M21_28 | M21 + RNA_30 | InfA | 733,542 | 56,827 | 35.301 | 0.537 | 412,565 | 31,022 | 36.087 | 0.489 |
| M21_28 | M21 + RNA_30 | InfB | 291,250 | 32,814 | 34.923 | 0.407 | 89,290 | 21,024 | 36.733 | 0.797 |
| M21_28 | M21 + RNA_30 | RNaseP | 1,015,274 | 24,659 | 35.036 | 0.155 | 752,796 | 71,052 | 34.977 | 0.263 |
| | | SARS-CoV- | | | | | | | | |
| M21_28 | M21 + RNA_30 | 2 | 418,270 | 25,523 | 35.320 | 0.445 | 38,942 | 13,514 | 37.210 | 1.159 |
| M21_28 | M21 + RNA_31 | InfA | 702,981 | 98,976 | 35.903 | 0.615 | 396,926 | 85,129 | 36.567 | 1.056 |
| M21_28 | M21 + RNA_31 | InfB | 297,621 | 37,412 | 35.141 | 0.571 | 121,453 | 2,161 | 35.949 | 0.004 |
| M21_28 | M21 + RNA_31 | RNaseP | 1,071,617 | 55,254 | 35.173 | 0.109 | 755,958 | 48,239 | 35.078 | 0.419 |
| M21_28 | M21 + RNA_31 | SARS-CoV- | 395,843 | 62,414 | 35.213 | 0.270 | 118,028 | 28,138 | 35.822 | 0.681 |
| | | 2 | | | | | | | | |
| M21_28 | M21 + RNA_32 | InfA | 785,503 | 17,739 | 35.152 | 0.525 | 362,731 | 26,352 | 36.830 | 0.318 |
| M21_28 | M21 + RNA_32 | InfB | 287,755 | 7,240 | 35.016 | 0.717 | 106,198 | 25,734 | 36.636 | 0.546 |
| M21_28 | M21 + RNA_32 | RNaseP | 1,088,474 | 71,666 | 34.884 | 0.135 | 747,348 | 66,922 | 35.317 | 0.573 |
| M21_28 | M21 + RNA_32 | SARS-CoV- | 446,718 | 42,716 | 34.865 | 0.795 | 177,719 | 41,527 | 35.974 | 0.263 |
| | | 2 | | | | | | | | |
| M21_28 | M21 + RNA_39 | InfA | 721,732 | 98,360 | 35.966 | 0.733 | 334,287 | 130,448 | 37.072 | 1.468 |
| M21_28 | M21 + RNA_39 | InfB | 279,639 | 23,892 | 35.209 | 0.738 | 84,157 | 26,430 | 36.923 | 0.833 |
| M21_28 | M21 + RNA_39 | RNaseP | 1,079,644 | 14,267 | 34.837 | 0.214 | 829,075 | 16,864 | 35.288 | 0.234 |
| | | SARS-CoV- | | | | | | | | |
| M21_28 | M21 + RNA_39 | 2 | 430,423 | 7,593 | 35.346 | 0.079 | 193,940 | 6,695 | 35.395 | 0.220 |
| M21_28 | M21 + RNA_40 | InfA | 754,767 | 68,319 | 35.513 | 0.654 | 252,155 | 47,238 | 37.968 | 0.698 |
| M21_28 | M21 + RNA_40 | InfB | 278,980 | 28,570 | 35.380 | 0.430 | 84,708 | 41,095 | 38.275 | 3.530 |
| M21_28 | M21 + RNA_40 | RNaseP | 1,176,683 | 50,759 | 34.820 | 0.184 | 824,694 | 131,808 | 35.049 | 0.329 |
| | | SARS-CoV- | | | | | | | | |
| M21_28 | M21 + RNA_40 | 2 | 422,755 | 35,821 | 35.584 | 0.059 | 145,003 | 63,642 | 36.034 | 0.596 |
| M21_28 | M21 + RNA_42 | InfA | 748,860 | 42,852 | 35.398 | 0.171 | 388,250 | 40,545 | 36.618 | 0.388 |
| M21_28 | M21 + RNA_42 | InfB | 301,783 | 13,818 | 35.025 | 0.151 | 126,609 | 20,043 | 35.957 | 0.562 |
| M21_28 | M21 + RNA_42 | RNaseP | 1,129,201 | 58,069 | 35.040 | 0.481 | 794,409 | 60,612 | 35.241 | 0.273 |
| | | SARS-CoV- | 421,196 | 11,235 | 35.438 | 0.199 | 170,377 | 18,300 | 35.731 | 0.267 |
| M21_28 | M21 + RNA_42 | 2 | | | | | | | | |
| M21_28 | M21 + RNA_43 | InfA | 796,872 | 48,826 | 35.551 | 0.488 | 362,522 | 102,422 | 37.155 | 1.182 |
| M21_28 | M21 + RNA_43 | InfB | 315,227 | 14,998 | 34.575 | 0.304 | 102,257 | 30,733 | 36.506 | 0.933 |
| M21_28 | M21 + RNA_43 | RNaseP | 1,153,161 | 86,560 | 34.627 | 0.220 | 709,243 | 87,092 | 35.485 | 0.482 |
| M21_28 | M21 + RNA_43 | SARS-CoV- | 478,206 | 20,401 | 35.060 | 0.127 | 191,885 | 24,505 | 35.552 | 0.301 |
| | | 2 | | | | | | | | |
| M21_28 | M21 + RNA_44 | InfA | 792,881 | 92,012 | 35.913 | 0.231 | 411,837 | 87,403 | 37.049 | 0.666 |
| M21_28 | M21 + RNA_44 | InfB | 301,427 | 27,709 | 35.306 | 0.357 | 109,552 | 21,894 | 36.866 | 0.986 |
| M21_28 | M21 + RNA_44 | RNaseP | 1,239,276 | 69,360 | 35.281 | 0.375 | 779,278 | 10,299 | 35.545 | 0.225 |
| M21_28 | M21 + RNA_44 | SARS-CoV- | 470,088 | 36,272 | 34.982 | 0.257 | 105,219 | 27,740 | 35.999 | 0.408 |
| | | 2 | | | | | | | | |
| M21_44 | M21 + RNA_9 | InfA | 716,961 | 49,939 | 35.514 | 0.761 | 475,806 | 17,397 | 36.396 | 0.232 |
| M21_44 | M21 + RNA_9 | InfB | 283,066 | 47,082 | 35.226 | 0.493 | 126,229 | 19,340 | 36.466 | 0.396 |
| M21_44 | M21 + RNA_9 | RNaseP | 1,061,497 | 66,272 | 35.176 | 0.225 | 792,285 | 15,666 | 35.504 | 0.138 |
| | | SARS-CoV- | | | | | | | | |
| M21_44 | M21 + RNA_9 | 2 | 383,440 | 11,546 | 35.861 | 0.054 | 158,241 | 13,276 | 36.111 | 0.406 |
| M21_44 | M21 + RNA_21 | InfA | 713,494 | 68,780 | 35.644 | 0.480 | 302,769 | 107,951 | 37.590 | 1.536 |
| M21_44 | M21 + RNA_21 | InfB | 259,152 | 27,352 | 34.988 | 0.490 | 108,577 | 15,949 | 36.593 | 0.302 |
| M21_44 | M21 + RNA_21 | RNaseP | 1,038,790 | 77,498 | 35.159 | 0.438 | 740,895 | 115,165 | 35.508 | 0.560 |
| M21_44 | M21 + RNA_21 | SARS-CoV- | 425,628 | 26,141 | 35.081 | 0.459 | 142,726 | 75,413 | 36.119 | 1.133 |
| | | 2 | | | | | | | | |
| M21_44 | M21 + RNA_25 | InfA | 776,792 | 14,420 | 35.056 | 0.234 | 368,648 | 26,699 | 36.678 | 0.178 |
| M21_44 | M21 + RNA_25 | InfB | 279,490 | 16,121 | 35.428 | 0.402 | 88,405 | 16,256 | 37.048 | 0.797 |
| M21_44 | M21 + RNA_25 | RNaseP | 1,081,146 | 22,272 | 34.701 | 0.165 | 821,558 | 14,209 | 35.163 | 0.255 |
| | | SARS-CoV- | 399,004 | 57,871 | 35.539 | 0.670 | 162,233 | 17,271 | 35.774 | 0.496 |
| M21_44 | M21 + RNA_25 | 2 | | | | | | | | |
| M21_44 | M21 + RNA_27 | InfA | 756,045 | 16,607 | 35.643 | 0.223 | 413,076 | 24,819 | 36.323 | 0.267 |
| M21_44 | M21 + RNA_27 | InfB | 276,157 | 26,414 | 35.183 | 0.368 | 117,624 | 18,983 | 36.027 | 0.650 |
| M21_44 | M21 + RNA_27 | RNaseP | 1,197,218 | 63,943 | 34.572 | 0.257 | 922,126 | 23,881 | 34.688 | 0.278 |
| M21_44 | M21 + RNA_27 | SARS-CoV- | 458,758 | 23,042 | 35.090 | 0.507 | 149,873 | 29,580 | 35.941 | 0.253 |
| | | 2 | | | | | | | | |
| M21_44 | M21 + RNA_28 | InfA | 741,801 | 34,214 | 35.348 | 0.504 | 373,598 | 86,135 | 36.312 | 0.919 |
| M21_44 | M21 + RNA_28 | InfB | 262,172 | 18,180 | 35.202 | 0.261 | 87,481 | 34,055 | 36.846 | 1.666 |
| M21_44 | M21 + RNA_28 | RNaseP | 1,158,911 | 32,007 | 34.592 | 0.322 | 767,886 | 56,581 | 34.938 | 0.040 |
| M21_44 | M21 + RNA_28 | SARS-CoV- | 442,378 | 46,780 | 34.826 | 0.102 | 58,373 | 41,144 | 36.490 | 1.737 |
| | | 2 | | | | | | | | |
| M21_44 | M21 + RNA_30 | InfA | 710,443 | 43,005 | 36.281 | 0.640 | 383,349 | 72,255 | 36.978 | 0.726 |
| M21_44 | M21 + RNA_30 | InfB | 310,387 | 16,804 | 35.063 | 0.230 | 120,022 | 14,523 | 36.236 | 0.649 |
| M21_44 | M21 + RNA_30 | RNaseP | 1,205,801 | 29,597 | 34.880 | 0.078 | 856,442 | 52,194 | 35.003 | 0.276 |
| M21_44 | M21 + RNA_30 | SARS-CoV- | 399,617 | 27,307 | 35.263 | 0.421 | 56,218 | 12,245 | 36.913 | 0.921 |
| | | 2 | | | | | | | | |
| M21_44 | M21 + RNA_31 | InfA | 772,397 | 60,427 | 35.638 | 0.757 | 476,613 | 25,993 | 36.181 | 0.327 |
| M21_44 | M21 + RNA_31 | InfB | 239,655 | 16,804 | 36.036 | 0.939 | 97,112 | 8,610 | 36.781 | 0.338 |
| M21_44 | M21 + RNA_31 | RNaseP | 1,187,999 | 23,934 | 34.966 | 0.396 | 913,030 | 58,104 | 35.217 | 0.230 |
| | | SARS-CoV- | | 49,403 | 35.187 | 0.586 | 121,738 | 17,937 | 35.715 | 0.700 |
| M21_44 | M21 + RNA_31 | 2 | 439,032 | | | | | | | |
| M21_44 | M21 + RNA_32 | InfA | 744,250 | 35,752 | 35.832 | 0.418 | 438,080 | 48,570 | 36.401 | 0.503 |
| M21_44 | M21 + RNA_32 | InfB | 270,961 | 33,703 | 35.320 | 0.507 | 136,411 | 21,239 | 36.143 | 0.664 |
| M21_44 | M21 + RNA_32 | RNaseP | 1,130,450 | 126,707 | 35.199 | 0.560 | 886,585 | 84,796 | 34.960 | 0.453 |
| M21_44 | M21 + RNA_32 | SARS-CoV- | 455,714 | 31,062 | 35.094 | 0.224 | 171,254 | 46,218 | 35.816 | 0.725 |
| | | 2 | | | | | | | | |
| M21_44 | M21 + RNA_39 | InfA | 735,784 | 49,898 | 35.646 | 0.132 | 413,879 | 64,905 | 35.993 | 0.536 |
| M21_44 | M21 + RNA_39 | InfB | 268,828 | 15,062 | 35.416 | 0.500 | 84,825 | 23,916 | 36.986 | 0.952 |
| M21_44 | M21 + RNA_39 | RNaseP | 1,139,113 | 27,009 | 34.981 | 0.196 | 861,969 | 61,901 | 35.178 | 0.087 |
| | | SARS-CoV- | 464,743 | 16,806 | 34.940 | 0.281 | 121,239 | 16,552 | 36.114 | 0.664 |
| M21_44 | M21 + RNA_39 | 2 | | | | | | | | |
| M21_44 | M21 + RNA_40 | InfA | 664,856 | 21,255 | 35.773 | 0.303 | 473,635 | 58,697 | 36.116 | 0.682 |
| M21_44 | M21 + RNA_40 | InfB | 275,896 | 27,544 | 35.031 | 0.981 | 121,000 | 31,422 | 36.567 | 0.856 |
| M21_44 | M21 + RNA_40 | RNaseP | 1,167,951 | 30,391 | 34.879 | 0.341 | 875,030 | 20,722 | 35.276 | 0.183 |
| | M21 + RNA_40 | SARS-CoV- | 442,310 | 20,599 | 34.901 | 0.341 | 160,533 | 31,529 | 36.249 | 0.471 |
| M21_44 | | 2 | | | | | | | | |
| M21_44 | M21 + RNA_42 | InfA | 761,859 | 22,968 | 35.538 | 0.339 | 435,693 | 27,206 | 35.970 | 0.377 |
| M21_44 | M21 + RNA_42 | InfB | 303,766 | 24,681 | 34.721 | 0.635 | 116,328 | 3,652 | 35.861 | 0.499 |
| M21_44 | M21 + RNA_42 | RNaseP | 1,150,653 | 70,985 | 34.961 | 0.201 | 823,343 | 85,583 | 35.074 | 0.207 |
| | | SARS-CoV- | 442,571 | 31,001 | 35.153 | 0.593 | 118,475 | 62,654 | 36.395 | 1.523 |
| M21_44 | M21 + RNA_42 | 2 | | | | | | | | |
| M21_44 | M21 + RNA_43 | InfA | 807,020 | 63,315 | 35.403 | 0.656 | 431,860 | 78,755 | 36.325 | 0.863 |
| M21_44 | M21 + RNA_43 | InfB | 276,459 | 18,182 | 35.364 | 0.348 | 122,677 | 17,962 | 35.949 | 0.564 |
| M21_44 | M21 + RNA_43 | RNaseP | 1,249,153 | 6,010 | 34.436 | 0.127 | 773,874 | 74,124 | 34.963 | 0.172 |
| | | SARS-CoV- | 483,204 | 44,050 | 34.746 | 0.300 | 150,085 | 24,339 | 36.006 | 0.433 |
| M21_44 | M21 + RNA_43 | 2 | | | | | | | | |
| M21_44 | M21 + RNA_44 | InfA | 694,333 | 84,308 | 36.542 | 0.767 | 452,948 | 89,984 | 36.530 | 0.924 |
| M21_44 | M21 + RNA_44 | InfB | 284,492 | 10,072 | 35.424 | 0.766 | 93,090 | 26,521 | 37.119 | 1.150 |
| M21_44 | M21 + RNA_44 | RNaseP | 1,221,893 | 104,953 | 34.492 | 0.195 | 910,046 | 38,328 | 35.045 | 0.324 |
| M21_44 | M21 + RNA_44 | SARS-CoV- | 446,697 | 22,411 | 35.157 | 0.501 | 72,956 | 29,606 | 36.741 | 0.747 |
| | | 2 | | | | | | | | |
| M21 + RNA_9 | M21 + RNA_21 | InfA | 697,348 | 26,788 | 35.466 | 0.465 | 469,293 | 86,742 | 36.491 | 0.711 |
| M21 + RNA_9 | M21 + RNA_21 | InfB | 258,815 | 20,451 | 35.463 | 0.362 | 126,582 | 31,452 | 36.210 | 0.641 |
| M21 + RNA_9 | M21 + RNA_21 | RNaseP | 1,054,405 | 128,159 | 35.284 | 0.558 | 824,697 | 92,069 | 35.150 | 0.141 |
| M21 + RNA_9 | M21 + RNA_21 | SARS-CoV- | 445,835 | 56,456 | 35.303 | 0.130 | 137,048 | 64,358 | 36.030 | 0.141 |
| | | 2 | | | | | | | | |
| M21 + RNA_9 | M21 + RNA_25 | InfA | 755,510 | 19,425 | 35.526 | 0.325 | 479,525 | 51,998 | 36.434 | 0.189 |
| M21 + RNA_9 | M21 + RNA_25 | InfB | 269,485 | 29,372 | 35.183 | 0.135 | 147,804 | 12,752 | 35.969 | 0.050 |
| M21 + RNA_9 | M21 + RNA_25 | RNaseP | 1,209,677 | 98,920 | 34.836 | 0.812 | 827,987 | 49,822 | 35.327 | 0.365 |
| M21 + RNA_9 | M21 + RNA_25 | SARS-CoV- | 472,169 | 21,260 | 35.339 | 0.166 | 186,910 | 40,460 | 35.715 | 0.416 |
| | | 2 | | | | | | | | |
| M21 + RNA_9 | M21 + RNA_27 | InfA | 685,768 | 40,134 | 35.507 | 0.989 | 495,228 | 18,114 | 36.325 | 0.270 |
| M21 + RNA_9 | M21 + RNA_27 | InfB | 265,697 | 43,799 | 34.807 | 0.216 | 118,851 | 2,767 | 36.483 | 0.216 |
| M21 + RNA_9 | M21 + RNA_27 | RNaseP | 1,131,669 | 219,790 | 34.753 | 0.448 | 889,981 | 38,600 | 35.129 | 0.250 |
| M21 + RNA_9 | M21 + RNA_27 | SARS-CoV- | 415,458 | 49,299 | 35.457 | 0.572 | 140,616 | 22,572 | 36.074 | 0.578 |
| | | 2 | | | | | | | | |
| M21 + RNA_9 | M21 + RNA_28 | InfA | 769,216 | 4,911 | 35.332 | 0.211 | 388,686 | 124,422 | 37.296 | 1.440 |
| M21 + RNA_9 | M21 + RNA_28 | InfB | 261,321 | 43,864 | 35.850 | 0.728 | 108,931 | 26,056 | 37.013 | 0.862 |
| M21 + RNA_9 | M21 + RNA_28 | RNaseP | 1,131,195 | 50,139 | 34.894 | 0.382 | 925,403 | 106,898 | 34.992 | 0.288 |
| M21 + RNA_9 | M21 + RNA_28 | SARS-CoV- | 432,741 | 41,671 | 35.738 | 0.394 | 144,823 | 39,802 | 36.137 | 0.646 |
| | | 2 | | | | | | | | |
| M21 + RNA_9 | M21 + RNA_30 | InfA | 701,019 | 32,441 | 35.836 | 0.421 | 568,184 | 94,893 | 35.927 | 0.501 |
| M21 + RNA_9 | M21 + RNA_30 | InfB | 282,941 | 54,534 | 35.218 | 0.800 | 125,009 | 18,952 | 36.411 | 0.572 |
| M21 + RNA_9 | M21 + RNA_30 | RNaseP | 1,123,130 | 66,430 | 34.953 | 0.410 | 900,107 | 52,318 | 35.250 | 0.350 |
| M21 + RNA_9 | M21 + RNA_30 | SARS-CoV- | 440,676 | 28,679 | 35.255 | 0.481 | 132,256 | 22,295 | 36.079 | 0.405 |
| | | 2 | | | | | | | | |
| M21 + RNA_9 | M21 + RNA_31 | InfA | 771,810 | 49,653 | 35.591 | 0.788 | 470,968 | 59,683 | 36.520 | 0.360 |
| M21 + RNA_9 | M21 + RNA_31 | InfB | 310,621 | 21,102 | 34.769 | 0.213 | 90,820 | 65,737 | 36.046 | 0.990 |
| M21 + RNA_9 | M21 + RNA_31 | RNaseP | 1,204,866 | 34,456 | 34.927 | 0.486 | 954,098 | 57,801 | 35.006 | 0.502 |
| M21 + RNA_9 | M21 + RNA_31 | SARS-CoV- | 498,201 | 16,334 | 34.796 | 0.301 | 108,217 | 45,394 | 36.714 | 1.304 |
| | | 2 | | | | | | | | |
| M21 + RNA_9 | M21 + RNA_32 | InfA | 689,951 | 120,100 | 36.077 | 0.852 | 520,827 | 32,749 | 36.228 | 0.302 |
| M21 + RNA_9 | M21 + RNA_32 | InfB | 307,388 | 13,333 | 34.969 | 0.264 | 160,760 | 20,324 | 35.767 | 0.754 |
| M21 + RNA_9 | M21 + RNA_32 | RNaseP | 1,223,083 | 49,272 | 34.488 | 0.324 | 847,942 | 28,648 | 35.598 | 0.321 |
| M21 + RNA_9 | M21 + RNA_32 | SARS-CoV- | 431,970 | 29,781 | 34.923 | 0.240 | 192,929 | 22,000 | 36.048 | 0.220 |
| | | 2 | | | | | | | | |
| M21 + RNA_9 | M21 + RNA_39 | InfA | 723,124 | 45,937 | 35.937 | 0.434 | 466,887 | 187,087 | 36.556 | 1.336 |
| M21 + RNA_9 | M21 + RNA_39 | InfB | 277,950 | 26,550 | 35.115 | 0.587 | 119,426 | 46,509 | 36.422 | 1.598 |
| M21 + RNA_9 | M21 + RNA_39 | RNaseP | 1,198,349 | 59,591 | 34.634 | 0.253 | 877,080 | 50,598 | 34.661 | 0.236 |
| M21 + RNA_9 | M21 + RNA_39 | SARS-CoV- | 444,971 | 10,368 | 35.254 | 0.217 | 121,994 | 69,551 | 37.336 | 2.001 |
| | | 2 | | | | | | | | |
| M21 + RNA_9 | M21 + RNA_40 | InfA | 704,397 | 44,033 | 36.234 | 0.161 | 510,731 | 143,631 | 36.826 | 1.279 |
| M21 + RNA_9 | M21 + RNA_40 | InfB | 301,194 | 12,127 | 35.035 | 0.164 | 152,943 | 19,639 | 36.166 | 0.464 |
| M21 + RNA_9 | M21 + RNA_40 | RNaseP | 1,195,224 | 87,216 | 35.143 | 0.431 | 950,196 | 31,357 | 35.271 | 0.109 |
| M21 + RNA_9 | M21 + RNA_40 | SARS-CoV- | 517,114 | 13,871 | 35.242 | 0.094 | 242,889 | 33,803 | 35.814 | 0.394 |
| | | 2 | | | | | | | | |
| M21 + RNA_9 | M21 + RNA_42 | InfA | 777,304 | 48,415 | 35.181 | 0.492 | 398,458 | 53,680 | 37.062 | 0.472 |
| M21 + RNA_9 | M21 + RNA_42 | InfB | 275,945 | 19,023 | 35.218 | 0.498 | 148,612 | 8,988 | 35.890 | 0.216 |
| M21 + RNA_9 | M21 + RNA_42 | RNaseP | 1,061,438 | 43,516 | 35.046 | 0.330 | 944,795 | 24,832 | 34.756 | 0.285 |
| M21 + RNA_9 | M21 + RNA_42 | SARS-CoV- | 443,963 | 6,648 | 35.235 | 0.351 | 165,060 | 59,297 | 35.722 | 0.442 |
| | | 2 | | | | | | | | |
| M21 + RNA_9 | M21 + RNA_43 | InfA | 741,577 | 34,020 | 35.835 | 0.279 | 438,618 | 113,828 | 36.788 | 0.841 |
| M21 + RNA_9 | M21 + RNA_43 | InfB | 290,741 | 23,079 | 34.905 | 0.388 | 137,058 | 7,879 | 36.089 | 0.306 |
| M21 + RNA_9 | M21 + RNA_43 | RNaseP | 1,178,709 | 36,725 | 34.822 | 0.199 | 815,213 | 134,584 | 35.271 | 0.732 |
| M21 + RNA_9 | M21 + RNA_43 | SARS-CoV- | 460,837 | 11,889 | 35.299 | 0.425 | 179,863 | 14,790 | 35.686 | 0.208 |
| | | 2 | | | | | | | | |
| M21 + RNA_9 | M21 + RNA_44 | InfA | 702,140 | 26,151 | 35.830 | 0.261 | 479,522 | 23,535 | 36.556 | 0.494 |
| M21 + RNA_9 | M21 + RNA_44 | InfB | 284,649 | 13,280 | 35.480 | 0.468 | 146,772 | 35,042 | 36.074 | 0.900 |
| M21 + RNA_9 | M21 + RNA_44 | RNaseP | 1,119,008 | 96,083 | 34.987 | 0.322 | 878,154 | 186,986 | 35.283 | 0.574 |
| M21 + RNA_9 | M21 + RNA_44 | SARS-CoV- | 387,072 | 38,842 | 35.446 | 0.482 | 166,482 | 11,815 | 35.489 | 0.261 |
| | | 2 | | | | | | | | |
| M21 + RNA_21 | M21 + RNA_25 | InfA | 732,958 | 20,647 | 35.352 | 0.198 | 342,896 | 68,319 | 37.351 | 0.989 |
| M21 + RNA_21 | M21 + RNA_25 | InfB | 294,399 | 7,916 | 34.779 | 0.453 | 113,650 | 34,299 | 36.617 | 0.858 |
| M21 + RNA_21 | M21 + RNA_25 | RNaseP | 1,142,297 | 29,350 | 34.739 | 0.366 | 787,593 | 22,816 | 35.060 | 0.336 |
| M21 + RNA_21 | M21 + RNA_25 | SARS-CoV- | 424,820 | 35,641 | 35.393 | 0.296 | 137,729 | 75,971 | 36.773 | 2.270 |
| | | 2 | | | | | | | | |
| M21 + RNA_21 | M21 + RNA_27 | InfA | 777,590 | 60,906 | 35.434 | 0.838 | 433,333 | 65,427 | 36.710 | 0.695 |
| M21 + RNA_21 | M21 + RNA_27 | InfB | 299,765 | 17,027 | 35.346 | 0.528 | 136,921 | 14,712 | 36.103 | 0.079 |
| M21 + RNA_21 | M21 + RNA_27 | RNaseP | 1,150,164 | 43,398 | 34.627 | 0.237 | 810,311 | 30,302 | 35.275 | 0.306 |
| M21 + RNA_21 | M21 + RNA_27 | SARS-CoV- | 408,867 | 15,381 | 35.355 | 0.428 | 205,296 | 26,690 | 35.374 | 0.585 |
| | | 2 | | | | | | | | |
| M21 + RNA_21 | M21 + RNA_28 | InfA | 763,844 | 56,458 | 35.326 | 0.563 | 250,779 | 109,138 | 38.467 | 2.654 |
| M21 + RNA_21 | M21 + RNA_28 | InfB | 282,773 | 13,674 | 35.236 | 0.253 | 97,535 | 8,495 | 36.434 | 0.183 |
| M21 + RNA_21 | M21 + RNA_28 | RNaseP | 1,099,468 | 41,745 | 35.010 | 0.308 | 719,223 | 81,069 | 35.275 | 0.521 |
| M21 + RNA_21 | M21 + RNA_28 | SARS-CoV- | 417,619 | 31,151 | 35.148 | 0.541 | 113,828 | 43,305 | 35.749 | 0.540 |
| | | 2 | | | | | | | | |
| M21 + RNA_21 | M21 + RNA_30 | InfA | 685,438 | 40,375 | 36.143 | 0.316 | 520,266 | 119,095 | 36.357 | 1.155 |
| M21 + RNA_21 | M21 + RNA_30 | InfB | 274,517 | 46,585 | 35.585 | 0.869 | 122,292 | 23,452 | 36.725 | 0.730 |
| M21 + RNA_21 | M21 + RNA_30 | RNaseP | 1,205,836 | 6,228 | 34.500 | 0.110 | 853,783 | 14,797 | 35.164 | 0.337 |
| M21 + RNA_21 | M21 + RNA_30 | SARS-CoV- | 417,524 | 22,491 | 35.271 | 0.439 | 127,644 | 14,276 | 35.674 | 0.115 |
| | | 2 | | | | | | | | |
| M21 + RNA_21 | M21 + RNA_31 | InfA | 660,464 | 44,007 | 36.202 | 0.826 | 496,445 | 37,951 | 36.305 | 0.316 |
| M21 + RNA_21 | M21 + RNA_31 | InfB | 268,371 | 27,172 | 35.119 | 0.085 | 141,741 | 24,804 | 36.285 | 0.645 |
| M21 + RNA_21 | M21 + RNA_31 | RNaseP | 1,121,204 | 159,901 | 34.908 | 0.263 | 914,247 | 50,627 | 35.025 | 0.196 |
| M21 + RNA_21 | M21 + RNA_31 | SARS-CoV- | 452,209 | 35,772 | 35.168 | 0.393 | 169,125 | 56,887 | 35.957 | 0.958 |
| | | 2 | | | | | | | | |
| M21 + RNA_21 | M21 + RNA_32 | InfA | 735,141 | 29,101 | 35.468 | 0.392 | 402,845 | 45,005 | 36.968 | 0.485 |
| M21 + RNA_21 | M21 + RNA_32 | InfB | 279,137 | 18,592 | 35.091 | 0.359 | 123,017 | 22,478 | 36.431 | 0.675 |
| M21 + RNA_21 | M21 + RNA_32 | RNaseP | 1,210,661 | 60,828 | 34.653 | 0.287 | 898,259 | 21,147 | 34.923 | 0.265 |
| M21 + RNA_21 | M21 + RNA_32 | SARS-CoV- | 446,980 | 21,856 | 35.205 | 0.500 | 207,447 | 30,229 | 35.078 | 0.185 |
| | | 2 | | | | | | | | |
| M21 + RNA_21 | M21 + RNA_39 | InfA | 713,962 | 36,202 | 35.826 | 0.401 | 364,401 | 62,245 | 36.616 | 0.892 |
| M21 + RNA_21 | M21 + RNA_39 | InfB | 292,159 | 24,206 | 35.022 | 0.128 | 97,597 | 42,794 | 37.180 | 2.688 |
| M21 + RNA_21 | M21 + RNA_39 | RNaseP | 1,232,055 | 20,723 | 34.655 | 0.027 | 853,920 | 50,020 | 35.102 | 0.480 |
| M21 + RNA_21 | M21 + RNA_39 | SARS-CoV- | 427,645 | 35,549 | 35.362 | 0.503 | 142,450 | 7,544 | 35.544 | 0.556 |
| | | 2 | | | | | | | | |
| M21 + RNA_21 | M21 + RNA_40 | InfA | 701,746 | 54,006 | 35.375 | 0.463 | 441,293 | 22,783 | 36.390 | 0.302 |
| M21 + RNA_21 | M21 + RNA_40 | InfB | 287,237 | 6,781 | 35.111 | 0.190 | 104,554 | 14,697 | 36.501 | 0.505 |
| M21 + RNA_21 | M21 + RNA_40 | RNaseP | 1,118,638 | 54,037 | 34.726 | 0.444 | 848,102 | 38,670 | 35.251 | 0.119 |
| M21 + RNA_21 | M21 + RNA_40 | SARS-CoV- | 343,750 | 59,843 | 35.652 | 0.520 | 132,154 | 25,945 | 36.251 | 0.462 |
| | | 2 | | | | | | | | |
| M21 + RNA_21 | M21 + RNA_42 | InfA | 618,383 | 53,422 | 36.445 | 0.463 | 347,957 | 150,167 | 37.369 | 1.548 |
| M21 + RNA_21 | M21 + RNA_42 | InfB | 279,744 | 15,887 | 35.163 | 0.240 | 136,260 | 20,014 | 36.152 | 0.364 |
| M21 + RNA_21 | M21 + RNA_42 | RNaseP | 1,101,767 | 89,397 | 34.561 | 0.346 | 798,267 | 39,757 | 35.093 | 0.526 |
| M21 + RNA_21 | M21 + RNA_42 | SARS-CoV- | 391,779 | 20,619 | 35.461 | 0.779 | 132,415 | 25,869 | 36.143 | 0.875 |
| | | 2 | | | | | | | | |
| M21 + RNA_21 | M21 + RNA_43 | InfA | 798,754 | 8,868 | 35.252 | 0.135 | 400,882 | 72,953 | 36.638 | 0.603 |
| M21 + RNA_21 | M21 + RNA_43 | InfB | 281,627 | 22,074 | 35.345 | 0.712 | 139,848 | 4,657 | 36.030 | 0.275 |
| M21 + RNA_21 | M21 + RNA_43 | RNaseP | 1,080,351 | 69,216 | 34.819 | 0.290 | 793,992 | 88,535 | 35.023 | 0.098 |
| M21 + RNA_21 | M21 + RNA_43 | SARS-CoV- | 419,735 | 53,165 | 35.698 | 0.965 | 154,506 | 28,238 | 35.740 | 0.198 |
| | | 2 | | | | | | | | |
| M21 + RNA_21 | M21 + RNA_44 | InfA | 798,495 | 25,103 | 34.948 | 0.492 | 395,578 | 91,319 | 36.976 | 0.847 |
| M21 + RNA_21 | M21 + RNA_44 | InfB | 276,057 | 19,495 | 35.769 | 0.660 | 128,991 | 6,985 | 36.336 | 0.262 |
| M21 + RNA_21 | M21 + RNA_44 | RNaseP | 1,159,922 | 46,808 | 34.890 | 0.097 | 844,101 | 67,158 | 35.077 | 0.185 |
| M21 + RNA_21 | M21 + RNA_44 | SARS-CoV- | 422,866 | 18,237 | 35.281 | 0.073 | 165,573 | 19,680 | 35.444 | 0.300 |
| | | 2 | | | | | | | | |
| M21 + RNA_25 | M21 + RNA_27 | InfA | 723,248 | 55,765 | 35.845 | 0.484 | 378,292 | 101,175 | 36.922 | 1.197 |
| M21 + RNA_25 | M21 + RNA_27 | InfB | 255,078 | 51,037 | 35.823 | 0.935 | 117,340 | 17,610 | 36.423 | 0.398 |
| M21 + RNA_25 | M21 + RNA_27 | RNaseP | 1,165,186 | 66,589 | 34.843 | 0.263 | 853,689 | 30,990 | 35.296 | 0.087 |
| M21 + RNA_25 | M21 + RNA_27 | SARS-CoV- | 437,768 | 16,458 | 35.207 | 0.335 | 145,177 | 47,827 | 36.504 | 1.105 |
| | | 2 | | | | | | | | |
| M21 + RNA_25 | M21 + RNA_28 | InfA | 769,335 | 26,824 | 35.406 | 0.141 | 420,780 | 58,425 | 36.338 | 0.488 |
| M21 + RNA_25 | M21 + RNA_28 | InfB | 268,712 | 63,166 | 35.560 | 0.659 | 95,155 | 9,178 | 36.728 | 0.222 |
| M21 + RNA_25 | M21 + RNA_28 | RNaseP | 1,160,718 | 43,413 | 34.828 | 0.461 | 816,902 | 52,934 | 35.237 | 0.314 |
| M21 + RNA_25 | M21 + RNA_28 | SARS-CoV- | 437,010 | 8,998 | 35.490 | 0.201 | 107,648 | 29,004 | 35.983 | 0.466 |
| | | 2 | | | | | | | | |
| M21 + RNA_25 | M21 + RNA_30 | InfA | 722,485 | 33,781 | 35.970 | 0.568 | 466,673 | 170,988 | 37.020 | 1.693 |
| M21 + RNA_25 | M21 + RNA_30 | InfB | 271,060 | 35,652 | 35.735 | 0.698 | 128,049 | 23,587 | 36.232 | 0.940 |
| M21 + RNA_25 | M21 + RNA_30 | RNaseP | 1,030,259 | 11,714 | 35.571 | 0.424 | 937,059 | 54,961 | 34.937 | 0.301 |
| M21 + RNA_25 | M21 + RNA_30 | SARS-CoV- | 426,636 | 39,979 | 35.697 | 0.291 | 61,587 | 16,415 | 37.276 | 1.421 |
| | | 2 | | | | | | | | |
| M21 + RNA_25 | M21 + RNA_31 | InfA | 614,965 | 97,285 | 36.684 | 0.846 | 441,586 | 74,493 | 36.354 | 0.674 |
| M21 + RNA_25 | M21 + RNA_31 | InfB | 290,982 | 29,366 | 35.284 | 0.297 | 127,646 | 38,608 | 36.204 | 1.138 |
| M21 + RNA_25 | M21 + RNA_31 | RNaseP | 1,145,659 | 31,393 | 35.064 | 0.381 | 936,549 | 37,665 | 35.058 | 0.318 |
| M21 + RNA_25 | M21 + RNA_31 | SARS-CoV- | 408,397 | 38,415 | 35.749 | 0.519 | 87,871 | 39,088 | 37.777 | 2.688 |
| | | 2 | | | | | | | | |
| M21 + RNA_25 | M21 + RNA_32 | InfA | 718,478 | 37,452 | 35.717 | 0.537 | 361,335 | 88,371 | 36.703 | 0.783 |
| M21 + RNA_25 | M21 + RNA_32 | InfB | 252,449 | 28,991 | 35.312 | 0.484 | 144,617 | 10,611 | 35.944 | 0.282 |
| M21 + RNA_25 | M21 + RNA_32 | RNaseP | 1,112,514 | 43,250 | 34.959 | 0.072 | 757,458 | 22,598 | 35.299 | 0.320 |
| M21 + RNA_25 | M21 + RNA_32 | SARS-CoV- | 405,788 | 42,485 | 35.672 | 0.452 | 136,588 | 27,005 | 35.981 | 1.067 |
| | | 2 | | | | | | | | |
| M21 + RNA_25 | M21 + RNA_39 | InfA | 706,981 | 48,827 | 35.538 | 0.249 | 291,286 | 113,938 | 37.404 | 1.732 |
| M21 + RNA_25 | M21 + RNA_39 | InfB | 272,106 | 10,972 | 35.179 | 0.557 | 110,697 | 5,723 | 36.280 | 0.330 |
| M21 + RNA_25 | M21 + RNA_39 | RNaseP | 1,084,821 | 78,037 | 35.217 | 0.464 | 842,964 | 46,456 | 35.017 | 0.299 |
| M21 + RNA_25 | M21 + RNA_39 | SARS-CoV- | 403,612 | 3,731 | 35.377 | 0.355 | 124,702 | 57,404 | 36.511 | 1.731 |
| | | 2 | | | | | | | | |
| M21 + RNA_25 | M21 + RNA_40 | InfA | 713,686 | 32,220 | 35.851 | 0.820 | 505,090 | 57,711 | 36.137 | 0.485 |
| M21 + RNA_25 | M21 + RNA_40 | InfB | 279,936 | 9,211 | 35.113 | 1.050 | 152,911 | 35,343 | 35.967 | 0.652 |
| M21 + RNA_25 | M21 + RNA_40 | RNaseP | 1,011,753 | 97,217 | 35.224 | 0.289 | 865,671 | 23,893 | 35.286 | 0.375 |
| M21 + RNA_25 | M21 + RNA_40 | SARS-CoV- | 407,923 | 11,368 | 35.051 | 0.176 | 189,922 | 22,990 | 35.862 | 0.144 |
| | | 2 | | | | | | | | |
| M21 + RNA_25 | M21 + RNA_42 | InfA | 711,981 | 26,753 | 35.645 | 0.479 | 357,589 | 55,861 | 36.730 | 0.357 |
| M21 + RNA_25 | M21 + RNA_42 | InfB | 268,222 | 27,180 | 35.500 | 0.630 | 122,216 | 34,244 | 36.043 | 1.060 |
| M21 + RNA_25 | M21 + RNA_42 | RNaseP | 1,017,798 | 38,160 | 35.216 | 0.077 | 816,997 | 27,745 | 35.128 | 0.314 |
| M21 + RNA_25 | M21 + RNA_42 | SARS-CoV- | 369,309 | 58,518 | 35.752 | 0.996 | 137,885 | 38,587 | 36.005 | 0.520 |
| | | 2 | | | | | | | | |
| M21 + RNA_25 | M21 + RNA_43 | InfA | 675,498 | 102,342 | 35.818 | 0.828 | 313,084 | 234,535 | 36.737 | 1.177 |
| M21 + RNA_25 | M21 + RNA_43 | InfB | 260,331 | 22,436 | 35.521 | 0.324 | 90,611 | 55,130 | 36.416 | 0.291 |
| M21 + RNA_25 | M21 + RNA_43 | RNaseP | 932,364 | 45,336 | 35.419 | 0.216 | 530,570 | 292,863 | 37.727 | 4.332 |
| M21 + RNA_25 | M21 + RNA_43 | SARS-CoV- | 380,523 | 52,847 | 35.975 | 0.896 | 115,188 | 58,184 | 37.477 | 2.084 |
| | | 2 | | | | | | | | |
| M21 + RNA_25 | M21 + RNA_44 | InfA | 751,009 | 5,719 | 35.907 | 0.331 | 438,588 | 27,573 | 36.683 | 0.367 |
| M21 + RNA_25 | M21 + RNA_44 | InfB | 312,585 | 5,820 | 35.079 | 0.313 | 114,086 | 4,133 | 36.736 | 0.509 |
| M21 + RNA_25 | M21 + RNA_44 | RNaseP | 1,087,863 | 19,513 | 35.186 | 0.209 | 873,121 | 106,763 | 35.085 | 0.366 |
| M21 + RNA_25 | M21 + RNA_44 | SARS-CoV- | 383,793 | 22,718 | 36.057 | 0.154 | 107,987 | 34,778 | 35.933 | 0.527 |
| | | 2 | | | | | | | | |
| M21 + RNA_27 | M21 + RNA_28 | InfA | 740,134 | 44,389 | 35.371 | 0.620 | 467,185 | 103,660 | 36.382 | 0.960 |
| M21 + RNA_27 | M21 + RNA_28 | InfB | 288,116 | 28,436 | 35.028 | 0.603 | 138,838 | 15,241 | 35.819 | 0.289 |
| M21 + RNA_27 | M21 + RNA_28 | RNaseP | 1,135,146 | 65,737 | 34.916 | 0.227 | 796,740 | 107,173 | 35.472 | 0.436 |
| M21 + RNA_27 | M21 + RNA_28 | SARS-CoV- | 411,542 | 44,020 | 35.444 | 0.255 | 67,871 | 16,704 | 37.154 | 0.612 |
| | | 2 | | | | | | | | |
| M21 + RNA_27 | M21 + RNA_30 | InfA | 780,701 | 32,982 | 35.438 | 0.808 | 431,904 | 61,863 | 36.965 | 0.665 |
| M21 + RNA_27 | M21 + RNA_30 | InfA | 723,225 | 13,850 | 35.865 | 0.052 | 352,248 | 116,236 | 37.301 | 1.150 |
| M21 + RNA_27 | M21 + RNA_30 | InfB | 322,472 | 7,730 | 35.160 | 0.609 | 92,110 | 32,477 | 38.003 | 1.721 |
| M21 + RNA_27 | M21 + RNA_30 | InfB | 269,613 | 15,031 | 35.264 | 0.460 | 118,932 | 23,330 | 36.097 | 0.780 |
| M21 + RNA_27 | M21 + RNA_30 | RNaseP | 1,140,899 | 48,081 | 34.880 | 0.205 | 890,114 | 47,025 | 34.868 | 0.180 |
| M21 + RNA_27 | M21 + RNA_30 | RNaseP | 994,534 | 76,447 | 35.417 | 0.307 | 822,927 | 82,572 | 35.000 | 0.646 |
| M21 + RNA_27 | M21 + RNA_30 | SARS-CoV- | 394,279 | 21,309 | 35.695 | 0.479 | 58,043 | 22,641 | 37.667 | 2.330 |
| | | 2 | | | | | | | | |
| M21 + RNA_27 | M21 + RNA_30 | SARS-CoV- | 367,192 | 47,236 | 35.643 | 0.551 | 22,158 | 4,913 | NA | NA |
| | | 2 | | | | | | | | |
| M21 + RNA_27 | M21 + RNA_31 | InfA | 740,948 | 85,636 | 35.487 | 0.780 | 489,314 | 81,373 | 36.031 | 0.810 |
| M21 + RNA_27 | M21 + RNA_31 | InfB | 280,557 | 12,176 | 35.702 | 0.433 | 147,071 | 21,267 | 35.787 | 0.643 |
| M21 + RNA_27 | M21 + RNA_31 | RNaseP | 1,089,601 | 63,027 | 35.121 | 0.405 | 861,454 | 45,297 | 35.050 | 0.246 |
| M21 + RNA_27 | M21 + RNA_31 | SARS-CoV- | 406,763 | 26,564 | 35.773 | 0.545 | 87,181 | 4,440 | 36.541 | 0.249 |
| | | 2 | | | | | | | | |
| M21 + RNA_27 | M21 + RNA_32 | InfA | 706,509 | 26,605 | 35.708 | 0.125 | 383,718 | 64,228 | 36.499 | 0.314 |
| M21 + RNA_27 | M21 + RNA_32 | InfB | 300,473 | 20,422 | 35.194 | 0.416 | 115,566 | 17,890 | 36.434 | 1.028 |
| M21 + RNA_27 | M21 + RNA_32 | RNaseP | 1,032,446 | 63,473 | 35.067 | 0.261 | 766,777 | 53,318 | 35.201 | 0.500 |
| M21 + RNA_27 | M21 + RNA_32 | SARS-CoV- | 390,714 | 4,475 | 35.623 | 0.486 | 136,914 | 38,611 | 35.929 | 0.310 |
| | | 2 | | | | | | | | |
| M21 + RNA_27 | M21 + RNA_39 | InfA | 800,736 | 42,071 | 35.046 | 0.457 | 417,311 | 123,248 | 36.357 | 1.226 |
| M21 + RNA_27 | M21 + RNA_39 | InfB | 307,140 | 12,046 | 34.680 | 0.647 | 116,804 | 11,035 | 36.102 | 0.146 |
| M21 + RNA_27 | M21 + RNA_39 | RNaseP | 1,116,466 | 60,582 | 34.840 | 0.287 | 704,481 | 88,082 | 35.850 | 0.632 |
| M21 + RNA_27 | M21 + RNA_39 | SARS-CoV- | 388,045 | 18,773 | 35.244 | 0.118 | 137,691 | 12,875 | 35.975 | 0.448 |
| | | 2 | | | | | | | | |
| M21 + RNA_27 | M21 + RNA_40 | InfA | 650,741 | 86,960 | 36.189 | 0.754 | 378,871 | 190,135 | 37.954 | 2.894 |
| M21 + RNA_27 | M21 + RNA_40 | InfB | 305,107 | 24,575 | 34.841 | 0.262 | 154,589 | 3,656 | 36.038 | 0.302 |
| M21 + RNA_27 | M21 + RNA_40 | RNaseP | 1,093,693 | 92,243 | 35.041 | 0.150 | 869,190 | 31,631 | 35.280 | 0.401 |
| M21 + RNA_27 | M21 + RNA_40 | SARS-CoV- | 417,186 | 6,489 | 35.175 | 0.197 | 196,826 | 27,506 | 35.970 | 0.202 |
| | | 2 | | | | | | | | |
| M21 + RNA_27 | M21 + RNA_42 | InfA | 747,186 | 77,509 | 35.660 | 0.412 | 438,884 | 114,270 | 36.724 | 0.857 |
| M21 + RNA_27 | M21 + RNA_42 | InfB | 268,659 | 48,307 | 35.747 | 1.158 | 109,308 | 44,866 | 37.056 | 2.214 |
| M21 + RNA_27 | M21 + RNA_42 | RNaseP | 1,075,475 | 113,243 | 34.955 | 0.290 | 828,466 | 139,433 | 35.675 | 0.784 |
| M21 + RNA_27 | M21 + RNA_42 | SARS-CoV- | 399,987 | 57,991 | 35.095 | 0.553 | 116,841 | 24,247 | 36.773 | 0.630 |
| | | 2 | | | | | | | | |
| M21 + RNA_27 | M21 + RNA_43 | InfA | 800,514 | 37,238 | 35.264 | 0.724 | 478,814 | 115,934 | 36.198 | 1.287 |
| M21 + RNA_27 | M21 + RNA_43 | InfB | 296,648 | 32,575 | 35.215 | 0.774 | 107,626 | 26,478 | 36.848 | 0.689 |
| M21 + RNA_27 | M21 + RNA_43 | RNaseP | 1,094,544 | 106,875 | 34.792 | 0.679 | 760,289 | 50,378 | 35.312 | 0.265 |
| M21 + RNA_27 | M21 + RNA_43 | SARS-CoV- | 428,462 | 46,650 | 35.469 | 0.381 | 136,024 | 39,070 | 36.399 | 0.589 |
| | | 2 | | | | | | | | |
| M21 + RNA_27 | M21 + RNA_44 | InfA | 812,537 | 41,622 | 35.471 | 0.566 | 490,615 | 73,229 | 36.589 | 0.642 |
| M21 + RNA_27 | M21 + RNA_44 | InfB | 274,747 | 36,977 | 35.769 | 0.808 | 116,794 | 16,135 | 36.451 | 0.802 |
| M21 + RNA_27 | M21 + RNA_44 | RNaseP | 1,106,794 | 78,284 | 34.964 | 0.609 | 865,427 | 30,842 | 35.123 | 0.426 |
| M21 + RNA_27 | M21 + RNA_44 | SARS-CoV- | 385,215 | 32,959 | 35.370 | 0.456 | 79,854 | 31,290 | 37.625 | 2.155 |
| | | 2 | | | | | | | | |
| M21 + RNA_28 | M21 + RNA_31 | InfA | 755,087 | 28,208 | 35.245 | 0.166 | 361,348 | 106,297 | 36.731 | 1.138 |
| M21 + RNA_28 | M21 + RNA_31 | InfB | 282,071 | 16,399 | 35.241 | 0.148 | 77,884 | 33,165 | 37.745 | 2.490 |
| M21 + RNA_28 | M21 + RNA_31 | RNaseP | 1,060,670 | 46,569 | 35.130 | 0.248 | 759,163 | 37,435 | 35.186 | 0.055 |
| M21 + RNA_28 | M21 + RNA_31 | SARS-CoV- | 404,696 | 19,563 | 35.334 | 0.252 | 63,394 | 13,089 | 37.008 | 1.049 |
| | | 2 | | | | | | | | |
| M21 + RNA_28 | M21 + RNA_32 | InfA | 707,840 | 81,509 | 35.946 | 1.168 | 302,199 | 92,177 | 37.604 | 1.273 |
| M21 + RNA_28 | M21 + RNA_32 | InfB | 274,118 | 8,287 | 35.529 | 0.263 | 130,176 | 23,210 | 35.901 | 0.712 |
| M21 + RNA_28 | M21 + RNA_32 | RNaseP | 941,420 | 22,724 | 35.450 | 0.166 | 682,180 | 13,257 | 35.365 | 0.168 |
| M21 + RNA_28 | M21 + RNA_32 | SARS-CoV- | 374,817 | 18,414 | 35.460 | 0.399 | 132,080 | 14,515 | 36.082 | 0.161 |
| | | 2 | | | | | | | | |
| M21 + RNA_28 | M21 + RNA_39 | InfA | 773,932 | 8,805 | 35.493 | 0.202 | 291,789 | 122,752 | 37.484 | 1.726 |
| M21 + RNA_28 | M21 + RNA_39 | InfB | 277,200 | 45,605 | 35.469 | 1.062 | 106,615 | 23,296 | 36.239 | 1.025 |
| M21 + RNA_28 | M21 + RNA_39 | RNaseP | 1,065,568 | 92,989 | 34.911 | 0.676 | 819,286 | 82,182 | 35.383 | 0.650 |
| M21 + RNA_28 | M21 + RNA_39 | SARS-CoV- | 440,792 | 47,371 | 35.071 | 0.752 | 106,208 | 13,866 | 36.086 | 0.389 |
| | | 2 | | | | | | | | |
| M21 + RNA_28 | M21 + RNA_40 | InfA | 792,578 | 43,246 | 35.168 | 0.257 | 340,861 | 85,063 | 37.393 | 0.904 |
| M21 + RNA_28 | M21 + RNA_40 | InfB | 290,920 | 13,205 | 35.413 | 0.063 | 123,636 | 35,998 | 36.258 | 1.415 |
| M21 + RNA_28 | M21 + RNA_40 | RNaseP | 1,151,109 | 95,207 | 34.734 | 0.430 | 890,752 | 3,060 | 35.040 | 0.205 |
| M21 + RNA_28 | M21 + RNA_40 | SARS-CoV- | 465,808 | 13,630 | 35.030 | 0.519 | 130,943 | 9,056 | 36.273 | 0.346 |
| | | 2 | | | | | | | | |
| M21 + RNA_28 | M21 + RNA_42 | InfA | 740,944 | 139,729 | 34.975 | 0.462 | 455,395 | 18,956 | 36.162 | 0.101 |
| M21 + RNA_28 | M21 + RNA_42 | InfB | 265,027 | 59,851 | 34.900 | 0.466 | 118,752 | 21,455 | 35.967 | 0.912 |
| M21 + RNA_28 | M21 + RNA_42 | RNaseP | 962,625 | 279,126 | 34.477 | 0.233 | 815,198 | 56,319 | 34.832 | 0.277 |
| M21 + RNA_28 | M21 + RNA_42 | SARS-CoV- | 399,378 | 46,211 | 35.238 | 0.343 | 82,442 | 14,957 | 36.259 | 0.801 |
| | | 2 | | | | | | | | |
| M21 + RNA_28 | M21 + RNA_43 | InfA | 779,474 | 34,345 | 35.633 | 0.484 | 421,451 | 46,717 | 36.842 | 0.517 |
| M21 + RNA_28 | M21 + RNA_43 | InfB | 293,558 | 15,159 | 35.290 | 0.329 | 138,077 | 7,871 | 35.663 | 0.268 |
| M21 + RNA_28 | M21 + RNA_43 | RNaseP | 1,163,393 | 41,862 | 34.818 | 0.338 | 788,190 | 50,422 | 35.344 | 0.252 |
| M21 + RNA_28 | M21 + RNA_43 | SARS-CoV- | 453,613 | 17,991 | 35.232 | 0.592 | 114,512 | 28,281 | 36.424 | 0.857 |
| | | 2 | | | | | | | | |
| M21 + RNA_28 | M21 + RNA_44 | InfA | 810,221 | 21,155 | 35.413 | 0.408 | 425,504 | 138,735 | 36.983 | 1.121 |
| M21 + RNA_28 | M21 + RNA_44 | InfB | 288,294 | 32,996 | 35.674 | 0.486 | 127,687 | 19,551 | 36.210 | 0.574 |
| M21 + RNA_28 | M21 + RNA_44 | RNaseP | 1,229,564 | 19,901 | 34.801 | 0.210 | 841,233 | 75,029 | 35.133 | 0.323 |
| M21 + RNA_28 | M21 + RNA_44 | SARS-CoV- | 445,035 | 17,276 | 35.043 | 0.190 | 79,018 | 21,752 | 36.763 | 1.139 |
| | | 2 | | | | | | | | |
| M21 + RNA_30 | M21 + RNA_31 | InfA | 843,872 | 48,532 | 34.924 | 0.311 | 433,208 | 13,129 | 36.592 | 0.036 |
| M21 + RNA_30 | M21 + RNA_31 | InfB | 265,147 | 31,068 | 35.884 | 0.761 | 102,551 | 28,601 | 36.650 | 1.260 |
| M21 + RNA_30 | M21 + RNA_31 | RNaseP | 1,223,644 | 62,377 | 34.607 | 0.180 | 895,206 | 13,997 | 34.744 | 0.324 |
| M21 + RNA_30 | M21 + RNA_31 | SARS-CoV- | 444,411 | 25,670 | 35.058 | 0.384 | 57,497 | 577 | 36.728 | 0.238 |
| | | 2 | | | | | | | | |
| M21 + RNA_30 | M21 + RNA_32 | InfA | 726,137 | 30,559 | 35.635 | 0.492 | 363,624 | 154,612 | 37.410 | 2.231 |
| M21 + RNA_30 | M21 + RNA_32 | InfB | 291,038 | 29,969 | 34.843 | 0.193 | 112,168 | 11,870 | 36.218 | 0.371 |
| M21 + RNA_30 | M21 + RNA_32 | RNaseP | 1,239,508 | 27,353 | 34.526 | 0.288 | 885,668 | 104,924 | 35.271 | 0.479 |
| M21 + RNA_30 | M21 + RNA_32 | SARS-CoV- | 443,281 | 23,369 | 35.042 | 0.465 | 119,373 | 30,746 | 35.894 | 0.994 |
| | | 2 | | | | | | | | |
| M21 + RNA_30 | M21 + RNA_39 | InfA | 750,568 | 26,201 | 35.316 | 0.388 | 328,950 | 31,901 | 36.867 | 0.248 |
| M21 + RNA_30 | M21 + RNA_39 | InfB | 277,552 | 23,294 | 34.975 | 0.335 | 83,427 | 58,195 | 35.813 | 0.196 |
| M21 + RNA_30 | M21 + RNA_39 | RNaseP | 1,071,619 | 75,447 | 34.943 | 0.273 | 806,136 | 38,417 | 35.173 | 0.303 |
| M21 + RNA_30 | M21 + RNA_39 | SARS-CoV- | 379,041 | 33,118 | 35.161 | 0.671 | 92,062 | 26,495 | 36.190 | 1.208 |
| | | 2 | | | | | | | | |
| M21 + RNA_30 | M21 + RNA_40 | InfA | 750,713 | 23,368 | 35.625 | 0.459 | 407,997 | 89,946 | 37.027 | 1.030 |
| M21 + RNA_30 | M21 + RNA_40 | InfB | 301,919 | 16,860 | 34.831 | 0.195 | 120,744 | 34,305 | 36.432 | 0.852 |
| M21 + RNA_30 | M21 + RNA_40 | RNaseP | 1,174,847 | 53,510 | 34.730 | 0.275 | 869,257 | 62,279 | 35.124 | 0.150 |
| M21 + RNA_30 | M21 + RNA_40 | SARS-CoV- | 450,057 | 37,031 | 34.713 | 0.578 | 123,282 | 7,379 | 35.867 | 0.065 |
| | | 2 | | | | | | | | |
| M21 + RNA_30 | M21 + RNA_42 | InfA | 805,689 | 71,970 | 35.521 | 0.635 | 512,807 | 79,228 | 36.349 | 0.627 |
| M21 + RNA_30 | M21 + RNA_42 | InfB | 309,903 | 15,151 | 35.034 | 0.216 | 110,982 | 28,028 | 36.588 | 1.049 |
| M21 + RNA_30 | M21 + RNA_42 | RNaseP | 1,238,226 | 50,183 | 34.741 | 0.444 | 942,221 | 54,213 | 34.915 | 0.312 |
| M21 + RNA_30 | M21 + RNA_42 | SARS-CoV- | 448,510 | 24,609 | 35.185 | 0.586 | 89,913 | 14,988 | 35.652 | 0.312 |
| | | 2 | | | | | | | | |
| M21 + RNA_30 | M21 + RNA_43 | InfA | 793,635 | 19,532 | 35.330 | 0.241 | 393,714 | 98,802 | 36.977 | 0.647 |
| M21 + RNA_30 | M21 + RNA_43 | InfB | 302,371 | 20,407 | 35.058 | 0.474 | 68,091 | 24,073 | 38.307 | 2.224 |
| M21 + RNA_30 | M21 + RNA_43 | RNaseP | 1,173,582 | 76,932 | 34.540 | 0.268 | 755,672 | 69,313 | 35.537 | 0.561 |
| M21 + RNA_30 | M21 + RNA_43 | SARS-CoV- | 465,323 | 34,249 | 35.295 | 0.682 | 84,325 | 14,262 | 36.438 | 0.511 |
| | | 2 | | | | | | | | |
| M21 + RNA_30 | M21 + RNA_44 | InfA | 762,606 | 37,221 | 35.443 | 0.097 | 446,954 | 97,839 | 36.686 | 0.830 |
| M21 + RNA_30 | M21 + RNA_44 | InfB | 296,270 | 12,645 | 34.960 | 0.204 | 110,467 | 25,588 | 36.736 | 0.650 |
| M21 + RNA_30 | M21 + RNA_44 | RNaseP | 1,157,044 | 136,692 | 35.236 | 0.426 | 889,973 | 155,577 | 34.920 | 0.074 |
| M21 + RNA_30 | M21 + RNA_44 | SARS-CoV- | 420,149 | 43,112 | 35.191 | 0.189 | 66,817 | 40,386 | 38.639 | 3.707 |
| | | 2 | | | | | | | | |
| M21 + RNA_31 | M21 + RNA_32 | InfA | 782,117 | 60,162 | 35.640 | 0.521 | 385,563 | 41,210 | 37.057 | 0.388 |
| M21 + RNA_31 | M21 + RNA_32 | InfB | 294,064 | 24,444 | 35.352 | 0.187 | 116,259 | 28,877 | 36.960 | 0.876 |
| M21 + RNA_31 | M21 + RNA_32 | RNaseP | 1,235,604 | 30,002 | 34.569 | 0.126 | 897,428 | 58,077 | 35.111 | 0.473 |
| M21 + RNA_31 | M21 + RNA_32 | SARS-CoV- | 470,540 | 22,284 | 35.255 | 0.343 | 175,881 | 39,698 | 35.940 | 0.972 |
| | | 2 | | | | | | | | |
| M21 + RNA_31 | M21 + RNA_39 | InfA | 753,355 | 25,092 | 35.691 | 0.164 | 424,651 | 33,134 | 36.563 | 0.657 |
| M21 + RNA_31 | M21 + RNA_39 | InfB | 290,978 | 32,270 | 35.060 | 0.832 | 138,825 | 20,297 | 36.238 | 0.569 |
| M21 + RNA_31 | M21 + RNA_39 | RNaseP | 1,269,632 | 71,311 | 34.521 | 0.229 | 980,469 | 39,044 | 34.863 | 0.270 |
| M21 + RNA_31 | M21 + RNA_39 | SARS-CoV- | 472,886 | 11,297 | 34.809 | 0.414 | 140,743 | 33,204 | 36.145 | 0.406 |
| | | 2 | | | | | | | | |
| M21 + RNA_31 | M21 + RNA_40 | InfA | 719,008 | 56,208 | 35.683 | 0.675 | 479,215 | 82,428 | 36.422 | 0.555 |
| M21 + RNA_31 | M21 + RNA_40 | InfB | 286,935 | 13,596 | 35.048 | 0.258 | 114,233 | 25,012 | 36.731 | 1.118 |
| M21 + RNA_31 | M21 + RNA_40 | RNaseP | 1,145,276 | 46,519 | 34.764 | 0.223 | 866,174 | 28,676 | 35.465 | 0.394 |
| M21 + RNA_31 | M21 + RNA_40 | SARS-CoV- | 438,680 | 28,114 | 35.341 | 0.345 | 197,505 | 49,355 | 35.836 | 0.459 |
| | | 2 | | | | | | | | |
| M21 + RNA_31 | M21 + RNA_42 | InfA | 737,286 | 48,322 | 35.553 | 0.475 | 451,477 | 57,179 | 36.345 | 0.414 |
| M21 + RNA_31 | M21 + RNA_42 | InfB | 272,398 | 11,778 | 35.481 | 0.661 | 117,266 | 21,213 | 36.408 | 0.608 |
| M21 + RNA_31 | M21 + RNA_42 | RNaseP | 1,145,447 | 6,711 | 34.811 | 0.211 | 815,853 | 94,490 | 35.813 | 0.973 |
| M21 + RNA_31 | M21 + RNA_42 | SARS-CoV- | 406,648 | 21,745 | 35.060 | 0.203 | 124,222 | 7,787 | 35.997 | 0.099 |
| | | 2 | | | | | | | | |
| M21 + RNA_31 | M21 + RNA_43 | InfA | 777,672 | 53,734 | 35.373 | 0.483 | 415,009 | 92,805 | 36.715 | 0.521 |
| M21 + RNA_31 | M21 + RNA_43 | InfB | 271,289 | 31,424 | 35.251 | 0.427 | 122,797 | 8,588 | 36.382 | 0.181 |
| M21 + RNA_31 | M21 + RNA_43 | RNaseP | 1,195,763 | 12,295 | 34.741 | 0.177 | 848,664 | 13,395 | 34.763 | 0.051 |
| M21 + RNA_31 | M21 + RNA_43 | SARS-CoV- | 450,594 | 20,904 | 35.484 | 0.294 | 137,740 | 8,115 | 35.896 | 0.257 |
| | | 2 | | | | | | | | |
| M21 + RNA_31 | M21 + RNA_44 | InfA | 742,427 | 27,658 | 36.008 | 0.783 | 439,638 | 22,958 | 36.708 | 0.215 |
| M21 + RNA_31 | M21 + RNA_44 | InfB | 311,881 | 7,159 | 34.808 | 0.260 | 122,358 | 20,999 | 36.530 | 0.512 |
| M21 + RNA_31 | M21 + RNA_44 | RNaseP | 1,294,083 | 87,886 | 34.643 | 0.248 | 992,034 | 27,824 | 34.948 | 0.242 |
| M21 + RNA_31 | M21 + RNA_44 | SARS-CoV- | 483,839 | 44,078 | 35.032 | 0.423 | 98,914 | 7,980 | 36.079 | 0.175 |
| | | 2 | | | | | | | | |
| M21 + RNA_32 | M21 + RNA_39 | InfA | 782,825 | 52,798 | 35.461 | 0.479 | 397,070 | 66,846 | 36.078 | 0.606 |
| M21 + RNA_32 | M21 + RNA_39 | InfB | 296,373 | 7,206 | 35.381 | 0.443 | 88,286 | 25,977 | 37.046 | 1.305 |
| M21 + RNA_32 | M21 + RNA_39 | RNaseP | 1,298,215 | 39,096 | 34.464 | 0.098 | 860,330 | 95,793 | 34.914 | 0.273 |
| M21 + RNA_32 | M21 + RNA_39 | SARS-CoV- | 484,652 | 14,242 | 35.286 | 0.522 | 89,073 | 34,136 | 37.363 | 1.752 |
| | | 2 | | | | | | | | |
| M21 + RNA_32 | M21 + RNA_40 | InfA | 743,482 | 60,705 | 35.854 | 0.920 | 407,460 | 92,450 | 36.705 | 0.736 |
| M21 + RNA_32 | M21 + RNA_40 | InfB | 287,758 | 49,300 | 35.461 | 0.953 | 129,394 | 26,779 | 36.095 | 0.940 |
| M21 + RNA_32 | M21 + RNA_40 | RNaseP | 1,162,113 | 96,838 | 35.032 | 0.422 | 917,303 | 51,543 | 35.240 | 0.330 |
| M21 + RNA_32 | M21 + RNA_40 | SARS-CoV- | 486,080 | 37,118 | 35.398 | 0.499 | 220,290 | 15,265 | 35.575 | 0.372 |
| | | 2 | | | | | | | | |
| M21 + RNA_32 | M21 + RNA_42 | InfA | 771,878 | 19,721 | 35.961 | 0.173 | 321,360 | 14,681 | 37.273 | 0.377 |
| M21 + RNA_32 | M21 + RNA_42 | InfB | 296,632 | 25,801 | 35.493 | 0.631 | 121,683 | 32,290 | 36.286 | 0.704 |
| M21 + RNA_32 | M21 + RNA_42 | RNaseP | 1,142,429 | 106,507 | 35.187 | 0.374 | 831,224 | 80,366 | 35.073 | 0.333 |
| M21 + RNA_32 | M21 + RNA_42 | SARS-CoV- | 462,792 | 54,247 | 35.084 | 0.264 | 187,859 | 18,451 | 35.464 | 0.105 |
| | | 2 | | | | | | | | |
| M21 + RNA_32 | M21 + RNA_43 | InfA | 756,221 | 44,690 | 35.514 | 0.222 | 427,588 | 113,676 | 36.779 | 1.315 |
| M21 + RNA_32 | M21 + RNA_43 | InfB | 275,248 | 12,906 | 35.254 | 0.355 | 105,692 | 26,291 | 36.608 | 0.920 |
| M21 + RNA_32 | M21 + RNA_43 | RNaseP | 1,123,040 | 62,095 | 34.938 | 0.420 | 851,221 | 60,935 | 35.037 | 0.461 |
| M21 + RNA_32 | M21 + RNA_43 | SARS-CoV- | 435,488 | 42,945 | 35.558 | 0.437 | 184,385 | 8,477 | 35.687 | 0.148 |
| | | 2 | | | | | | | | |
| M21 + RNA_32 | M21 + RNA_44 | InfA | 780,540 | 20,582 | 35.607 | 0.380 | 416,757 | 16,496 | 36.726 | 0.142 |
| M21 + RNA_32 | M21 + RNA_44 | InfB | 300,957 | 16,722 | 35.155 | 0.456 | 117,296 | 21,140 | 36.635 | 0.727 |
| M21 + RNA_32 | M21 + RNA_44 | RNaseP | 1,196,270 | 33,328 | 35.185 | 0.021 | 905,904 | 67,538 | 35.006 | 0.049 |
| M21 + RNA_32 | M21 + RNA_44 | SARS-CoV- | 441,447 | 28,383 | 35.455 | 0.607 | 135,203 | 32,342 | 36.117 | 0.531 |
| | | 2 | | | | | | | | |
| M21 + RNA_39 | M21 + RNA_40 | InfA | 718,889 | 40,721 | 35.495 | 0.436 | 346,976 | 50,660 | 36.282 | 0.660 |
| M21 + RNA_39 | M21 + RNA_40 | InfB | 298,594 | 19,274 | 34.789 | 0.330 | 129,618 | 7,388 | 35.645 | 0.164 |
| M21 + RNA_39 | M21 + RNA_40 | RNaseP | 1,081,718 | 34,671 | 35.092 | 0.445 | 809,243 | 127,810 | 34.986 | 0.300 |
| M21 + RNA_39 | M21 + RNA_40 | SARS-CoV- | 418,797 | 20,979 | 35.383 | 0.551 | 101,668 | 32,824 | 36.636 | 1.191 |
| | | 2 | | | | | | | | |
| M21 + RNA_39 | M21 + RNA_42 | InfA | 842,976 | 3,452 | 35.028 | 0.340 | 402,272 | 25,336 | 36.535 | 0.389 |
| M21 + RNA_39 | M21 + RNA_42 | InfB | 305,873 | 11,341 | 34.943 | 0.136 | 87,630 | 12,444 | 37.073 | 0.916 |
| M21 + RNA_39 | M21 + RNA_42 | RNaseP | 1,318,429 | 18,765 | 34.559 | 0.056 | 909,746 | 101,513 | 35.325 | 0.398 |
| M21 + RNA_39 | M21 + RNA_42 | SARS-CoV- | 503,379 | 31,195 | 34.753 | 0.393 | 91,950 | 73,954 | 36.065 | 0.363 |
| | | 2 | | | | | | | | |
| M21 + RNA_39 | M21 + RNA_43 | InfA | 778,932 | 9,142 | 35.370 | 0.105 | 384,466 | 138,397 | 36.868 | 1.627 |
| M21 + RNA_39 | M21 + RNA_43 | InfB | 318,060 | 36,109 | 34.948 | 0.452 | 101,980 | 24,229 | 36.636 | 0.994 |
| M21 + RNA_39 | M21 + RNA_43 | RNaseP | 1,262,380 | 44,513 | 34.855 | 0.211 | 923,839 | 10,101 | 34.731 | 0.110 |
| M21 + RNA_39 | M21 + RNA_43 | SARS-CoV- | 496,182 | 34,073 | 35.353 | 0.408 | 71,902 | 36,795 | 38.640 | 2.436 |
| | | 2 | | | | | | | | |
| M21 + RNA_39 | M21 + RNA_44 | InfA | 694,092 | 114,804 | 36.287 | 1.037 | 388,844 | 19,620 | 36.823 | 0.136 |
| M21 + RNA_39 | M21 + RNA_44 | InfB | 257,435 | 14,532 | 35.937 | 0.265 | 145,819 | 33,505 | 35.939 | 0.749 |
| M21 + RNA_39 | M21 + RNA_44 | RNaseP | 1,236,028 | 14,467 | 34.939 | 0.467 | 948,751 | 148,377 | 35.161 | 0.514 |
| M21 + RNA_39 | M21 + RNA_44 | SARS-CoV- | 455,904 | 27,924 | 35.576 | 0.496 | 138,794 | 19,064 | 35.762 | 0.263 |
| | | 2 | | | | | | | | |
| M21 + RNA_40 | M21 + RNA_42 | InfA | 838,706 | 41,733 | 35.132 | 0.242 | 346,713 | 96,229 | 37.556 | 1.312 |
| M21 + RNA_40 | M21 + RNA_42 | InfB | 293,980 | 12,054 | 35.555 | 0.525 | 140,817 | 26,556 | 36.090 | 0.640 |
| M21 + RNA_40 | M21 + RNA_42 | RNaseP | 1,217,799 | 39,398 | 35.156 | 0.269 | 913,431 | 40,804 | 34.947 | 0.101 |
| M21 + RNA_40 | M21 + RNA_42 | SARS-CoV- | 507,886 | 19,460 | 34.863 | 0.331 | 182,825 | 41,585 | 36.147 | 0.796 |
| | | 2 | | | | | | | | |
| M21 + RNA_40 | M21 + RNA_43 | InfA | 753,481 | 50,601 | 35.651 | 0.079 | 458,432 | 52,679 | 36.440 | 0.490 |
| M21 + RNA_40 | M21 + RNA_43 | InfB | 278,421 | 10,305 | 34.907 | 0.644 | 123,643 | 26,267 | 36.214 | 0.626 |
| M21 + RNA_40 | M21 + RNA_43 | RNaseP | 1,135,219 | 32,110 | 34.965 | 0.183 | 799,776 | 51,935 | 35.170 | 0.560 |
| M21 + RNA_40 | M21 + RNA_43 | SARS-CoV- | 483,121 | 17,659 | 35.554 | 0.255 | 231,946 | 33,365 | 35.485 | 0.744 |
| | | 2 | | | | | | | | |
| M21 + RNA_40 | M21 + RNA_44 | InfA | 774,849 | 46,280 | 35.763 | 0.601 | 532,800 | 107,474 | 36.295 | 0.840 |
| M21 + RNA_40 | M21 + RNA_44 | InfB | 307,636 | 14,013 | 35.090 | 0.239 | 153,330 | 10,668 | 36.049 | 0.272 |
| M21 + RNA_40 | M21 + RNA_44 | RNaseP | 1,233,941 | 42,154 | 35.038 | 0.286 | 912,791 | 30,910 | 35.233 | 0.391 |
| M21 + RNA_40 | M21 + RNA_44 | SARS-CoV- | 422,843 | 19,406 | 35.584 | 0.285 | 190,969 | 25,532 | 35.498 | 0.481 |
| | | 2 | | | | | | | | |
| M21 + RNA_42 | M21 + RNA_43 | InfA | 695,834 | 40,875 | 36.020 | 0.564 | 489,748 | 107,913 | 36.153 | 1.134 |
| M21 + RNA_42 | M21 + RNA_43 | InfB | 295,205 | 33,689 | 34.751 | 0.726 | 131,290 | 27,944 | 36.047 | 0.730 |
| M21 + RNA_42 | M21 + RNA_43 | RNaseP | 1,210,355 | 78,083 | 34.641 | 0.258 | 829,571 | 95,526 | 35.420 | 0.469 |
| M21 + RNA_42 | M21 + RNA_43 | SARS-CoV- | 511,911 | 27,158 | 34.849 | 0.135 | 142,510 | 36,215 | 36.285 | 0.736 |
| | | 2 | | | | | | | | |
| M21 + RNA_42 | M21 + RNA_44 | InfA | 719,762 | 64,445 | 35.698 | 0.482 | 433,466 | 32,046 | 36.569 | 0.514 |
| M21 + RNA_42 | M21 + RNA_44 | InfB | 289,031 | 25,261 | 35.275 | 0.459 | 112,069 | 17,026 | 36.421 | 0.580 |
| M21 + RNA_42 | M21 + RNA_44 | RNaseP | 1,212,106 | 9,836 | 34.989 | 0.188 | 943,690 | 60,995 | 34.966 | 0.225 |
| M21 + RNA_42 | M21 + RNA_44 | SARS-CoV- | 426,871 | 4,363 | 35.138 | 0.224 | 106,381 | 26,821 | 36.096 | 0.889 |
| | | 2 | | | | | | | | |
| M21 + RNA_43 | M21 + RNA_44 | InfA | 815,541 | 18,420 | 35.345 | 0.754 | 473,484 | 8,143 | 36.672 | 0.123 |
| M21 + RNA_43 | M21 + RNA_44 | InfB | 281,729 | 55,144 | 35.345 | 0.934 | 107,945 | 14,879 | 36.867 | 0.446 |
| M21 + RNA_43 | M21 + RNA_44 | RNaseP | 1,247,368 | 53,000 | 34.631 | 0.145 | 958,956 | 72,300 | 34.939 | 0.415 |
| M21 + RNA_43 | M21 + RNA_44 | SARS-CoV- | 453,290 | 41,868 | 35.067 | 0.222 | 144,129 | 50,939 | 35.702 | 0.832 |
| | | 2 | | | | | | | | |
| RNA Aptamer | | InfA | 757,259 | 33,324 | 35.343 | 0.341 | 179,024 | 30,749 | 38.141 | 0.638 |
| Only | | | | | | | | | | |
| RNA Aptamer | | InfB | 237,038 | 15,562 | 35.503 | 0.267 | 17,429 | 2,789 | NA | NA |
| Only | | | | | | | | | | |
| RNA Aptamer | | RNaseP | 1,052,139 | 42,467 | 35.301 | 0.392 | 372,869 | 43,395 | 35.636 | 0.786 |
| Only | | | | | | | | | | |
| RNA Aptamer | | SARS-CoV- | 335,970 | 49,293 | 35.892 | 0.408 | 23,616 | 1,531 | NA | NA |
| Only | | 2 | | | | | | | | |
| No Aptamer | | InfA | 704,276 | 27,887 | 35.704 | 0.482 | 65,598 | 2,338 | 42.096 | 0.462 |
| No Aptamer | | InfB | 297,504 | 27,397 | 34.685 | 0.785 | 16,944 | 2,473 | NA | NA |
| No Aptamer | | RNaseP | 1,007,289 | 12,637 | 35.112 | 0.295 | 408,839 | 9,002 | 35.262 | 0.407 |
| No Aptamer | | SARS-CoV- | 415,278 | 28,422 | 35.048 | 0.088 | 29,069 | 9,303 | 39.917 | 2.906 |
| | | 2 | | | | | | | | |
| No Template | | InfA | 7,224 | 1,309 | NA | NA | 4,110 | 2,080 | NA | NA |
| Control | | | | | | | | | | |
| No Template | | InfB | 3,051 | 630 | NA | NA | 702 | 369 | NA | NA |
| Control | | | | | | | | | | |
| No Template | | RNaseP | 6,754 | 994 | NA | NA | 2,731 | 474 | NA | NA |
| Control | | | | | | | | | | |
| No Template | | SARS-CoV- | 3,396 | 553 | NA | NA | 803 | 143 | NA | NA |
| Control | | 2 | | | | | | | | |

TABLE 7
Evaluation of room temperature stability at 0 hours, 4 hours, and 12 hours following template addition to PrimeTime ™
One-Step RT-qPCR Master Mix (Integrated DNA Technologies) with single or combinations of DNA aptamers at various
concentrations with and without RNA Aptamer.

| | | | | | 0 h Endpoint | | | | 4 h Endpoint | | | | 12 h Endpoint | | | |
|---|---|---|---|---|---|---|---|---|---|---|---|---|---|---|---|---|
| | | | | | Fluorescence | | 0 h Cycle | | Fluorescence | | 4 h Cycle | | Fluorescence | | | |
| | | | | | Delta | Delta | Threshold | | Delta | Delta | Threshold | | Delta | Delta | 12 h CycleThreshold | |
| Aptamer | Aptamer | RNA | | Target | Rn | Rn | Ct | Ct | Rn | Rn | Ct | Ct | Rn | Rn | | |
| 1 | 2 | Aptamer | Ratio | Name | Mean | SD | Mean | SD | Mean | SD | Mean | SD | Mean | SD | Ct Mean | Ct SD |
| M21_44 | | No | 1:01 | InfA | 709838 | 39882 | 35.023 | 0.293 | 29261 | 17267 | 42.568 | 0.257 | 472 | 468 | NA | NA |
| M21_44 | | No | 1:01 | InfB | 232714 | 17837 | 34.904 | 0.582 | 5542 | 2718 | NA | NA | −236 | 25 | NA | NA |
| M21_44 | | No | 1:01 | RNaseP | 831238 | 68977 | 33.692 | 0.24 | 195319 | 69183 | 34.593 | 0.899 | 17750 | 11890 | NA | NA |
| M21_44 | | No | 1:01 | SARS- | 293357 | 74438 | 34.783 | 0.429 | 19738 | 3593 | 38.351 | 2.801 | 215 | 101 | NA | NA |
| | | | | CoV-2 | | | | | | | | | | | | |
| M21_44 | M21 + | No | 1:01: | InfA | 720988 | 14857 | 34.929 | 0.181 | 117476 | 7013 | 36.857 | 0.371 | 5912 | 2128 | NA | NA |
| | RNA_ | | 01 | | | | | | | | | | | | | |
| | 31 | | | | | | | | | | | | | | | |
| M21_44 | M21 + | No | 1:01: | InfB | 257861 | 14879 | 34.884 | 0.711 | 29898 | 6567 | 36.557 | 1.652 | 254 | 95 | NA | NA |
| | RNA_ | | 01 | | | | | | | | | | | | | |
| | 31 | | | | | | | | | | | | | | | |
| M21_44 | M21 + | No | 1:01: | RNaseP | 943663 | 46165 | 33.372 | 0.441 | 458177 | 67883 | 33.443 | 0.151 | 112733 | 8250 | 35.164 | 0.583 |
| | RNA_ | | 01 | | | | | | | | | | | | | |
| | 31 | | | | | | | | | | | | | | | |
| M21_44 | M21 + | No | 1:01: | SARS- | 370948 | 31566 | 34.814 | 0.766 | 25548 | 14030 | 35.179 | 1.127 | 260 | 135 | NA | NA |
| | RNA_ | | 01 | CoV-2 | | | | | | | | | | | | |
| | 31 | | | | | | | | | | | | | | | |
| M21_44 | | Yes | 01: | InfA | 728735 | 25927 | 35.401 | 0.402 | 111221 | 33476 | 37.866 | 1.803 | 5565 | 2648 | NA | NA |
| | | | 03.3 | | | | | | | | | | | | | |
| M21_44 | | Yes | 01: | InfB | 276677 | 6571 | 34.464 | 0.28 | 7967 | 3919 | NA | NA | −151 | 198 | NA | NA |
| | | | 03.3 | | | | | | | | | | | | | |
| M21_44 | | Yes | 01: | RNaseP | 990056 | 71612 | 33.689 | 0.734 | 191988 | 11951 | 34.039 | 0.262 | 40886 | 2539 | NA | NA |
| | | | 03.3 | | | | | | | | | | | | | |
| M21_44 | | Yes | 01: | SARS- | 410062 | 24492 | 34.518 | 0.357 | 4218 | 3844 | NA | NA | 80 | 171 | NA | NA |
| | | | 03.3 | CoV-2 | | | | | | | | | | | | |
| M21_44 | M21 + | Yes | 1:1:1: | InfA | 730467 | 44611 | 35.249 | 0.746 | 134072 | 27188 | 37.924 | 0.867 | 35983 | 14929 | 40.322 | NA |
| | RNA_ | | 3.3 | | | | | | | | | | | | | |
| | 31 | | | | | | | | | | | | | | | |
| M21_44 | M21 + | Yes | 1:1:1: | InfB | 270170 | 41008 | 34.634 | 0.255 | 3084 | 674 | NA | NA | 200 | 58 | NA | NA |
| | RNA_ | | 3.3 | | | | | | | | | | | | | |
| | 31 | | | | | | | | | | | | | | | |
| M21_44 | M21 + | Yes | 1:1:1: | RNaseP | 1012387 | 10622 | 33.692 | 0.23 | 289902 | 23592 | 33.341 | 0.334 | 63272 | 16375 | 37.864 | 1.739 |
| | RNA_ | | 3.3 | | | | | | | | | | | | | |
| | 31 | | | | | | | | | | | | | | | |
| M21_44 | M21 + | Yes | 1:1:1: | SARS- | 412277 | 6406 | 34.22 | 0.577 | 17545 | 10604 | 36.699 | 0.748 | 157 | 97 | NA | NA |
| | RNA_ | | 3.3 | CoV-2 | | | | | | | | | | | | |
| | 31 | | | | | | | | | | | | | | | |
| M21_44 | | No | 1:02 | InfA | 676180 | 64156 | 35.807 | 0.703 | 97194 | 19756 | 37.516 | 0.747 | 15093 | 3969 | NA | NA |
| M21_44 | | No | 1:02 | InfB | 285684 | 9656 | 34.406 | 0.095 | 17923 | 8568 | 35.848 | NA | 76 | 153 | NA | NA |
| M21_44 | | No | 1:02 | RNaseP | 1016977 | 22949 | 33.76 | 0.158 | 360257 | 50535 | 34.048 | 0.094 | 83770 | 16181 | 36.876 | 1.704 |
| M21_44 | | No | 1:02 | SARS- | 417088 | 35196 | 33.92 | 0.47 | 29478 | 6166 | 36.253 | 0.762 | 684 | 254 | NA | NA |
| | | | | CoV-2 | | | | | | | | | | | | |
| M21_44 | M21 + | No | 1:02: | InfA | 742719 | 24247 | 34.96 | 0.338 | 237055 | 101425 | 36.859 | 1.629 | 25611 | 3581 | NA | NA |
| | RNA_ | | 02 | | | | | | | | | | | | | |
| | 31 | | | | | | | | | | | | | | | |
| M21_44 | M21 + | No | 1:02: | InfB | 304865 | 5595 | 34.183 | 0.302 | 50337 | 24070 | 38.573 | 4.951 | 3274 | 2624 | NA | NA |
| | RNA_ | | 02 | | | | | | | | | | | | | |
| | 31 | | | | | | | | | | | | | | | |
| M21_44 | M21 + | No | 1:02: | RNaseP | 1037535 | 13135 | 33.658 | 0.281 | 573049 | 22943 | 34.083 | 0.491 | 246969 | 30331 | 34.498 | 0.632 |
| | RNA_ | | 02 | | | | | | | | | | | | | |
| | 31 | | | | | | | | | | | | | | | |
| M21_44 | M21 + | No | 1:02: | SARS- | 400243 | 8042 | 34.353 | 0.902 | 57020 | 6056 | 34.96 | 0.382 | 331 | 981 | NA | NA |
| | RNA_ | | 02 | CoV-2 | | | | | | | | | | | | |
| | 31 | | | | | | | | | | | | | | | |
| M21_44 | | Yes | 02: | InfA | 791895 | 23806 | 34.992 | 0.428 | 100953 | 16713 | 38.041 | 1.082 | 18814 | 2676 | NA | NA |
| | | | 03.3 | | | | | | | | | | | | | |
| M21_44 | | Yes | 02: | InfB | 303030 | 23600 | 34.069 | 0.312 | 8207 | 5548 | NA | NA | 144 | 179 | NA | NA |
| | | | 03.3 | | | | | | | | | | | | | |
| M21_44 | | Yes | 02: | RNaseP | 989885 | 4348 | 33.821 | 0.072 | 194497 | 24160 | 34.224 | 0.586 | 46855 | 6833 | 41.922 | 1.598 |
| | | | 03.3 | | | | | | | | | | | | | |
| M21_44 | | Yes | 02: | SARS- | 356018 | 33283 | 34.77 | 0.362 | 13400 | 3005 | 41.791 | 2.505 | 148 | 89 | NA | NA |
| | | | 03.3 | CoV-2 | | | | | | | | | | | | |
| M21_44 | M21 + | Yes | 1:2:2: | InfA | 776749 | 19462 | 34.662 | 0.304 | 160706 | 48180 | 38.125 | 1.442 | 7119 | 5601 | NA | NA |
| | RNA_ | | 3.3 | | | | | | | | | | | | | |
| | 31 | | | | | | | | | | | | | | | |
| M21_44 | M21 + | Yes | 1:2:2: | InfB | 306115 | 19498 | 33.935 | 0.675 | 24677 | 7798 | 39.882 | 3.266 | 325 | 115 | NA | NA |
| | RNA_ | | 3.3 | | | | | | | | | | | | | |
| | 31 | | | | | | | | | | | | | | | |
| M21_44 | M21 + | Yes | 1:2:2: | RNaseP | 916151 | 66143 | 34.11 | 0.303 | 311038 | 27938 | 33.696 | 0.158 | 80822 | 4280 | 37.09 | 0.718 |
| | RNA_ | | 3.3 | | | | | | | | | | | | | |
| | 31 | | | | | | | | | | | | | | | |
| M21_44 | M21 + | Yes | 1:2:2: | SARS- | 391250 | 45446 | 34.303 | 0.913 | 36452 | 17489 | 36.379 | 1.911 | 612 | 133 | NA | NA |
| | RNA_ | | 3.3 | CoV-2 | | | | | | | | | | | | |
| | 31 | | | | | | | | | | | | | | | |
| M21_44 | | No | 1:05 | InfA | 288260 | 313296 | 39.88 | 6.792 | 100510 | 87740 | 38.304 | 3.396 | 5929 | 13498 | NA | NA |
| M21_44 | | No | 1:05 | InfB | 151715 | 104580 | 38.366 | 3.372 | 42561 | 22164 | 35.98 | 3.111 | −1885 | 1473 | NA | NA |
| M21_44 | | No | 1:05 | RNaseP | 452819 | 333272 | 38.782 | 4.594 | 368663 | 108838 | 34.983 | 1.357 | 129873 | 80528 | 37.799 | 3.656 |
| M21_44 | | No | 1:05 | SARS- | 163400 | 116945 | 38.938 | 3.437 | 27548 | 6458 | 34.269 | 5.18 | 1419 | 2719 | NA | NA |
| | | | | CoV-2 | | | | | | | | | | | | |
| M21_44 | M21 + | No | 1:05: | InfA | 796325 | 24250 | 34.653 | 0.234 | 324247 | 114842 | 36.244 | 1.278 | 46333 | 10650 | 42.183 | 2.29 |
| | RNA_ | | 05 | | | | | | | | | | | | | |
| | 31 | | | | | | | | | | | | | | | |
| M21_44 | M21 + | No | 1:05: | InfB | 278865 | 17082 | 34.699 | 0.45 | 55470 | 14414 | 36.838 | 0.87 | 3513 | 4938 | NA | NA |
| | RNA_ | | 05 | | | | | | | | | | | | | |
| | 31 | | | | | | | | | | | | | | | |
| M21_44 | M21 + | No | 1:05: | RNaseP | 938820 | 50465 | 33.951 | 0.635 | 557356 | 28867 | 34.688 | 0.463 | 229478 | 57676 | 35.254 | 1.047 |
| | RNA_ | | 05 | | | | | | | | | | | | | |
| | 31 | | | | | | | | | | | | | | | |
| M21_44 | M21 + | No | 1:05: | SARS- | 400471 | 21336 | 34.163 | 0.393 | 83116 | 12892 | 35.239 | 0.501 | 2643 | 2469 | NA | NA |
| | RNA_ | | 05 | CoV-2 | | | | | | | | | | | | |
| | 31 | | | | | | | | | | | | | | | |
| M21_44 | | Yes | 05: | InfA | 728486 | 32735 | 34.968 | 0.208 | 262890 | 102753 | 36.532 | 1.608 | 25680 | 8659 | 44.957 | NA |
| | | | 03.3 | | | | | | | | | | | | | |
| M21_44 | | Yes | 05: | InfB | 264796 | 14429 | 34.757 | 0.266 | 17934 | 5873 | 41.336 | 5.171 | 101 | 119 | NA | NA |
| | | | 03.3 | | | | | | | | | | | | | |
| M21_44 | | Yes | 05: | RNaseP | 940991 | 39953 | 33.717 | 0.408 | 323137 | 54154 | 33.798 | 0.428 | 102099 | 30774 | 36.199 | 1.704 |
| | | | 03.3 | | | | | | | | | | | | | |
| M21_44 | | Yes | 05: | SARS- | 411734 | 10149 | 34.117 | 0.305 | 14155 | 18852 | 34.922 | NA | 105 | 279 | NA | NA |
| | | | 03.3 | CoV-2 | | | | | | | | | | | | |
| M21_44 | M21 + | Yes | 1:5:5: | InfA | 622263 | 41117 | 35.246 | 0.315 | 233432 | 24209 | 37.441 | 0.65 | 39131 | 28828 | 41.219 | 1.423 |
| | RNA_ | | 3.3 | | | | | | | | | | | | | |
| | 31 | | | | | | | | | | | | | | | |
| M21_44 | M21 + | Yes | 1:5:5: | InfB | 216429 | 38522 | 34.776 | 0.931 | 27235 | 4557 | 38.432 | 1.735 | 1490 | 1570 | NA | NA |
| | RNA_ | | 3.3 | | | | | | | | | | | | | |
| | 31 | | | | | | | | | | | | | | | |
| M21_44 | M21 + | Yes | 1:5:5: | RNaseP | 935583 | 43338 | 33.463 | 0.662 | 346197 | 37190 | 33.996 | 0.395 | 173346 | 19443 | 34.401 | 0.519 |
| | RNA_ | | 3.3 | | | | | | | | | | | | | |
| | 31 | | | | | | | | | | | | | | | |
| M21_44 | M21 + | Yes | 1:5:5: | SARS- | 379082 | 24308 | 33.797 | 0.243 | 36216 | 9668 | 36.497 | 1.422 | 522 | 986 | NA | NA |
| | RNA_ | | 3.3 | CoV-2 | | | | | | | | | | | | |
| | 31 | | | | | | | | | | | | | | | |
| M21 + | | No | 1:01 | InfA | 650085 | 19680 | 35.298 | 0.399 | 92085 | 54146 | 36.341 | 1.505 | 377 | 127 | NA | NA |
| RNA_9 | | | | | | | | | | | | | | | | |
| M21 + | | No | 1:01 | InfB | 242311 | 13573 | 34.875 | 0.356 | 7189 | 4487 | NA | NA | −415 | 94 | NA | NA |
| RNA_9 | | | | | | | | | | | | | | | | |
| M21 + | | No | 1:01 | RNaseP | 850666 | 83696 | 34.035 | 0.152 | 373676 | 40862 | 33.56 | 0.374 | 57969 | 5861 | 40.498 | 2.136 |
| RNA_9 | | | | | | | | | | | | | | | | |
| M21 + | | No | 1:01 | SARS- | 345452 | 17797 | 34.699 | 0.142 | 14593 | 2552 | 41.011 | 3.035 | −153 | 103 | NA | NA |
| RNA_9 | | | | CoV-2 | | | | | | | | | | | | |
| M21 + | M21 + | No | 1:01: | InfA | 695147 | 70079 | 35.237 | 0.603 | 199774 | 35399 | 36.321 | 0.732 | 25460 | 18028 | 40.211 | NA |
| RNA_9 | RNA_ | | 01 | | | | | | | | | | | | | |
| | 27 | | | | | | | | | | | | | | | |
| M21 + | M21 + | No | 1:01: | InfB | 244344 | 34017 | 35.255 | 1.3 | 47489 | 10069 | 35.753 | 0.845 | 20 | 350 | NA | NA |
| RNA_9 | RNA_ | | 01 | | | | | | | | | | | | | |
| | 27 | | | | | | | | | | | | | | | |
| M21 + | M21 + | No | 1:01: | RNaseP | 989066 | 38084 | 33.656 | 0.234 | 573734 | 12209 | 33.668 | 0.419 | 185653 | 19293 | 34.616 | 0.495 |
| RNA_9 | RNA_ | | 01 | | | | | | | | | | | | | |
| | 27 | | | | | | | | | | | | | | | |
| M21 + | M21 + | No | 1:01: | SARS- | 400719 | 3263 | 34.157 | 0.312 | 63121 | 37703 | 34.691 | 0.5 | 116 | 361 | NA | NA |
| RNA_9 | RNA_ | | 01 | CoV-2 | | | | | | | | | | | | |
| | 27 | | | | | | | | | | | | | | | |
| M21 + | | Yes | 01: | InfA | 733795 | 46643 | 34.802 | 0.691 | 211618 | 17404 | 36.596 | 0.329 | 11041 | 7805 | NA | NA |
| RNA_9 | | | 03.3 | | | | | | | | | | | | | |
| M21 + | | Yes | 01: | InfB | 292756 | 3610 | 34.158 | 0.105 | 8409 | 5264 | NA | NA | −433 | 132 | NA | NA |
| RNA_9 | | | 03.3 | | | | | | | | | | | | | |
| M21 + | | Yes | 01: | RNaseP | 944777 | 32847 | 33.727 | 0.215 | 270214 | 14386 | 33.608 | 0.509 | 55760 | 6977 | 41.297 | 2.744 |
| RNA_9 | | | 03.3 | | | | | | | | | | | | | |
| M21 + | | Yes | 01: | SARS- | 407352 | 16499 | 34.447 | 0.18 | 10540 | 5767 | 40.457 | NA | −24 | 386 | NA | NA |
| RNA_9 | | | 03.3 | CoV-2 | | | | | | | | | | | | |
| M21 + | M21 + | Yes | 1:1:1: | InfA | 697646 | 43591 | 35.556 | 0.899 | 97437 | 28902 | 39.757 | 2.075 | 32071 | 17984 | 41.125 | NA |
| RNA_9 | RNA_ | | 3.3 | | | | | | | | | | | | | |
| | 27 | | | | | | | | | | | | | | | |
| M21 + | M21 + | Yes | 1:1:1: | InfB | 262222 | 13563 | 34.265 | 0.65 | 14863 | 10836 | 36.553 | NA | 201 | 47 | NA | NA |
| RNA_9 | RNA_ | | 3.3 | | | | | | | | | | | | | |
| | 27 | | | | | | | | | | | | | | | |
| M21 + | M21 + | Yes | 1:1:1: | RNaseP | 1009111 | 60338 | 33.46 | 0.453 | 294807 | 58842 | 33.929 | 0.882 | 90008 | 36449 | 38.379 | 4.472 |
| RNA_9 | RNA_ | | 3.3 | | | | | | | | | | | | | |
| | 27 | | | | | | | | | | | | | | | |
| M21 + | M21 + | Yes | 1:1:1: | SARS- | 431598 | 43497 | 34.251 | 0.688 | 15391 | 11984 | 39.082 | 3.701 | 132 | 191 | NA | NA |
| RNA_9 | RNA_ | | 3.3 | CoV-2 | | | | | | | | | | | | |
| | 27 | | | | | | | | | | | | | | | |
| M21 + | | No | 1:02 | InfA | 706806 | 56631 | 35.23 | 0.629 | 241037 | 42394 | 36.496 | 0.548 | 31866 | 9694 | 41.902 | NA |
| RNA_9 | | | | | | | | | | | | | | | | |
| M21 + | | No | 1:02 | InfB | 275887 | 14508 | 34.585 | 0.48 | 60341 | 15348 | 36.113 | 0.512 | 5357 | 2815 | NA | NA |
| RNA_9 | | | | | | | | | | | | | | | | |
| M21 + | | No | 1:02 | RNaseP | 1074412 | 49642 | 33.664 | 0.221 | 605962 | 65649 | 33.992 | 0.26 | 301642 | 12459 | 34.003 | 0.052 |
| RNA_9 | | | | | | | | | | | | | | | | |
| M21 + | | No | 1:02 | SARS- | 442041 | 9978 | 34.317 | 0.297 | 81494 | 23187 | 35.127 | 0.306 | 8981 | 6445 | 39.541 | NA |
| RNA_9 | | | | CoV-2 | | | | | | | | | | | | |
| M21 + | M21 + | No | 1:02: | InfA | 725742 | 39985 | 35.017 | 0.365 | 274089 | 50896 | 36.981 | 1.024 | 33749 | 11574 | 41.526 | NA |
| RNA_9 | RNA_ | | 02 | | | | | | | | | | | | | |
| | 27 | | | | | | | | | | | | | | | |
| M21 + | M21 + | No | 1:02: | InfB | 256994 | 52066 | 34.7 | 1.058 | 85780 | 9690 | 35.917 | 0.368 | 2142 | 4058 | NA | NA |
| RNA_9 | RNA_ | | 02 | | | | | | | | | | | | | |
| | 27 | | | | | | | | | | | | | | | |
| M21 + | M21 + | No | 1:02: | RNaseP | 1007750 | 44464 | 33.88 | 0.349 | 637829 | 44140 | 34.294 | 0.135 | 302422 | 56988 | 34.984 | 0.731 |
| RNA_9 | RNA_ | | 02 | | | | | | | | | | | | | |
| | 27 | | | | | | | | | | | | | | | |
| M21 + | M21 + | No | 1:02: | SARS- | 427849 | 26489 | 33.989 | 0.825 | 69562 | 25206 | 36.313 | 0.875 | 15107 | 10648 | 36.896 | 0.38 |
| RNA_9 | RNA_ | | 02 | CoV-2 | | | | | | | | | | | | |
| | 27 | | | | | | | | | | | | | | | |
| M21 + | | Yes | 02: | InfA | 685173 | 17339 | 35.505 | 0.407 | 213677 | 66325 | 37.126 | 1.327 | 23617 | 14072 | 43.244 | NA |
| RNA_9 | | | 03.3 | | | | | | | | | | | | | |
| M21 + | | Yes | 02: | InfB | 279264 | 20185 | 34.743 | 0.333 | 15666 | 6321 | 38.8 | NA | −7 | 289 | NA | NA |
| RNA_9 | | | 03.3 | | | | | | | | | | | | | |
| M21 + | | Yes | 02: | RNaseP | 959241 | 19927 | 34.135 | 0.355 | 309070 | 15496 | 33.803 | 0.155 | 86533 | 34965 | 35.884 | 1.439 |
| RNA_9 | | | 03.3 | | | | | | | | | | | | | |
| M21 + | | Yes | 02: | SARS- | 387226 | 31994 | 34.617 | 0.726 | 26813 | 8938 | 37.403 | 2.218 | 174 | 471 | NA | NA |
| RNA_9 | | | 03.3 | CoV-2 | | | | | | | | | | | | |
| M21 + | M21 + | Yes | 1:2:2: | InfA | 698288 | 52406 | 35.309 | 0.412 | 213301 | 53320 | 37.172 | 0.949 | 11167 | 15550 | NA | NA |
| RNA_9 | RNA_ | | 3.3 | | | | | | | | | | | | | |
| | 27 | | | | | | | | | | | | | | | |
| M21 + | M21 + | Yes | 1:2:2: | InfB | 231446 | 4511 | 35.558 | 0.573 | 24363 | 4713 | 39.282 | 1.88 | 834 | 234 | NA | NA |
| RNA_9 | RNA_ | | 3.3 | | | | | | | | | | | | | |
| | 27 | | | | | | | | | | | | | | | |
| M21 + | M21 + | Yes | 1:2:2: | RNaseP | 810324 | 37603 | 34.364 | 0.212 | 374526 | 31500 | 33.649 | 0.301 | 144324 | 18846 | 34.995 | 0.656 |
| RNA_9 | RNA_ | | 3.3 | | | | | | | | | | | | | |
| | 27 | | | | | | | | | | | | | | | |
| M21 + | M21 + | Yes | 1:2:2: | SARS- | 390373 | 13076 | 34.293 | 0.533 | 42839 | 11799 | 35.738 | 0.784 | 4807 | 3695 | NA | NA |
| RNA_9 | RNA_ | | 3.3 | CoV-2 | | | | | | | | | | | | |
| | 27 | | | | | | | | | | | | | | | |
| M21 + | | No | 1:05 | InfA | 708798 | 30326 | 35.472 | 0.575 | 274173 | 199083 | 35.907 | 0.924 | 31922 | 25422 | 40.038 | NA |
| RNA_9 | | | | | | | | | | | | | | | | |
| M21 + | | No | 1:05 | InfB | 273175 | 21238 | 34.868 | 0.473 | 64905 | 45509 | 36.039 | 0.155 | 7258 | 5654 | NA | NA |
| RNA_9 | | | | | | | | | | | | | | | | |
| M21 + | | No | 1:05 | RNaseP | 925689 | 30416 | 33.742 | 0.341 | 516377 | 286024 | 35.227 | 2.738 | 304229 | 36905 | 34.859 | 0.465 |
| RNA_9 | | | | | | | | | | | | | | | | |
| M21 + | | No | 1:05 | SARS- | 375208 | 9105 | 34.73 | 0.401 | 94716 | 35248 | 35.516 | 0.119 | 12340 | 9991 | 38.825 | 2.106 |
| RNA_9 | | | | CoV-2 | | | | | | | | | | | | |
| M21 + | M21 + | No | 1:05: | InfA | 731952 | 35133 | 35.224 | 0.716 | 366355 | 106399 | 36.584 | 0.726 | 66287 | 22638 | 40.807 | 2.076 |
| RNA_9 | RNA_ | | 05 | | | | | | | | | | | | | |
| | 27 | | | | | | | | | | | | | | | |
| M21 + | M21 + | No | 1:05: | InfB | 294133 | 1614 | 34.29 | 0.219 | 48842 | 34835 | 36.666 | 0.238 | 7144 | 6586 | NA | NA |
| RNA_9 | RNA_ | | 05 | | | | | | | | | | | | | |
| | 27 | | | | | | | | | | | | | | | |
| M21 + | M21 + | No | 1:05: | RNaseP | 1029056 | 48611 | 33.793 | 0.037 | 661317 | 45036 | 34.479 | 0.198 | 443322 | 56499 | 34.132 | 0.378 |
| RNA_9 | RNA_ | | 05 | | | | | | | | | | | | | |
| | 27 | | | | | | | | | | | | | | | |
| M21 + | M21 + | No | 1:05: | SARS- | 426483 | 15345 | 34.664 | 0.181 | 112956 | 43624 | 35.633 | 0.651 | 15150 | 13717 | 36.532 | NA |
| RNA_9 | RNA_ | | 05 | CoV-2 | | | | | | | | | | | | |
| | 27 | | | | | | | | | | | | | | | |
| M21 + | | Yes | 05: | InfA | 685606 | 86323 | 35.211 | 0.685 | 291814 | 53492 | 36.613 | 0.47 | 17264 | 24367 | 42.153 | NA |
| RNA_9 | | | 03.3 | | | | | | | | | | | | | |
| M21 + | | Yes | 05: | InfB | 274724 | 10755 | 34.541 | 0.335 | 27540 | 13889 | 40.642 | 4.426 | −185 | 369 | NA | NA |
| RNA_9 | | | 03.3 | | | | | | | | | | | | | |
| M21 + | | Yes | 05: | RNaseP | 984957 | 30004 | 33.755 | 0.354 | 314876 | 19166 | 34.385 | 0.308 | 203736 | 33177 | 34.177 | 0.761 |
| RNA_9 | | | 03.3 | | | | | | | | | | | | | |
| M21 + | | Yes | 05: | SARS | 396109 | 36584 | 34.436 | 0.379 | 53463 | 15760 | 35.862 | 0.994 | 4902 | 2918 | NA | NA |
| RNA_9 | | | 03.3 | CoV-2 | | | | | | | | | | | | |
| M21 + | M21 + | Yes | 1:5:5: | InfA | 691518 | 37494 | 35.124 | 0.688 | 368672 | 56117 | 36.262 | 0.741 | 32899 | 44883 | 39.738 | NA |
| RNA_9 | RNA_ | | 3.3 | | | | | | | | | | | | | |
| | 27 | | | | | | | | | | | | | | | |
| M21 + | M21 + | Yes | 1:5:5: | InfB | 264135 | 4325 | 34.584 | 0.247 | 42473 | 8690 | 36.366 | 1.049 | 3155 | 3903 | NA | NA |
| RNA_9 | RNA_ | | 3.3 | | | | | | | | | | | | | |
| | 27 | | | | | | | | | | | | | | | |
| M21 + | M21 + | Yes | 1:5:5: | RNaseP | 967104 | 49622 | 33.666 | 0.723 | 445265 | 46576 | 33.865 | 0.254 | 228672 | 28823 | 34.431 | 0.622 |
| RNA_9 | RNA_ | | 3.3 | | | | | | | | | | | | | |
| | 27 | | | | | | | | | | | | | | | |
| M21 + | M21 + | Yes | 1:5:5: | SARS- | 366342 | 54265 | 34.981 | 1.17 | 65115 | 20747 | 35.717 | 1.44 | 11086 | 4331 | 40.762 | NA |
| RNA_9 | RNA_ | | 3.3 | CoV-2 | | | | | | | | | | | | |
| | 27 | | | | | | | | | | | | | | | |
| M21 + | | No | 1:01 | InfA | 513823 | 276037 | 37.459 | 3.866 | 56376 | 10860 | 39.532 | 0.968 | 1301 | 2525 | NA | NA |
| RNA_30 | | | | | | | | | | | | | | | | |
| M21 + | | No | 1:01 | InfB | 187448 | 70931 | 36.323 | 2.313 | 2561 | 2197 | NA | NA | −343 | 49 | NA | NA |
| RNA_30 | | | | | | | | | | | | | | | | |
| M21 + | | No | 1:01 | RNaseP | 748843 | 196393 | 35.484 | 2.026 | 265099 | 73902 | 34.358 | 1.194 | 33128 | 10336 | NA | NA |
| RNA_30 | | | | | | | | | | | | | | | | |
| M21 + | | No | 1:01 | SARS- | 283567 | 86524 | 35.784 | 1.302 | 128 | 246 | NA | NA | −160 | 98 | NA | NA |
| RNA_30 | | | | CoV-2 | | | | | | | | | | | | |
| M21 + | M21 + | No | 1:01: | InfA | 688647 | 16936 | 35.384 | 0.104 | 164414 | 42320 | 37.458 | 1.042 | 12618 | 10204 | NA | NA |
| RNA_9 | RNA_ | | 01 | | | | | | | | | | | | | |
| | 30 | | | | | | | | | | | | | | | |
| M21 + | M21 + | No | 1:01: | InfB | 258895 | 19057 | 35.005 | 0.483 | 49263 | 13643 | 36.235 | 1.374 | 1464 | 1135 | NA | NA |
| RNA_9 | RNA_ | | 01 | | | | | | | | | | | | | |
| | 30 | | | | | | | | | | | | | | | |
| M21 + | M21 + | No | 1:01: | RNaseP | 921218 | 40912 | 34.455 | 0.261 | 584006 | 28527 | 33.916 | 0.194 | 250133 | 35874 | 34.192 | 0.771 |
| RNA_9 | RNA_ | | 01 | | | | | | | | | | | | | |
| | 30 | | | | | | | | | | | | | | | |
| M21 + | M21 + | No | 1:01: | SARS- | 366476 | 12371 | 34.679 | 0.477 | 27890 | 5148 | 36.11 | 1.181 | 546 | 332 | NA | NA |
| RNA_9 | RNA_ | | 01 | CoV-2 | | | | | | | | | | | | |
| | 30 | | | | | | | | | | | | | | | |
| M21 + | | Yes | 01: | InfA | 668734 | 41465 | 35.39 | 0.153 | 134621 | 51408 | 38.029 | 1.705 | 21356 | 2292 | NA | NA |
| RNA_30 | | | 03.3 | | | | | | | | | | | | | |
| M21 + | | Yes | 01: | InfB | 245967 | 32572 | 34.804 | 0.856 | 7827 | 2779 | NA | NA | −152 | 173 | NA | NA |
| RNA_30 | | | 03.3 | | | | | | | | | | | | | |
| M21 + | | Yes | 01: | RNaseP | 1008611 | 10749 | 33.825 | 0.268 | 249813 | 48823 | 33.82 | 0.498 | 51223 | 4274 | 42.509 | 1.554 |
| RNA_30 | | | 03.3 | | | | | | | | | | | | | |
| M21 + | | Yes | 01: | SARS- | 393396 | 58528 | 34.23 | 0.39 | 5061 | 3781 | NA | NA | −239 | 201 | NA | NA |
| RNA_30 | | | 03.3 | CoV-2 | | | | | | | | | | | | |
| M21 + | M21 + | Yes | 1:1:1: | InfA | 652231 | 77320 | 35.81 | 0.719 | 217410 | 11814 | 36.549 | 0.292 | 16636 | 8440 | NA | NA |
| RNA_9 | RNA_ | | 3.3 | | | | | | | | | | | | | |
| | 30 | | | | | | | | | | | | | | | |
| M21 + | M21 + | Yes | 1:1:1: | InfB | 269123 | 10187 | 34.578 | 0.238 | 8047 | 3752 | NA | NA | 103 | 84 | NA | NA |
| RNA_9 | RNA_ | | 3.3 | | | | | | | | | | | | | |
| | 30 | | | | | | | | | | | | | | | |
| M21 + | M21 + | Yes | 1:1:1: | RNaseP | 1022951 | 2861 | 33.558 | 0.268 | 279652 | 18891 | 33.953 | 0.159 | 71980 | 20634 | 36.86 | 1.17 |
| RNA_9 | RNA_ | | 3.3 | | | | | | | | | | | | | |
| | 30 | | | | | | | | | | | | | | | |
| M21 + | M21 + | Yes | 1:1:1: | SARS- | 422253 | 20431 | 34.26 | 0.551 | 13491 | 5054 | 36.995 | NA | 119 | 492 | NA | NA |
| RNA_9 | RNA_ | | 3.3 | CoV-2 | | | | | | | | | | | | |
| | 30 | | | | | | | | | | | | | | | |
| M21 + | | No | 1:02 | InfA | 702459 | 36799 | 35.634 | 0.515 | 205936 | 64646 | 37.25 | 1.072 | 7967 | 8117 | NA | NA |
| RNA_30 | | | | | | | | | | | | | | | | |
| M21 + | | No | 1:02 | InfB | 262130 | 48393 | 35.248 | 0.944 | 32924 | 2637 | 37.725 | 0.511 | 1036 | 98 | NA | NA |
| RNA_30 | | | | | | | | | | | | | | | | |
| M21 + | | No | 1:02 | RNaseP | 1103098 | 63503 | 33.567 | 0.422 | 634748 | 25844 | 33.641 | 0.233 | 316885 | 7277 | 34 | 0.045 |
| RNA_30 | | | | | | | | | | | | | | | | |
| M21 + | | No | 1:02 | SARS- | 444736 | 50153 | 34.193 | 0.427 | 18732 | 8009 | 35.622 | 0.332 | −1209 | 390 | NA | NA |
| RNA_30 | | | | CoV-2 | | | | | | | | | | | | |
| M21 + | M21 + | No | 1:02: | InfA | 681616 | 28847 | 35.829 | 0.48 | 323364 | 90323 | 36.347 | 1.069 | 32827 | 24964 | 41.61 | 2.491 |
| RNA_9 | RNA_ | | 02 | | | | | | | | | | | | | |
| | 30 | | | | | | | | | | | | | | | |
| M21 + | M21 + | No | 1:02: | InfB | 276388 | 20127 | 34.873 | 0.349 | 88332 | 27168 | 35.451 | 0.898 | 6409 | 4300 | NA | NA |
| RNA_9 | RNA_ | | 02 | | | | | | | | | | | | | |
| | 30 | | | | | | | | | | | | | | | |
| M21 + | M21 + | No | 1:02: | RNaseP | 1026825 | 34805 | 33.952 | 0.338 | 693864 | 46340 | 33.592 | 0.189 | 341644 | 19840 | 34.084 | 0.165 |
| RNA_9 | RNA_ | | 02 | | | | | | | | | | | | | |
| | 30 | | | | | | | | | | | | | | | |
| M21 + | M21 + | No | 1:02: | SARS- | 463035 | 13299 | 34.102 | 0.104 | 62563 | 15123 | 35.062 | 0.116 | 271 | 862 | NA | NA |
| RNA_9 | RNA_ | | 02 | CoV-2 | | | | | | | | | | | | |
| | 30 | | | | | | | | | | | | | | | |
| M21 + | | Yes | 02: | InfA | 776757 | 36402 | 34.66 | 0.63 | 130113 | 36815 | 38.552 | 1.452 | 14415 | 10155 | NA | NA |
| RNA_30 | | | 03.3 | | | | | | | | | | | | | |
| M21 + | | Yes | 02: | InfB | 285188 | 22669 | 35.056 | 0.758 | 9582 | 4850 | NA | NA | −115 | 433 | NA | NA |
| RNA_30 | | | 03.3 | | | | | | | | | | | | | |
| M21 + | | Yes | 02: | RNaseP | 1042581 | 71145 | 33.731 | 0.088 | 338933 | 43188 | 33.481 | 0.261 | 109071 | 23713 | 35.987 | 1.522 |
| RNA_30 | | | 03.3 | | | | | | | | | | | | | |
| M21 + | | Yes | 02: | SARS- | 416604 | 23648 | 34.845 | 0.752 | 2837 | 3228 | NA | NA | −204 | 285 | NA | NA |
| RNA_30 | | | 03.3 | CoV-2 | | | | | | | | | | | | |
| M21 + | M21 + | Yes | 1:2:2: | InfA | 210253 | 205205 | 40.28 | 4.798 | 257225 | 131319 | 37.604 | 2.644 | 1117 | 1276 | NA | NA |
| RNA_9 | RNA_ | | 3.3 | | | | | | | | | | | | | |
| | 30 | | | | | | | | | | | | | | | |
| M21 + | M21 + | Yes | 1:2:2: | InfB | 100449 | 96816 | 35.788 | NA | 23708 | 4343 | 39.935 | 3.08 | 250 | 379 | NA | NA |
| RNA_9 | RNA_ | | 3.3 | | | | | | | | | | | | | |
| | 30 | | | | | | | | | | | | | | | |
| M21 + | M21 + | Yes | 1:2:2: | RNaseP | 331190 | 365813 | 38.358 | 6.036 | 380013 | 35360 | 33.796 | 0.197 | 113462 | 65693 | 34.808 | 0.243 |
| RNA_9 | RNA_ | | 3.3 | | | | | | | | | | | | | |
| | 30 | | | | | | | | | | | | | | | |
| M21 + | M21 + | Yes | 1:2:2: | SARS- | 148517 | 117447 | 38.606 | 3.72 | 25847 | 18031 | 35.959 | 0.058 | 2130 | 2386 | NA | NA |
| RNA_9 | RNA_ | | 3.3 | CoV-2 | | | | | | | | | | | | |
| | 30 | | | | | | | | | | | | | | | |
| M21 + | | No | 1:05 | InfA | 464649 | 282275 | 35.976 | 0.84 | 233602 | 155631 | 36.299 | 0.604 | 22928 | 27387 | 40.196 | NA |
| RNA_30 | | | | | | | | | | | | | | | | |
| M21 + | | No | 1:05 | InfB | 195369 | 113169 | 35.097 | 0.44 | 27447 | 19749 | 37.851 | 0.902 | −1441 | 2270 | NA | NA |
| RNA_30 | | | | | | | | | | | | | | | | |
| M21 + | | No | 1:05 | RNaseP | 677360 | 336902 | 36.212 | 3.504 | 409298 | 226955 | 36.151 | 3.583 | 175130 | 54595 | 36.43 | 1.929 |
| RNA_30 | | | | | | | | | | | | | | | | |
| M21 + | | No | 1:05 | SARS- | 264067 | 151482 | 37.562 | 4.15 | 11993 | 918 | NA | NA | −1248 | 418 | NA | NA |
| RNA_30 | | | | CoV-2 | | | | | | | | | | | | |
| M21 + | M21 + | No | 1:05: | InfA | 633222 | 43423 | 36.273 | 0.372 | 476601 | 29760 | 35.774 | 0.338 | 83396 | 23479 | 39.46 | 1.428 |
| RNA_9 | RNA_ | | 05 | | | | | | | | | | | | | |
| | 30 | | | | | | | | | | | | | | | |
| M21 + | M21 + | No | 1:05: | InfB | 257376 | 10669 | 35.289 | 0.525 | 90785 | 41576 | 36.508 | 1.907 | 7201 | 6660 | NA | NA |
| RNA_9 | RNA_ | | 05 | | | | | | | | | | | | | |
| | 30 | | | | | | | | | | | | | | | |
| M21 + | M21 + | No | 1:05: | RNaseP | 1014759 | 28566 | 33.734 | 0.084 | 698316 | 53083 | 34.415 | 0.498 | 344496 | 44909 | 35.161 | 0.561 |
| RNA_9 | RNA_ | | 05 | | | | | | | | | | | | | |
| | 30 | | | | | | | | | | | | | | | |
| M21 + | M21 + | No | 1:05: | SARS- | 386343 | 15410 | 34.92 | 0.456 | 107805 | 18960 | 34.937 | 0.081 | 7785 | 4838 | 43.142 | NA |
| RNA_9 | RNA_ | | 05 | CoV-2 | | | | | | | | | | | | |
| | 30 | | | | | | | | | | | | | | | |
| M21 + | | Yes | 05: | InfA | 698799 | 46928 | 35.182 | 0.657 | 256878 | 52935 | 37.04 | 0.789 | 34704 | 17975 | 42.437 | 1.399 |
| RNA_30 | | | 03.3 | | | | | | | | | | | | | |
| M21 + | | Yes | 05: | InfB | 274684 | 18105 | 34.508 | 0.509 | 27153 | 6720 | 38.445 | 2.338 | 133 | 394 | NA | NA |
| RNA_30 | | | 03.3 | | | | | | | | | | | | | |
| M21 + | | Yes | 05: | RNaseP | 985399 | 38309 | 33.718 | 0.556 | 398615 | 24190 | 33.818 | 0.061 | 161610 | 37151 | 34.944 | 0.859 |
| RNA_30 | | | 03.3 | | | | | | | | | | | | | |
| M21 + | | Yes | 05: | SARS- | 382088 | 21016 | 34.501 | 0.544 | 6014 | 3948 | NA | NA | −706 | 215 | NA | NA |
| RNA_30 | | | 03.3 | CoV-2 | | | | | | | | | | | | |
| M21 + | M21 + | Yes | 1:5:5: | InfA | 634250 | 98288 | 35.404 | 0.731 | 313113 | 90715 | 36.628 | 1.051 | 26070 | 37779 | 40.233 | NA |
| RNA_9 | RNA_ | | 3.3 | | | | | | | | | | | | | |
| | 30 | | | | | | | | | | | | | | | |
| M21 + | M21 + | Yes | 1:5:5: | InfB | 259277 | 29355 | 34.634 | 0.201 | 29796 | 6966 | 38.352 | 1.783 | 1152 | 521 | NA | NA |
| RNA_9 | RNA_ | | 3.3 | | | | | | | | | | | | | |
| | 30 | | | | | | | | | | | | | | | |
| M21 + | M21 + | Yes | 1:5:5: | RNaseP | 994977 | 58166 | 33.702 | 0.624 | 394351 | 14684 | 33.945 | 0.059 | 161269 | 50912 | 35.517 | 1.662 |
| RNA_9 | RNA_ | | 3.3 | | | | | | | | | | | | | |
| | 30 | | | | | | | | | | | | | | | |
| M21 + | M21 + | Yes | 1:5:5: | SARS- | 401232 | 31372 | 33.937 | 0.248 | 19133 | 5077 | 39.305 | 0.554 | 3181 | 3986 | NA | NA |
| RNA_9 | RNA_ | | 3.3 | CoV-2 | | | | | | | | | | | | |
| | 30 | | | | | | | | | | | | | | | |
| M21 + | | No | 1:01 | InfA | 559061 | 177772 | 36.365 | 2.226 | 27350 | 9292 | 43.099 | NA | 343 | 982 | NA | NA |
| RNA_31 | | | | | | | | | | | | | | | | |
| M21 + | | No | 1:01 | InfB | 205028 | 70442 | 35.993 | 1.886 | 3542 | 2913 | NA | NA | −473 | 63 | NA | NA |
| RNA_31 | | | | | | | | | | | | | | | | |
| M21 + | | No | 1:01 | RNaseP | 835678 | 176387 | 34.705 | 1.761 | 228589 | 137367 | 37.289 | 6.195 | 34005 | 7858 | NA | NA |
| RNA_31 | | | | | | | | | | | | | | | | |
| M21 + | | No | 1:01 | SARS- | 330795 | 106403 | 35.21 | 1.565 | 10793 | 1401 | NA | NA | −197 | 133 | NA | NA |
| RNA_31 | | | | CoV-2 | | | | | | | | | | | | |
| M21 + | M21 + | No | 1:01: | InfA | 690213 | 34639 | 35.337 | 0.445 | 216721 | 63626 | 36.048 | 1.331 | 16723 | 8608 | NA | NA |
| RNA_31 | RNA_ | | 01 | | | | | | | | | | | | | |
| | 44 | | | | | | | | | | | | | | | |
| M21 + | M21 + | No | 1:01: | InfB | 271526 | 28912 | 35.109 | 0.527 | 43079 | 4980 | 36.014 | 0.592 | 572 | 62 | NA | NA |
| RNA_31 | RNA_ | | 01 | | | | | | | | | | | | | |
| | 44 | | | | | | | | | | | | | | | |
| M21 + | M21 + | No | 1:01: | RNaseP | 946972 | 73726 | 34.514 | 0.513 | 515410 | 30783 | 34.104 | 0.697 | 173536 | 58593 | 35.108 | 1.456 |
| RNA_31 | RNA_ | | 01 | | | | | | | | | | | | | |
| | 44 | | | | | | | | | | | | | | | |
| M21 + | M21 + | No | 1:01: | SARS- | 394206 | 36440 | 34.952 | 0.778 | 50760 | 28029 | 35.044 | 0.803 | 1113 | 1711 | NA | NA |
| RNA_31 | RNA_ | | 01 | CoV-2 | | | | | | | | | | | | |
| | 44 | | | | | | | | | | | | | | | |
| M21 + | | Yes | 01: | InfA | 683484 | 43772 | 35.152 | 0.554 | 154984 | 38715 | 37.415 | 1.034 | 9927 | 7400 | NA | NA |
| RNA_31 | | | 03.3 | | | | | | | | | | | | | |
| M21 + | | Yes | 01: | InfB | 223392 | 23768 | 35.568 | 0.61 | 14449 | 3028 | 41.95 | NA | −164 | 810 | NA | NA |
| RNA_31 | | | 03.3 | | | | | | | | | | | | | |
| M21 + | | Yes | 01: | RNaseP | 952466 | 30277 | 33.766 | 0.272 | 240072 | 16858 | 33.958 | 0.673 | 56143 | 9213 | 39.449 | 1.148 |
| RNA_31 | | | 03.3 | | | | | | | | | | | | | |
| M21 + | | Yes | 01: | SARS- | 377532 | 41750 | 34.621 | 0.984 | 11065 | 8393 | 37.737 | NA | −271 | 864 | NA | NA |
| RNA_31 | | | 03.3 | CoV-2 | | | | | | | | | | | | |
| M21 + | M21 + | Yes | 1:1:1: | InfA | 723233 | 70133 | 34.876 | 0.785 | 141703 | 45387 | 38.57 | 1.914 | 10281 | 13396 | NA | NA |
| RNA_31 | RNA_ | | 3.3 | | | | | | | | | | | | | |
| | 44 | | | | | | | | | | | | | | | |
| M21 + | M21 + | Yes | 1:1:1: | InfB | 295358 | 20839 | 34.166 | 0.298 | 15082 | 2812 | 41.455 | NA | 35 | 531 | NA | NA |
| RNA_31 | RNA_ | | 3.3 | | | | | | | | | | | | | |
| | 44 | | | | | | | | | | | | | | | |
| M21 + | M21 + | Yes | 1:1:1: | RNaseP | 1078172 | 78248 | 33.613 | 0.902 | 252182 | 11406 | 34.262 | 0.362 | 90471 | 34648 | 35.212 | 0.411 |
| RNA_31 | RNA_ | | 3.3 | | | | | | | | | | | | | |
| | 44 | | | | | | | | | | | | | | | |
| M21 + | M21 + | Yes | 1:1:1: | SARS- | 450465 | 1380 | 34.424 | 0.472 | 12878 | 10335 | 36.453 | NA | −565 | 340 | NA | NA |
| RNA_31 | RNA_ | | 3.3 | CoV-2 | | | | | | | | | | | | |
| | 44 | | | | | | | | | | | | | | | |
| M21 + | | No | 1:02 | InfA | 724686 | 9083 | 35.427 | 0.224 | 237208 | 21036 | 36.341 | 0.428 | 15570 | 8660 | NA | NA |
| RNA_31 | | | | | | | | | | | | | | | | |
| M21 + | | No | 1:02 | InfB | 277596 | 2858 | 34.831 | 0.232 | 66366 | 14314 | 35.582 | 0.705 | 3089 | 3128 | NA | NA |
| RNA_31 | | | | | | | | | | | | | | | | |
| M21 + | | No | 1:02 | RNaseP | 1041927 | 41929 | 33.962 | 0.352 | 671694 | 36302 | 33.543 | 0.239 | 294434 | 26897 | 33.915 | 0.278 |
| RNA_31 | | | | | | | | | | | | | | | | |
| M21 + | | No | 1:02 | SARS- | 467738 | 51961 | 34.118 | 0.426 | 32209 | 19086 | 35.563 | 1.131 | −1053 | 93 | NA | NA |
| RNA_31 | | | | CoV-2 | | | | | | | | | | | | |
| M21 + | M21 + | No | 1:02: | InfA | 691536 | 21704 | 35.438 | 0.712 | 383374 | 58535 | 35.524 | 0.4 | 24010 | 19706 | 41.471 | NA |
| RNA_31 | RNA_ | | 02 | | | | | | | | | | | | | |
| | 44 | | | | | | | | | | | | | | | |
| M21 + | M21 + | No | 1:02: | InfB | 286966 | 20246 | 34.45 | 0.185 | 67319 | 15043 | 36.276 | 1.185 | 7367 | 6085 | 44.725 | NA |
| RNA_31 | RNA_ | | 02 | | | | | | | | | | | | | |
| | 44 | | | | | | | | | | | | | | | |
| M21 + | M21 + | No | 1:02: | RNaseP | 1056589 | 24389 | 33.729 | 0.55 | 644429 | 26104 | 34.12 | 0.218 | 334624 | 66047 | 34.216 | 0.735 |
| RNA_31 | RNA_ | | 02 | | | | | | | | | | | | | |
| | 44 | | | | | | | | | | | | | | | |
| M21 + | M21 + | No | 1:02: | SARS- | 398432 | 30170 | 34.854 | 0.174 | 68602 | 1548 | 35.175 | 0.234 | 6013 | 4420 | NA | NA |
| RNA_31 | RNA_ | | 02 | CoV-2 | | | | | | | | | | | | |
| | 44 | | | | | | | | | | | | | | | |
| M21 + | | Yes | 02: | InfA | 730439 | 34994 | 35.055 | 0.504 | 167444 | 29406 | 37.591 | 0.572 | 22507 | 8814 | NA | NA |
| RNA_31 | | | 03.3 | | | | | | | | | | | | | |
| M21 + | | Yes | 02: | InfB | 257412 | 10758 | 35.389 | 0.404 | 10195 | 7540 | 41.839 | NA | 522 | 166 | NA | NA |
| RNA_31 | | | 03.3 | | | | | | | | | | | | | |
| M21 + | | Yes | 02: | RNaseP | 995449 | 44362 | 33.959 | 0.119 | 327122 | 40212 | 33.683 | 0.108 | 120684 | 12725 | 35.096 | 0.645 |
| RNA_31 | | | 03.3 | | | | | | | | | | | | | |
| M21 + | | Yes | 02: | SARS- | 408516 | 8844 | 34.542 | 0.25 | 11863 | 761 | NA | NA | −7 | 204 | NA | NA |
| RNA_31 | | | 03.3 | CoV-2 | | | | | | | | | | | | |
| M21 + | M21 + | Yes | 1:2:2: | InfA | 713124 | 19115 | 35.289 | 0.072 | 188610 | 75949 | 37.929 | 1.641 | 2790 | 2179 | NA | NA |
| RNA_31 | RNA_ | | 3.3 | | | | | | | | | | | | | |
| | 44 | | | | | | | | | | | | | | | |
| M21 + | M21 + | Yes | 1:2:2: | InfB | 235633 | 18302 | 35.319 | 0.22 | 20523 | 2162 | 40.658 | 1.942 | 413 | 422 | NA | NA |
| RNA_31 | RNA_ | | 3.3 | | | | | | | | | | | | | |
| | 44 | | | | | | | | | | | | | | | |
| M21 + | M21 + | Yes | 1:2:2: | RNaseP | 889554 | 41271 | 34.133 | 0.442 | 380809 | 39513 | 33.693 | 0.25 | 75161 | 49529 | 36.413 | 0.083 |
| RNA_31 | RNA_ | | 3.3 | | | | | | | | | | | | | |
| | 44 | | | | | | | | | | | | | | | |
| M21 + | M21 + | Yes | 1:2:2: | SARS- | 342586 | 35975 | 34.938 | 0.44 | 32658 | 6035 | 36.359 | 0.608 | 29 | 433 | NA | NA |
| RNA_31 | RNA_ | | 3.3 | CoV-2 | | | | | | | | | | | | |
| | 44 | | | | | | | | | | | | | | | |
| M21 + | | No | 1:05 | InfA | 658104 | 122886 | 35.477 | 1.5 | 249527 | 102706 | 36.975 | 1.522 | 15268 | 15741 | 43.91 | NA |
| RNA_31 | | | | | | | | | | | | | | | | |
| M21 + | | No | 1:05 | InfB | 267458 | 31289 | 34.996 | 0.622 | 56876 | 34078 | 38.283 | 4.23 | 1402 | 2016 | NA | NA |
| RNA_31 | | | | | | | | | | | | | | | | |
| M21 + | | No | 1:05 | RNaseP | 979830 | 91382 | 33.779 | 0.653 | 549796 | 20156 | 34.333 | 0.182 | 183536 | 104157 | 34.426 | 0.469 |
| RNA_31 | | | | | | | | | | | | | | | | |
| M21 + | | No | 1:05 | SARS- | 370670 | 22905 | 34.97 | 0.748 | 40402 | 6997 | 35.817 | 0.604 | 2430 | 3043 | NA | NA |
| RNA_31 | | | | CoV-2 | | | | | | | | | | | | |
| M21 + | M21 + | No | 1:05: | InfA | 757471 | 45938 | 35.184 | 0.624 | 276340 | 9001 | 37.103 | 1.531 | 8837 | 12622 | NA | NA |
| RNA_31 | RNA_ | | 05 | | | | | | | | | | | | | |
| | 44 | | | | | | | | | | | | | | | |
| M21 + | M21 + | No | 1:05: | InfB | 294417 | 25060 | 34.902 | 0.286 | 92939 | 27792 | 35.813 | 0.853 | 8129 | 1284 | NA | NA |
| RNA_31 | RNA_ | | 05 | | | | | | | | | | | | | |
| | 44 | | | | | | | | | | | | | | | |
| M21 + | M21 + | No | 1:05: | RNaseP | 1098022 | 80577 | 33.349 | 0.571 | 718619 | 28368 | 33.818 | 0.351 | 280988 | 64766 | 35.297 | 0.665 |
| RNA_31 | RNA_ | | 05 | | | | | | | | | | | | | |
| | 44 | | | | | | | | | | | | | | | |
| M21 + | M21 + | No | 1:05: | SARS- | 377200 | 44729 | 34.935 | 0.704 | 82803 | 16162 | 35.526 | 0.715 | −554 | 923 | NA | NA |
| RNA_31 | RNA_ | | 05 | CoV-2 | | | | | | | | | | | | |
| | 44 | | | | | | | | | | | | | | | |
| M21 + | | Yes | 05: | InfA | 747828 | 68152 | 34.663 | 1.035 | 225473 | 74847 | 37.421 | 1.424 | 18591 | 13028 | NA | NA |
| RNA_31 | | | 03.3 | | | | | | | | | | | | | |
| M21 + | | Yes | 05: | InfB | 282161 | 15679 | 34.413 | 0.346 | 16482 | 5337 | 38.687 | NA | 440 | 838 | NA | NA |
| RNA_31 | | | 03.3 | | | | | | | | | | | | | |
| M21 + | | Yes | 05: | RNaseP | 1043744 | 66093 | 33.819 | 0.456 | 371691 | 15580 | 33.628 | 0.197 | 151271 | 32832 | 35.02 | 1.247 |
| RNA_31 | | | 03.3 | | | | | | | | | | | | | |
| M21 + | | Yes | 05: | SARS- | 441411 | 18807 | 34.103 | 0.62 | 46298 | 19509 | 35.665 | 1.258 | 218 | 317 | NA | NA |
| RNA_31 | | | 03.3 | CoV-2 | | | | | | | | | | | | |
| M21 + | M21 + | Yes | 1:5:5: | InfA | 705048 | 39161 | 35.225 | 0.201 | 258539 | 74699 | 37.36 | 1.259 | 13748 | 19747 | 43.573 | NA |
| RNA_31 | RNA_ | | 3.3 | | | | | | | | | | | | | |
| | 44 | | | | | | | | | | | | | | | |
| M21 + | M21 + | Yes | 1:5:5: | InfB | 256631 | 20236 | 34.484 | 0.359 | 15992 | 2610 | 40.639 | NA | 51 | 192 | NA | NA |
| RNA_31 | RNA_ | | 3.3 | | | | | | | | | | | | | |
| | 44 | | | | | | | | | | | | | | | |
| M21 + | M21 + | Yes | 1:5:5: | RNaseP | 1095965 | 38193 | 33.417 | 0.177 | 466956 | 19697 | 33.752 | 0.304 | 223282 | 6470 | 34.469 | 0.09 |
| RNA_31 | RNA_ | | 3.3 | | | | | | | | | | | | | |
| | 44 | | | | | | | | | | | | | | | |
| M21 + | M21 + | Yes | 1:5:5: | SARS- | 430949 | 14365 | 34.019 | 0.213 | 53075 | 13855 | 35.805 | 1.48 | 6541 | 5647 | 42.805 | NA |
| RNA_31 | RNA_ | | 3.3 | CoV-2 | | | | | | | | | | | | |
| | 44 | | | | | | | | | | | | | | | |
| M21 + N | | No | 1:01 | InfA | 601186 | 86666 | 35.719 | 1.212 | 84233 | 45940 | 37.328 | 1.238 | 7939 | 3970 | NA | NA |
| RA_44 | | | | | | | | | | | | | | | | |
| M21 + | | No | 1:01 | InfB | 238384 | 11270 | 34.858 | 0.562 | 21531 | 11692 | 35.568 | NA | 111 | 85 | NA | NA |
| RNA_44 | | | | | | | | | | | | | | | | |
| M21 + | | No | 1:01 | RNaseP | 911024 | 46906 | 34.002 | 0.579 | 270672 | 177650 | 34.13 | 0.336 | 127483 | 27427 | 35.469 | 1.321 |
| RNA_44 | | | | | | | | | | | | | | | | |
| M21 + | | No | 1:01 | SARS- | 296595 | 64666 | 35.359 | 1.1 | 16303 | 9523 | 38.277 | 3.539 | −5 | 484 | NA | NA |
| RNA_44 | | | | CoV-2 | | | | | | | | | | | | |
| M21 + | M21 + | No | 1:01: | InfA | 641798 | 81501 | 35.429 | 0.133 | 190829 | 55453 | 35.68 | 0.8 | 7849 | 5357 | NA | NA |
| RNA_43 | RNA_ | | 01 | | | | | | | | | | | | | |
| | 44 | | | | | | | | | | | | | | | |
| M21 + | M21 + | No | 1:01: | InfB | 234619 | 93002 | 35.153 | 1.716 | 30512 | 7789 | 36.998 | 2.21 | −167 | 80 | NA | NA |
| RNA_43 | RNA_ | | 01 | | | | | | | | | | | | | |
| | 44 | | | | | | | | | | | | | | | |
| M21 + | M21 + | No | 1:01: | RNaseP | 1005801 | 105518 | 33.847 | 0.435 | 452873 | 40919 | 33.571 | 0.121 | 131461 | 19118 | 34.767 | 0.854 |
| RNA_43 | RNA_ | | 01 | | | | | | | | | | | | | |
| | 44 | | | | | | | | | | | | | | | |
| M21 + | M21 + | No | 1:01: | SARS- | 351152 | 67361 | 34.695 | 0.853 | 31001 | 6618 | 35.591 | 1.323 | −68 | 223 | NA | NA |
| RNA_43 | RNA_ | | 01 | CoV-2 | | | | | | | | | | | | |
| | 44 | | | | | | | | | | | | | | | |
| M21 + | | Yes | 01: | InfA | 738535 | 15426 | 34.536 | 0.26 | 186424 | 31342 | 36.591 | 0.69 | 11210 | 8236 | NA | NA |
| RNA_44 | | | 03.3 | | | | | | | | | | | | | |
| M21 + | | Yes | 01: | InfB | 247012 | 16727 | 34.784 | 0.348 | 12255 | 3642 | NA | NA | 444 | 261 | NA | NA |
| RNA_44 | | | 03.3 | | | | | | | | | | | | | |
| M21 + | | Yes | 01: | RNaseP | 1022458 | 71247 | 33.691 | 0.496 | 298566 | 28682 | 33.418 | 0.534 | 72654 | 5891 | 37.34 | 0.382 |
| RNA_44 | | | 03.3 | | | | | | | | | | | | | |
| M21 + | | Yes | 01: | SARS- | 401588 | 5492 | 34.548 | 0.317 | 11525 | 4988 | 39.673 | NA | −441 | 402 | NA | NA |
| RNA_44 | | | 03.3 | CoV-2 | | | | | | | | | | | | |
| M21 + | M21 + | Yes | 1:1:1: | InfA | 709606 | 42036 | 35 | 0.413 | 126446 | 6756 | 38.246 | 0.155 | 14308 | 2298 | NA | NA |
| RNA_43 | RNA_ | | 3.3 | | | | | | | | | | | | | |
| | 44 | | | | | | | | | | | | | | | |
| M21 + | M21 + | Yes | 1:1:1: | InfB | 244897 | 32212 | 35.04 | 0.712 | 4897 | 4510 | NA | NA | −88 | 318 | NA | NA |
| RNA_43 | RNA_ | | 3.3 | | | | | | | | | | | | | |
| | 44 | | | | | | | | | | | | | | | |
| M21 + | M21 + | Yes | 1:1:1: | RNaseP | 1061750 | 73823 | 33.326 | 0.454 | 300555 | 35355 | 33.747 | 0.39 | 103917 | 15635 | 35.818 | 1.025 |
| ARN_43 | RNA_ | | 3.3 | | | | | | | | | | | | | |
| | 44 | | | | | | | | | | | | | | | |
| M21 + | M21 + | Yes | 1:1:1: | SARS- | 418846 | 30187 | 33.829 | 0.302 | 25916 | 2350 | 36.608 | 0.805 | 440 | 351 | NA | NA |
| RNA_43 | RNA_ | | 3.3 | CoV-2 | | | | | | | | | | | | |
| | 44 | | | | | | | | | | | | | | | |
| M21 + | | No | 1:02 | InfA | 725492 | 20084 | 35.026 | 0.319 | 182991 | 108079 | 36.136 | 0.049 | 12796 | 13907 | NA | NA |
| RNA_44 | | | | | | | | | | | | | | | | |
| M21 + | | No | 1:02 | InfB | 287049 | 11066 | 34.596 | 0.348 | 47502 | 16109 | 36.344 | 1.065 | 763 | 434 | NA | NA |
| RNA_44 | | | | | | | | | | | | | | | | |
| M21 + | | No | 1:02 | RNaseP | 1096420 | 16721 | 33.638 | 0.311 | 509341 | 111665 | 34.013 | 0.263 | 280009 | 45869 | 34.429 | 0.438 |
| RNA_44 | | | | | | | | | | | | | | | | |
| M21 + | | No | 1:02 | SARS- | 442639 | 31758 | 34.421 | 0.353 | 48184 | 26623 | 35.956 | 2.114 | −90 | 602 | NA | NA |
| RNA_44 | | | | CoV-2 | | | | | | | | | | | | |
| M21 + | M21 + | No | 1:02: | InfA | 721374 | 38909 | 34.878 | 0.296 | 213362 | 51364 | 36.856 | 0.822 | 14380 | 12301 | NA | NA |
| RNA_43 | RNA_ | | 02 | | | | | | | | | | | | | |
| | 44 | | | | | | | | | | | | | | | |
| M21 + | M21 + | No | 1:02: | InfB | 265322 | 14050 | 34.966 | 0.401 | 58366 | 11544 | 36.118 | 0.599 | 1952 | 2612 | NA | NA |
| RNA_43 | RNA_ | | 02 | | | | | | | | | | | | | |
| | 44 | | | | | | | | | | | | | | | |
| M21 + | M21 + | No | 1:02: | RNaseP | 1123829 | 64675 | 33.497 | 0.308 | 599827 | 35233 | 33.638 | 0.154 | 123045 | 94832 | 34.739 | 0.125 |
| RNA_43 | RNA_ | | 02 | | | | | | | | | | | | | |
| | 44 | | | | | | | | | | | | | | | |
| M21 + | M21 + | No | 1:02: | SARS- | 464400 | 28453 | 33.85 | 0.724 | 66185 | 17813 | 35.199 | 0.701 | −409 | 865 | NA | NA |
| RNA_43 | RNA_ | | 02 | CoV-2 | | | | | | | | | | | | |
| | 44 | | | | | | | | | | | | | | | |
| M21 + | | Yes | 02: | InfA | 742694 | 9354 | 34.944 | 0.381 | 121239 | 17166 | 38.624 | 0.543 | 4891 | 3570 | NA | NA |
| RNA_44 | | | 03.3 | | | | | | | | | | | | | |
| M21 + | | Yes | 02: | InfB | 302652 | 9839 | 34.279 | 0.157 | 12807 | 8399 | 40.878 | NA | 27 | 321 | NA | NA |
| RNA_44 | | | 03.3 | | | | | | | | | | | | | |
| M21 + | | Yes | 02: | RNaseP | 1067965 | 112904 | 33.703 | 0.457 | 345062 | 38271 | 33.596 | 0.478 | 123320 | 14126 | 35.296 | 0.674 |
| RNA_44 | | | 03.3 | | | | | | | | | | | | | |
| M21 + | | Yes | 02: | SARS- | 438495 | 38056 | 34.218 | 0.267 | 26357 | 3478 | 36.716 | 1.052 | 20 | 571 | NA | NA |
| RNA_44 | | | 03.3 | CoV-2 | | | | | | | | | | | | |
| M21 + | M21 + | Yes | 1:2:2: | InfA | 717452 | 29902 | 35.045 | 0.361 | 229799 | 88360 | 37.333 | 1.494 | 22225 | 29982 | 41.031 | NA |
| RNA_43 | RNA_ | | 3.3 | | | | | | | | | | | | | |
| | 44 | | | | | | | | | | | | | | | |
| M21 + | M21 + | Yes | 1:2:2: | InfB | 246184 | 12896 | 35.169 | 0.187 | 33953 | 7966 | 37.156 | 2.025 | −752 | 866 | NA | NA |
| RNA_43 | RNA_ | | 3.3 | | | | | | | | | | | | | |
| | 44 | | | | | | | | | | | | | | | |
| M21 + | M21 + | Yes | 1:2:2: | RNaseP | 886323 | 42566 | 33.931 | 0.515 | 373425 | 52005 | 33.752 | 0.42 | 80225 | 79252 | 34.333 | NA |
| RNA_43 | RNA_ | | 3.3 | | | | | | | | | | | | | |
| | 44 | | | | | | | | | | | | | | | |
| M21 + | M21 + | Yes | 1:2:2: | SARS- | 391433 | 21187 | 34.362 | 0.172 | 23808 | 20660 | 36.417 | 2.066 | −1204 | 945 | NA | NA |
| RNA_43 | RNA_ | | 3.3 | CoV-2 | | | | | | | | | | | | |
| | 44 | | | | | | | | | | | | | | | |
| M21 + | | No | 1:05 | InfA | 572322 | 134418 | 36.208 | 1.475 | 175242 | 106623 | 36.805 | 0.744 | 25081 | 19925 | 41.863 | NA |
| RNA_44 | | | | | | | | | | | | | | | | |
| M21 + | | No | 1:05 | InfB | 238486 | 29998 | 35.575 | 0.907 | 34734 | 15397 | 39.673 | 4.633 | 3372 | 2063 | NA | NA |
| RNA_44 | | | | | | | | | | | | | | | | |
| M21 + | | No | 1:05 | RNaseP | 869003 | 100448 | 34.701 | 1.121 | 426169 | 264453 | 36.743 | 4.748 | 327295 | 83606 | 34.4 | 0.827 |
| RNA_44 | | | | | | | | | | | | | | | | |
| M21 + | | No | 1:05 | SARS- | 322075 | 76722 | 35.403 | 1.297 | 34479 | 22368 | 35.509 | 0.531 | 1539 | 3343 | NA | NA |
| RNA_44 | | | | CoV-2 | | | | | | | | | | | | |
| M21 + | M21 + | No | 1:05: | InfA | 686833 | 16995 | 35.737 | 0.315 | 349741 | 42858 | 36.557 | 0.274 | 91015 | 28417 | 39.468 | 1.868 |
| RNA_43 | RNA_ | | 05 | | | | | | | | | | | | | |
| | 44 | | | | | | | | | | | | | | | |
| M21 + | M21 + | No | 1:05: | InfB | 265574 | 15574 | 34.936 | 0.564 | 47379 | 34638 | 36.606 | 0.23 | 12604 | 10454 | 37.886 | NA |
| RNA_43 | RNA_ | | 05 | | | | | | | | | | | | | |
| | 44 | | | | | | | | | | | | | | | |
| M21 + | M21 + | No | 1:05: | RNaseP | 985689 | 68529 | 33.768 | 0.235 | 606724 | 55178 | 34.311 | 0.296 | 348674 | 52049 | 34.946 | 0.751 |
| RNA_43 | RNA_ | | 05 | | | | | | | | | | | | | |
| | 44 | | | | | | | | | | | | | | | |
| M21 + | M21 + | No | 1:05: | SARS- | 394402 | 52861 | 34.032 | 0.218 | 101329 | 27727 | 35.23 | 0.485 | 9527 | 8321 | 39.572 | NA |
| RNA_43 | RNA_ | | 05 | CoV-2 | | | | | | | | | | | | |
| | 44 | | | | | | | | | | | | | | | |
| M21 + | | Yes | 05: | InfA | 667159 | 60600 | 35.298 | 0.803 | 243734 | 64118 | 37.139 | 0.891 | 53648 | 28540 | 40.874 | 2.691 |
| RNA_44 | | | 03.3 | | | | | | | | | | | | | |
| M21 + | | Yes | 05: | InfB | 243930 | 18021 | 34.974 | 0.497 | 25968 | 2513 | 38.497 | 0.172 | 388 | 819 | NA | NA |
| RNA_44 | | | 03.3 | | | | | | | | | | | | | |
| M21 + | | Yes | 05: | RNaseP | 972241 | 47663 | 34.134 | 0.267 | 365061 | 15258 | 34.173 | 0.235 | 156831 | 19805 | 35.239 | 0.637 |
| RNA_44 | | | 03.3 | | | | | | | | | | | | | |
| M21 + | | Yes | 05: | SARS- | 393575 | 3796 | 34.01 | 0.214 | 26059 | 11346 | 37.109 | 1.4 | 2363 | 2336 | NA | NA |
| RNA_44 | | | 03.3 | CoV-2 | | | | | | | | | | | | |
| M21 + | M21 + | Yes | 1:5:5: | InfA | 733822 | 68931 | 34.842 | 0.611 | 251376 | 42312 | 37.139 | 1.063 | 56323 | 71358 | 38.308 | NA |
| RNA_43 | RNA_ | | 3.3 | | | | | | | | | | | | | |
| | 44 | | | | | | | | | | | | | | | |
| M21 + | M21 + | Yes | 1:5:5: | InfB | 238900 | 13266 | 35.233 | 0.458 | 35347 | 6783 | 36.715 | 0.869 | 674 | 276 | NA | NA |
| RNA_43 | RNA_ | | 3.3 | | | | | | | | | | | | | |
| | 44 | | | | | | | | | | | | | | | |
| M21 + | M21 + | Yes | 1:5:5: | RNaseP | 1050153 | 58021 | 33.348 | 0.689 | 423658 | 44847 | 33.974 | 0.528 | 133996 | 9786 | 36.126 | 0.507 |
| RNA_43 | RNA_ | | 3.3 | | | | | | | | | | | | | |
| | 44 | | | | | | | | | | | | | | | |
| M21 + | M21 + | Yes | 1:5:5: | SARS- | 395799 | 27160 | 34.17 | 0.54 | 49618 | 11992 | 36.127 | 0.874 | 5537 | 2349 | NA | NA |
| RNA_43 | RNA_ | | 3.3 | CoV-2 | | | | | | | | | | | | |
| | 44 | | | | | | | | | | | | | | | |
| RNA | | | | InfA | 395357 | 248934 | 34.802 | 0.235 | 92322 | 7778 | 38.74 | 0.247 | −26311 | 44309 | NA | NA |
| Aptamer | | | | | | | | | | | | | | | | |
| RNA | | | | InfB | 136479 | 60846 | 37.595 | 3.631 | 4108 | 3252 | NA | NA | −1214 | 1646 | NA | NA |
| Aptamer | | | | | | | | | | | | | | | | |
| RNA | | | | RNaseP | 519827 | 249661 | 35.769 | 2.943 | 173171 | 25659 | 34.436 | 0.819 | 46172 | 24055 | 39.096 | 0.207 |
| Aptamer | | | | | | | | | | | | | | | | |
| RNA | | | | SARS- | 242550 | 146896 | 33.597 | 0.478 | 4546 | 3650 | NA | NA | −1584 | 2187 | NA | NA |
| Aptamer | | | | CoV-2 | | | | | | | | | | | | |
| No | | | | InfA | 544792 | 122318 | 34.611 | 0.928 | 18429 | 14547 | 43.071 | NA | 849 | 153 | NA | NA |
| Aptamer | | | | | | | | | | | | | | | | |
| No | | | | InfB | 195301 | 20116 | 34.853 | 0.324 | 1522 | 392 | NA | NA | −51 | 113 | NA | NA |
| Aptamer | | | | | | | | | | | | | | | | |
| No | | | | RNaseP | 680868 | 105144 | 34.18 | 0.975 | 192478 | 19186 | 33.881 | 0.348 | 11387 | 6038 | NA | NA |
| Aptamer | | | | | | | | | | | | | | | | |
| No | | | | SARS- | 285334 | 57028 | 34.21 | 0.483 | 12044 | 1716 | 44.673 | NA | 152 | 109 | NA | NA |
| Aptamer | | | | CoV-2 | | | | | | | | | | | | |
| NTC | | | | InfA | 63394 | 1342 | NA | NA | 2184 | 359 | NA | NA | 2238 | 214 | NA | NA |
| NTC | | | | InfB | 39127 | 342 | NA | NA | 780 | 119 | NA | NA | 279 | 329 | NA | NA |
| NTC | | | | RNaseP | 55740 | 881 | NA | NA | 1287 | 294 | NA | NA | 874 | 280 | NA | NA |
| NTC | | | | SARS- | 37452 | 778 | NA | NA | 518 | 80 | NA | NA | 430 | 227 | NA | NA |
| | | | | CoV-2 | | | | | | | | | | | | |

TABLE 8
Evaluation of room temperature stability at 4 hours following template addition
to PrimeTime^{TM} One-Step RT-qPCR Master Mix (Integrated DNA Technologies)
with combinations of DNA aptamers with various 3′ modifications.

| | | | | 4 h Endpoint | | 4 h Cycle | |
|---|---|---|---|---|---|---|---|
| | | | | Fluorescence | | Threshold | |
| 3′ | | | Target | Delta Rn | Delta Rn | Ct | Ct |
| Modification | Aptamer 1 | Aptamer 2 | Name | Mean | SD | Mean | SD |
| | M21 + RNA_9 | | InfA | 355,244.219 | 90,619.731 | 34.419 | 0.578 |
| | M21 + RNA_9 | | InfB | 78,874.861 | 38,721.520 | 32.577 | 0.968 |
| | M21 + RNA_9 | | RNaseP | 640,228.164 | 84,420.229 | 32.789 | 0.472 |
| | M21 + RNA_9 | | SARS- | 122,653.741 | 34,002.976 | 33.227 | 0.756 |
| | | | CoV-2 | | | | |
| 3′ Phosphate | M21 + RNA_9 | | InfA | 483,156.255 | 126,990.194 | 33.958 | 0.810 |
| 3′ Phosphate | M21 + RNA_9 | | InfB | 97,783.915 | 28,045.129 | 32.657 | 0.670 |
| 3′ Phosphate | M21 + RNA_9 | | RNaseP | 714,568.844 | 57,694.593 | 32.761 | 0.487 |
| 3′ Phosphate | M21 + RNA_9 | | SARS- | 140,808.558 | 42,520.756 | 34.013 | 0.840 |
| | | | CoV-2 | | | | |
| C3 | M21 + RNA_9 | | InfA | 465,342.764 | 185,201.555 | 33.638 | 0.736 |
| C3 | M21 + RNA_9 | | InfB | 99,997.390 | 21,453.013 | 32.358 | 0.740 |
| C3 | M21 + RNA_9 | | RNaseP | 684,048.776 | 77,851.057 | 32.820 | 0.436 |
| C3 | M21 + RNA_9 | | SARS- | 141,091.394 | 35,063.558 | 33.622 | 0.535 |
| | | | CoV-2 | | | | |
| ddC | M21 + RNA_9 | | InfA | 311,277.557 | 91,443.221 | 34.411 | 0.728 |
| ddC | M21 + RNA_9 | | InfB | 78,335.229 | 15,453.955 | 31.697 | 3.330 |
| ddC | M21 + RNA_9 | | RNaseP | 632,371.021 | 81,581.819 | 32.863 | 0.457 |
| ddC | M21 + RNA_9 | | SARS- | 96,245.596 | 29,978.505 | 33.112 | 2.006 |
| | | | CoV-2 | | | | |
| InvdT | M21 + RNA_9 | | InfA | 426,349.242 | 78,771.750 | 33.819 | 0.421 |
| InvdT | M21 + RNA_9 | | InfB | 84,472.822 | 31,967.987 | 32.466 | 1.286 |
| InvdT | M21 + RNA_9 | | RNaseP | 668,755.286 | 93,118.340 | 32.674 | 0.230 |
| InvdT | M21 + RNA_9 | | SARS- | 72,060.135 | 44,062.219 | 33.993 | 1.008 |
| | | | CoV-2 | | | | |
| amino | M21 + RNA_9 | | InfA | 166,533.445 | 61,182.438 | 35.042 | 1.011 |
| amino | M21 + RNA_9 | | InfB | 41,929.839 | 20,352.371 | 32.933 | 1.063 |
| amino | M21 + RNA_9 | | RNaseP | 607,241.328 | 60,393.631 | 32.745 | 0.362 |
| amino | M21 + RNA_9 | | SARS- | 90,212.097 | 22,391.398 | 33.201 | 0.435 |
| | | | CoV-2 | | | | |
| | M21 + RNA_27 | | InfA | 314,842.146 | 71,060.436 | 33.626 | 0.625 |
| | M21 + RNA_27 | | InfB | 51,924.106 | 17,033.436 | 32.291 | 0.835 |
| | M21 + RNA_27 | | RNaseP | 615,728.229 | 39,789.914 | 32.445 | 0.356 |
| | M21 + RNA_27 | | SARS- | 100,477.290 | 32,130.599 | 33.081 | 0.565 |
| | | | CoV-2 | | | | |
| 3′ Phosphate | M21 + RNA_27 | | InfA | 605,009.768 | 68,729.552 | 32.383 | 0.358 |
| 3′ Phosphate | M21 + RNA_27 | | InfB | 71,695.784 | 27,093.974 | 32.592 | 0.741 |
| 3′ Phosphate | M21 + RNA_27 | | RNaseP | 655,666.068 | 67,847.520 | 32.755 | 0.531 |
| 3′ Phosphate | M21 + RNA_27 | | SARS- | 115,907.324 | 28,481.881 | 33.626 | 0.481 |
| | | | CoV-2 | | | | |
| C3 | M21 + RNA_27 | | InfA | 473,083.615 | 73,005.437 | 33.294 | 0.439 |
| C3 | M21 + RNA_27 | | InfB | 84,439.844 | 20,995.571 | 32.340 | 0.831 |
| C3 | M21 + RNA_27 | | RNaseP | 653,936.297 | 81,306.118 | 32.772 | 0.488 |
| C3 | M21 + RNA_27 | | SARS- | 128,319.675 | 43,555.224 | 33.641 | 0.756 |
| | | | CoV-2 | | | | |
| ddC | M21 + RNA_27 | | InfA | 364,387.624 | 79,485.796 | 33.713 | 0.343 |
| ddC | M21 + RNA_27 | | InfB | 61,810.231 | 24,557.999 | 32.590 | 0.796 |
| ddC | M21 + RNA_27 | | RNaseP | 634,432.057 | 71,442.466 | 32.729 | 0.305 |
| ddC | M21 + RNA_27 | | SARS- | 118,180.504 | 36,017.249 | 33.423 | 0.249 |
| | | | CoV-2 | | | | |
| InvdT | M21 + RNA_27 | | InfA | 478,816.003 | 41,479.044 | 33.326 | 0.292 |
| InvdT | M21 + RNA_27 | | InfB | 69,745.480 | 21,268.860 | 32.808 | 0.715 |
| InvdT | M21 + RNA_27 | | RNaseP | 697,137.260 | 80,253.266 | 32.600 | 0.459 |
| InvdT | M21 + RNA_27 | | SARS- | 126,341.896 | 18,798.166 | 33.600 | 0.341 |
| | | | CoV-2 | | | | |
| amino | M21 + RNA_27 | | InfA | 240,472.238 | 84,932.465 | 34.839 | 0.861 |
| amino | M21 + RNA_27 | | InfB | 79,811.833 | 20,331.305 | 32.308 | 0.671 |
| amino | M21 + RNA_27 | | RNaseP | 660,952.021 | 72,141.280 | 32.799 | 0.509 |
| amino | M21 + RNA_27 | | SARS- | 118,377.116 | 26,943.998 | 33.644 | 0.472 |
| | | | CoV-2 | | | | |
| | M21 + RNA_9 | M21 + RNA_27 | InfA | 546,403.708 | 81,239.433 | 33.670 | 0.536 |
| | M21 + RNA_9 | M21 + RNA_27 | InfB | 108,502.412 | 25,515.805 | 32.563 | 0.805 |
| | M21 + RNA_9 | M21 + RNA_27 | RNaseP | 729,200.599 | 78,395.000 | 32.625 | 0.357 |
| | M21 + RNA_9 | M21 + RNA_27 | SARS- | 136,821.112 | 24,427.541 | 33.596 | 0.374 |
| | | | CoV-2 | | | | |
| Phos | M21 + RNA_9 | M21 + RNA_27 | InfA | 660,597.344 | 61,883.468 | 32.687 | 0.352 |
| Phos | M21 + RNA_9 | M21 + RNA_27 | InfB | 107,341.939 | 19,690.414 | 32.210 | 0.534 |
| Phos | M21 + RNA_9 | M21 + RNA_27 | RNaseP | 745,331.625 | 78,503.344 | 32.575 | 0.377 |
| Phos | M21 + RNA_9 | M21 + RNA_27 | SARS- | 164,761.066 | 46,794.169 | 33.315 | 0.576 |
| | | | CoV-2 | | | | |
| C3 | M21 + RNA_9 | M21 + RNA_27 | InfA | 567,978.518 | 67,620.698 | 33.340 | 0.542 |
| C3 | M21 + RNA_9 | M21 + RNA_27 | InfB | 89,101.962 | 30,009.868 | 32.851 | 0.941 |
| C3 | M21 + RNA_9 | M21 + RNA_27 | RNaseP | 690,268.615 | 59,597.526 | 32.775 | 0.337 |
| C3 | M21 + RNA_9 | M21 + RNA_27 | SARS- | 142,136.844 | 35,625.547 | 33.145 | 2.046 |
| | | | CoV-2 | | | | |
| ddC | M21 + RNA_9 | M21 + RNA_27 | InfA | 522,672.068 | 62,412.793 | 33.605 | 0.408 |
| ddC | M21 + RNA_9 | M21 + RNA_27 | InfB | 99,201.552 | 23,160.549 | 32.085 | 0.850 |
| ddC | M21 + RNA_9 | M21 + RNA_27 | RNaseP | 749,531.563 | 77,297.016 | 32.585 | 0.475 |
| ddC | M21 + RNA_9 | M21 + RNA_27 | SARS- | 126,760.973 | 51,777.091 | 33.699 | 0.806 |
| | | | CoV-2 | | | | |
| InvdT | M21 + RNA_9 | M21 + RNA_27 | InfA | 608,693.536 | 98,137.034 | 33.041 | 0.351 |
| InvdT | M21 + RNA_9 | M21 + RNA_27 | InfB | 77,570.815 | 41,985.792 | 32.853 | 1.399 |
| InvdT | M21 + RNA_9 | M21 + RNA_27 | RNaseP | 728,575.411 | 88,691.044 | 32.779 | 0.337 |
| InvdT | M21 + RNA_9 | M21 + RNA_27 | SARS- | 127,922.338 | 30,170.937 | 33.673 | 0.516 |
| | | | CoV-2 | | | | |
| amino | M21 + RNA_9 | M21 + RNA_27 | InfA | 368,601.818 | 76,883.636 | 33.925 | 1.010 |
| amino | M21 + RNA_9 | M21 + RNA_27 | InfB | 98,582.964 | 29,458.022 | 31.678 | 1.765 |
| amino | M21 + RNA_9 | M21 + RNA_27 | RNaseP | 705,930.445 | 83,083.938 | 32.588 | 0.386 |
| amino | M21 + RNA_9 | M21 + RNA_27 | SARS- | 134,238.793 | 47,390.309 | 33.023 | 2.157 |
| | | | CoV-2 | | | | |
| | No Aptamer 1 | | InfA | 9,979.732 | 8,372.444 | 37.849 | 4.124 |
| | No Aptamer 1 | | InfB | 712.015 | 329.608 | NA | NA |
| | No Aptamer 1 | | RNaseP | 175,382.745 | 22,640.161 | 31.945 | 0.387 |
| | No Aptamer 1 | | SARS- | 5,244.930 | 4,093.441 | 33.201 | 1.100 |
| | | | CoV-2 | | | | |
| | No Aptamer 2 | | InfA | 10,772.267 | 7,026.130 | 38.561 | 1.576 |
| | No Aptamer 2 | | InfB | 933.751 | 376.262 | 38.718 | 4.708 |
| | No Aptamer 2 | | RNaseP | 196,756.880 | 37,492.949 | 32.685 | 2.456 |
| | No Aptamer 2 | | SARS- | 8,767.410 | 5,351.093 | 33.119 | 1.262 |
| | | | CoV-2 | | | | |
| | RNA Aptamer 1 | | InfA | 39,796.037 | 17,657.965 | 36.516 | 1.239 |
| | RNA Aptamer 1 | | InfB | 899.204 | 668.567 | 43.032 | 2.635 |
| | RNA Aptamer 1 | | RNaseP | 189,358.603 | 28,064.254 | 32.063 | 0.250 |
| | RNA Aptamer 1 | | SARS- | 2,707.698 | 3,532.563 | 35.850 | 0.613 |
| | | | CoV-2 | | | | |
| | RNA Aptamer 2 | | InfA | 63,546.240 | 19,588.554 | 35.791 | 0.857 |
| | RNA Aptamer 2 | | InfB | 1,266.749 | 760.294 | 41.488 | 3.833 |
| | RNA Aptamer 2 | | RNaseP | 190,506.962 | 14,456.900 | 32.441 | 0.224 |
| | RNA Aptamer 2 | | SARS- | 10,237.001 | 7,852.122 | 33.441 | 2.787 |
| | | | CoV-2 | | | | |
| | No Template Control | | InfA | 2,834.733 | 2,764.725 | NA | NA |
| | No Template Control | | InfB | 414.246 | 491.685 | NA | NA |
| | No Template Control | | RNaseP | 863.870 | 93.325 | NA | NA |
| | No Template Control | | SARS- | 420.613 | 78.202 | NA | NA |
| | | | CoV-2 | | | | |

TABLE 9
Evaluation of impact of aptamer binding period on room temperature stability
6 hours following template addition to PrimeTime ™ One-Step
RT-qPCR Master Mix (Integrated DNA Technologies) with DNA aptamers.

| | | | | 4 h Endpoint | | 4 h Cycle | |
|---|---|---|---|---|---|---|---|
| | | | | Fluorescence | | Threshold | |
| | Incubation | Target | | Delta Rn | Delta Rn | Ct | Ct |
| Aptamer | Time | Name | Reporter | Mean | SD | Mean | SD |
| M21 + RNA_9_3Phos | 15 min | InfA | FAM | 51,490.482 | 35,637.197 | 30.414 | 0.629 |
| M21 + RNA_9_3Phos | 20 min | InfA | FAM | 34,307.244 | 70,952.636 | 30.792 | 0.911 |
| M21 + RNA_9_3Phos | 30 min | InfA | FAM | 187,873.737 | 41,080.389 | 30.704 | 0.275 |
| M21 + RNA_9_3Phos | 15 min | InfB | VIC | 23,080.780 | 21,803.034 | 32.415 | 0.623 |
| M21 + RNA_9_3Phos | 20 min | InfB | VIC | 141,038.227 | 58,481.066 | 33.377 | 0.659 |
| M21 + RNA_9_3Phos | 30 min | InfB | VIC | 42,779.654 | 11,047.218 | 32.802 | 0.395 |
| M21 + RNA_9_3Phos | 15 min | RNaseP | CY5 | 440,815.083 | 65,252.815 | 30.137 | 0.436 |
| M21 + RNA_9_3Phos | 20 min | RNaseP | CY5 | 286,493.096 | 88,757.362 | 30.129 | 0.350 |
| M21 + RNA_9_3Phos | 30 min | RNaseP | CY5 | 172,363.232 | 90,176.446 | 30.119 | 0.204 |
| M21 + RNA_9_3Phos | 15 min | SARS- | ROX | 61,557.468 | 10,268.997 | 30.890 | 1.151 |
| | | CoV-2 | | | | | |
| M21 + RNA_9_3Phos | 20 min | SARS- | ROX | 432,512.542 | 102,071.517 | 30.795 | 0.762 |
| | | CoV-2 | | | | | |
| M21 + RNA_9_3Phos | 30 min | SARS- | ROX | 229,719.633 | 40,895.936 | 30.607 | 0.675 |
| | | CoV-2 | | | | | |
| No Template | 15 min | InfA | FAM | 12,653.224 | 17,776.441 | 37.006 | 4.145 |
| Control | | | | | | | |
| No Template | 20 min | InfA | FAM | −708.664 | 32,011.252 | 36.164 | 0.934 |
| Control | | | | | | | |
| No Template | 30 min | InfA | FAM | 2,833.458 | 4,687.256 | 33.980 | 0.591 |
| Control | | | | | | | |
| No Template | 15 min | InfB | VIC | −2,911.318 | 1,562.622 | NA | NA |
| Control | | | | | | | |
| No Template | 20 min | InfB | VIC | 574.902 | 5,029.759 | NA | NA |
| Control | | | | | | | |
| No Template | 30 min | InfB | VIC | −20,236.627 | 2,331.285 | NA | NA |
| Control | | | | | | | |
| No Template | 15 min | RNaseP | CY5 | 1,494.207 | 1,046.782 | 37.968 | NA |
| Control | | | | | | | |
| No Template | 20 min | RNaseP | CY5 | −3,164.546 | 2,302.200 | NA | NA |
| Control | | | | | | | |
| No Template | 30 min | RNaseP | CY5 | 58,954.068 | 73,607.860 | 35.953 | 0.297 |
| Control | | | | | | | |
| No Template | 15 min | SARS- | ROX | −9,960.893 | 17,308.404 | 9.894 | NA |
| Control | | CoV-2 | | | | | |
| No Template | 20 min | SARS- | ROX | 7,202.016 | 11,013.176 | NA | NA |
| Control | | CoV-2 | | | | | |
| No Template | 30 min | SARS- | ROX | −4,526.254 | 3,439.226 | NA | NA |
| Control | | CoV-2 | | | | | |
| RTv2 | | InfA | FAM | 73,149.721 | 29,999.603 | 29.621 | 1.337 |
| RTv2 | | InfB | VIC | 34,011.723 | 10,524.364 | 18.248 | 2.728 |
| RTv2 | | RNaseP | CY5 | 241,692.357 | 32,517.422 | 30.163 | 0.388 |
| RTv2 | | SARS- | ROX | 55,224.291 | 13,755.365 | 28.692 | 4.960 |
| | | CoV-2 | | | | | |

TABLE 10
Evaluation of room temperature stability 6 hours following template addition to PrimeTime ™ One-
Step RT-qPCR Master Mix (Integrated DNA Technologies) with combinations DNA aptamers.

| | | 6 h Endpoint Fluorescence | | | |
|---|---|---|---|---|---|
| | Target | Delta Rn | Delta Rn | 6 h Cycle Threshold | |
| Sample Name | Name | Mean | SD | Ct Mean | Ct SD |
| M21 + RNA_9_3Phos | InfA | 152354.874 | 48896.095 | 30.824 | 0.620 |
| M21 + RNA_9_3Phos | InfB | 69327.251 | 14637.147 | 32.646 | 0.259 |
| M21 + RNA_9_3Phos | RNaseP | 443856.125 | 64834.665 | 31.469 | 0.465 |
| M21 + RNA_9_3Phos | SARS- | 213940.531 | 33093.173 | 31.962 | 0.378 |
| | CoV-2 | | | | |
| M21 + RNA_9_3Phos + M21_1 | InfA | 100136.603 | 40766.472 | 30.080 | 0.332 |
| M21 + RNA_9_3Phos + M21_1 | InfB | 27030.936 | 12605.595 | 32.573 | 0.740 |
| M21 + RNA_9_3Phos + M21_1 | RNaseP | 346971.500 | 15178.359 | 31.451 | 0.123 |
| M21 + RNA_9_3Phos + M21_1 | SARS- | 117898.706 | 30615.055 | 31.500 | 0.774 |
| | CoV-2 | | | | |
| M21 + RNA_9_3Phos + M21_2 | InfA | 52640.743 | 17015.827 | 31.154 | 0.424 |
| M21 + RNA_9_3Phos + M21_2 | InfB | 45899.570 | 12583.399 | 32.335 | 0.475 |
| M21 + RNA_9_3Phos + M21_2 | RNaseP | 385397.115 | 15444.911 | 31.406 | 0.046 |
| M21 + RNA_9_3Phos + M21_2 | SARS- | 144026.797 | 11551.277 | 30.776 | 1.232 |
| | CoV-2 | | | | |
| M21 + RNA_9_3Phos + M21_3 | InfA | 120367.867 | 10112.955 | 30.388 | 0.224 |
| M21 + RNA_9_3Phos + M21_3 | InfB | 36903.234 | 12378.270 | 32.786 | 0.334 |
| M21 + RNA_9_3Phos + M21_3 | RNaseP | 405931.531 | 33304.813 | 31.493 | 0.227 |
| M21 + RNA_9_3Phos + M21_3 | SARS- | 132322.833 | 19024.494 | 32.176 | 0.616 |
| | CoV-2 | | | | |
| M21 + RNA_9_3Phos + M21_4 | InfA | 167774.714 | 33527.423 | 29.602 | 0.680 |
| M21 + RNA_9_3Phos + M21_4 | InfB | 39703.816 | 8061.611 | 33.133 | 0.317 |
| M21 + RNA_9_3Phos + M21_4 | RNaseP | 427925.198 | 78329.479 | 31.529 | 0.677 |
| M21 + RNA_9_3Phos + M21_4 | SARS- | 182593.370 | 13931.908 | 30.745 | 0.867 |
| | CoV-2 | | | | |
| M21 + RNA_9_3Phos + M21_5 | InfA | 93673.224 | 23446.164 | 30.004 | 0.545 |
| M21 + RNA_9_3Phos + M21_5 | InfB | 31591.831 | 21545.078 | 32.776 | 1.065 |
| M21 + RNA_9_3Phos + M21_5 | RNaseP | 393099.906 | 30341.391 | 31.071 | 0.412 |
| M21 + RNA_9_3Phos + M21_5 | SARS- | 108299.969 | 33739.365 | 31.115 | 0.786 |
| | CoV-2 | | | | |
| M21 + RNA_9_3Phos + M21_6 | InfA | 90303.697 | 24488.654 | 30.242 | 0.220 |
| M21 + RNA_9_3Phos + M21_6 | InfB | 34700.757 | 8256.181 | 32.454 | 0.122 |
| M21 + RNA_9_3Phos + M21_6 | RNaseP | 370132.354 | 28500.813 | 31.472 | 0.396 |
| M21 + RNA_9_3Phos + M21_6 | SARS- | 123228.995 | 22555.568 | 31.401 | 0.419 |
| | CoV-2 | | | | |
| M21 + RNA_9_3Phos + M21_7 | InfA | 130220.518 | 44550.581 | 30.036 | 1.253 |
| M21 + RNA_9_3Phos + M21_7 | InfB | 28885.318 | 12052.398 | 33.090 | 0.903 |
| M21 + RNA_9_3Phos + M21_7 | RNaseP | 393561.688 | 34519.612 | 31.232 | 0.251 |
| M21 + RNA_9_3Phos + M21_7 | SARS- | 114973.201 | 27560.900 | 32.238 | 0.255 |
| | CoV-2 | | | | |
| M21 + RNA_9_3Phos + M21_8 | InfA | 104922.961 | 18967.967 | 30.015 | 0.140 |
| M21 + RNA_9_3Phos + M21_8 | InfB | 20765.375 | 14315.509 | 33.302 | 0.938 |
| M21 + RNA_9_3Phos + M21_8 | RNaseP | 321889.406 | 38173.884 | 31.766 | 0.463 |
| M21 + RNA_9_3Phos + M21_8 | SARS- | 133088.185 | 34017.083 | 31.617 | 0.692 |
| | CoV-2 | | | | |
| M21 + RNA_9_3Phos + M21_9 | InfA | 124430.706 | 40225.860 | 30.357 | 0.700 |
| M21 + RNA_9_3Phos + M21_9 | InfB | 44849.944 | 7497.741 | 32.605 | 0.324 |
| M21 + RNA_9_3Phos + M21_9 | RNaseP | 328095.083 | 49232.192 | 31.680 | 0.447 |
| M21 + RNA_9_3Phos + M21_9 | SARS- | 156236.234 | 21202.595 | 31.097 | 0.853 |
| | CoV-2 | | | | |
| M21 + RNA_9_3Phos + M21_10 | InfA | 110094.753 | 19405.130 | 30.382 | 0.497 |
| M21 + RNA_9_3Phos + M21_10 | InfB | 28522.099 | 7902.952 | 33.204 | 0.409 |
| M21 + RNA_9_3Phos + M21_10 | RNaseP | 393978.146 | 48200.348 | 31.368 | 0.308 |
| M21 + RNA_9_3Phos + M21_10 | SARS- | 145583.164 | 12367.314 | 31.838 | 0.321 |
| | CoV-2 | | | | |
| M21 + RNA_9_3Phos + M21_11 | InfA | 131998.641 | 13962.398 | 30.295 | 0.259 |
| M21 + RNA_9_3Phos + M21_11 | InfB | 25782.915 | 6567.146 | 33.246 | 0.561 |
| M21 + RNA_9_3Phos + M21_11 | RNaseP | 383023.646 | 41700.461 | 31.359 | 0.384 |
| M21 + RNA_9_3Phos + M21_11 | SARS- | 99476.169 | 19787.451 | 32.109 | 0.362 |
| | CoV-2 | | | | |
| M21 + RNA_9_3Phos + M21_12 | InfA | 95009.553 | 28156.064 | 30.758 | 0.617 |
| M21 + RNA_9_3Phos + M21_12 | InfB | 39438.381 | 13420.421 | 32.708 | 0.527 |
| M21 + RNA_9_3Phos + M21_12 | RNaseP | 402150.292 | 20090.572 | 31.260 | 0.119 |
| M21 + RNA_9_3Phos + M21_12 | SARS- | 131899.469 | 36456.395 | 31.023 | 0.699 |
| | CoV-2 | | | | |
| M21 + RNA_9_3Phos + M21_13 | InfA | 74083.016 | 10691.186 | 30.845 | 0.534 |
| M21 + RNA_9_3Phos + M21_13 | InfB | 29593.214 | 5126.772 | 32.983 | 0.205 |
| M21 + RNA_9_3Phos + M21_13 | RNaseP | 367471.865 | 42509.103 | 31.340 | 0.313 |
| M21 + RNA_9_3Phos + M21_13 | SARS- | 116556.625 | 11604.862 | 30.849 | 0.708 |
| | CoV-2 | | | | |
| M21 + RNA_9_3Phos + M21_14 | InfA | 124022.349 | 27937.037 | 30.317 | 0.603 |
| M21 + RNA_9_3Phos + M21_14 | InfB | 24662.737 | 12001.335 | 33.228 | 0.807 |
| M21 + RNA_9_3Phos + M21_14 | RNaseP | 371572.375 | 33708.253 | 31.524 | 0.312 |
| M21 + RNA_9_3Phos + M21_14 | SARS- | 121126.307 | 31938.107 | 31.838 | 0.504 |
| | CoV-2 | | | | |
| M21 + RNA_9_3Phos + M21_15 | InfA | 140539.063 | 5042.386 | 30.152 | 0.143 |
| M21 + RNA_9_3Phos + M21_15 | InfB | 35990.015 | 7085.749 | 32.793 | 0.187 |
| M21 + RNA_9_3Phos + M21_15 | RNaseP | 378401.125 | 65053.843 | 31.473 | 0.248 |
| M21 + RNA_9_3Phos + M21_15 | SARS- | 121932.635 | 50704.266 | 32.170 | 0.883 |
| | CoV-2 | | | | |
| M21 + RNA_9_3Phos + M21_16 | InfA | 86972.945 | 34792.707 | 30.893 | 0.934 |
| M21 + RNA_9_3Phos + M21_16 | InfB | 45971.496 | 11843.969 | 32.522 | 0.349 |
| M21 + RNA_9_3Phos + M21_16 | RNaseP | 348380.406 | 22561.503 | 31.579 | 0.113 |
| M21 + RNA_9_3Phos + M21_16 | SARS- | 121066.117 | 42322.172 | 32.272 | 0.561 |
| | CoV-2 | | | | |
| M21 + RNA_9_3Phos + M21_17 | InfA | 107620.057 | 17062.676 | 29.780 | 0.252 |
| M21 + RNA_9_3Phos + M21_17 | InfB | 31487.754 | 13943.339 | 32.432 | 0.675 |
| M21 + RNA_9_3Phos + M21_17 | RNaseP | 328929.208 | 38609.443 | 31.221 | 0.433 |
| M21 + RNA_9_3Phos + M21_17 | SARS- | 113679.617 | 9254.515 | 30.968 | 0.625 |
| | CoV-2 | | | | |
| M21 + RNA_9_3Phos + M21_18 | InfA | 95923.284 | 8420.084 | 30.568 | 0.177 |
| M21 + RNA_9_3Phos + M21_18 | InfB | 33481.817 | 4562.663 | 32.842 | 0.328 |
| M21 + RNA_9_3Phos + M21_18 | RNaseP | 415160.063 | 30374.534 | 31.261 | 0.377 |
| M21 + RNA_9_3Phos + M21_18 | SARS- | 115320.711 | 27502.072 | 31.994 | 0.602 |
| | CoV-2 | | | | |
| M21 + RNA_9_3Phos + M21_19 | InfA | 99392.941 | 66559.876 | 32.673 | 4.234 |
| M21 + RNA_9_3Phos + M21_19 | InfB | 44862.395 | 6582.222 | 32.762 | 0.151 |
| M21 + RNA_9_3Phos + M21_19 | RNaseP | 409664.563 | 34581.675 | 31.266 | 0.237 |
| M21 + RNA_9_3Phos + M21_19 | SARS- | 143326.615 | 43019.861 | 31.360 | 0.780 |
| | CoV-2 | | | | |
| M21 + RNA_9_3Phos + M21_20 | InfA | 135051.029 | 24678.433 | 30.184 | 0.383 |
| M21 + RNA_9_3Phos + M21_20 | InfB | 31624.573 | 30827.319 | 33.220 | 1.812 |
| M21 + RNA_9_3Phos + M21_20 | RNaseP | 346119.552 | 31125.571 | 31.569 | 0.277 |
| M21 + RNA_9_3Phos + M21_20 | SARS- | 179296.479 | 15145.598 | 31.373 | 0.352 |
| | CoV-2 | | | | |
| M21 + RNA_9_3Phos + M21_21 | InfA | 86176.576 | 25671.946 | 31.266 | 0.497 |
| M21 + RNA_9_3Phos + M21_21 | InfB | 57012.148 | 9438.740 | 32.349 | 0.294 |
| M21 + RNA_9_3Phos + M21_21 | RNaseP | 437029.083 | 11161.483 | 31.307 | 0.296 |
| M21 + RNA_9_3Phos + M21_21 | SARS- | 188593.443 | 48372.608 | 31.804 | 0.508 |
| | CoV-2 | | | | |
| M21 + RNA_9_3Phos + M21_22 | InfA | 81885.240 | 19238.381 | 26.298 | 7.750 |
| M21 + RNA_9_3Phos + M21_22 | InfB | 23703.949 | 6820.989 | 27.383 | 9.528 |
| M21 + RNA_9_3Phos + M21_22 | RNaseP | 363873.198 | 28012.317 | 30.707 | 0.929 |
| M21 + RNA_9_3Phos + M21_22 | SARS- | 97610.507 | 43347.279 | 26.777 | 8.578 |
| | CoV-2 | | | | |
| M21 + RNA_9_3Phos + M21_23 | InfA | 133267.992 | 25532.842 | 30.096 | 0.607 |
| M21 + RNA_9_3Phos + M21_23 | InfB | 27849.275 | 9354.368 | 33.042 | 0.331 |
| M21 + RNA_9_3Phos + M21_23 | RNaseP | 394840.031 | 38489.557 | 31.318 | 0.175 |
| M21 + RNA_9_3Phos + M21_23 | SARS- | 133784.398 | 25204.337 | 30.942 | 1.071 |
| | CoV-2 | | | | |
| M21 + RNA_9_3Phos + M21_24 | InfA | 60102.109 | 27708.838 | 31.624 | 1.207 |
| M21 + RNA_9_3Phos + M21_24 | InfB | 30570.679 | 20085.574 | 33.475 | 1.242 |
| M21 + RNA_9_3Phos + M21_24 | RNaseP | 395350.167 | 39561.108 | 31.426 | 0.261 |
| M21 + RNA_9_3Phos + M21_24 | SARS- | 134123.940 | 4098.829 | 31.960 | 0.325 |
| | CoV-2 | | | | |
| M21 + RNA_9_3Phos + M21_25 | InfA | 136101.396 | 18619.171 | 30.218 | 0.459 |
| M21 + RNA_9_3Phos + M21_25 | InfB | 42749.060 | 4595.267 | 32.305 | 0.137 |
| M21 + RNA_9_3Phos + M21_25 | RNaseP | 410507.750 | 32310.570 | 31.190 | 0.320 |
| M21 + RNA_9_3Phos + M21_25 | SARS- | 114221.784 | 8082.820 | 31.733 | 0.502 |
| | CoV-2 | | | | |
| M21 + RNA_9_3Phos + M21_26 | InfA | 135605.974 | 22337.288 | 30.245 | 0.306 |
| M21 + RNA_9_3Phos + M21_26 | InfB | 52469.696 | 14877.036 | 32.667 | 0.678 |
| M21 + RNA_9_3Phos + M21_26 | RNaseP | 353486.063 | 35584.802 | 31.736 | 0.277 |
| M21 + RNA_9_3Phos + M21_26 | SARS- | 161243.115 | 9358.382 | 31.406 | 0.164 |
| | CoV-2 | | | | |
| M21 + RNA_9_3Phos + M21_27 | InfA | 96390.268 | 34835.011 | 30.469 | 0.800 |
| M21 + RNA_9_3Phos + M21_27 | InfB | 35570.353 | 3192.017 | 32.919 | 0.078 |
| M21 + RNA_9_3Phos + M21_27 | RNaseP | 437371.094 | 50022.839 | 31.251 | 0.357 |
| M21 + RNA_9_3Phos + M21_27 | SARS- | 139103.974 | 5150.464 | 31.588 | 0.354 |
| | CoV-2 | | | | |
| M21 + RNA_9_3Phos + M21_28 | InfA | 100684.526 | 38184.262 | 30.705 | 0.695 |
| M21 + RNA_9_3Phos + M21_28 | InfB | 26412.067 | 6236.689 | 33.411 | 0.239 |
| M21 + RNA_9_3Phos + M21_28 | RNaseP | 416535.927 | 29212.131 | 31.272 | 0.237 |
| M21 + RNA_9_3Phos + M21_28 | SARS- | 160603.271 | 32527.188 | 30.111 | 2.005 |
| | CoV-2 | | | | |
| M21 + RNA_9_3Phos + M21_29 | InfA | 158766.724 | 46547.942 | 27.086 | 4.803 |
| M21 + RNA_9_3Phos + M21_29 | InfB | 25110.936 | 9745.169 | 33.272 | 1.329 |
| M21 + RNA_9_3Phos + M21_29 | RNaseP | 436993.073 | 82494.689 | 30.819 | 0.073 |
| M21 + RNA_9_3Phos + M21_29 | SARS- | 105067.461 | 45242.211 | 24.464 | 8.804 |
| | CoV-2 | | | | |
| M21 + RNA_9_3Phos + M21_30 | InfA | 106462.661 | 7561.049 | 26.472 | 6.968 |
| M21 + RNA_9_3Phos + M21_30 | InfB | 36123.786 | 11124.252 | 32.117 | 1.027 |
| M21 + RNA_9_3Phos + M21_30 | RNaseP | 352763.500 | 67424.779 | 31.204 | 0.277 |
| M21 + RNA_9_3Phos + M21_30 | SARS- | 102871.263 | 14918.855 | 27.580 | 8.042 |
| | CoV-2 | | | | |
| M21 + RNA_9_3Phos + M21_31 | InfA | 88453.413 | 42975.454 | 29.433 | 2.652 |
| M21 + RNA_9_3Phos + M21_31 | InfB | 23679.601 | 6561.225 | 33.199 | 0.429 |
| M21 + RNA_9_3Phos + M21_31 | RNaseP | 397696.635 | 14834.395 | 31.019 | 0.239 |
| M21 + RNA_9_3Phos + M21_31 | SARS- | 136815.531 | 25482.397 | 28.293 | 5.743 |
| | CoV-2 | | | | |
| M21 + RNA_9_3Phos + M21_32 | InfA | 62423.911 | 24538.779 | 31.163 | 0.577 |
| M21 + RNA_9_3Phos + M21_32 | InfB | 19795.396 | 5544.336 | 33.123 | 0.144 |
| M21 + RNA_9_3Phos + M21_32 | RNaseP | 343336.552 | 23532.344 | 31.516 | 0.306 |
| M21 + RNA_9_3Phos + M21_32 | SARS- | 90553.281 | 19611.382 | 31.562 | 0.775 |
| | CoV-2 | | | | |
| M21 + RNA_9_3Phos + M21_33 | InfA | 102430.237 | 8843.163 | 30.720 | 0.288 |
| M21 + RNA_9_3Phos + M21_33 | InfB | 48618.868 | 11159.513 | 32.525 | 0.366 |
| M21 + RNA_9_3Phos + M21_33 | RNaseP | 343396.500 | 36722.496 | 31.725 | 0.246 |
| M21 + RNA_9_3Phos + M21_33 | SARS- | 132596.896 | 17443.032 | 31.657 | 0.720 |
| | CoV-2 | | | | |
| M21 + RNA_9_3Phos + M21_34 | InfA | 98614.602 | 32331.418 | 31.087 | 0.352 |
| M21 + RNA_9_3Phos + M21_34 | InfB | 51599.846 | 2325.918 | 32.554 | 0.127 |
| M21 + RNA_9_3Phos + M21_34 | RNaseP | 408504.177 | 37991.402 | 31.473 | 0.349 |
| M21 + RNA_9_3Phos + M21_34 | SARS- | 182800.510 | 7033.534 | 31.629 | 0.298 |
| | CoV-2 | | | | |
| M21 + RNA_9_3Phos + M21_35 | InfA | 210091.063 | 70631.444 | 27.566 | 3.274 |
| M21 + RNA_9_3Phos + M21_35 | InfB | 58526.072 | 5482.038 | 32.104 | 0.904 |
| M21 + RNA_9_3Phos + M21_35 | RNaseP | 391589.813 | 30741.409 | 31.266 | 0.284 |
| M21 + RNA_9_3Phos + M21_35 | SARS- | 177449.578 | 63642.293 | 27.133 | 8.522 |
| | CoV-2 | | | | |
| M21 + RNA_9_3Phos + M21_36 | InfA | 113309.039 | 23533.495 | 30.300 | 1.364 |
| M21 + RNA_9_3Phos + M21_36 | InfB | 35794.915 | 3874.583 | 32.875 | 0.218 |
| M21 + RNA_9_3Phos + M21_36 | RNaseP | 454706.740 | 68308.343 | 31.167 | 0.244 |
| M21 + RNA_9_3Phos + M21_36 | SARS- | 136457.029 | 44658.981 | 27.572 | 6.524 |
| | CoV-2 | | | | |
| M21 + RNA_9_3Phos + M21_37 | InfA | 82313.914 | 20298.097 | 30.940 | 0.452 |
| M21 + RNA_9_3Phos + M21_37 | InfB | 35460.572 | 18272.162 | 32.878 | 0.973 |
| M21 + RNA_9_3Phos + M21_37 | RNaseP | 378039.365 | 17432.519 | 31.535 | 0.417 |
| M21 + RNA_9_3Phos + M21_37 | SARS- | 173147.974 | 7734.837 | 30.902 | 0.474 |
| | CoV-2 | | | | |
| M21 + RNA_9_3Phos + M21_38 | InfA | 126813.292 | 15352.826 | 30.334 | 0.644 |
| M21 + RNA_9_3Phos + M21_38 | InfB | 45804.078 | 21868.743 | 32.673 | 1.032 |
| M21 + RNA_9_3Phos + M21_38 | RNaseP | 402478.750 | 77905.455 | 31.406 | 0.518 |
| M21 + RNA_9_3Phos + M21_38 | SARS- | 143094.156 | 15481.827 | 31.716 | 0.645 |
| | CoV-2 | | | | |
| M21 + RNA_9_3Phos + M21_39 | InfA | 163311.396 | 35809.974 | 30.360 | 0.610 |
| M21 + RNA_9_3Phos + M21_39 | InfB | 39103.279 | 14910.147 | 33.093 | 0.752 |
| M21 + RNA_9_3Phos + M21_39 | RNaseP | 415712.594 | 35553.648 | 31.313 | 0.309 |
| M21 + RNA_9_3Phos + M21_39 | SARS- | 168415.083 | 39881.580 | 31.825 | 0.473 |
| | CoV-2 | | | | |
| M21 + RNA_9_3Phos + M21_40 | InfA | 134033.878 | 33627.191 | 30.358 | 0.663 |
| M21 + RNA_9_3Phos + M21_40 | InfB | 41278.348 | 12011.728 | 32.666 | 0.442 |
| M21 + RNA_9_3Phos + M21_40 | RNaseP | 329641.198 | 12390.346 | 31.583 | 0.156 |
| M21 + RNA_9_3Phos + M21_40 | SARS- | 115070.117 | 22306.398 | 31.953 | 0.382 |
| | CoV-2 | | | | |
| M21 + RNA_9_3Phos + M21_41 | InfA | 121813.122 | 9411.590 | 30.508 | 0.503 |
| M21 + RNA_9_3Phos + M21_41 | InfB | 54020.224 | 16891.961 | 32.379 | 0.650 |
| M21 + RNA_9_3Phos + M21_41 | RNaseP | 395285.229 | 32418.703 | 31.493 | 0.271 |
| M21 + RNA_9_3Phos + M21_41 | SARS- | 134418.122 | 31269.511 | 29.339 | 4.728 |
| | CoV-2 | | | | |
| M21 + RNA_9_3Phos + M21_42 | InfA | 124267.570 | 17482.859 | 29.539 | 2.543 |
| M21 + RNA_9_3Phos + M21_42 | InfB | 36949.426 | 19619.012 | 32.942 | 1.115 |
| M21 + RNA_9_3Phos + M21_42 | RNaseP | 395232.250 | 27512.862 | 31.474 | 0.379 |
| M21 + RNA_9_3Phos + M21_42 | SARS- | 197003.865 | 16089.528 | 27.768 | 6.689 |
| | CoV-2 | | | | |
| M21 + RNA_9_3Phos + M21_43 | InfA | 116172.139 | 50849.547 | 30.841 | 1.063 |
| M21 + RNA_9_3Phos + M21_43 | InfB | 31077.954 | 13261.149 | 33.198 | 0.939 |
| M21 + RNA_9_3Phos + M21_43 | RNaseP | 454430.708 | 27494.588 | 31.131 | 0.403 |
| M21 + RNA_9_3Phos + M21_43 | SARS- | 171220.096 | 41300.989 | 31.136 | 0.961 |
| | CoV-2 | | | | |
| M21 + RNA_9_3Phos + M21_44 | InfA | 141513.076 | 40385.547 | 30.304 | 0.598 |
| M21 + RNA_9_3Phos + M21_44 | InfB | 43881.432 | 11088.805 | 32.452 | 0.608 |
| M21 + RNA_9_3Phos + M21_44 | RNaseP | 346563.302 | 28293.170 | 31.796 | 0.203 |
| M21 + RNA_9_3Phos + M21_44 | SARS- | 191045.135 | 27833.870 | 30.772 | 0.302 |
| | CoV-2 | | | | |
| M21 + RNA_9_3Phos + M21_45 | InfA | 113663.456 | 23383.206 | 30.223 | 0.469 |
| M21 + RNA_9_3Phos + M21_45 | InfB | 26173.765 | 16827.327 | 32.984 | 0.849 |
| M21 + RNA_9_3Phos + M21_45 | RNaseP | 398376.167 | 44344.803 | 31.302 | 0.270 |
| M21 + RNA_9_3Phos + M21_45 | SARS- | 117529.130 | 8249.782 | 31.655 | 0.744 |
| | CoV-2 | | | | |
| M21 + RNA_9_3Phos + M21_46 | InfA | 115635.423 | 40046.695 | 30.435 | 1.292 |
| M21 + RNA_9_3Phos + M21_46 | InfB | 38296.459 | 9439.912 | 32.916 | 0.318 |
| M21 + RNA_9_3Phos + M21_46 | RNaseP | 408700.052 | 34959.677 | 31.685 | 0.119 |
| M21 + RNA_9_3Phos + M21_46 | SARS- | 147107.036 | 11021.284 | 31.612 | 0.357 |
| | CoV-2 | | | | |
| M21 + RNA_9_3Phos + M21_47 | InfA | 125246.695 | 16433.337 | 30.258 | 0.370 |
| M21 + RNA_9_3Phos + M21_47 | InfB | 56815.645 | 18419.057 | 32.163 | 0.227 |
| M21 + RNA_9_3Phos + M21_47 | RNaseP | 430351.438 | 22703.387 | 31.194 | 0.250 |
| M21 + RNA_9_3Phos + M21_47 | SARS- | 124276.544 | 40067.561 | 31.563 | 1.028 |
| | CoV-2 | | | | |
| M21 + RNA_9_3Phos + M21_48 | InfA | 108865.701 | 39603.086 | 30.809 | 0.332 |
| M21 + RNA_9_3Phos + M21_48 | InfB | 42968.212 | 10463.940 | 32.794 | 0.553 |
| M21 + RNA_9_3Phos + M21_48 | RNaseP | 364619.760 | 43040.881 | 31.640 | 0.364 |
| M21 + RNA_9_3Phos + M21_48 | SARS- | 106418.806 | 40776.789 | 32.304 | 0.997 |
| | CoV-2 | | | | |
| M21 + RNA_9_3Phos + M21_49 | InfA | 110802.865 | 43515.869 | 29.972 | 0.966 |
| M21 + RNA_9_3Phos + M21_49 | InfB | 23740.660 | 20969.674 | 33.055 | 1.192 |
| M21 + RNA_9_3Phos + M21_49 | RNaseP | 382802.719 | 14078.171 | 31.488 | 0.237 |
| M21 + RNA_9_3Phos + M21_49 | SARS- | 123185.240 | 38379.828 | 30.853 | 0.883 |
| | CoV-2 | | | | |
| M21 + RNA_9_3Phos + M21_50 | InfA | 139033.328 | 24651.087 | 30.286 | 0.343 |
| M21 + RNA_9_3Phos + M21_50 | InfB | 41963.307 | 12698.012 | 32.811 | 0.512 |
| M21 + RNA_9_3Phos + M21_50 | RNaseP | 435268.625 | 55204.773 | 31.230 | 0.304 |
| M21 + RNA_9_3Phos + M21_50 | SARS- | 167482.234 | 6255.743 | 31.028 | 0.818 |
| | CoV-2 | | | | |
| M21 + RNA_9_3Phos + M21 + RNA_1 | InfA | 219431.438 | 39259.269 | 30.329 | 0.850 |
| M21 + RNA_9_3Phos + M21 + RNA_1 | InfB | 57880.760 | 12527.772 | 32.901 | 0.452 |
| M21 + RNA_9_3Phos + M21 + RNA_1 | RNaseP | 403498.000 | 30777.650 | 31.471 | 0.317 |
| M21 + RNA_9_3Phos + M21 + RNA_1 | SARS- | 210294.349 | 13897.248 | 32.134 | 0.723 |
| | CoV-2 | | | | |
| M21 + RNA_9_3Phos + M21 + RNA_2 | InfA | 155108.581 | 30844.394 | 30.318 | 0.569 |
| M21 + RNA_9_3Phos + M21 + RNA_2 | InfB | 46744.008 | 1586.369 | 33.012 | 0.082 |
| M21 + RNA_9_3Phos + M21 + RNA_2 | RNaseP | 372431.156 | 27076.682 | 31.688 | 0.255 |
| M21 + RNA_9_3Phos + M21 + RNA_2 | SARS- | 113553.453 | 25348.753 | 32.319 | 0.547 |
| | CoV-2 | | | | |
| M21 + RNA_9_3Phos + M21 + RNA_3 | InfA | 158514.503 | 59056.643 | 30.223 | 0.718 |
| M21 + RNA_9_3Phos + M21 + RNA_3 | InfB | 49479.169 | 14838.742 | 32.734 | 0.603 |
| M21 + RNA_9_3Phos + M21 + RNA_3 | RNaseP | 359465.021 | 24887.643 | 31.583 | 0.050 |
| M21 + RNA_9_3Phos + M21 + RNA_3 | SARS- | 139568.159 | 17129.571 | 31.222 | 1.690 |
| | CoV-2 | | | | |
| M21 + RNA_9_3Phos + M21 + RNA_4 | InfA | 141490.065 | 16051.631 | 30.067 | 0.329 |
| M21 + RNA_9_3Phos + M21 + RNA_4 | InfB | 37370.632 | 7655.544 | 33.210 | 0.396 |
| M21 + RNA_9_3Phos + M21 + RNA_4 | RNaseP | 381288.750 | 52071.422 | 31.485 | 0.393 |
| M21 + RNA_9_3Phos + M21 + RNA_4 | SARS- | 120378.477 | 23290.707 | 31.913 | 0.258 |
| | CoV-2 | | | | |
| M21 + RNA_9_3Phos + M21 + RNA_5 | InfA | 164468.526 | 57532.732 | 30.725 | 0.899 |
| M21 + RNA_9_3Phos + M21 + RNA_5 | InfB | 68453.155 | 21919.992 | 32.802 | 0.408 |
| M21 + RNA_9_3Phos + M21 + RNA_5 | RNaseP | 351543.740 | 26304.423 | 31.697 | 0.106 |
| M21 + RNA_9_3Phos + M21 + RNA_5 | SARS- | 121864.607 | 50625.129 | 32.904 | 1.212 |
| | CoV-2 | | | | |
| M21 + RNA_9_3Phos + M21 + RNA_6 | InfA | 123660.641 | 37569.003 | 30.762 | 0.970 |
| M21 + RNA_9_3Phos + M21 + RNA_6 | InfB | 52734.452 | 15498.469 | 32.753 | 0.358 |
| M21 + RNA_9_3Phos + M21 + RNA_6 | RNaseP | 392474.219 | 6763.761 | 31.743 | 0.135 |
| M21 + RNA_9_3Phos + M21 + RNA_6 | SARS- | 148406.536 | 2095.093 | 31.705 | 0.790 |
| | CoV-2 | | | | |
| M21 + RNA_9_3Phos + M21 + RNA_7 | InfA | 126917.896 | 25932.581 | 30.977 | 0.311 |
| M21 + RNA_9_3Phos + M21 + RNA_7 | InfB | 47837.738 | 9698.419 | 32.960 | 0.249 |
| M21 + RNA_9_3Phos + M21 + RNA_7 | RNaseP | 316638.193 | 46589.116 | 32.079 | 0.321 |
| M21 + RNA_9_3Phos + M21 + RNA_7 | SARS- | 134350.589 | 42858.335 | 32.698 | 0.496 |
| | CoV-2 | | | | |
| M21 + RNA_9_3Phos + M21 + RNA_8 | InfA | 115944.008 | 11218.090 | 30.641 | 0.707 |
| M21 + RNA_9_3Phos + M21 + RNA_8 | InfB | 43450.406 | 8142.139 | 32.949 | 0.424 |
| M21 + RNA_9_3Phos + M21 + RNA_8 | RNaseP | 248783.000 | 35791.698 | 32.719 | 0.477 |
| M21 + RNA_9_3Phos + M21 + RNA_8 | SARS- | 151987.427 | 46531.707 | 32.329 | 0.841 |
| | CoV-2 | | | | |
| M21 + RNA_9_3Phos + M21 + RNA_9 | InfA | 162857.531 | 22242.401 | 30.474 | 0.226 |
| M21 + RNA_9_3Phos + M21 + RNA_9 | InfB | 72481.380 | 10535.402 | 32.597 | 0.452 |
| M21 + RNA_9_3Phos + M21 + RNA_9 | RNaseP | 403029.250 | 1717.571 | 31.555 | 0.237 |
| M21 + RNA_9_3Phos + M21 + RNA_9 | SARS- | 185983.010 | 27958.069 | 31.436 | 0.720 |
| | CoV-2 | | | | |
| M21 + RNA_9_3Phos + M21 + RNA_19 | InfA | 131819.932 | 6176.348 | 30.787 | 0.118 |
| M21 + RNA_9_3Phos + M21 + RNA_19 | InfB | 58919.014 | 15067.121 | 32.822 | 0.741 |
| M21 + RNA_9_3Phos + M21 + RNA_19 | RNaseP | 415696.063 | 38365.243 | 31.578 | 0.376 |
| M21 + RNA_9_3Phos + M21 + RNA_19 | SARS- | 178685.630 | 34848.071 | 31.744 | 0.296 |
| | CoV-2 | | | | |
| M21 + RNA_9_3Phos + M21 + RNA_10 | InfA | 150534.612 | 14867.092 | 30.363 | 0.241 |
| M21 + RNA_9_3Phos + M21 + RNA_10 | InfB | 57399.909 | 16477.550 | 32.560 | 0.701 |
| M21 + RNA_9_3Phos + M21 + RNA_10 | RNaseP | 388256.021 | 5369.381 | 31.518 | 0.311 |
| M21 + RNA_9_3Phos + M21 + RNA_10 | SARS- | 141410.391 | 51490.637 | 32.078 | 0.814 |
| | CoV-2 | | | | |
| M21 + RNA_9_3Phos + M21 + RNA_11 | InfA | 163580.094 | 30267.230 | 30.540 | 0.638 |
| M21 + RNA_9_3Phos + M21 + RNA_11 | InfB | 46986.242 | 11116.430 | 33.219 | 0.673 |
| M21 + RNA_9_3Phos + M21 + RNA_11 | RNaseP | 416950.458 | 22064.182 | 31.492 | 0.276 |
| M21 + RNA_9_3Phos + M21 + RNA_11 | SARS- | 180996.620 | 24081.489 | 31.912 | 0.220 |
| | CoV-2 | | | | |
| M21 + RNA_9_3Phos + M21 + RNA_12 | InfA | 176457.833 | 15586.863 | 30.298 | 0.183 |
| M21 + RNA_9_3Phos + M21 + RNA_12 | InfB | 67284.204 | 13570.899 | 32.698 | 0.393 |
| M21 + RNA_9_3Phos + M21 + RNA_12 | RNaseP | 418596.208 | 23375.603 | 31.517 | 0.043 |
| M21 + RNA_9_3Phos + M21 + RNA_12 | SARS- | 202840.307 | 25942.674 | 31.426 | 1.093 |
| | CoV-2 | | | | |
| M21 + RNA_9_3Phos + M21 + RNA_13 | InfA | 128227.997 | 42150.376 | 30.385 | 0.655 |
| M21 + RNA_9_3Phos + M21 + RNA_13 | InfB | 51989.801 | 959.518 | 32.528 | 0.256 |
| M21 + RNA_9_3Phos + M21 + RNA_13 | RNaseP | 409782.948 | 42120.668 | 31.351 | 0.179 |
| M21 + RNA_9_3Phos + M21 + RNA_13 | SARS- | 144025.628 | 29505.578 | 30.941 | 1.816 |
| | CoV-2 | | | | |
| M21 + RNA_9_3Phos + M21 + RNA_14 | InfA | 119995.732 | 29771.853 | 30.448 | 0.462 |
| M21 + RNA_9_3Phos + M21 + RNA_14 | InfB | 40895.952 | 6693.571 | 32.854 | 0.288 |
| M21 + RNA_9_3Phos + M21 + RNA_14 | RNaseP | 356766.667 | 21090.657 | 31.726 | 0.244 |
| M21 + RNA_9_3Phos + M21 + RNA_14 | SARS- | 140582.997 | 35153.922 | 31.905 | 0.380 |
| | CoV-2 | | | | |
| M21 + RNA_9_3Phos + M21 + RNA_15 | InfA | 126071.638 | 13938.574 | 30.418 | 0.335 |
| M21 + RNA_9_3Phos + M21 + RNA_15 | InfB | 22835.604 | 20002.123 | 33.129 | 0.260 |
| M21 + RNA_9_3Phos + M21 + RNA_15 | RNaseP | 361477.594 | 31033.631 | 31.441 | 0.375 |
| M21 + RNA_9_3Phos + M21 + RNA_15 | SARS- | 151912.315 | 60281.567 | 31.610 | 0.992 |
| | CoV-2 | | | | |
| M21 + RNA_9_3Phos + M21 + RNA_16 | InfA | 90348.612 | 27647.223 | 31.144 | 0.575 |
| M21 + RNA_9_3Phos + M21 + RNA_16 | InfB | 40892.757 | 22072.745 | 32.927 | 1.000 |
| M21 + RNA_9_3Phos + M21 + RNA_16 | RNaseP | 386618.448 | 75366.683 | 31.653 | 0.580 |
| M21 + RNA_9_3Phos + M21 + RNA_16 | SARS- | 179958.984 | 22476.504 | 31.754 | 0.334 |
| | CoV-2 | | | | |
| M21 + RNA_9_3Phos + M21 + RNA_17 | InfA | 194003.000 | 46530.197 | 29.778 | 0.430 |
| M21 + RNA_9_3Phos + M21 + RNA_17 | InfB | 37871.143 | 3740.978 | 33.286 | 0.163 |
| M21 + RNA_9_3Phos + M21 + RNA_17 | RNaseP | 405849.521 | 29659.757 | 31.533 | 0.306 |
| M21 + RNA_9_3Phos + M21 + RNA_17 | SARS- | 164680.081 | 68019.213 | 31.921 | 1.232 |
| | CoV-2 | | | | |
| M21 + RNA_9_3Phos + M21 + RNA_18 | InfA | 95283.465 | 29560.738 | 31.245 | 0.539 |
| M21 + RNA_9_3Phos + M21 + RNA_18 | InfB | 59804.434 | 16695.854 | 32.569 | 0.580 |
| M21 + RNA_9_3Phos + M21 + RNA_18 | RNaseP | 448359.938 | 42981.326 | 31.357 | 0.265 |
| M21 + RNA_9_3Phos + M21 + RNA_18 | SARS- | 192543.885 | 31196.191 | 32.087 | 0.402 |
| | CoV-2 | | | | |
| M21 + RNA_9_3Phos + M21 + RNA_20 | InfA | 103278.126 | 32487.855 | 30.903 | 0.812 |
| M21 + RNA_9_3Phos + M21 + RNA_20 | InfB | 37481.609 | 2992.734 | 33.046 | 0.229 |
| M21 + RNA_9_3Phos + M21 + RNA_20 | RNaseP | 559877.521 | 10986.102 | 30.939 | 0.102 |
| M21 + RNA_9_3Phos + M21 + RNA_20 | SARS- | 169330.859 | 7796.673 | 30.987 | 0.685 |
| | CoV-2 | | | | |
| M21 + RNA_9_3Phos + M21 + RNA_21 | InfA | 147889.917 | 43797.958 | 30.867 | 0.608 |
| M21 + RNA_9_3Phos + M21 + RNA_21 | InfB | 59140.227 | 20875.212 | 32.854 | 1.024 |
| M21 + RNA_9_3Phos + M21 + RNA_21 | RNaseP | 432913.875 | 5507.416 | 31.432 | 0.019 |
| M21 + RNA_9_3Phos + M21 + RNA_21 | SARS- | 163706.953 | 25173.461 | 31.841 | 0.363 |
| | CoV-2 | | | | |
| M21 + RNA_9_3Phos + M21 + RNA_22 | InfA | 198262.656 | 20430.757 | 29.758 | 0.453 |
| M21 + RNA_9_3Phos + M21 + RNA_22 | InfB | 50701.240 | 2919.906 | 32.593 | 0.185 |
| M21 + RNA_9_3Phos + M21 + RNA_22 | RNaseP | 369579.938 | 32492.028 | 31.708 | 0.261 |
| M21 + RNA_9_3Phos + M21 + RNA_22 | SARS- | 142614.471 | 14699.847 | 31.721 | 0.524 |
| | CoV-2 | | | | |
| M21 + RNA_9_3Phos + M21 + RNA_23 | InfA | 114512.570 | 22483.888 | 30.540 | 0.397 |
| M21 + RNA_9_3Phos + M21 + RNA_23 | InfB | 28035.074 | 3521.412 | 33.158 | 0.102 |
| M21 + RNA_9_3Phos + M21 + RNA_23 | RNaseP | 387673.490 | 29727.142 | 31.409 | 0.269 |
| M21 + RNA_9_3Phos + M21 + RNA_23 | SARS- | 142443.419 | 21887.673 | 31.780 | 0.461 |
| | CoV-2 | | | | |
| M21 + RNA_9_3Phos + M21 + RNA_24 | InfA | 108718.161 | 20595.009 | 31.003 | 0.221 |
| M21 + RNA_9_3Phos + M21 + RNA_24 | InfB | 46293.469 | 6963.442 | 32.827 | 0.346 |
| M21 + RNA_9_3Phos + M21 + RNA_24 | RNaseP | 431042.250 | 21203.030 | 31.175 | 0.159 |
| M21 + RNA_9_3Phos + M21 + RNA_24 | SARS- | 165207.953 | 12139.270 | 31.940 | 0.347 |
| | CoV-2 | | | | |
| M21 + RNA_9_3Phos + M21 + RNA_25 | InfA | 111725.439 | 33926.212 | 30.675 | 0.752 |
| M21 + RNA_9_3Phos + M21 + RNA_25 | InfB | 61887.129 | 10244.719 | 32.517 | 0.389 |
| M21 + RNA_9_3Phos + M21 + RNA_25 | RNaseP | 457555.656 | 34316.861 | 31.225 | 0.179 |
| M21 + RNA_9_3Phos + M21 + RNA_25 | SARS- | 178595.208 | 57642.019 | 31.677 | 1.263 |
| | CoV-2 | | | | |
| M21 + RNA_9_3Phos + M21 + RNA_26 | InfA | 192839.807 | 1629.061 | 30.545 | 0.270 |
| M21 + RNA_9_3Phos + M21 + RNA_26 | InfB | 67987.751 | 28016.372 | 32.725 | 1.069 |
| M21 + RNA_9_3Phos + M21 + RNA_26 | RNaseP | 416549.219 | 25440.482 | 31.390 | 0.160 |
| M21 + RNA_9_3Phos + M21 + RNA_26 | SARS- | 199806.672 | 21999.948 | 31.851 | 0.413 |
| | CoV-2 | | | | |
| M21 + RNA_9_3Phos + M21 + RNA_27 | InfA | 122170.930 | 6540.727 | 30.658 | 0.954 |
| M21 + RNA_9_3Phos + M21 + RNA_27 | InfB | 66603.589 | 22462.857 | 32.421 | 0.348 |
| M21 + RNA_9_3Phos + M21 + RNA_27 | RNaseP | 392217.427 | 75950.469 | 31.747 | 0.507 |
| M21 + RNA_9_3Phos + M21 + RNA_27 | SARS- | 177900.745 | 20346.032 | 31.036 | 0.656 |
| | CoV-2 | | | | |
| M21 + RNA_9_3Phos + M21 + RNA_28 | InfA | 145017.089 | 24488.121 | 26.471 | 4.699 |
| M21 + RNA_9_3Phos + M21 + RNA_28 | InfB | 55469.464 | 9509.197 | 28.218 | 7.216 |
| M21 + RNA_9_3Phos + M21 + RNA_28 | RNaseP | 389616.198 | 61335.219 | 31.022 | 0.393 |
| M21 + RNA_9_3Phos + M21 + RNA_28 | SARS- | 219697.172 | 34410.079 | 21.380 | 9.105 |
| | CoV-2 | | | | |
| M21 + RNA_9_3Phos + M21 + RNA_29 | InfA | 193099.270 | 132971.378 | 30.318 | 0.154 |
| M21 + RNA_9_3Phos + M21 + RNA_29 | InfB | 72405.422 | 4454.155 | 32.846 | 0.074 |
| M21 + RNA_9_3Phos + M21 + RNA_29 | RNaseP | 403070.344 | 15477.607 | 31.903 | 0.104 |
| M21 + RNA_9_3Phos + M21 + RNA_29 | SARS- | 302996.422 | 61240.845 | 32.042 | 0.700 |
| | CoV-2 | | | | |
| M21 + RNA_9_3Phos + M21 + RNA_30 | InfA | 169755.760 | 6826.768 | 30.289 | 0.553 |
| M21 + RNA_9_3Phos + M21 + RNA_30 | InfB | 55163.178 | 6654.527 | 32.717 | 0.366 |
| M21 + RNA_9_3Phos + M21 + RNA_30 | RNaseP | 455538.896 | 58555.268 | 31.468 | 0.493 |
| M21 + RNA_9_3Phos + M21 + RNA_30 | SARS- | 170642.276 | 23306.645 | 31.572 | 0.540 |
| | CoV-2 | | | | |
| M21 + RNA_9_3Phos + M21 + RNA_31 | InfA | 147548.404 | 25823.095 | 30.548 | 0.446 |
| M21 + RNA_9_3Phos + M21 + RNA_31 | InfB | 32680.459 | 23134.095 | 33.847 | 2.075 |
| M21 + RNA_9_3Phos + M21 + RNA_31 | RNaseP | 453542.167 | 78694.251 | 31.031 | 0.315 |
| M21 + RNA_9_3Phos + M21 + RNA_31 | SARS- | 168865.039 | 36072.614 | 32.067 | 0.189 |
| | CoV-2 | | | | |
| M21 + RNA_9_3Phos + M21 + RNA_32 | InfA | 180510.573 | 30120.254 | 30.362 | 0.521 |
| M21 + RNA_9_3Phos + M21 + RNA_32 | InfB | 47050.020 | 8514.383 | 33.163 | 0.225 |
| M21 + RNA_9_3Phos + M21 + RNA_32 | RNaseP | 412663.281 | 54216.508 | 31.590 | 0.325 |
| M21 + RNA_9_3Phos + M21 + RNA_32 | SARS- | 156584.740 | 29008.909 | 32.622 | 0.278 |
| | CoV-2 | | | | |
| M21 + RNA_9_3Phos + M21 + RNA_33 | InfA | 164686.224 | 10717.403 | 30.197 | 0.229 |
| M21 + RNA_9_3Phos + M21 + RNA_33 | InfB | 50967.799 | 6082.659 | 32.694 | 0.257 |
| M21 + RNA_9_3Phos + M21 + RNA_33 | RNaseP | 382571.458 | 21367.449 | 31.546 | 0.234 |
| M21 + RNA_9_3Phos + M21 + RNA_33 | SARS- | 134695.049 | 38523.131 | 31.217 | 0.516 |
| | CoV-2 | | | | |
| M21 + RNA_9_3Phos + M21 + RNA_34 | InfA | 175997.753 | 69032.954 | 30.334 | 0.539 |
| M21 + RNA_9_3Phos + M21 + RNA_34 | InfB | 49202.137 | 16299.425 | 32.888 | 0.465 |
| M21 + RNA_9_3Phos + M21 + RNA_34 | RNaseP | 447044.813 | 56128.068 | 31.280 | 0.486 |
| M21 + RNA_9_3Phos + M21 + RNA_34 | SARS- | 132538.867 | 19834.143 | 31.725 | 1.031 |
| | CoV-2 | | | | |
| M21 + RNA_9_3Phos + M21 + RNA_35 | InfA | 121441.742 | 32655.001 | 30.676 | 0.524 |
| M21 + RNA_9_3Phos + M21 + RNA_35 | InfB | 56292.475 | 15091.997 | 32.449 | 0.648 |
| M21 + RNA_9_3Phos + M21 + RNA_35 | RNaseP | 358342.583 | 60406.757 | 31.488 | 0.638 |
| M21 + RNA_9_3Phos + M21 + RNA_35 | SARS- | 198855.938 | 9703.312 | 30.714 | 0.621 |
| | CoV-2 | | | | |
| M21 + RNA_9_3Phos + M21 + RNA_36 | InfA | 132014.100 | 51983.674 | 30.737 | 0.921 |
| M21 + RNA_9_3Phos + M21 + RNA_36 | InfB | 43481.223 | 2093.470 | 33.109 | 0.115 |
| M21 + RNA_9_3Phos + M21 + RNA_36 | RNaseP | 422428.094 | 50170.916 | 31.482 | 0.563 |
| M21 + RNA_9_3Phos + M21 + RNA_36 | SARS- | 178371.005 | 27056.042 | 31.812 | 0.113 |
| | CoV-2 | | | | |
| M21 + RNA_9_3Phos + M21 + RNA_37 | InfA | 137940.138 | 37679.465 | 30.538 | 0.363 |
| M21 + RNA_9_3Phos + M21 + RNA_37 | InfB | 37309.160 | 31088.265 | 33.717 | 2.271 |
| M21 + RNA_9_3Phos + M21 + RNA_37 | RNaseP | 486985.635 | 43930.771 | 31.159 | 0.345 |
| M21 + RNA_9_3Phos + M21 + RNA_37 | SARS- | 167364.771 | 16113.237 | 30.849 | 0.481 |
| | CoV-2 | | | | |
| M21 + RNA_9_3Phos + M21 + RNA_38 | InfA | 94154.633 | 28178.215 | 30.858 | 0.874 |
| M21 + RNA_9_3Phos + M21 + RNA_38 | InfB | 31316.992 | 19434.911 | 33.411 | 1.535 |
| M21 + RNA_9_3Phos + M21 + RNA_38 | RNaseP | 413722.479 | 38596.964 | 31.263 | 0.226 |
| M21 + RNA_9_3Phos + M21 + RNA_38 | SARS- | 128285.294 | 39038.621 | 31.945 | 0.665 |
| | CoV-2 | | | | |
| M21 + RNA_9_3Phos + M21 + RNA_39 | InfA | 112297.940 | 43654.836 | 30.862 | 0.939 |
| M21 + RNA_9_3Phos + M21 + RNA_39 | InfB | 52403.333 | 8299.018 | 32.574 | 0.303 |
| M21 + RNA_9_3Phos + M21 + RNA_39 | RNaseP | 428202.250 | 91201.194 | 31.186 | 0.556 |
| M21 + RNA_9_3Phos + M21 + RNA_39 | SARS- | 166681.859 | 23510.363 | 31.688 | 0.130 |
| | CoV-2 | | | | |
| M21 + RNA_9_3Phos + M21 + RNA_40 | InfA | 150785.820 | 34306.687 | 30.750 | 0.580 |
| M21 + RNA_9_3Phos + M21 + RNA_40 | InfB | 58292.250 | 15288.081 | 32.748 | 0.576 |
| M21 + RNA_9_3Phos + M21 + RNA_40 | RNaseP | 358328.375 | 12497.851 | 31.866 | 0.189 |
| M21 + RNA_9_3Phos + M21 + RNA_40 | SARS- | 207678.464 | 31325.299 | 32.194 | 0.500 |
| | CoV-2 | | | | |
| M21 + RNA_9_3Phos + M21 + RNA_41 | InfA | 151583.862 | 14543.495 | 30.230 | 0.247 |
| M21 + RNA_9_3Phos + M21 + RNA_41 | InfB | 43458.535 | 1433.914 | 32.855 | 0.177 |
| M21 + RNA_9_3Phos + M21 + RNA_41 | RNaseP | 453278.708 | 16442.287 | 31.394 | 0.102 |
| M21 + RNA_9_3Phos + M21 + RNA_41 | SARS- | 200076.901 | 12287.231 | 31.265 | 0.529 |
| | CoV-2 | | | | |
| M21 + RNA_9_3Phos + M21 + RNA_42 | InfA | 139403.240 | 25485.814 | 30.674 | 0.591 |
| M21 + RNA_9_3Phos + M21 + RNA_42 | InfB | 58682.686 | 14031.202 | 32.567 | 0.423 |
| M21 + RNA_9_3Phos + M21 + RNA_42 | RNaseP | 386128.729 | 53882.174 | 31.645 | 0.250 |
| M21 + RNA_9_3Phos + M21 + RNA_42 | SARS- | 177322.594 | 21970.739 | 31.719 | 0.524 |
| | CoV-2 | | | | |
| M21 + RNA_9_3Phos + M21 + RNA_43 | InfA | 168853.792 | 15025.913 | 27.952 | 3.988 |
| M21 + RNA_9_3Phos + M21 + RNA_43 | InfB | 57696.517 | 11221.314 | 31.862 | 0.613 |
| M21 + RNA_9_3Phos + M21 + RNA_43 | RNaseP | 423473.927 | 47955.599 | 30.889 | 0.535 |
| M21 + RNA_9_3Phos + M21 + RNA_43 | SARS- | 207552.828 | 26783.363 | 26.414 | 8.177 |
| | CoV-2 | | | | |
| M21 + RNA_9_3Phos + M21 + RNA_44 | InfA | 89195.440 | 7374.455 | 31.705 | 0.162 |
| M21 + RNA_9_3Phos + M21 + RNA_44 | InfB | 67544.658 | 2713.924 | 32.482 | 0.317 |
| M21 + RNA_9_3Phos + M21 + RNA_44 | RNaseP | 440260.198 | 25155.016 | 31.535 | 0.142 |
| M21 + RNA_9_3Phos + M21 + RNA_44 | SARS- | 198712.521 | 19265.716 | 32.088 | 0.351 |
| | CoV-2 | | | | |
| M21 + RNA_9_3Phos + M21 + RNA_45 | InfA | 145595.719 | 4795.634 | 30.570 | 0.351 |
| M21 + RNA_9_3Phos + M21 + RNA_45 | InfB | 57881.164 | 12328.779 | 32.496 | 0.559 |
| M21 + RNA_9_3Phos + M21 + RNA_45 | RNaseP | 426696.594 | 65086.477 | 31.525 | 0.502 |
| M21 + RNA_9_3Phos + M21 + RNA_45 | SARS- | 186189.661 | 12179.498 | 31.927 | 0.474 |
| | CoV-2 | | | | |
| M21 + RNA_9_3Phos + M21 + RNA_46 | InfA | 133250.846 | 39299.616 | 30.583 | 0.255 |
| M21 + RNA_9_3Phos + M21 + RNA_46 | InfB | 61693.033 | 14459.375 | 32.191 | 0.454 |
| M21 + RNA_9_3Phos + M21 + RNA_46 | RNaseP | 397203.417 | 74573.658 | 31.553 | 0.362 |
| M21 + RNA_9_3Phos + M21 + RNA_46 | SARS- | 157758.070 | 34607.772 | 31.953 | 0.965 |
| | CoV-2 | | | | |
| M21 + RNA_9_3Phos + M21 + RNA_47 | InfA | 177397.867 | 49002.167 | 30.628 | 0.772 |
| M21 + RNA_9_3Phos + M21 + RNA_47 | InfB | 84699.047 | 10672.376 | 32.350 | 0.256 |
| M21 + RNA_9_3Phos + M21 + RNA_47 | RNaseP | 438477.156 | 29616.175 | 31.397 | 0.210 |
| M21 + RNA_9_3Phos + M21 + RNA_47 | SARS- | 210102.870 | 19497.557 | 31.902 | 0.585 |
| | CoV-2 | | | | |
| M21 + RNA_9_3Phos + M21 + RNA_48 | InfA | 129832.331 | 5784.485 | 30.634 | 0.277 |
| M21 + RNA_9_3Phos + M21 + RNA_48 | InfB | 42468.255 | 7950.759 | 33.189 | 0.422 |
| M21 + RNA_9_3Phos + M21 + RNA_48 | RNaseP | 408273.323 | 23959.455 | 31.588 | 0.229 |
| M21 + RNA_9_3Phos + M21 + RNA_48 | SARS- | 200516.911 | 48422.062 | 31.666 | 0.488 |
| | CoV-2 | | | | |
| M21 + RNA_9_3Phos + M21 + RNA_49 | InfA | 159803.320 | 39717.308 | 30.368 | 0.776 |
| M21 + RNA_9_3Phos + M21 + RNA_49 | InfB | 52374.133 | 10230.851 | 32.944 | 0.558 |
| M21 + RNA_9_3Phos + M21 + RNA_49 | RNaseP | 374071.323 | 30603.116 | 31.874 | 0.056 |
| M21 + RNA_9_3Phos + M21 + RNA_49 | SARS- | 160433.633 | 41109.360 | 31.042 | 2.056 |
| | CoV-2 | | | | |
| M21 + RNA_9_3Phos + M21 + RNA_50 | InfA | 166785.943 | 26186.814 | 30.395 | 0.345 |
| M21 + RNA_9_3Phos + M21 + RNA_50 | InfB | 29637.728 | 16527.846 | 33.458 | 1.148 |
| M21 + RNA_9_3Phos + M21 + RNA_50 | RNaseP | 433924.094 | 42716.244 | 31.560 | 0.395 |
| M21 + RNA_9_3Phos + M21 + RNA_50 | SARS- | 167920.469 | 27743.801 | 31.967 | 0.351 |
| | CoV-2 | | | | |
| RTv2 | InfA | 59478.632 | 38655.341 | 30.055 | 1.350 |
| RTv2 | InfB | 25640.642 | 3453.920 | 16.823 | 2.790 |
| RTv2 | RNaseP | 254518.021 | 58398.753 | 31.440 | 0.468 |
| RTv2 | SARS- | 45524.533 | 16340.656 | 31.013 | 1.095 |
| | CoV-2 | | | | |
| NTC | InfA | 7584.040 | 15637.739 | 36.655 | 4.048 |
| NTC | InfB | −19358.718 | 3169.781 | NA | NA |
| NTC | RNaseP | 10802.277 | 18593.993 | 35.784 | NA |
| NTC | SARS- | 5254.715 | 8643.013 | 15.404 | 9.034 |
| | CoV-2 | | | | |

TABLE 11
Evaluation of room temperature stability 6 hours following template addition
to PrimeTime ™ One-Step RT-qPCR Master Mix (Integrated DNA Technologies)
with pairs of DNA aptamers across a gradient of concentrations.

| | | 6 h Endpoint | | 6 h Cycle | |
|---|---|---|---|---|---|
| | | Fluorescence | | Threshold | |
| | | | Delta Rn | | Ct |
| Sample Name | Target Name | Delta Rn Mean | SD | Ct Mean | SD |
| M21 + RNA_9_3Phos + M21 + | InfA | 57902.957 | 21204.874 | 29.941 | 1.212 |
| RNA_11_3Phos_1:2:2 | | | | | |
| M21 + RNA_9_3Phos + M21 + | InfB | 15731.878 | 23793.126 | 32.767 | 0.449 |
| RNA_11_3Phos_1:2:2 | | | | | |
| M21 + RNA_9_3Phos + M21 + | RNaseP | 211621.025 | 72220.309 | 30.671 | 0.441 |
| RNA_11_3Phos_1:2:2 | | | | | |
| M21 + RNA_9_3Phos + M21 + | SARS-CoV- | 36586.868 | 37745.047 | 31.187 | 0.673 |
| RNA_11_3Phos_1:2:2 | 2 | | | | |
| M21 + RNA_9_3Phos + M21 + | InfA | 75478.735 | 27036.330 | 30.369 | 0.334 |
| RNA_11_3Phos_1:3:3 | | | | | |
| M21 + RNA_9_3Phos + M21 + | InfB | 24322.547 | 12102.095 | 32.728 | 0.257 |
| RNA_11_3Phos_1:3:3 | | | | | |
| M21 + RNA_9_3Phos + M21 + | RNaseP | 280798.867 | 47653.422 | 30.612 | 0.542 |
| RNA_11_3Phos_1:3:3 | | | | | |
| M21 + RNA_9_3Phos + M21 + | SARS-CoV- | 39603.277 | 28417.111 | 31.143 | 0.629 |
| RNA_11_3Phos_1:3:3 | 2 | | | | |
| M21 + RNA_9_3Phos + M21 + | InfA | 76963.469 | 27886.449 | 30.146 | 0.864 |
| RNA_11_3Phos_1:4:4 | | | | | |
| M21 + RNA_9_3Phos + M21 + | InfB | 23685.979 | 26229.018 | 33.321 | 0.556 |
| RNA_11_3Phos_1:4:4 | | | | | |
| M21 + RNA_9_3Phos + M21 + | RNaseP | 279326.349 | 106784.193 | 30.766 | 0.380 |
| RNA_11_3Phos_1:4:4 | | | | | |
| M21 + RNA_9_3Phos + M21 + | SARS-CoV- | 47726.294 | 61975.893 | 31.328 | 0.714 |
| RNA_11_3Phos_1:4:4 | 2 | | | | |
| M21 + RNA_9_3Phos + M21 + | InfA | 314286.617 | 93395.673 | 30.325 | 0.646 |
| RNA_11_3Phos_1:6:6 | | | | | |
| M21 + RNA_9_3Phos + M21 + | InfB | 96376.381 | 23507.336 | 32.887 | 0.381 |
| RNA_11_3Phos_1:6:6 | | | | | |
| M21 + RNA_9_3Phos + M21 + | RNaseP | 419018.870 | 59393.386 | 31.157 | 0.296 |
| RNA_11_3Phos_1:6:6 | | | | | |
| M21 + RNA_9_3Phos + M21 + | SARS-CoV- | 221852.973 | 96225.007 | 32.130 | |
| RNA_11_3Phos_1:6:6 | 2 | | | | 1.256 |
| M21 + RNA_9_3Phos + M21 + | InfA | 256122.474 | 61744.359 | 30.817 | 1.090 |
| RNA_11_3Phos_1:8:8 | | | | | |
| M21 + RNA_9_3Phos + M21 + | InfB | 89325.825 | 22415.003 | 33.069 | 0.328 |
| RNA_11_3Phos_1:8:8 | | | | | |
| M21 + RNA_9_3Phos + M21 + | RNaseP | 386175.245 | 46937.140 | 31.460 | 0.264 |
| RNA_11_3Phos_1:8:8 | | | | | |
| M21 + RNA_9_3Phos + M21 + | SARS-CoV- | 262299.865 | 81134.142 | 31.485 | 1.244 |
| RNA_11_3Phos_1:8:8 | 2 | | | | |
| M21 + RNA_9_3Phos + M21 + | InfA | 123196.104 | 28637.032 | 29.652 | 0.492 |
| RNA_47_3Phos_1:2:2 | | | | | |
| M21 + RNA_9_3Phos + M21 + | InfB | 5116.375 | 13696.597 | 33.637 | 0.311 |
| RNA_47_3Phos_1:2:2 | | | | | |
| M21 + RNA_9_3Phos + M21 + | RNaseP | 307286.414 | 85037.985 | 30.461 | 0.314 |
| RNA_47_3Phos_1:2:2 | | | | | |
| M21 + RNA_9_3Phos + M21 + | SARS-CoV- | 53357.642 | 48794.160 | 30.962 | 0.493 |
| RNA_47_3Phos_1:2:2 | 2 | | | | |
| M21 + RNA_9_3Phos + M21 + | InfA | 102541.661 | 36266.161 | 30.691 | 0.443 |
| RNA_47_3Phos_1:3:3 | | | | | |
| M21 + RNA_9_3Phos + M21 + | InfB | 20995.291 | 8674.359 | 33.697 | 0.597 |
| RNA_47_3Phos_1:3:3 | | | | | |
| M21 + RNA_9_3Phos + M21 + | RNaseP | 334448.411 | 43702.491 | 31.022 | 0.208 |
| RNA_47_3Phos_1:3:3 | | | | | |
| M21 + RNA_9_3Phos + M21 + | SARS-CoV- | 74761.389 | 37211.620 | 31.165 | 1.450 |
| RNA_47_3Phos_1:3:3 | 2 | | | | |
| M21 + RNA_9_3Phos + M21 + | InfA | 198580.939 | 70957.955 | 30.272 | 0.459 |
| RNA_47_3Phos_1:4:4 | | | | | |
| M21 + RNA_9_3Phos + M21 + | InfB | 41261.454 | 24119.594 | 33.624 | 0.965 |
| RNA_47_3Phos_1:4:4 | | | | | |
| M21 + RNA_9_3Phos + M21 + | RNaseP | 432668.531 | 66603.241 | 30.506 | 0.219 |
| RNA_47_3Phos_1:4:4 | | | | | |
| M21 + RNA_9_3Phos + M21 + | SARS-CoV- | 143650.169 | 93433.261 | 31.068 | 0.932 |
| RNA_47_3Phos_1:4:4 | 2 | | | | |
| M21 + RNA_9_3Phos + M21 + | InfA | 227597.242 | 36825.956 | 30.460 | 0.385 |
| RNA_47_3Phos_1:6:6 | | | | | |
| M21 + RNA_9_3Phos + M21 + | InfB | 93982.986 | 8091.507 | 32.758 | 0.199 |
| RNA_47_3Phos_1:6:6 | | | | | |
| M21 + RNA_9_3Phos + M21 + | RNaseP | 486858.849 | 32755.660 | 30.861 | 0.186 |
| RNA_47_3Phos_1:6:6 | | | | | |
| M21 + RNA_9_3Phos + M21 + | SARS-CoV- | 214435.980 | 61930.884 | 31.561 | 0.545 |
| RNA_47_3Phos_1:6:6 | 2 | | | | |
| M21 + RNA_9_3Phos + M21 + | InfA | 216937.904 | 121212.931 | 31.130 | 1.278 |
| RNA_47_3Phos_1:8:8 | | | | | |
| M21 + RNA_9_3Phos + M21 + | InfB | 81810.556 | 34276.797 | 33.384 | 0.455 |
| RNA_47_3Phos_1:8:8 | | | | | |
| M21 + RNA_9_3Phos + M21 + | RNaseP | 478202.922 | 96698.884 | 30.927 | 0.287 |
| RNA_47_3Phos_1:8:8 | | | | | |
| M21 + RNA_9_3Phos + M21 + | SARS-CoV- | 279116.575 | 126523.428 | 31.055 | 1.198 |
| RNA_47_3Phos_1:8:8 | 2 | | | | |
| M21 + RNA_9_3Phos_1:4 | InfA | 64632.210 | 15045.922 | 30.215 | 0.516 |
| M21 + RNA_9_3Phos_1:4 | InfB | 8972.886 | 12688.552 | 33.156 | 0.729 |
| M21 + RNA_9_3Phos_1:4 | RNaseP | 254635.898 | 70776.250 | 30.784 | 0.260 |
| M21 + RNA_9_3Phos_1:4 | SARS-CoV- | 59710.252 | 25497.657 | 28.830 | 2.485 |
| | 2 | | | | |
| M21 + RNA_9_3Phos_1:4 − NTC | InfA | 21571.603 | 26793.452 | 33.871 | 0.942 |
| M21 + RNA_9_3Phos_1:4 − NTC | InfB | 17374.673 | 41077.294 | NA | NA |
| M21 + RNA_9_3Phos_1:4 − NTC | RNaseP | 3496.172 | 5563.014 | 38.335 | NA |
| M21 + RNA_9_3Phos_1:4 − NTC | SARS-CoV- | −2442.818 | 1139.297 | NA | NA |
| | 2 | | | | |
| RTv2 | InfA | 62688.528 | 28722.471 | 29.337 | 1.594 |
| RTv2 | InfB | 27696.333 | 8728.790 | 16.162 | 2.417 |
| RTv2 | RNaseP | 257125.375 | 50435.772 | 30.750 | 0.463 |
| RTv2 | SARS-CoV- | 53779.510 | 13971.625 | 29.519 | 2.070 |
| | 2 | | | | |

TABLE 12
Evaluation of room temperature stability 6 hours following template addition
to PrimeTime ™ One-Step RT-qPCR Master Mix (Integrated DNA Technologies)
with a combination of three DNA aptamers across a gradient of concentrations.

| | | 6 h Endpoint | | | |
|---|---|---|---|---|---|
| | | Fluorescence | | | |
| | Target | Delta Rn | Delta Rn | 6 h Cycle Threshold | |
| Sample Name | Name | Mean | SD | Ct Mean | Ct SD |
| M21 + RNA_9_3Phos_1:4 | InfA | 81139.95 | 26017.91 | 30.28598 | 0.515973 |
| M21 + RNA_9_3Phos_1:4 | InfB | 17176.86 | 27738.74 | 33.18984 | 0.631217 |
| M21 + RNA_9_3Phos_1:4 | RNaseP | 308819.4 | 93091.12 | 30.6277 | 0.545308 |
| M21 + RNA_9_3Phos_1:4 | SARS- | 116760.3 | 82966.29 | 30.47775 | 1.514591 |
| | CoV-2 | | | | |
| M21 + RNA_9_3Phos + M21 + | InfA | 124632.9 | 138993.8 | 29.91861 | 0.898909 |
| RNA_11_3Phos_1:6:6 | | | | | |
| M21 + RNA_9_3Phos + M21 + | InfB | 26817.33 | 55812.34 | 33.76754 | 1.196403 |
| RNA_11_3Phos_1:6:6 | | | | | |
| M21 + RNA_9_3Phos + M21 + | RNaseP | 250737.8 | 168985.4 | 30.85549 | 0.596518 |
| RNA_11_3Phos_1:6:6 | | | | | |
| M21 + RNA_9_3Phos + M21 + | SARS- | 118308.4 | 132705 | 31.77511 | 0.610517 |
| RNA_11_3Phos_1:6:6 | CoV-2 | | | | |
| M21 + RNA_9_3Phos + M21 + | InfA | 68984 | 45701.7 | 30.3885 | 0.737852 |
| RNA_47_3Phos_1:6:6 | | | | | |
| M21 + RNA_9_3Phos + M21 + | InfB | 23390.57 | 27413.76 | 33.04554 | 0.473545 |
| RNA_47_3Phos_1:6:6 | | | | | |
| M21 + RNA_9_3Phos + M21 + | RNaseP | 301790.4 | 120777.6 | 30.56288 | 0.256889 |
| RNA_47_3Phos_1:6:6 | | | | | |
| M21 + RNA_9_3Phos + M21 + | SARS- | 52538.86 | 34508.04 | 31.67067 | 1.085319 |
| RNA_47_3Phos_1:6:6 | CoV-2 | | | | |
| M21 + RNA_9_3Phos + M21 + RNA_11_3Phos + | InfA | 169889.2 | 49169.22 | 30.91689 | 0.57753 |
| M21 + RNA_47_3Phos_1:4:4:4 | | | | | |
| M21 + RNA_9_3Phos + M21 + RNA_11_3Phos + | InfB | 72536.46 | 34118.6 | 33.95918 | 1.119249 |
| M21 + RNA_47_3Phos_1:4:4:4 | | | | | |
| M21 + RNA_9_3Phos + M21 + RNA_11_3Phos + | RNaseP | 394486.8 | 84826.84 | 31.21974 | 0.426439 |
| M21 + RNA_47_3Phos_1:4:4:4 | | | | | |
| M21 + RNA_9_3Phos + M21 + RNA_11_3Phos + | SARS- | 233405.2 | 103924.2 | 31.7012 | 1.164687 |
| M21 + RNA_47_3Phos_1:4:4:4 | CoV-2 | | | | |
| M21 + RNA_9_3Phos + M21 + RNA_11_3Phos + | InfA | 144453.9 | 123233.5 | 30.79 | 1.204923 |
| M21 + RNA_47_3Phos_1:5:5:5 | | | | | |
| M21 + RNA_9_3Phos + M21 + RNA_11_3Phos + | InfB | 50111.36 | 49963.1 | 33.83173 | 0.819159 |
| M21 + RNA_47_3Phos_1:5:5:5 | | | | | |
| M21 + RNA_9_3Phos + M21 + RNA_11_3Phos + | RNaseP | 366448.9 | 167688.5 | 31.07451 | 0.567504 |
| M21 + RNA_47_3Phos_1:5:5:5 | | | | | |
| M21 + RNA_9_3Phos + M21 + RNA_11_3Phos + | SARS- | 167003.9 | 144151.9 | 32.03743 | 0.910019 |
| M21 + RNA_47_3Phos_1:5:5:5 | CoV-2 | | | | |
| M21 + RNA_9_3Phos + M21 + RNA_11_3Phos + | InfA | 283467.7 | 94346.8 | 30.61128 | 0.473064 |
| M21 + RNA_47_3Phos_1:6:6:6 | | | | | |
| M21 + RNA_9_3Phos + M21 + RNA_11_3Phos + | InfB | 96911.84 | 10747.53 | 33.86855 | 0.166928 |
| M21 + RNA_47_3Phos_1:6:6:6 | | | | | |
| M21 + RNA_9_3Phos + M21 + RNA_11_3Phos + | RNaseP | 465985.9 | 31066.94 | 30.92971 | 0.402605 |
| M21 + RNA_47_3Phos_1:6:6:6 | | | | | |
| M21 + RNA_9_3Phos + M21 + RNA_11_3Phos + | SARS- | 372819.5 | 65962.1 | 31.78149 | 0.889624 |
| M21 + RNA_47_3Phos_1:6:6:6 | CoV-2 | | | | |
| RTv2 | InfA | 60410.03 | 40280.93 | 27.91564 | 3.079899 |
| RTv2 | InfB | 22475.86 | 3916.669 | 20.93457 | 4.232099 |
| RTv2 | RNaseP | 272923.2 | 51560.68 | 30.87224 | 0.2852 |
| RTv2 | SARS- | 47214.41 | 12213.99 | 30.94508 | 1.737713 |
| | CoV-2 | | | | |
| M21 + RNA_9_3Phos_1:4 − NTC | InfA | 26257.09 | 29856.9 | 33.69143 | 0.994545 |
| M21 + RNA_9_3Phos_1:4 − NTC | InfB | −21930.3 | 7276.14 | NA | NA |
| M21 + RNA_9_3Phos_1:4 − NTC | RNaseP | 739.2834 | 113.4568 | NA | NA |
| M21 + RNA_9_3Phos_1:4 − NTC | SARS- | −2072.37 | 1963.313 | 11.31761 | NA |
| | CoV-2 | | | | |

All references, including publications, patent applications, and patents, cited herein are hereby incorporated by reference to the same extent as if each reference were individually and specifically indicated to be incorporated by reference and were set forth in its entirety herein.

The use of the terms “a” and “an” and “the” and “at least one” and similar referents in the context of describing the invention (especially in the context of the following claims) are to be construed to cover both the singular and the plural, unless otherwise indicated herein or clearly contradicted by context. The use of the term “at least one” followed by a list of one or more items (for example, “at least one of A and B”) is to be construed to mean one item selected from the listed items (A or B) or any combination of two or more of the listed items (A and B), unless otherwise indicated herein or clearly contradicted by context. The terms “comprising.” “having,” “including,” and “containing” are to be construed as open-ended terms (i.e., meaning “including, but not limited to,”) unless otherwise noted. Recitation of ranges of values herein are merely intended to serve as a shorthand method of referring individually to each separate value falling within the range, unless otherwise indicated herein, and each separate value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein, is intended merely to better illuminate the invention and does not pose a limitation on the scope of the invention unless otherwise claimed. No language in the specification should be construed as indicating any non-claimed element as essential to the practice of the invention.

Preferred aspects of this invention are described herein, including the best mode known to the inventors for carrying out the invention. Variations of those preferred aspects may become apparent to those of ordinary skill in the art upon reading the foregoing description. The inventors expect skilled artisans to employ such variations as appropriate, and the inventors intend for the invention to be practiced otherwise than as specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

Unless otherwise defined, all technical and scientific terms used herein have the same meaning as commonly understood by one of ordinary skill in the art. For example, any nomenclatures used in connection with, and techniques of biochemistry, molecular biology, immunology, microbiology, genetics, cell and tissue culture, and protein and nucleic acid chemistry described herein are well known and commonly used in the art. In case of conflict, the present disclosure, including definitions, will control. Exemplary methods and materials are described below; although methods and materials similar or equivalent to those described herein can be used in practice or testing of the embodiments and aspects described herein.

As used herein, the terms “amino acid,” “nucleotide,” “polynucleotide,” “vector,” “polypeptide.” and “protein” have their common meanings as would be understood by a biochemist of ordinary skill in the art. Standard single letter nucleotides (A, C, G, T, U) and standard single letter amino acids (A, C, D, E, F, G, H, I, K, L, M, N, P, Q, R, S, T. V, W, or Y) are used herein.

As used herein, the terms such as “include,” “including,” “contain,” “containing,” “having,” and the like mean “comprising.” The present disclosure also contemplates other embodiments “comprising.” “consisting of,” and “consisting essentially of,” the embodiments or elements presented herein, whether explicitly set forth or not.

As used herein, the term “or” can be conjunctive or disjunctive.

As used herein, the term “substantially” means to a great or significant extent, but not completely.

As used herein, the term “about” or “approximately” as applied to one or more values of interest, refers to a value that is similar to a stated reference value, or within an acceptable error range for the particular value as determined by one of ordinary skill in the art, which will depend in part on how the value is measured or determined, such as the limitations of the measurement system. In one aspect, the term “about” refers to any values, including both integers and fractional components that are within a variation of up to #10% of the value modified by the term “about.” Alternatively, “about” can mean within 3 or more standard deviations, per the practice in the art. Alternatively, such as with respect to biological systems or processes, the term “about” can mean within an order of magnitude, in some embodiments within 5-fold, and in some embodiments within 2-fold, of a value. As used herein, the symbol “˜” means “about” or “approximately.”

All ranges disclosed herein include both end points as discrete values as well as all integers and fractions specified within the range. For example, a range of 0.1-2.0 includes 0.1, 0.2, 0.3, 0.4 . . . 2.0. If the end points are modified by the term “about.” the range specified is expanded by a variation of up to #10% of any value within the range or within 3 or more standard deviations, including the end points.

|0117] As used herein, the term “hybridization” refers to the process of combining two complementary single-stranded nucleic acid molecules and allowing them to form a single double-stranded hybrid molecule through base pairing.

As used herein, the term “target enrichment” with respect to a nucleic acid is intended to refer to increasing the relative concentration of particular nucleic acid species in the sample.

As used herein, the term “nucleic acid” may refer to DNA, RNA, dsDNA, dsRNA, ssDNA, ssRNA, or hybrids of DNA/RNA complexes or sequences obtained from any source, containing target and non-target sequences. For example, a nucleic acid sample can be obtained from artificial sources or by chemical synthesis, or from viruses, prokaryotic cells including microbes, or eukaryotic cells. Biological samples may be vertebrate, including human or excluding humans, invertebrates, plants, microbes, viruses, mycoplasma, fungi, or Archaea. A nucleic acid sample may comprise whole genomic sequences, portions of the genomic sequence, chromosomal sequences, mitochondrial sequences, PCR products, whole genome amplification products or products of other amplification protocol, such as but not limited to, cDNA sequences, mRNA sequences, whole transcriptome sequences, exons, or intronic. These examples are not to be construed as limiting the sample types applicable to aspects described herein.

By a polynucleotide having a nucleotide sequence at least, for example, 90-99% “identical” to a reference nucleotide sequence (e.g., SEQ ID NO: 2) is intended that the nucleotide sequence of the polynucleotide be identical to the reference sequence except that the polynucleotide sequence can include up to about 10 to 1 point mutations, additions, or deletions per each 100 nucleotides of the reference nucleotide sequence without affecting the function or activity of the polynucleotide sequence.

In other words, to obtain a polynucleotide having a nucleotide sequence about at least 90-99% identical to a reference nucleotide sequence, up to 10% of the nucleotides in the reference sequence can be deleted, added, or substituted, with another nucleotide, or a number of nucleotides up to 10% of the total nucleotides in the reference sequence can be inserted into the reference sequence. These mutations of the reference sequence can occur at the 5′- or 3′-terminal positions of the reference nucleotide sequence or anywhere between those terminal positions, interspersed either individually among nucleotides in the reference sequence or in one or more contiguous groups within the reference sequence.

As noted above, two or more polynucleotide sequences can be compared by determining their percent identity. The percent identity of two sequences is generally described as the number of exact matches between two aligned sequences divided by the length of the shorter sequence and multiplied by 100. An approximate alignment for nucleic acid sequences is provided by the local homology algorithm of Smith and Waterman, Advances in Applied Mathematics. 2 (4): 82-489 (1981).

The polynucleotides described herein include those comprising mutations, variations, substitutions, additions, deletions, and particular examples of the polynucleotides described herein. In addition, the polynucleotides can be ribonucleotides (RNA), deoxyribonucleotides (DNA), or combinations thereof, where the sequence comprise one or more ribonucleotides, or deoxyribonucleotides. The sequences can comprise ribonucleotides with for example ribothymidine nucleotides instead of uridine nucleotides to maintain consonance with the analogous DNA sequence.

It will be apparent to one of ordinary skill in the relevant art that suitable modifications and adaptations to the compositions, formulations, methods, processes, and applications described herein can be made without departing from the scope of any embodiments or aspects thereof. The compositions and methods provided are exemplary and are not intended to limit the scope of any of the specified embodiments. All of the various embodiments, aspects, and options disclosed herein can be combined in any variations or iterations. The scope of the compositions, formulations, methods, and processes described herein include all actual or potential combinations of embodiments, aspects, options, examples, and preferences herein described. The exemplary compositions and formulations described herein may omit any component, substitute any component disclosed herein, or include any component disclosed elsewhere herein. The ratios of the mass of any component of any of the compositions or formulations disclosed herein to the mass of any other component in the formulation or to the total mass of the other components in the formulation are hereby disclosed as if they were expressly disclosed. Should the meaning of any terms in any of the patents or publications incorporated by reference conflict with the meaning of the terms used in this disclosure, the meanings of the terms or phrases in this disclosure are controlling. Furthermore, the foregoing discussion discloses and describes merely exemplary embodiments.

1. A DNA aptamer comprising a nucleotide sequence that has at least 95% sequence identity to at least one of SEQ ID NOs: 2-101.

2. The DNA aptamer of claim 1, wherein the DNA aptamer consists of from about 50 to about 90 nucleotides.

3. The DNA aptamer of claim 1, comprising a nucleotide sequence that has at least 95% sequence identity to at least one of SEQ ID NOs: 29 (M21_28), 45 (M21_44), 60 (M21+RNA_9), 72 (M21+RNA_21), 76 (M21+RNA_25), 78 (M21+RNA_27), 79 (M21+RNA_28), 81 (M21+RNA_30), 82 (M21+RNA_31), 83 (M21+RNA_32), 90 (M21+RNA_39), 91 (M21+RNA_40), 93 (M21+RNA_42), 94 (M21+RNA_43), 95 (M21+RNA_44), 121 (M21+RNA_9_3Phos), 134 (M21+RNA_11_3Phos), and 135 (M21+RNA_47_3Phos).

4. The DNA aptamer of claim 1, comprising a nucleotide sequence that has at least 95% sequence identity to at least one of SEQ ID NOs: 121 (M21+RNA_9_3Phos), 134 (M21+RNA_11_3Phos), and 135 (M21+RNA_47_3Phos).

5. The DNA aptamer of claim 1, wherein the DNA aptamer comprises at least one 1-20 bp truncation.

6. The DNA aptamer of claim 1, wherein the DNA aptamer comprises 3′-phosphorylation.

7. The DNA aptamer of claim 1, wherein the DNA aptamer has binding affinity for a reverse transcriptase.

8. The DNA aptamer of claim 1, wherein the DNA aptamer inhibits a reverse transcriptase at from about 20° C. to about 30° C.

9. The DNA aptamer of claim 1, wherein the DNA aptamer fails to inhibit a reverse transcriptase at from about 45° C. to about 55° C.

10. The DNA aptamer of claim 7, wherein the reverse transcriptase is derived from Moloney murine leukemia virus (MMLV), avian myeloblastosis virus (AMV), or human immunodeficiency virus type 1 (HIV-1).

11. The DNA aptamer of claim 7, wherein the reverse transcriptase comprises a nucleotide sequence that has at least 95% sequence identity to SEQ ID NO: 132.

12. A composition comprising at least one DNA aptamer of claim 1.

13. The composition of claim 12, wherein the at least one DNA aptamer is polyacrylamide gel electrophoresis (PAGE)-purified.

14. The composition of claim 12, wherein the composition further comprises a reverse transcriptase.

15. The composition of claim 12, wherein the composition further comprises deoxynucleotide triphosphates (dNTPs), a buffer, or a combination thereof.

16. The composition of claim 12, wherein the DNA aptamer is present in the composition at a reverse transcriptase: aptamer ratio of from about 1:2 to about 1:8.

17. The composition of claim 12, wherein the composition comprises a first DNA aptamer (aptamer1), a second DNA aptamer (aptamer2), and optionally a third DNA aptamer (aptamer3).

18. A kit comprising the DNA aptamers of claim 1.

19. A method for amplifying a target DNA sequence, the method comprising:
a. providing a composition of claim 12, the composition further comprising at least one oligonucleotide primer, and at least one sample comprising at least one target DNA sequence, wherein the composition is at a temperature of from about 20° C. to about 30° C., and wherein the reverse transcriptase is bound to the at least one DNA aptamer;
b. elevating the temperature of the composition to release the reverse transcriptase from the at least one DNA aptamer;
c. hybridizing the at least one oligonucleotide primer to the at least one sample comprising at least one target DNA sequence; and
d. initiating DNA polymerase activity and extending the primer with the DNA polymerase to provide additional target DNA sequences.

20. A method for identifying at least one DNA aptamer that inhibits at least one reverse transcriptase, the method comprising:
a. providing a DNA aptamer pool comprising DNA aptamers;
b. allowing the DNA aptamers to be in proximity to the at least one reverse transcriptase in the presence of a RNA aptamer;
c. separating DNA aptamers with high affinity for the at least one reverse transcriptase from the DNA aptamers with low affinity for the at least one reverse transcriptase; and
d. amplifying the DNA aptamers with high affinity for the at least one reverse transcriptase to obtain an enriched DNA aptamer pool, wherein the at least one DNA aptamer that inhibits at least one reverse transcriptase may be identified from the enriched DNA aptamer pool.


SEQUENCE LISTING (135 sequences)

SEQ ID NO: 1 - RNA, 41 nt, synthetic construct
  source 1..41: mol_type = other RNA
cttaccacgcgctcttaactgctagcgccatggccaaaact

SEQ ID NO: 2 - DNA, 82 bp, synthetic construct
  source 1..82: mol_type = other DNA
gttcagtccctacggcgctaactataggtgaagtaactaaacacgtaattcttaattgactagccaccgtgctacaaccaag

SEQ ID NO: 3 - DNA, 82 bp, synthetic construct
  source 1..82: mol_type = other DNA
gttcagtccctacggcgctaacgaagggagttagagggcgctaatgatatgcacaacgaaatgccaccgtgctacaaccaag

SEQ ID NO: 4 - DNA, 82 bp, synthetic construct
  source 1..82: mol_type = other DNA
gttcagtccctacggcgctaaccaaatggtcgtaatagcctcaacatgtagttctcaatttcgccaccgtgctacaaccaag

SEQ ID NO: 5 - DNA, 82 bp, synthetic construct
  source 1..82: mol_type = other DNA
gttcagtccctacggcgctaactttctccgacatttttttaggaagggggtagctcagttgggccaccgtgctacaaccaag

SEQ ID NO: 6 - DNA, 82 bp, synthetic construct
  source 1..82: mol_type = other DNA
gttcagtccctacggcgctaaccggcaggcgaaacaacttaaacgagtagttcttcaattacgccaccgtgctacaaccaag

SEQ ID NO: 7 - DNA, 82 bp, synthetic construct; identical to SEQ ID NO: 6
  source 1..82: mol_type = other DNA

SEQ ID NO: 8 - DNA, 82 bp, synthetic construct
  source 1..82: mol_type = other DNA
gttcagtccctacggcgctaacttggcggatatagtcacaacatggagttctcaattggcccgccaccgtgctacaaccaag

SEQ ID NO: 9 - DNA, 82 bp, synthetic construct
  source 1..82: mol_type = other DNA
gttcagtccctacggcgctaacagtatggcgtaatagtaatcccatgaagatctcatttgttgccaccgtgctacaaccaag

SEQ ID NO: 10 - DNA, 82 bp, synthetic construct
  source 1..82: mol_type = other DNA
gttcagtccctacggcgctaacttggaatgcatgcagtaacttgaacacggaatcctcgacggccaccgtgctacaaccaag

SEQ ID NO: 11 - DNA, 82 bp, synthetic construct
  source 1..82: mol_type = other DNA
gttcagtccctacggcgctaacatgtagatgcgtaataaattcaccatgaagttcttaattagccaccgtgctacaaccaag

SEQ ID NO: 12 - DNA, 82 bp, synthetic construct
  source 1..82: mol_type = other DNA
gttcagtccctacggcgctaacttagatacgaattacggccgccgctaccaggcctagggcagccaccgtgctacaaccaag

SEQ ID NO: 13 - DNA, 82 bp, synthetic construct
  source 1..82: mol_type = other DNA
gttcagtccctacggcgctaactggaacgcggaatagccaaacatggaggtctttactttgtgccaccgtgctacaaccaag

SEQ ID NO: 14 - DNA, 82 bp, synthetic construct
  source 1..82: mol_type = other DNA
gttcagtccctacggcgctaaccaccgtggagtagattaacacggaattctcaattagcgatgccaccgtgctacaaccaag

SEQ ID NO: 15 - DNA, 82 bp, synthetic construct
  source 1..82: mol_type = other DNA
gttcagtccctacggcgctaaccagcgcaaaaactctgtcttccaatgttaattttagtagggccaccgtgctacaaccaag

SEQ ID NO: 16 - DNA, 82 bp, synthetic construct
  source 1..82: mol_type = other DNA
gttcagtccctacggcgctaaccttcatggccgtttagtcaaacaaaaaggctctcagtttcgccaccgtgctacaaccaag

SEQ ID NO: 17 - DNA, 82 bp, synthetic construct
  source 1..82: mol_type = other DNA
gttcagtccctacggcgctaacctgtgacgagggacgaaccctgccgtgcggccggatttaagccaccgtgctacaaccaag

SEQ ID NO: 18 - DNA, 82 bp, synthetic construct
  source 1..82: mol_type = other DNA
gttcagtccctacggcgctaacagttggtcaaatagcctcaacatgagattcttaattggacgccaccgtgctacaaccaag

SEQ ID NO: 19 - DNA, 82 bp, synthetic construct
  source 1..82: mol_type = other DNA
gttcagtccctacggcgctaactggcataatagataaaacatggcgatcttatttgatgcacgccaccgtgctacaaccaag

SEQ ID NO: 20 - DNA, 82 bp, synthetic construct
  source 1..82: mol_type = other DNA
gttcagtccctacggcgctaactctcgagcggcacaactataacgtggagttctcaattagtgccaccgtgctacaaccaag

SEQ ID NO: 21 - DNA, 82 bp, synthetic construct
  source 1..82: mol_type = other DNA
gttcagtccctacggcgctaacgaagggagttagagggcgcacatgaaagacaacaaataatgccaccgtgctacaaccaag

SEQ ID NO: 22 - DNA, 82 bp, synthetic construct
  source 1..82: mol_type = other DNA
gttcagtccctacggcgctaacaagcaagtacacggcgatttcaatcgctgaaccctcattagccaccgtgctacaaccaag

SEQ ID NO: 23 - DNA, 82 bp, synthetic construct
  source 1..82: mol_type = other DNA
gttcagtccctacggcgctaactcagtggaatcgagccgcatcgagacagggatcagctcaggccaccgtgctacaaccaag

SEQ ID NO: 24 - DNA, 82 bp, synthetic construct
  source 1..82: mol_type = other DNA
gttcagtccctacggcgctaacgaagggagttagagggcgcacaaaaacggtgtattatgcagccaccgtgctacaaccaag

SEQ ID NO: 25 - DNA, 82 bp, synthetic construct
  source 1..82: mol_type = other DNA
gttcagtccctacggcgctaacgaagggagttagagggcgtcatcaaagtatcaaccaggtggccaccgtgctacaaccaag

SEQ ID NO: 26 - DNA, 82 bp, synthetic construct
  source 1..82: mol_type = other DNA
gttcagtccctacggcgctaacgaagggagttagagggcgctattctgcttgatccaacaaggccaccgtgctacaaccaag

SEQ ID NO: 27 - DNA, 82 bp, synthetic construct
  source 1..82: mol_type = other DNA
gttcagtccctacggcgctaacccctacgaaaaaatatcagcctttcagttgtttgattcgtgccaccgtgctacaaccaag

SEQ ID NO: 28 - DNA, 82 bp, synthetic construct
  source 1..82: mol_type = other DNA
gttcagtccctacggcgctaacgaagggagttagagggcgtccactacactgcaccggccatgccaccgtgctacaaccaag

SEQ ID NO: 29 - DNA, 82 bp, synthetic construct
  source 1..82: mol_type = other DNA
gttcagtccctacggcgctaactggctggcgaagtaactatccacggagatctttatttgtggccaccgtgctacaaccaag

SEQ ID NO: 30 - DNA, 82 bp, synthetic construct
  source 1..82: mol_type = other DNA
gttcagtccctacggcgctaacctcagcattaaattgcacactgcccagcggccggatttaagccaccgtgctacaaccaag

SEQ ID NO: 31 - DNA, 82 bp, synthetic construct
  source 1..82: mol_type = other DNA
gttcagtccctacggcgctaacttagaaaaaggcgaagtcgcgacacacaatacttaacgccgccaccgtgctacaaccaag

SEQ ID NO: 32 - DNA, 82 bp, synthetic construct
  source 1..82: mol_type = other DNA
gttcagtccctacggcgctaacaggcgtttagccaaacaataagcatatcatcgaccggggggccaccgtgctacaaccaag

SEQ ID NO: 33 - DNA, 82 bp, synthetic construct
  source 1..82: mol_type = other DNA
gttcagtccctacggcgctaaccttcaggcgaagtaaccttgccacggagttcttaattcccgccaccgtgctacaaccaag

SEQ ID NO: 34 - DNA, 82 bp, synthetic construct
  source 1..82: mol_type = other DNA
gttcagtccctacggcgctaactactcaaggaagcatcactcgcatttttcagtcgccgatagccaccgtgctacaaccaag

SEQ ID NO: 35 - DNA, 82 bp, synthetic construct
  source 1..82: mol_type = other DNA
gttcagtccctacggcgctaaccacttggcaaatattcggagcatgtcggtcttacttgtatgccaccgtgctacaaccaag

SEQ ID NO: 36 - DNA, 82 bp, synthetic construct
  source 1..82: mol_type = other DNA
gttcagtccctacggcgctaacataactcacaactgccgagcggccggatttaagctctaatgccaccgtgctacaaccaag

SEQ ID NO: 37 - DNA, 82 bp, synthetic construct
  source 1..82: mol_type = other DNA
gttcagtccctacggcgctaactaactccagcaggtgtttaaatagggattagtccagctctgccaccgtgctacaaccaag

SEQ ID NO: 38 - DNA, 82 bp, synthetic construct
  source 1..82: mol_type = other DNA
gttcagtccctacggcgctaacccttggcacagcagcctaaacgcggaggtcttacttagatgccaccgtgctacaaccaag

SEQ ID NO: 39 - DNA, 83 bp, synthetic construct
  source 1..83: mol_type = other DNA
gttcagtccctacggcgctaactactgcctaacggccggatttaagttatccagctgagcggtgccaccgtgctacaaccaag

SEQ ID NO: 40 - DNA, 82 bp, synthetic construct
  source 1..82: mol_type = other DNA
gttcagtccctacggcgctaacgggaacaagaaatcagagtttcgataagcattcggcaagagccaccgtgctacaaccaag

SEQ ID NO: 41 - DNA, 82 bp, synthetic construct
  source 1..82: mol_type = other DNA
gttcagtccctacggcgctaacctaataaggcaaatatatcacatgtcgttctcaattcagtgccaccgtgctacaaccaag

SEQ ID NO: 42 - DNA, 82 bp, synthetic construct
  source 1..82: mol_type = other DNA
gttcagtccctacggcgctaactggatttggcgaagcagccccaacgcggagttatcaattggccaccgtgctacaaccaag

SEQ ID NO: 43 - DNA, 82 bp, synthetic construct
  source 1..82: mol_type = other DNA
gttcagtccctacggcgctaactgcggtaatgttagttagaatgctctaccgggtgagccaagccaccgtgctacaaccaag

SEQ ID NO: 44 - DNA, 82 bp, synthetic construct
  source 1..82: mol_type = other DNA
gttcagtccctacggcgctaactctggttgctagtacaattaaaacacgtcgatctcatttagccaccgtgctacaaccaag

SEQ ID NO: 45 - DNA, 82 bp, synthetic construct
  source 1..82: mol_type = other DNA
gttcagtccctacggcgctaacgattgcctttgaggacggcttactccttagcggaagaccagccaccgtgctacaaccaag

SEQ ID NO: 46 - DNA, 82 bp, synthetic construct
  source 1..82: mol_type = other DNA
gttcagtccctacggcgctaacgtttcgaaaaaggggatcattgagcgtagggcaattctgagccaccgtgctacaaccaag

SEQ ID NO: 47 - DNA, 82 bp, synthetic construct
  source 1..82: mol_type = other DNA
gttcagtccctacggcgctaaccaaacactacctgccctgcggccggatttaagctttcctagccaccgtgctacaaccaag

SEQ ID NO: 48 - DNA, 82 bp, synthetic construct
  source 1..82: mol_type = other DNA
gttcagtccctacggcgctaactatatgatgcctttggggacggctgaatgcagagagggacgccaccgtgctacaaccaag

SEQ ID NO: 49 - DNA, 82 bp, synthetic construct
  source 1..82: mol_type = other DNA
gttcagtccctacggcgctaacggagggagttagagggcgcctccatgcatattagtggtgagccaccgtgctacaaccaag

SEQ ID NO: 50 - DNA, 82 bp, synthetic construct
  source 1..82: mol_type = other DNA
gttcagtccctacggcgctaactgtcgagtgggttaagtatacaactgcgacaactagatttgccaccgtgctacaaccaag

SEQ ID NO: 51 - DNA, 82 bp, synthetic construct
  source 1..82: mol_type = other DNA
gttcagtccctacggcgctaaccagtcgggcgtaatagcgaacatgaagttctttaattagtgccaccgtgctacaaccaag

SEQ ID NO: 52 - DNA, 82 bp, synthetic construct
  source 1..82: mol_type = other DNA
gttcagtccctacggcgctaacgtcgtggagtagccctaacacggaagtattactagggaccgccaccgtgctacaaccaag

SEQ ID NO: 53 - DNA, 82 bp, synthetic construct
  source 1..82: mol_type = other DNA
gttcagtccctacggcgctaacgatgtgcaacaagattgcctttgaggatggcttatattttgccaccgtgctacaaccaag

SEQ ID NO: 54 - DNA, 82 bp, synthetic construct
  source 1..82: mol_type = other DNA
gttcagtccctacggcgctaactacccgcgaagtagcttatacacgtagttctcaatagtttgccaccgtgctacaaccaag

SEQ ID NO: 55 - DNA, 82 bp, synthetic construct
  source 1..82: mol_type = other DNA
gttcagtccctacggcgctaactcgatccccactctaccggcaactgctcggtcgggcatctgccaccgtgctacaaccaag

SEQ ID NO: 56 - DNA, 82 bp, synthetic construct
  source 1..82: mol_type = other DNA
gttcagtccctacggcgctaaccgttcgcactcattgcctttggggtacggcttatgagatcgccaccgtgctacaaccaag

SEQ ID NO: 57 - DNA, 82 bp, synthetic construct
  source 1..82: mol_type = other DNA
gttcagtccctacggcgctaactacaagggcttctaccggacacttatcggtttctgtatgtgccaccgtgctacaaccaag

SEQ ID NO: 58 - DNA, 82 bp, synthetic construct
  source 1..82: mol_type = other DNA
gttcagtccctacggcgctaacgattattaggtgctctattgcctttggggacggcttataagccaccgtgctacaaccaag

SEQ ID NO: 59 - DNA, 82 bp, synthetic construct
  source 1..82: mol_type = other DNA
gttcagtccctacggcgctaacggctatgccgccttttggtatcgtatgttgagcgacctaggccaccgtgctacaaccaag

SEQ ID NO: 60 - DNA, 82 bp, synthetic construct
  source 1..82: mol_type = other DNA
gttcagtccctacggcgctaaccgattgcctttgaggtatggcttatcgtttttgactcgccgccaccgtgctacaaccaag

SEQ ID NO: 61 - DNA, 82 bp, synthetic construct
  source 1..82: mol_type = other DNA
gttcagtccctacggcgctaaccagttgaggagtctaccggggaatatcggtgcatttggtcgccaccgtgctacaaccaag

SEQ ID NO: 62 - DNA, 82 bp, synthetic construct
  source 1..82: mol_type = other DNA
gttcagtccctacggcgctaacggtgaccttttcctaaaaggagtttggctttgcgattttggccaccgtgctacaaccaag

SEQ ID NO: 63 - DNA, 82 bp, synthetic construct
  source 1..82: mol_type = other DNA
gttcagtccctacggcgctaacttcgatcctctctgcccctggtgggcccctgagtaacggagccaccgtgctacaaccaag

SEQ ID NO: 64 - DNA, 82 bp, synthetic construct
  source 1..82: mol_type = other DNA
gttcagtccctacggcgctaacataccatcggtttgcctttgaggacggcttaacctatgctgccaccgtgctacaaccaag

SEQ ID NO: 65 - DNA, 82 bp, synthetic construct
  source 1..82: mol_type = other DNA
gttcagtccctacggcgctaacacgaaactccttcagagaattgttcacatgtcaccttggcgccaccgtgctacaaccaag

SEQ ID NO: 66 - DNA, 82 bp, synthetic construct
  source 1..82: mol_type = other DNA
gttcagtccctacggcgctaacaatggtttcctccttgcctttggggtacggcttaggagaagccaccgtgctacaaccaag

SEQ ID NO: 67 - DNA, 82 bp, synthetic construct
  source 1..82: mol_type = other DNA
gttcagtccctacggcgctaaccttattttcgacgcctttggggtacggcttcaaaaatatcgccaccgtgctacaaccaag

SEQ ID NO: 68 - DNA, 82 bp, synthetic construct
  source 1..82: mol_type = other DNA
gttcagtccctacggcgctaaccaaaccggatgccgattgcctttggggtacggcctactcggccaccgtgctacaaccaag

SEQ ID NO: 69 - DNA, 82 bp, synthetic construct
  source 1..82: mol_type = other DNA
gttcagtccctacggcgctaacagaaagcgaattgcctttggggtatggcttattggctgccgccaccgtgctacaaccaag

SEQ ID NO: 70 - DNA, 82 bp, synthetic construct
  source 1..82: mol_type = other DNA
gttcagtccctacggcgctaacacacagaggaaacaactgccccaccggccggatttaaggtgccaccgtgctacaaccaag

SEQ ID NO: 71 - DNA, 82 bp, synthetic construct
  source 1..82: mol_type = other DNA
gttcagtccctacggcgctaactaagcgttttccaattttcgctaaaattcgcaggcataaggccaccgtgctacaaccaag

SEQ ID NO: 72 - DNA, 82 bp, synthetic construct
  source 1..82: mol_type = other DNA
gttcagtccctacggcgctaactcctctctgccatgcggccggatttaagcattcataaactgccaccgtgctacaaccaag

SEQ ID NO: 73 - DNA, 82 bp, synthetic construct
  source 1..82: mol_type = other DNA
gttcagtccctacggcgctaacggtttgaaattttgcctttggggatggctttaattttcaggccaccgtgctacaaccaag

SEQ ID NO: 74 - DNA, 82 bp, synthetic construct
  source 1..82: mol_type = other DNA
gttcagtccctacggcgctaactggccagtgaaatagcttttacatggaattctcaatttgcgccaccgtgctacaaccaag

SEQ ID NO: 75 - DNA, 82 bp, synthetic construct
  source 1..82: mol_type = other DNA
gttcagtccctacggcgctaaccctcgcggaatagacttacatgaagatcttatttagtgacgccaccgtgctacaaccaag

SEQ ID NO: 76 - DNA, 82 bp, synthetic construct
  source 1..82: mol_type = other DNA
gttcagtccctacggcgctaacgtgaacatagcctttggtgtacggcttatgcttctctcttgccaccgtgctacaaccaag

SEQ ID NO: 77 - DNA, 82 bp, synthetic construct
  source 1..82: mol_type = other DNA
gttcagtccctacggcgctaacaagtcctggtacgcaccttgccaatgttactggcgctctagccaccgtgctacaaccaag

SEQ ID NO: 78 - DNA, 82 bp, synthetic construct
  source 1..82: mol_type = other DNA
gttcagtccctacggcgctaacgtgcgattgcctttggggtatggcttatcatacgcagccggccaccgtgctacaaccaag

SEQ ID NO: 79 - DNA, 82 bp, synthetic construct
  source 1..82: mol_type = other DNA
gttcagtccctacggcgctaaccatagcaggattagtggagttgcctttggggatggcttaggccaccgtgctacaaccaag

SEQ ID NO: 80 - DNA, 82 bp, synthetic construct
  source 1..82: mol_type = other DNA
gttcagtccctacggcgctaacgcttatatgtggcatggtacagggattgtccgagagttaggccaccgtgctacaaccaag

SEQ ID NO: 81 - DNA, 82 bp, synthetic construct
  source 1..82: mol_type = other DNA
gttcagtccctacggcgctaactctatcaatggacttgcctttggggatggctttgtctattgccaccgtgctacaaccaag

SEQ ID NO: 82 - DNA, 82 bp, synthetic construct
  source 1..82: mol_type = other DNA
gttcagtccctacggcgctaactaagattacggtgatcattgcctttggggatggcttactggccaccgtgctacaaccaag

SEQ ID NO: 83 - DNA, 82 bp, synthetic construct
  source 1..82: mol_type = other DNA
gttcagtccctacggcgctaacactccctgcccgacggccggatttaagtcgtctatggtgagccaccgtgctacaaccaag

SEQ ID NO: 84 - DNA, 82 bp, synthetic construct
  source 1..82: mol_type = other DNA
gttcagtccctacggcgctaacgaggttctccacctttccatataggtctcatatagtgtgcgccaccgtgctacaaccaag

SEQ ID NO: 85 - DNA, 82 bp, synthetic construct
  source 1..82: mol_type = other DNA
gttcagtccctacggcgctaacgttggctaaactagctctgccttccgatgttaatagttgggccaccgtgctacaaccaag

SEQ ID NO: 86 - DNA, 82 bp, synthetic construct
  source 1..82: mol_type = other DNA
gttcagtccctacggcgctaactcctcgaacgacgataacttttggggacagtttttcgtaggccaccgtgctacaaccaag

SEQ ID NO: 87 - DNA, 82 bp, synthetic construct
  source 1..82: mol_type = other DNA
gttcagtccctacggcgctaacggttactctactcgtaggcctttgaggatggccaaacgaagccaccgtgctacaaccaag

SEQ ID NO: 88 - DNA, 82 bp, synthetic construct
  source 1..82: mol_type = other DNA
gttcagtccctacggcgctaacccatttatactgccaccgcggccggatttaagcggcagaagccaccgtgctacaaccaag

SEQ ID NO: 89 - DNA, 82 bp, synthetic construct
  source 1..82: mol_type = other DNA
gttcagtccctacggcgctaacatttcggggtataagttgggtccagagaaatgtgaccttcgccaccgtgctacaaccaag

SEQ ID NO: 90 - DNA, 82 bp, synthetic construct
  source 1..82: mol_type = other DNA
gttcagtccctacggcgctaacccatactactgccccacggccggatttaagtgttaggaatgccaccgtgctacaaccaag

SEQ ID NO: 91 - DNA, 82 bp, synthetic construct
  source 1..82: mol_type = other DNA
gttcagtccctacggcgctaacactgcccgacggccggatttaagtcatagctcttcctgtggccaccgtgctacaaccaag

SEQ ID NO: 92 - DNA, 82 bp, synthetic construct
  source 1..82: mol_type = other DNA
gttcagtccctacggcgctaacagcttggagacaacagagctatgatcatcgtttgttcaacgccaccgtgctacaaccaag

SEQ ID NO: 93 - DNA, 82 bp, synthetic construct
  source 1..82: mol_type = other DNA
gttcagtccctacggcgctaaccagccgtgatcctggcctttggggtacggcctatgatctcgccaccgtgctacaaccaag

SEQ ID NO: 94 - DNA, 82 bp, synthetic construct
  source 1..82: mol_type = other DNA
gttcagtccctacggcgctaacaggattagtggagcggcctgttcgtcctcgatggataacagccaccgtgctacaaccaag

SEQ ID NO: 95 - DNA, 82 bp, synthetic construct
  source 1..82: mol_type = other DNA
gttcagtccctacggcgctaacccgctatcaaatatttgcctttggggatggcttaatatttgccaccgtgctacaaccaag

SEQ ID NO: 96 - DNA, 82 bp, synthetic construct
  source 1..82: mol_type = other DNA
gttcagtccctacggcgctaacaattgcctttggggtatggcttattgtgcatgatcgaaaagccaccgtgctacaaccaag

SEQ ID NO: 97 - DNA, 82 bp, synthetic construct
  source 1..82: mol_type = other DNA
gttcagtccctacggcgctaacagcattgcgaagtagctatacacgaagttctttaattggcgccaccgtgctacaaccaag

SEQ ID NO: 98 - DNA, 82 bp, synthetic construct
  source 1..82: mol_type = other DNA
gttcagtccctacggcgctaacttctgcccgaccggccggatttaaggtcatccgattgttagccaccgtgctacaaccaag

SEQ ID NO: 99 - DNA, 82 bp, synthetic construct
  source 1..82: mol_type = other DNA
gttcagtccctacggcgctaacaatgtatatctcggtacgctacgagtactaaccttgccatgccaccgtgctacaaccaag

SEQ ID NO: 100 - DNA, 82 bp, synthetic construct
  source 1..82: mol_type = other DNA
gttcagtccctacggcgctaactctctcctgcatgaagcacccgcgcctatcggggtgcggcgccaccgtgctacaaccaag

SEQ ID NO: 101 - DNA, 82 bp, synthetic construct
  source 1..82: mol_type = other DNA
gttcagtccctacggcgctaactgccatgcggccggatttaagcatcttagtatcagtcagagccaccgtgctacaaccaag

SEQ ID NO: 102 - DNA, 25 bp, synthetic construct
  source 1..25: mol_type = other DNA
caagaccaatcytgtcacctctgac

SEQ ID NO: 103 - DNA, 25 bp, synthetic construct
  source 1..25: mol_type = other DNA
caagaccaatyctgtcacctytgac

SEQ ID NO: 104 - DNA, 23 bp, synthetic construct
  source 1..23: mol_type = other DNA
gcattytggacaaavcgtctacg

SEQ ID NO: 105 - DNA, 23 bp, synthetic construct
  source 1..23: mol_type = other DNA
gcattttggataaagcgtctacg

SEQ ID NO: 106 - DNA, 15 bp, synthetic construct
  source 1..15: mol_type = other DNA
  misc_feature 1: note = The 5&apos; phosphate of the cytosine is attached through the linker N,N-diethyl-4-(4-nitronaphthalen-1-ylazo)-phenylamine to another nucleotide sequence 5&apos; TGCAGTCCT 3&apos;, which has a dye attached at the 5&apos; thymine.
  misc_feature 15: note = The 3&apos; phosphate of the guanine is attached to a Iowa BlackTM FQ quencher.
cgctcactgggcacg

SEQ ID NO: 107 - DNA, 22 bp, synthetic construct
  source 1..22: mol_type = other DNA
tcctcaaytcactcttcgagcg

SEQ ID NO: 108 - DNA, 21 bp, synthetic construct
  source 1..21: mol_type = other DNA
cggtgctcttgaccaaattgg

SEQ ID NO: 109 - DNA, 18 bp, synthetic construct
  source 1..18: mol_type = other DNA
  misc_feature 1: note = The 5&apos; phosphate of the guanine is attached through the linker N,N-diethyl-4-(4-nitronaphthalen-1-ylazo)-phenylamine to another nucleotide sequence 5&apos; CCAATTCGA 3&apos;, which has a Yakima YellowTM dye attached at the 5&apos; cytosine.
  misc_feature 18: note = The 3&apos; phosphate of the guanine is attached to a Iowa BlackTM FQ quencher.
gcagctgaaactgcggtg

SEQ ID NO: 110 - DNA, 23 bp, synthetic construct
  source 1..23: mol_type = other DNA
ctgcagatttggatgatttctcc

SEQ ID NO: 111 - DNA, 25 bp, synthetic construct
  source 1..25: mol_type = other DNA
ccttgtgtggtctgcatgagtttag

SEQ ID NO: 112 - DNA, 21 bp, synthetic construct
  source 1..21: mol_type = other DNA
  misc_feature 1: note = The 5&apos; phosphate of the adenine is attached through a quencher to another nucleotide sequence 5&apos; ATTGCAACA 3&apos;, which has a Texas RedTM-X attached at the 5&apos; adenine.
  misc_feature 21: note = The 3&apos; phosphate of the cytosine is attached to Iowa BlackTM FQ quencher.
atccatgagcagtgctgactc

SEQ ID NO: 113 - DNA, 19 bp, synthetic construct
  source 1..19: mol_type = other DNA
agatttggacctgcgagcg

SEQ ID NO: 114 - DNA, 20 bp, synthetic construct
  source 1..20: mol_type = other DNA
gagcggctgtctccacaagt

SEQ ID NO: 115 - DNA, 14 bp, synthetic construct
  source 1..14: mol_type = other DNA
  misc_feature 1: note = The 5&apos; phosphate of the guanine is attached through a quencher to another nucleotide sequence 5&apos; TTCTGACCT 3&apos;, which has a red dye attached at the 5&apos; thymine.
  misc_feature 14: note = The 3&apos; phosphate of the guanine is attached to Iowa Black TM FQ quencher.
gaaggctctgcgcg

SEQ ID NO: 116 - RNA, 106 nt, synthetic construct
  source 1..106: mol_type = other RNA
caagaccaatcttgtcacctctgactaagggaattttaggatttgtgttcacgctcaccgtgcccagtgagcgaggactgcagcgtagacgctttgtccaaaatgc

SEQ ID NO: 117 - RNA, 103 nt, synthetic construct
  source 1..103: mol_type = other RNA
tcctcaattcactcttcgagcgtcttaatgaaggacattcaaagccaattcgagcagctgaaactgcggtgggagtcttatcccaatttggtcaagagcaccg

SEQ ID NO: 118 - RNA, 92 nt, synthetic construct
  source 1..92: mol_type = other RNA
ctgcagatttggatgatttctccaaacaattgcaacaatccatgagcagtgctgactcaactcaggcctaaactcatgcagaccacacaagg

SEQ ID NO: 119 - RNA, 65 nt, synthetic construct
  source 1..65: mol_type = other RNA
agatttggacctgcgagcgggttctgacctgaaggctctgcgcggacttgtggagacagccgctc

SEQ ID NO: 120 - DNA, 82 bp, synthetic construct; identical to SEQ ID NO: 60
  source 1..82: mol_type = other DNA

SEQ ID NO: 121 - DNA, 82 bp, synthetic construct; identical to SEQ ID NO: 60
  source 1..82: mol_type = other DNA
  misc_feature 82: note = The guanine is attached to 3&apos; phosphorylation.

SEQ ID NO: 122 - DNA, 82 bp, synthetic construct; identical to SEQ ID NO: 60
  source 1..82: mol_type = other DNA
  misc_feature 82: note = The guanine is attached to a 3&apos; C3 Spacer.

SEQ ID NO: 123 - DNA, 82 bp, synthetic construct; identical to SEQ ID NO: 60
  source 1..82: mol_type = other DNA
  misc_feature 82: note = The guanine is attached to a 3&apos; Dideoxy-C.

SEQ ID NO: 124 - DNA, 82 bp, synthetic construct; identical to SEQ ID NO: 60
  source 1..82: mol_type = other DNA
  misc_feature 82: note = The guanine is attached to a 3&apos; inverted dT.

SEQ ID NO: 125 - DNA, 82 bp, synthetic construct; identical to SEQ ID NO: 60
  source 1..82: mol_type = other DNA
  misc_feature 82: note = The guanine is attached to 3&apos; amino modifer.

SEQ ID NO: 126 - DNA, 82 bp, synthetic construct; identical to SEQ ID NO: 78
  source 1..82: mol_type = other DNA

SEQ ID NO: 127 - DNA, 82 bp, synthetic construct; identical to SEQ ID NO: 78
  source 1..82: mol_type = other DNA
  misc_feature 82: note = The guanine is attached to 3&apos; phosphorylation.

SEQ ID NO: 128 - DNA, 82 bp, synthetic construct; identical to SEQ ID NO: 78
  source 1..82: mol_type = other DNA
  misc_feature 82: note = The guanine is attached to 3&apos; C3 Spacer.

SEQ ID NO: 129 - DNA, 82 bp, synthetic construct; identical to SEQ ID NO: 78
  source 1..82: mol_type = other DNA
  misc_feature 82: note = The guanine is attached to 3&apos; Dideoxy-C.

SEQ ID NO: 130 - DNA, 82 bp, synthetic construct; identical to SEQ ID NO: 78
  source 1..82: mol_type = other DNA
  misc_feature 82: note = The guanine is attached to 3&apos; inverted dT.

SEQ ID NO: 131 - DNA, 82 bp, synthetic construct; identical to SEQ ID NO: 78
  source 1..82: mol_type = other DNA
  misc_feature 82: note = The guanine is attached to 3&apos; amino modifier.

SEQ ID NO: 132 - DNA, 2052 bp, synthetic construct
  source 1..2052: mol_type = other DNA
atgactttaaatattgaggatgagcatcgtttacatgagacatcaaaagaacccgacgtgagcttagggtcaacgtggctttctgacttcccccaggcgtgggcggagactggcggaatggggttagctgtccgccaagcaccgttgatcatcccgttaaaggcaacgtctacacctgtctctatcaaacagtaccccatgagtcgtgaggcccgcctggggattaagccacatattcgtcgcttgctggaccaggggatcttggtcccatgtcaatctccgtggaacaccccccttaaacccgtgaaaaagccaggtacaaacgattatcgtccagttcaagatcttcgcgaggtcaacaaacgcgtagaagacatccatccgactgtacctaatccttataatctgttatcaggcctgcccccatcgcaccaatggtatacagtattagacttgaaagacgcgttcttttgcctgcgtctgcacccaacgtctcagccgctgtttgcgttcgaatggcgtgatcctgaaatgggaatttcgggtcagttaacctggactcgtctgccccagggctttaaaaacagccccacattgttcgatgaagcacttcaccgtgacttagcagacttccgtatccaacacccagacttaattctgttacagtatgttgacgaccttttgttggcggcaacgtctgaacttgactgtcagcaaggcacacgcgcgttattacaaacgttaggtaacttaggatatcgtgcgtccgcgaaaaaggcgcaaatttgtcaaaaacaggtaaagtaccttgggtatttgctgaaagaaggtcaacgttggctgactgatgcgcgtaaggagaccgtaatggggcagcctacgcctaagacgccacgccagttgcgtgaatttttgggcacagcgggattctgtcgtttatggattcctgggttcgctgaaatggctgcacccctgtaccccttaacaaaaacagggacgcttttcaactgggggccagaccagcaaaaggcgtatcaggagatcaaacaagctttgttgaccgcacccgcgttgggtcttccggatttaaccaagccctttgagctgttcgttgatgaaaaacagggatatgcaaaaggagtattaacccaaaagttaggcccgtggcgtcgccctgttgcttacttgagtaaaaaattggatcctgtcgcagcaggatggccaccgtgcttgcgtatggtcgcggcaattgccgttttgacaaaggatgcaggtaagttgacgatgggtcaacccttagtaatcttggctccacatgctgtagaagcgttagtaaagcagcccccagaccgctggctttctaatgcgcgcatgacccactatcaggcgcttctgcttgatacggatcgtgtacaatttggaccagttgtagctttgaatccagctactttgcttccccttccagaagaaggacttcagcacaattgtttagatattctggccgaggcacatgggacgcgccctgatttgacggatcagccactgcctgatgccgaccatacatggtatactggcggcagtagtcttcttcaagaggggcaacgcaaggcgggagcagccgtcactacggagaccgaagttatctgggccaaagcgttacccgcgggaacatccgcgcaacgtgcacagttaatcgctctgacacaggccctgaagatggcagagggcaaaaagttgaatgtctacaccaactcacgttatgcttttgcaacagcgcatatccatggcgaaatttaccgccgccgtggtctgctgactagtgagggtaaggaaattaaaaataaagatgagattcttgcgttgttaaaagctttattcttaccaaaacgcctttcgatcattcattgcccggggcatcaaaagggtcactcagcggaggctcgtggaaaccgtatggcggaccaagctgcccgtaaggcggcgatcacagagaccccggatacatcaacgctgttgatcgaaaacagctctccctacactagcgagcatttt

SEQ ID NO: 133 - protein, 704 aa, synthetic construct
MGSSHHHHHHSSGLVPRGSHMTLNIEDEHRLHETSKEPDVSLGSTWLSDFPQAWAETGGMGLAVRQAPLIIPLKATSTPVSIKQYPMSREARLGIKPHIRRLLDQGILVPCQSPWNTPLKPVKKPGTNDYRPVQDLREVNKRVEDIHPTVPNPYNLLSGLPPSHQWYTVLDLKDAFFCLRLHPTSQPLFAFEWRDPEMGISGQLTWTRLPQGFKNSPTLFDEALHRDLADFRIQHPDLILLQYVDDLLLAATSELDCQQGTRALLQTLGNLGYRASAKKAQICQKQVKYLGYLLKEGQRWLTDARKETVMGQPTPKTPRQLREFLGTAGFCRLWIPGFAEMAAPLYPLTKTGTLFNWGPDQQKAYQEIKQALLTAPALGLPDLTKPFELFVDEKQGYAKGVLTQKLGPWRRPVAYLSKKLDPVAAGWPPCLRMVAAIAVLTKDAGKLTMGQPLVILAPHAVEALVKQPPDRWLSNARMTHYQALLLDTDRVQFGPVVALNPATLLPLPEEGLQHNCLDILAEAHGTRPDLTDQPLPDADHTWYTGGSSLLQEGQRKAGAAVTTETEVIWAKALPAGTSAQRAQLIALTQALKMAEGKKLNVYTNSRYAFATAHIHGEIYRRRGLLTSEGKEIKNKDEILALLKALFLPKRLSIIHCPGHQKGHSAEARGNRMADQAARKAAITETPDTSTLLIENSSPYTSEHF

SEQ ID NO: 134 - DNA, 82 bp, synthetic construct; identical to SEQ ID NO: 62
  source 1..82: mol_type = other DNA
  misc_feature 82: note = The guanine is attached to 3&apos; phosphorylation.

SEQ ID NO: 135 - DNA, 82 bp, synthetic construct; identical to SEQ ID NO: 98
  source 1..82: mol_type = other DNA
  misc_feature 82: note = The guanine is attached to 3&apos; phosphorylation.